(19) United States
(12) Patent Application Publication
(10) Pub. No.: US 2024/0252631 A1
(43) Pub. Date: Aug. 1, 2024

(54) METHODS OF USING ACTIVIN RECEPTOR TYPE II SIGNALING INHIBITORS

(71) Applicant: Keros Therapeutics, Inc., Lexington, MA (US)

(72) Inventors: Jasbir S. SEEHRA, Lexington, MA (US); Jennifer Lachey, Lincoln, MA (US); Christopher R. Rovaldi, Swampscott, MA (US); Elissa Furutani, Belmont, MA (US)

(21) Appl. No.: 18/443,987

(22) Filed: Feb. 16, 2024

Related U.S. Application Data
(63) Continuation of application No. PCT/US22/40920, filed on Aug. 19, 2022.
(60) Provisional application No. 63/235,096, filed on Aug. 19, 2021.
(60) Provisional application No. 63/287,823, filed on Dec. 9, 2021.
(60) Provisional application No. 63/350,706, filed on Jun. 9, 2022.
(60) Provisional application No. 63/354,158, filed on Jun. 21, 2022.

(51) Int. Cl. A61K 39/395; A61K 31/519; A61K 45/06; A61P 7/06

(52) U.S. Cl. CPC A61K 39/3955; A61K 31/519; A61K 45/06; A61P 7/06

(57) ABSTRACT

The invention features methods of treating a subject receiving a cytopenia-associated myelofibrosis treatment by co-administering an activin receptor type II (ActRII) signaling inhibitor. The ActRII signaling inhibitor may be an antibody that binds to an ActRII ligand, an ActRII antibody, or an ActRII ligand trap.

### SEQUENCE LISTING

The instant application contains a Sequence Listing which has been submitted electronically in XML file format and is hereby incorporated by reference in its entirety. Said XML copy, created on Feb. 14, 2024, is named 51184-041006_Sequence_Listing_2_14_24. xml and is 1,204,409 bytes in size.

### BACKGROUND OF THE INVENTION

Myelofibrosis is a chronic myeloproliferative malignancy characterized by clonal proliferation of myeloid cells and megakaryocytic hyperplasia/dysplasia resulting in bone marrow fibrosis and osteosclerosis. It can present as a de novo disorder (primary myelofibrosis, PMF) or evolve from polycythemia vera (post-PV MF), essential thrombocythemia (post-ET MF), myelodysplastic syndrome (MDS), lupus, or other hematologic and solid tumors. Myeloproliferative neoplasms arise from a single somatically mutated hematopoietic stem cell progenitor that clonally expands and gives rise to virtually all myeloid cells and B and natural killer cells. It is characterized by bone marrow fibrosis, ineffective hematopoiesis, splenomegaly, extramedullary hematopoiesis, constitutional symptoms, and shortened survival. There are no curative medical therapies for patients with myelofibrosis, but JAK inhibitors, such as ruxolitinib (JAKAFI®/JAKAVI®), fedratinib (INREBIC®), and pacritinib (VONJO™) have been shown to reduce spleen volume and improve symptoms associated with MF. However, JAK inhibitors interfere with normal hematopoiesis and treatment with ruxolitinib and fedratinib is complicated by the development of anemia and thrombocytopenia, which can lead to dose reductions and reduced adherence, thereby limiting the number of patients able to remain on JAK inhibitors.

Accordingly, there exists a need for a new therapeutic approach to prevent or reduce the development of cytopenias in subjects treated with JAK inhibitors.

### SUMMARY OF THE INVENTION

The invention provides methods of co-administering an activin receptor type II (ActRII) signaling inhibitor and a cytopenia-associated myelofibrosis treatment, which can be used to treat myelofibrosis, polycythemia vera, or steroid-refractory graft versus host disease or to treat a cytopenia in a subject having any of these conditions. These methods can also be used to mitigate the adverse reactions associated with treatment with a cytopenia-associated myelofibrosis treatment and can improve treatment adherence, treatment duration, maintain dose intensity, or increase the dose of a cytopenia-associated myelofibrosis treatment, or decrease episodes of cytopenia, transfusion burden, bleeding events, infections, and treatment interruptions or discontinuations for a cytopenia-associated myelofibrosis treatment. The ActRII signaling inhibitor can be an antibody that binds to an ActRII ligand, an anti-ActRII antibody, or an ActRII ligand trap, and exemplary cytopenia-associated myelofibrosis treatments include ruxolitinib (JAKAFI®/JAKAVI®), fedratinib (INREBIC®), pacritinib (VONJO™), and imetelstat.

Exemplary embodiments of the invention are described in the enumerated paragraphs below.
E1. A method of treating a subject having myelofibrosis, comprising administering in combination to the subject an effective amount of a cytopenia-associated myelofibrosis treatment and an ActRII signaling inhibitor.
E2. The method of E1, wherein the cytopenia-associated myelofibrosis treatment and the ActRII signaling inhibitor are administered in combination after the subject has been identified as having a cytopenia.
E3. The method of E1, wherein the cytopenia-associated myelofibrosis treatment and the ActRII signaling inhibitor are administered in combination before the subject develops a cytopenia (e.g., to prevent or reduce the development of a cytopenia).
E4. A method of treating a subject with a myelofibrosis that has been identified as having a cytopenia, comprising administering in combination to the subject an effective amount of a cytopenia-associated myelofibrosis treatment and an ActRII signaling inhibitor.
E5. A method of treating a cytopenia in a subject diagnosed as having myelofibrosis, comprising administering in combination to the subject an effective amount of a cytopenia-associated myelofibrosis treatment and an ActRII signaling inhibitor.
E6. A method of treating a subject having polycythemia vera (e.g., an adult subject who has had an inadequate response to or is intolerant of hydroxyurea), comprising administering in combination to the subject an effective amount of a cytopenia-associated myelofibrosis treatment and an ActRII signaling inhibitor.
E7. A method of treating a subject having steroid-refractory graft-versus-host disease (e.g., acute graft-versus-host disease), comprising administering in combination to the subject an effective amount of a cytopenia-associated myelofibrosis treatment and an ActRII signaling inhibitor.
E8. The method of E6 or E7, wherein the cytopenia-associated myelofibrosis treatment and the ActRII signaling inhibitor are administered in combination after the subject has been identified as having a cytopenia.
E9. The method of E6 or E7, wherein the cytopenia-associated myelofibrosis treatment and the ActRII signaling inhibitor are administered in combination before the subject develops a cytopenia (e.g., to prevent or reduce the development of a cytopenia).
E10. A method of treating a cytopenia in a subject diagnosed as having polycythemia vera, comprising administering in combination to the subject an effective amount of a cytopenia-associated myelofibrosis treatment and an ActRII signaling inhibitor.
E11. A method of treating a cytopenia in a subject diagnosed as having steroid-refractory graft-versus-host disease, comprising administering in combination to the subject an effective amount of a cytopenia-associated myelofibrosis treatment and an ActRII signaling inhibitor.
E12. A method of treating a subject receiving treatment with a cytopenia-associated myelofibrosis treatment, comprising administering in combination to the subject an effective amount of a cytopenia-associated myelofibrosis treatment and an ActRII signaling inhibitor.
E13. A method of improving adherence to treatment with a cytopenia-associated myelofibrosis treatment in a subject in need thereof, comprising administering in combination the cytopenia-associated myelofibrosis treatment and an ActRII signaling inhibitor.
E14. A method of increasing the dose of a cytopenia-associated myelofibrosis treatment administered to a subject in need thereof, comprising administering in combination the cytopenia-associated myelofibrosis treatment and an ActRII signaling inhibitor (e.g., the subject can take a higher dose when the two agents are co-administered than when the cytopenia-associated myelofibrosis treatment is administered alone).
E15. A method of increasing treatment duration for a cytopenia-associated myelofibrosis treatment in a subject in need thereof, comprising administering in combination the cytopenia-associated myelofibrosis treatment and an ActRII signaling inhibitor (e.g., the subject can continue to take the cytopenia-associated myelofibrosis treatment for a longer period of time when the two agents are co-administered than when the cytopenia-associated myelofibrosis treatment is administered alone).
E16. A method of maintaining dose intensity for a cytopenia-associated myelofibrosis treatment in a subject in need thereof, comprising administering in combination the cytopenia-associated myelofibrosis treatment and an ActRII signaling inhibitor (e.g., the subject does not need to reduce the dose of the cytopenia-associated myelofibrosis treatment when the two agents are co-administered or requires fewer or smaller dose reductions than when the cytopenia-associated myelofibrosis treatment is administered alone).
E17. A method of decreasing episodes of cytopenia associated with a cytopenia-associated myelofibrosis treatment in a subject in need thereof, comprising administering in combination the cytopenia-associated myelofibrosis treatment and an ActRII signaling inhibitor.
E18. A method of decreasing transfusion burden in a subject treated with a cytopenia-associated myelofibrosis treatment, comprising administering in combination the cytopenia-associated myelofibrosis treatment and an ActRII signaling inhibitor.
E19. A method of decreasing bleeding events in a subject treated with a cytopenia-associated myelofibrosis treatment, comprising administering in combination the cytopenia-associated myelofibrosis treatment and an ActRII signaling inhibitor.
E20. A method of decreasing infections in a subject treated with a cytopenia-associated myelofibrosis treatment, comprising administering in combination the cytopenia-associated myelofibrosis treatment and an ActRII signaling inhibitor.
E21. A method of decreasing treatment interruptions or discontinuations for a cytopenia-associated myelofibrosis treatment in a subject in need thereof, comprising administering in combination the cytopenia-associated myelofibrosis treatment and an ActRII signaling inhibitor.
E22. A method of resuming treatment with a cytopenia-associated myelofibrosis treatment in a subject who developed a myelofibrosis treatment-associated cytopenia (e.g., after a treatment discontinuation), comprising administering in combination the cytopenia-associated myelofibrosis treatment and an ActRII signaling inhibitor.
E23. A method of promoting transfusion independence in a subject treated with a cytopenia-associated myelofibrosis treatment, comprising administering in combination the cytopenia-associated myelofibrosis treatment and an ActRII signaling inhibitor.
E24. The method of any one of E1-E5 and E12-E23, wherein the subject has medium- or high-risk myelofibrosis.
E25. The method of any one of E1-E5 and E12-E24, wherein the subject has primary myelofibrosis (PMF), post-essential thrombocythemia myelofibrosis (post-ET MF), or post-polycythemia vera myelofibrosis (post-PV MF).
E26. The method of any one of E6, E8-E10, and E12-E24, wherein the subject has polycythemia vera.
E27. The method of any one of E7-E9 and E11-E24, wherein the subject has steroid-refractory graft-versus-host disease.
E28. The method of any one of E1-E27, wherein the cytopenia-associated myelofibrosis treatment is a JAK inhibitor or Imetelstat
E29. The method of E28, wherein the JAK inhibitor is Ruxolitinib, Fedratinib, or Pacritinib.
E30. The method of any one of E1, E6, E7, and E12-E29, wherein the subject has a cytopenia.
E31. The method of any one of E1, E6, E7, and E12-E30, wherein the subject is identified as having a cytopenia prior to administration of the ActRII signaling inhibitor.
E32. The method of any one of E1, E6, E7, and E12-E30, wherein the method further comprises identifying the subject as having a cytopenia prior to administration of the ActRII signaling inhibitor.
E33. The method of any one of E2-E5, E8-E11, E17, and E29-E32, wherein the cytopenia is anemia.
E34. The method of any one of E2-E5, E8-E11, E17, and E29-E33, wherein the cytopenia is thrombocytopenia.
E35. The method of any one of E2-E5, E8-E11, E17, and E29-E34, wherein the cytopenia is neutropenia.
E36. The method of any one of E1-E35, wherein the ActRII signaling inhibitor is an activin A antibody or an antigen binding fragment thereof.
E37. The method of E36, wherein the activin A antibody is garetosmab.
E38. The method of E36, wherein the activin A antibody or an antigen binding fragment thereof has a heavy chain variable region (HCVR) sequence having at least 90% (e.g., at least 91%, 92%, 93%, 94%, 95%, 96%, 97%, 98%, 99%, or 100%) sequence identity to a HCVR sequence in Table 1 and a light chain variable region (LCVR) sequence having at least 90% (e.g., at least 91%, 92%, 93%, 94%, 95%, 96%, 97%, 98%, 99%, or 100%) sequence identity to a LCVR sequence in Table 1 (e.g., an HCVR sequence in Table 1 and an LCVR sequence in Table 1, such as an HCVR sequence and an LCVR sequence from the same row of Table 1).
E39. The method of E36 or E38, wherein the activin A antibody or an antigen binding fragment thereof has a light chain CDR1, CDR2, and CDR3 and a heavy chain CDR1, CDR2, and CDR3 listed in Table 2 (e.g., a light chain CDR1, CDR2, and CDR3 sequence and a heavy chain CDR1, CDR2, and CDR3 sequence from the same row of Table 2).
E40. The method of any one of E1-E35, wherein the ActRII signaling inhibitor is a myostatin antibody or an antigen binding fragment thereof.
E41. The method of E40, wherein the myostatin antibody is domagrozumab, landogrozumab, trevogrumab, or SRK-015.
E42. The method of E40, wherein the myostatin antibody or an antigen binding fragment thereof has a HCVR sequence having at least 90% (e.g., at least 91%, 92%, 93%, 94%, 95%, 96%, 97%, 98%, 99%, or 100%) sequence identity to a HCVR sequence in Table 3 and a LCVR sequence having at least 90% (e.g., at least 91%, 92%, 93%, 94%, 95%, 96%, 97%, 98%, 99%, or 100%) sequence identity to a LCVR sequence in Table 3 (e.g., a HCVR sequence in Table 3 and a LCVR sequence in Table 3, such as an HCVR sequence and an LCVR sequence from the same row of Table 3 or an HCVR sequence of any one of SEQ ID NOs: 448-476 and an LCVR sequence of any one of SEQ ID NOs: 477-486).
E43. The method of E40 or E42, wherein the myostatin antibody or an antigen binding fragment thereof has a light chain CDR1, CDR2, and CDR3 and a heavy chain CDR1, CDR2, and CDR3 listed in Table 4, Table 5, or Table 6 (e.g., a light chain CDR1, CDR2, and CDR3 sequence and a heavy chain CDR1, CDR2, and CDR3 sequence from the same row of Table 4).
E44. The method of any one of E40, E42, and E43, wherein the myostatin antibody or an antigen binding fragment thereof has a heavy chain and light chain sequence having at least 90% sequence identity (e.g., 90%, 91%, 92%, 93%, 94%, 95%, 95%, 97%, 98%, 99% or 100% sequence identity) to a heavy chain and light chain sequence provided in Table 7 (e.g., a heavy chain and light chain sequence from the same row of Table 7).
E45. The method of any one of E1-E35, wherein the ActRII signaling inhibitor is an ActRII antibody or an antigen binding fragment thereof.
E46. The method of E45, wherein the ActRII antibody is bimagrumab, CSJ089, CQI876, or CDD861.
E47. The method of E45, wherein the ActRII antibody or an antigen binding fragment thereof has a HCVR sequence having at least 90% (e.g., at least 91%, 92%, 93%, 94%, 95%, 96%, 97%, 98%, 99%, or 100%) sequence identity to a HCVR sequence in Table 8 and a LCVR sequence having at least 90% (e.g., at least 91%, 92%, 93%, 94%, 95%, 96%, 97%, 98%, 99%, or 100%) sequence identity to a LCVR sequence in Table 8 (e.g., a HCVR sequence in Table 8 and a LCVR sequence in Table 8, such as an HCVR sequence and an LCVR sequence from the same row of Table 8).
E48. The method of E45 or E47, wherein the ActRII antibody or an antigen binding fragment thereof has a light chain CDR1, CDR2, and CDR3 and a heavy chain CDR1, CDR2, and CDR3 listed in Table 9 (e.g., a light chain CDR1, CDR2, and CDR3 sequence and a heavy chain CDR1, CDR2, and CDR3 sequence from the same row of Table 9).
E49. The method of any one of E45, E47, and E49, wherein the ActRII antibody or an antigen binding fragment thereof has a heavy chain and light chain sequence having at least 90% sequence identity (e.g., 90%, 91%, 92%, 93%, 94%, 95%, 95%, 97%, 98%, 99% or 100% sequence identity) to a heavy chain and light chain sequence provided in Table 10 (e.g., a heavy chain and light chain sequence from the same row of Table 10).
E50. The method of any one of E1-E35, wherein the ActRII signaling inhibitor is an ActRII ligand trap.
E51. The method of E50, wherein the ActRII ligand trap is an ActRIIA ligand trap.
E52. The method of E51, wherein the ActRIIA ligand trap is a composition of Table 18 (e.g., a polypeptide, nucleic acid molecule, vector, or pharmaceutical composition of Table 18).
E53. The method of E51, wherein the ActRIIA ligand trap comprises an extracellular portion of wild-type ActRIIA (e.g., SEQ ID NO: 73 or SEQ ID NO: 729).
E54. The method of E51, wherein the ActRIIA ligand trap is sotatercept.
E55. The method of E50, wherein the ActRII ligand trap is an ActRIIB ligand trap.
E56. The method of E55, wherein the ActRIIB ligand trap comprises an extracellular portion of wild-type ActRIIB (e.g., SEQ ID NO: 74 or a portion thereof).
E57. The method of E55, wherein the ActRIIB ligand trap is BIIB110, ALG-802, luspatercept, ramatercept, or ACE-2494.
E58. The method of E55, wherein the ActRIIB ligand trap is a composition of Table 19 (e.g., a polypeptide, nucleic acid molecule, vector, or pharmaceutical composition of Table 19).
E59. The method of E55, wherein the ActRIIB ligand trap comprises the sequence of any one of SEQ ID NOs: 745-750 (e.g., the sequence of any one of SEQ ID NOs: 745-750 fused to a moiety, such as an Fc domain or an Fc domain monomer, by way of a linker).
E60. The method of E50, wherein the ActRII ligand trap is an ActRII chimera ligand trap.
E61. The method of E60, wherein the ActRII chimera ligand trap is a composition of Table 20 or Table 21 (e.g., a polypeptide, nucleic acid molecule, vector, or pharmaceutical composition of Table 20 or Table 21).
E62. The method of any one of E1-E35, wherein the ActRII signaling inhibitor is an activin B antibody or an antigen binding fragment thereof.
E63. The method of E62, wherein the activin B antibody or an antigen binding fragment thereof has a HCVR having at least 90% (e.g., at least 91%, 92%, 93%, 94%, 95%, 96%, 97%, 98%, 99%, or 100%) sequence identity to SEQ ID NO: 494 and a LCVR having at least 90% (e.g., at least 91%, 92%, 93%, 94%, 95%, 96%, 97%, 98%, 99%, or 100%) sequence identity to SEQ ID NO: 495.
E64. The method of any one of E1-E35, wherein the ActRII signaling inhibitor is a GDF-11 antibody or an antigen binding fragment thereof.
E65. The method of any one of E1-E64, wherein the method further includes evaluating red cell or platelet parameters after administration of the ActRII signaling inhibitor.
E66. The method of any one of E1-E65, wherein the method leads to an increase in hemoglobin of ≥1.5 g/dL (e.g., an increase in hemoglobin of ≥1.5 g/dL for at least 2 weeks, 4 weeks, 6 weeks, 8 weeks, 10 weeks, 12 weeks, 14 weeks, 16 weeks, 20 weeks, 24, weeks, 26 weeks, 1 year, 2 years or more during treatment with an ActRII signaling inhibitor compared to baseline or pretreatment measurements).
E67. The method of any one of E1-E66, wherein the method leads to a reduction in transfusion burden during a treatment period (e.g., a reduction in RBC units transfused during a treatment period of 2 weeks, 4 weeks, 6 weeks, 8 weeks, 10 weeks, 12 weeks, 14 weeks, 16 weeks, 20 weeks, 24, weeks, 26 weeks, 1 year, 2 years or more with an ActRII signaling inhibitor compared to baseline in the 8 weeks preceding treatment).
E68. The method of any one of E1-E67, wherein the subject achieves transfusion independence for at least twelve weeks during treatment (e.g., compared to pretreatment transfusion data from the 12 weeks directly preceding treatment).
E69. The method of any one of E1-E68, wherein the ActRII signaling inhibitor is administered in an amount sufficient to increase red blood cell levels, increase hemoglobin levels, increase red blood cell production, increase red blood cell count, increase hematocrit, reduce transfusion burden, promote transfusion independence, increase mean corpuscular volume, increase mean corpuscular hemoglobin, increase reticulocyte cell hemoglobin, increase erythropoietin levels, increase thrombopoietin levels, increase the maturation and/or differentiation of erythroid progenitors (e.g., early- and/or late-stage erythroid progenitors), increase late-stage erythroid precursor maturation, recruit early-stage progenitors into the erythroid lineage, increase reticulocytes, increase proerythroblast numbers, reduce the accumulation of red blood cell progenitor cells, increase the number of early-stage erythroid precursors and/or progenitors, promote the progression of erythroid precursors and/or progenitors through erythropoiesis, treat anemia, increase platelet levels, increase platelet volume, increase immature platelet fraction, increase proplatelets, increase platelet production, increase platelet count, increase or induce megakaryocyte differentiation and/or maturation, increase megakaryocyte progenitor renewal, reduce the accumulation of platelet progenitor cells, improve blood clotting, reduce bleeding events, reduce bleeding in the skin, treat thrombocytopenia, increase neutrophil levels, increase neutrophil production, increase neutrophil count, increase or induce the differentiation and/or maturation of progenitor cells into neutrophils, treat neutropenia, reduce susceptibility to infection, affect myostatin, activin A, activin B, and/or BMP9 signaling in the subject, or reduce or inhibit the binding of activin A, activin B, and/or myostatin to their receptors (e.g., their endogenous receptors).
E70. The method of any one of E1-E5 and E12-E69, wherein the ActRII signaling inhibitor is administered in an amount sufficient to reduce spleen volume, reduce bone marrow fibrosis, reduce osteosclerosis, improve bone marrow fibrosis grade, or reduce high platelet levels.
E71. The method of any one of E1-E70, wherein the method does not cause a vascular complication in the subject.
E72. The method of E71, wherein the method does not increase vascular permeability or leakage.
E73. The method of any one of E1-E72, wherein the subject is a human.

### Definitions

To facilitate the understanding of this invention, a number of terms are defined below. Terms defined herein have meanings as commonly understood by a person of ordinary skill in the areas relevant to the invention. Terms such as “a,” “an,” and “the” are not intended to refer to only a singular entity but include the general class of which a specific example may be used for illustration. The terminology herein is used to describe specific embodiments of the invention, but their usage does not limit the invention, except as outlined in the claims.

As used herein, the term “about” refers to a value that is within 10% above or below the value being described.

As used herein, any values provided in a range of values include both the upper and lower bounds, and any values contained within the upper and lower bounds.

As used herein, “administration” refers to providing or giving a subject a therapeutic agent (e.g., an ActRII signaling inhibitor described herein), by any effective route. Exemplary routes of administration are described herein below.

The term “antibody” is used in the broadest sense and specifically covers intact monoclonal antibodies, polyclonal antibodies, multispecific antibodies (e.g., bispecific antibodies) formed from at least two intact antibodies, and antibody fragments so long as they exhibit the desired biological activity.

“Antibody fragments” include a portion of an intact antibody, preferably the antigen binding or variable region of the intact antibody. Examples of antibody fragments include Fab, Fab′, F(ab′)2, and Fv fragments; diabodies; linear antibodies (Zapata et al. Protein Eng. 8(10):1057-1062 (1995)); single-chain antibody molecules; and multispecific antibodies formed from antibody fragments.

As used herein, the term “extracellular activin receptor type IIA (ActRIIA) variant” refers to a peptide including a soluble, extracellular portion of the single transmembrane receptor, ActRIIA, that has at least one amino acid substitution relative to a wild-type extracellular ActRIIA (e.g., bold portion of the sequence of SEQ ID NO: 75 shown below). The sequence of the wild-type, human ActRIIA precursor protein is shown below (SEQ ID NO: 75), in which the signal peptide is italicized and the extracellular portion is bold.

Wild-type, human ActRIIA precursor protein (SEQ ID NO: 75):

MGAAAKLAFAVFLISCSSGAILGRSETQECLFFNANWEKDRTNQTGVEP
CYGDKDKRRHCFATWKNISGSIEIVKQGCWLDDINCYDRTDCVEKKDSP
EVYFCCCEGNMCNEKFSYFPEMEVTQPTSNPVTPKPPYYNILLYSLVPL
MLIAGIVICAFWVYRHHKMAYPPVLVPTQDPGPPPPSPLLGLKPLQLLE
VKARGRFGCVWKAQLLNEYVAVKIFPIQDKQSWQNEYEVYSLPGMKHEN
ILQFIGAEKRGTSVDVDLWLITAFHEKGSLSDFLKANVVSWNELCHIAE
TMARGLAYLHEDIPGLKDGHKPAISHRDIKSKNVLLKNNLTACIADFGL
ALKFEAGKSAGDTHGQVGTRRYMAPEVLEGAINFQRDAFLRIDMYAMGL
VLWELASRCTAADGPVDEYMLPFEEEIGQHPSLEDMQEVVVHKKKRPVL
RDYWQKHAGMAMLCETIEECWDHDAEARLSAGCVGERITQMQRLTNIIT
TEDIVTVVTMVTNVDFPPKESSL

An extracellular ActRIIA variant may have a sequence of any one of SEQ ID NOs: 1-72. In particular embodiments, an extracellular ActRIIA variant has a sequence of any one of SEQ ID NOs: 6-72 (Table 12). In some embodiments, an extracellular ActRIIA variant may have at least 85% (e.g., at least 85%, 87%, 90%, 92%, 95%, 97%, or greater) amino acid sequence identity to the sequence of a wild-type extracellular ActRIIA (SEQ ID NO: 73).

As used herein, the term “cytopenia-associated myelofibrosis treatment” refers to a drug that is either approved for the treatment of myelofibrosis or that is in clinical development for the treatment of myelofibrosis and that has as an adverse reaction the development of a cytopenia (e.g., anemia, thrombocytopenia, or neutropenia).

As used herein, the term “linker” refers to a linkage between two elements, e.g., peptides or protein domains. An ActRII ligand trap described herein may include an extracellular portion of ActRIIA, ActRIIB, a variant thereof, or a chimera thereof (e.g., an extracellular ActRIIA variant having a sequence of any one of SEQ ID NOs: 1-72 fused to a moiety. The moiety may increase stability or improve pharmacokinetic properties of the polypeptide. The moiety (e.g., Fc domain monomer, Fc domain, an albumin-binding peptide, a fibronectin domain, or a human serum albumin) may be fused to the polypeptide by way of a linker. A linker can be a covalent bond or a spacer. The term “bond” refers to a chemical bond, e.g., an amide bond or a disulfide bond, or any kind of bond created from a chemical reaction, e.g., chemical conjugation. The term “spacer” refers to a moiety (e.g., a polyethylene glycol (PEG) polymer) or an amino acid sequence (e.g., a 1-200 amino acid sequence) occurring between two elements, e.g., peptides or protein domains, to provide space and/or flexibility between the two elements. An amino acid spacer is part of the primary sequence of a polypeptide (e.g., fused to the spaced peptides via the polypeptide backbone). The formation of disulfide bonds, e.g., between two hinge regions that form an Fc domain, is not considered a linker.

As used herein, the term “Fc domain” refers to a dimer of two Fc domain monomers. An Fc domain has at least 80% sequence identity (e.g., at least 85%, 90%, 95%, 97%, or 100% sequence identity) to a human Fc domain that includes at least a C_{H}2 domain and a C_{H}3 domain. An Fc domain monomer includes second and third antibody constant domains (C_{H}2 and C_{H}3). In some embodiments, the Fc domain monomer also includes a hinge domain. An Fc domain does not include any portion of an immunoglobulin that is capable of acting as an antigen-recognition region, e.g., a variable domain or a complementarity determining region (CDR). In the wild-type Fc domain, the two Fc domain monomers dimerize by the interaction between the two C_{H}3 antibody constant domains, as well as one or more disulfide bonds that form between the hinge domains of the two dimerizing Fc domain monomers. In some embodiments, an Fc domain may be mutated to lack effector functions, typical of a “dead Fc domain.” In certain embodiments, each of the Fc domain monomers in an Fc domain includes amino acid substitutions in the C_{H}2 antibody constant domain to reduce the interaction or binding between the Fc domain and an Fcγ receptor. In some embodiments, the Fc domain contains one or more amino acid substitutions that reduce or inhibit Fc domain dimerization. An Fc domain can be any immunoglobulin antibody isotype, including IgG, IgE, IgM, IgA, or IgD. Additionally, an Fc domain can be an lgG subtype (e.g., IgG1, IgG2a, IgG2b, IgG3, or IgG4). The Fc domain can also be a non-naturally occurring Fc domain, e.g., a recombinant Fc domain.

As used herein, the term “albumin-binding peptide” refers to an amino acid sequence of 12 to 16 amino acids that has affinity for and functions to bind serum albumin. An albumin-binding peptide can be of different origins, e.g., human, mouse, or rat. In some embodiments, an albumin-binding peptide has the sequence DICLPRWGCLW (SEQ ID NO: 83).

As used herein, the term “fibronectin domain” refers to a high molecular weight glycoprotein of the extracellular matrix, or a fragment thereof, that binds to, e.g., membrane-spanning receptor proteins such as integrins and extracellular matrix components such as collagens and fibrins. In some embodiments, a fibronectin domain is a fibronectin type Ill domain (SEQ ID NO: 82) having amino acids 610-702 of the sequence of UniProt ID NO: P02751. In other embodiments, a fibronectin domain is an adnectin protein.

As used herein, the term “human serum albumin” refers to the albumin protein present in human blood plasma. Human serum albumin is the most abundant protein in the blood. It constitutes about half of the blood serum protein. In some embodiments, a human serum albumin has the sequence of UniProt ID NO: P02768 (SEQ ID NO: 81).

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell, e.g., a human red blood cell, platelet, neutrophil, or muscle cell).

As used herein, the term “fused” is used to describe the combination or attachment of two or more elements, components, or protein domains, e.g., peptides or polypeptides, by means including chemical conjugation, recombinant means, and chemical bonds, e.g., amide bonds. For example, two single peptides in tandem series can be fused to form one contiguous protein structure, e.g., a polypeptide, through chemical conjugation, a chemical bond, a peptide linker, or any other means of covalent linkage. In some embodiments of an ActRII ligand trap described herein, extracellular portion of ActRIIA, ActRIIB, a variant thereof, or a chimera thereof (e.g., an extracellular ActRIIA variant having the sequence of any one of SEQ ID NOs: 1-72 (e.g., SEQ ID NOs: 6-72)) may be fused in tandem series to the N- or C-terminus of a moiety (e.g., Fc domain monomer (e.g., the sequence of SEQ ID NO: 97), an Fc domain (e.g., the sequence of SEQ ID NO: 84 or SEQ ID NO: 79), an albumin-binding peptide (e.g., the sequence of SEQ ID NO: 83), a fibronectin domain (e.g., the sequence of SEQ ID NO: 82), or a human serum albumin (e.g., the sequence of SEQ ID NO: 81)) by way of a linker. For example, an extracellular ActRIIA variant is fused to a moiety (e.g., an Fc domain monomer, an Fc domain, an albumin-binding peptide, a fibronectin domain, or a human serum albumin) by way of a peptide linker, in which the N-terminus of the peptide linker is fused to the C-terminus of the extracellular ActRIIA variant through a chemical bond, e.g., a peptide bond, and the C-terminus of the peptide linker is fused to the N-terminus of the moiety (e.g., Fc domain monomer, Fc domain, albumin-binding peptide, fibronectin domain, or human serum albumin) through a chemical bond, e.g., a peptide bond.

As used herein, the term “C-terminal extension” refers to the addition of one or more amino acids to the C-terminus of an extracellular ActRIIA variant (e.g., an extracellular ActRIIA variant having the sequence of any one of SEQ ID NOs: 1-70 (e.g., SEQ ID NOs: 6-70)). The C-terminal extension can be one or more amino acids, such as 1-6 amino acids (e.g., 1, 2, 3, 4, 5, 6 or more amino acids). The C-terminal extension may include amino acids from the corresponding position of wild-type ActRIIA. Exemplary C-terminal extensions are the amino acid sequence NP (a two amino acid C-terminal extension) and the amino acid sequence NPVTPK (SEQ ID NO: 78) (a six amino acid C-terminal extension). Any amino acid sequence that does not disrupt the activity of the polypeptide can be used. SEQ ID NO: 71, which is the sequence of SEQ ID NO: 69 with a C-terminal extension of NP, and SEQ ID NO: 72, which is the sequence of SEQ ID NO: 69 with a C-terminal extension of NPVTPK (SEQ ID NO: 78), represent two of the possible ways that a polypeptide of the invention can be modified to include a C-terminal extension.

As used herein, the term “percent (%) identity” refers to the percentage of amino acid (or nucleic acid) residues of a candidate sequence, e.g., an extracellular ActRIIA variant, that are identical to the amino acid (or nucleic acid) residues of a reference sequence, e.g., a wild-type extracellular ActRIIA (e.g., SEQ ID NO: 73), after aligning the sequences and introducing gaps, if necessary, to achieve the maximum percent identity (i.e., gaps can be introduced in one or both of the candidate and reference sequences for optimal alignment and non-homologous sequences can be disregarded for comparison purposes). Alignment for purposes of determining percent identity can be achieved in various ways that are within the skill in the art, for instance, using publicly available computer software such as BLAST, ALIGN, or Megalign (DNASTAR) software. Those skilled in the art can determine appropriate parameters for measuring alignment, including any algorithms needed to achieve maximal alignment over the full length of the sequences being compared. In some embodiments, the percent amino acid (or nucleic acid) sequence identity of a given candidate sequence to, with, or against a given reference sequence (which can alternatively be phrased as a given candidate sequence that has or includes a certain percent amino acid (or nucleic acid) sequence identity to, with, or against a given reference sequence) is calculated as follows:

$$100 \times ( fraction of A / B )$$

where A is the number of amino acid (or nucleic acid) residues scored as identical in the alignment of the candidate sequence and the reference sequence, and where B is the total number of amino acid (or nucleic acid) residues in the reference sequence. In some embodiments where the length of the candidate sequence does not equal to the length of the reference sequence, the percent amino acid (or nucleic acid) sequence identity of the candidate sequence to the reference sequence would not equal to the percent amino acid (or nucleic acid) sequence identity of the reference sequence to the candidate sequence.

In particular embodiments, a reference sequence aligned for comparison with a candidate sequence may show that the candidate sequence exhibits from 50% to 100% identity across the full length of the candidate sequence or a selected portion of contiguous amino acid (or nucleic acid) residues of the candidate sequence. The length of the candidate sequence aligned for comparison purpose is at least 30%, e.g., at least 40%, e.g., at least 50%, 60%, 70%, 80%, 90%, or 100% of the length of the reference sequence. When a position in the candidate sequence is occupied by the same amino acid (or nucleic acid) residue as the corresponding position in the reference sequence, then the molecules are identical at that position.

As used herein, the term “serum half-life” refers to, in the context of administering a therapeutic protein to a subject, the time required for plasma concentration of the protein in the subject to be reduced by half. The protein can be redistributed or cleared from the bloodstream, or degraded, e.g., by proteolysis. Serum half-life comparisons can be made by comparing the serum half-life of Fc fusion proteins.

As used herein, the term “affinity” or “binding affinity” refers to the strength of the binding interaction between two molecules. Generally, binding affinity refers to the strength of the sum total of non-covalent interactions between a molecule and its binding partner, such as an extracellular ActRIIA variant and BMP9 or activin A. Unless indicated otherwise, binding affinity refers to intrinsic binding affinity, which reflects a 1:1 interaction between members of a binding pair. The binding affinity between two molecules is commonly described by the dissociation constant (K_{D}) or the affinity constant (K_{A}). Two molecules that have low binding affinity for each other generally bind slowly, tend to dissociate easily, and exhibit a large K_{D}. Two molecules that have high affinity for each other generally bind readily, tend to remain bound longer, and exhibit a small K_{D}. The K_{D} of two interacting molecules may be determined using methods and techniques well known in the art, e.g., surface plasmon resonance. K_{D} is calculated as the ratio of k_{off}/kₒₙ.

As used herein, the phrase “affecting myostatin, activin A, activin B, and/or BMP9 signaling” means changing the binding of myostatin, activin A, activin B, and/or BMP9 to their receptors, e.g., ActRIIA, ActRIIB, and/or BMPRII (e.g., ActRIIA, e.g., endogenous ActRIIA). In some embodiments, a polypeptide including an extracellular ActRIIA variant described herein reduces or inhibits the binding of myostatin, activin A, activin B, and/or BMP9 to their receptors, e.g., ActRIIA, ActRIIB, and/or BMPRII (e.g., ActRIIA, e.g., endogenous ActRIIA).

As used herein, the terms “increasing” and “decreasing” refer to modulating resulting in, respectively, greater or lesser amounts, of function, expression, or activity of a metric relative to a reference. For example, subsequent to administration of a polypeptide of the invention including an extracellular ActRIIA variant in a method described herein, the amount of a marker of a metric (e.g., hemoglobin levels, red blood cell count, hematocrit, reticulocyte count, platelet count, or transfusion burden) as described herein may be increased or decreased in a subject relative to the amount of the marker prior to administration. Generally, the metric is measured subsequent to administration at a time that the administration has had the recited effect, e.g., at least one week, one month, 3 months, or 6 months, after a treatment regimen has begun.

As used herein, the terms “increase red blood cell levels” and “promote red blood cell formation” refer to clinically observable metrics, such as hematocrit, red blood cell counts, and hemoglobin measurements, and are intended to be neutral as to the mechanism by which such changes occur. The terms “red blood cell formation” and “red blood cell production” refer to the generation of red blood cells, such as the process of erythropoiesis in which red blood cells are produced in the bone marrow.

As used herein, the term “anemia” refers to any abnormality in hemoglobin or red blood cells that leads to reduced oxygen levels in the blood. Anemia can be associated with abnormal production, processing, or performance of erythrocytes and/or hemoglobin. The term anemia refers to any reduction in the number of red blood cells and/or level of hemoglobin in blood relative to normal blood levels. For example, a subject having a hemoglobin level ≤10 g/dL or receiving red blood cell (RBC) transfusions can be identified as having anemia.

As used herein, the terms “increase platelet levels” and “promote platelet formation” refer to clinically observable metrics, such as platelet counts, and are intended to be neutral as to the mechanism by which such changes occur. The terms “platelet formation” and “platelet production” refer to the generation of platelets, such as the process in which platelets are produced from megakaryocytes.

As used herein, the terms “increase neutrophil levels” and “promote neutrophil formation” refer to clinically observable metrics, such as neutrophil counts, and are intended to be neutral as to the mechanism by which such changes occur. The terms “neutrophil formation” and “neutrophil production” refer to the generation of neutrophils such as the process in which neutrophils are produced in the bone marrow.

As used herein, the term “thrombocytopenia” refers to a condition in which the blood contains a lower than normal number of platelets, which may be due to a deficiency in platelet production, accumulation of platelets within an enlarged spleen, or the destruction of platelets. Normal blood platelet levels range from about 150,000 to 450,000 per microliter blood in humans. A platelet count of less than 150,000 platelets per microliter is lower than normal. Bleeding can occur after a relatively minor injury if the platelet count falls below 50,000 platelets per microliter of blood, and serious bleeding may occur without any recognized injury if the platelet count falls below 10,000 to 20,000 platelets per microliter of blood.

As used herein, the term “neutropenia” refers to a condition in which the blood contains an abnormally low number of neutrophils. The typical lower limit of the neutrophil count is about 1500 cells per microliter of blood. Below this level, the risk of infection increases. Neutropenia severity is classified as: mild (1000 to 1500 neutrophils per microliter of blood), moderate (500 to 1000 neutrophils per microliter of blood), and severe (below 500 neutrophils per microliter of blood). Neutropenia has many causes, but they typically fall into two main categories: destruction or depletion of neutrophils faster than the bone marrow can produce new neutrophils, or reduced production of neutrophils in the bone marrow.

As used herein, the term “ineffective hematopoiesis” refers to the failure to produce fully mature hematopoietic cells (e.g., the failure to produce red blood cells, platelets, and neutrophils). Ineffective hematopoiesis may be due to single or multiple defects, such as abnormal proliferation and/or differentiation of progenitor cells (e.g., an excessive production of progenitors that are unable to complete differentiation), that can lead to a hyperproliferation or a shortage of progenitor cells.

As used herein, the terms “erythropoiesis stimulating agent” and “ESA” refer to a class of drugs that act on the proliferation stage of red blood cell development by expanding the pool of early-stage progenitor cells. Examples of erythropoiesis-stimulating agents are epoetin alfa and darbepoetin alfa.

As used herein, the term “vascular complication” refers to a vascular disorder or any damage to the blood vessels, such as damage to the blood vessel walls. Damage to the blood vessel walls may cause an increase in vascular permeability or leakage. The term “vascular permeability or leakage” refers to the capacity of the blood vessel walls to allow the flow of small molecules, proteins, and cells in and out of blood vessels. An increase in vascular permeability or leakage may be caused by an increase in the gaps (e.g., an increase in the size and/or number of the gaps) between endothelial cells that line the blood vessel walls and/or thinning of the blood vessel walls.

As used herein, the term “polypeptide” describes a single polymer in which the monomers are amino acid residues which are covalently conjugated together through amide bonds. A polypeptide is intended to encompass any amino acid sequence, either naturally occurring, recombinant, or synthetically produced.

As used herein, the term “homodimer” refers to a molecular construct formed by two identical macromolecules, such as proteins or nucleic acids. The two identical monomers may form a homodimer by covalent bonds or non-covalent bonds. For example, an Fc domain may be a homodimer of two Fc domain monomers if the two Fc domain monomers contain the same sequence. In another example, a polypeptide described herein including an extracellular ActRIIA variant fused to an Fc domain monomer may form a homodimer through the interaction of two Fc domain monomers, which form an Fc domain in the homodimer.

As used herein, the term “heterodimer” refers to a molecular construct formed by two different macromolecules, such as proteins or nucleic acids. The two monomers may form a heterodimer by covalent bonds or non-covalent bonds. For example, a polypeptide described herein including an extracellular ActRIIA variant fused to an Fc domain monomer may form a heterodimer through the interaction of two Fc domain monomers, each fused to a different ActRIIA variant, which form an Fc domain in the heterodimer.

As used herein, the term “host cell” refers to a vehicle that includes the necessary cellular components, e.g., organelles, needed to express proteins from their corresponding nucleic acids. The nucleic acids are typically included in nucleic acid vectors that can be introduced into the host cell by conventional techniques known in the art (transformation, transfection, electroporation, calcium phosphate precipitation, direct microinjection, etc.). A host cell may be a prokaryotic cell, e.g., a bacterial cell, or a eukaryotic cell, e.g., a mammalian cell (e.g., a CHO cell or a HEK293 cell).

As used herein, the term “therapeutically effective amount” refers an amount of a polypeptide, nucleic acid, or vector of the invention or a pharmaceutical composition containing a polypeptide, nucleic acid, or vector of the invention effective in achieving the desired therapeutic effect in treating a patient having a disease or condition, such as a cytopenia (e.g., anemia, thrombocytopenia, or neutropenia) associated with myelofibrosis or myelofibrosis treatment. In particular, the therapeutically effective amount of the polypeptide, nucleic acid, or vector avoids adverse side effects.

As used herein, the term “pharmaceutical composition” refers to a medicinal or pharmaceutical formulation that includes an active ingredient as well as excipients and diluents to enable the active ingredient suitable for the method of administration. The pharmaceutical composition of the present invention includes pharmaceutically acceptable components that are compatible with the polypeptide, nucleic acid, or vector. The pharmaceutical composition may be in tablet or capsule form for oral administration or in aqueous form for intravenous or subcutaneous administration.

As used herein, the term “pharmaceutically acceptable carrier or excipient” refers to an excipient or diluent in a pharmaceutical composition. The pharmaceutically acceptable carrier must be compatible with the other ingredients of the formulation and not deleterious to the recipient. In the present invention, the pharmaceutically acceptable carrier or excipient must provide adequate pharmaceutical stability to a polypeptide described herein (e.g., an ActRII signaling inhibitor, such as an ActRII ligand trap including an extracellular ActRIIA variant), the nucleic acid molecule(s) encoding the polypeptide, or a vector containing such nucleic acid molecule(s). The nature of the carrier or excipient differs with the mode of administration. For example, for intravenous administration, an aqueous solution carrier is generally used; for oral administration, a solid carrier is preferred.

As used herein, the term “treating and/or preventing” refers to the treatment and/or prevention of a disease or condition, e.g., a cytopenia (e.g., anemia, thrombocytopenia, or neutropenia) associated with myelofibrosis or myelofibrosis treatment, using methods and compositions of the invention. Generally, treating a disease or condition, e.g., a cytopenia (e.g., anemia, thrombocytopenia, or neutropenia) associated with myelofibrosis or myelofibrosis treatment e, occurs after a subject has developed the disease or condition. Preventing a disease or condition, e.g., a cytopenia (e.g., anemia, thrombocytopenia, or neutropenia) associated with myelofibrosis or myelofibrosis treatment, refers to steps or procedures taken when a subject is at risk of developing the disease or condition. The subject may show signs or mild symptoms that are judged by a physician to be indications or risk factors for developing the disease or condition, have another disease or condition associated with development of the disease or condition, be undergoing treatment that may cause the disease or condition, or have a family history or genetic predisposition of developing the disease or condition, but has not yet developed the disease or condition.

As used herein, the term “subject” refers to a mammal, e.g., preferably a human. Mammals include, but are not limited to, humans and domestic and farm animals, such as monkeys (e.g., a cynomolgus monkey), mice, dogs, cats, horses, and cows, etc.

### DESCRIPTION OF THE DRAWINGS

FIG. 1 is a series of graphs showing the effect of ActRIIA/B-mFc (SEQ ID NO: 69 fused to a mouse Fc domain by way of a linker) on thrombopoiesis. As shown in FIG. 1, ActRIIA/B-mFc increased circulating platelet numbers and bone marrow megakaryocyte progenitors within 12 hours post-dose. Data are represented as mean±SEM. Statistical analysis was performed using a Student T-Test; *p<0.05; **** p<0.0001.

FIG. 2 is a series of graphs showing that treatment with ActRIIA/B-mFc had a direct effect on megakaryocyte maturation. Treatment of eleven-week-old C57/BI6 mice with ActRIIA/B-mFc (10 mg/kg via subcutaneous administration), led to an increase in the number of CD41+ megakaryocyte progenitors at 12 hours post-dose (left) and an increase in the number of polyploid megakaryocytes by 24 hours post-dose (right). Data are represented as mean±SEM.

FIGS. 3A-3C are a series of graphs showing that treatment with ActRIIA/B-mFc accelerated recovery of platelet numbers following platelet depletion compared to vehicle treatment. Eleven-week-old mice were treated with either anti-GP1bα (0.08 mg/kg, Efferet) or IgG control. At day 4 after treatment, the anti-GP1bα treated group was further divided to receive either vehicle or ActRIIA/B-mFc (7.5 mg/kg) treatment. Platelets were measured at the indicated time points post anti-GP1bα dosing. At day 10 post-treatment, mice were euthanized and bone marrow cells were harvested. As shown in FIG. 3A, mice treated with ActRIIA/B-mFc exhibited accelerated recovery of platelet numbers following platelet depletion compared to vehicle treated mice in this mouse model of immune thrombocytopenia. Additionally, as shown in FIGS. 3B-3C, there was a 25% increase in the number of CD41⁺ megakaryocyte progenitors in the bone marrow of the ActRIIA/B-mFc-treated group compared to the vehicle-treated group and a higher 4N ploidy level at day 10 after platelet depletion. For platelet data, statistical analysis was performed with repeat measures mixed effect modeling. Individual comparisons shown are from a Tukey post-test. For CD41 data, statistical analysis was performed with 1-Way ANOVA and individual comparisons calculated using a Tukey post-test. * p<0.05; ** p<0.01; *** p<0.001; **** p<0.0001.

FIG. 4 is a series of graphs showing that treatment with a single dose of ActRIIA/B-mFc resulted in increased circulating platelets for at least 85 days. Eleven-week-old C57BL/6 mice were treated with a single dose of either vehicle or ActRIIA/B-mFc (10 mg/kg) by subcutaneous administration. Separate cohorts of mice from both dosing groups were sampled for whole blood at study day 37, 51 and 85, and platelet counts were determined using a veterinary hematology analyzer (Heska Element HT5). Data are shown as mean±SEM. Statistical analysis was performed using a Student T-Test; * p≤0.05; ** p≤0.01; *** p≤0.001; **** p≤0.0001.

FIG. 5 is a graph showing that ex-vivo treatment with ActRIIA/B-mFc reversed activin-mediated changes in megakaryocyte precursors. Bone marrow cells from 11-week-old C57BI/6 mice were isolated and treated with Activin A (5 mg/kg), ActRIIA/B-mFc (10 mg/kg), or a combination of both for six days. Cells were harvested after six days and analyzed using flow cytometry (N=2). Error bars=SEM.

FIG. 6 is a graph showing that treatment with an anti-activin A antibody increased platelets in wild-type mice. Ten-week-old C57BI/6 male mice were dosed with either TBS (vehicle), anti-activin A (5 mg/kg), or ActRIIA/B-mFc (10 mg/kg) via intraperitoneal administration. Twenty-four hours post-dose whole blood was sampled, and platelet counts were determined using a veterinary hematology analyzer (Hematrue). Data are represented as mean±SEM. Statistical analysis was performed using a one-way ANOVA, ** p<0.01, *** p<0.001.

FIG. 7 is a series of graphs showing that platelet number and platelet volume were increased in the TPO^{high} model of myelofibrosis and that treatment with ActRIIA/B-mFc attenuated the expansion of platelets. Seven-week-old C57BI/6 albino mice (B6(Cg)-Tyr, Jackson Laboratory) were teil-vein injected with 0.75 mg/kg thrombopoietin (TPO) expressing plasmid cloned into pLEV113 plasmid (Lake Pharma). The injection was done in a hydrodynamic approach in which 100 ml/kg volume was injected in a short period of time (6-10 seconds). On day 3 after TPO injection, mice were divided into 2 groups receiving either vehicle (TBS) or ActRIIA/B-mFc (7.5 mg/kg), twice weekly via IP injection. Mice were sacrificed on day 14 after TPO injection. Hematological parameters were analyzed by measured using the Heska Element HT5 veterinary hematology analyzer. N=10-12 mice/group. Results are presented as mean±SEM. Statistical analysis was performed by one-way ANOVA. ****=p<0.0001.

FIG. 8 is a series of graphs showing that ActRIIA/B-mFc improved TPO-induced anemia. The TPO^{high} model of myelofibrosis was anemic after 14 days of TPO expression and exhibited reduced red blood cells, hemoglobin, hematocrit, and mean corpuscular hemoglobin concentration. Treatment with ActRIIA/B-mFc was associated with a significant improvement in RBC metrics and appeared to reduce the development of anemia in this model. N=10-12 mice/group. Results are presented as mean±SEM. Statistical analysis was performed by one-way ANOVA. **=p<0.01; ***=p<0.001; ****=p<0.0001.

FIG. 9 is a graph showing that ActRIIA/B-mFc reduced TPO-induced splenic extra medullary hematopoiesis. In the TPO^{high} model of myelofibrosis, the expansion in megakaryocyte growth and proliferation reduces the capacity of bone marrow for hematopoiesis, inducing compensatory extra medullary hematopoiesis in the liver and spleen. These data show a significant reduction in splenomegaly in mice treated with ActRIIA/B-mFc, indicating reduced splenic extra medullary hematopoiesis, likely due to a reduced requirement for this compensatory process. N=10-12 mice/group. Results are presented as mean±SEM. Statistical analysis was performed by one-way ANOVA. *=p<0.05; ****=p<0.0001.

FIG. 10 is a series of graphs showing that ActRIIA/B-mFc reduced the TPO-mediated increase in white blood cells and lymphocytes. The TPO^{high} model of myelofibrosis exhibited an increase in white blood cells, neutrophils, and lymphocytes. Treatment with ActRIIA/B-mFc reduced the increase in white blood cells and lymphocytes in TPO^{high} mice. N=10-12 mice/group. Results are presented as mean±SEM. Statistical analysis was performed by one-way ANOVA. ***=p<0.001; ****=p<0.0001.

FIG. 11 is a graph showing that ActRIIA/B-mFc increased body weight in ruxolitinib treated mice. Ten- to twelve-week-old, female, C57BI/6 mice were dosed orally with either vehicle (n=10) or ruxolitinib at 90 mg/kg (n=20) or 120 mg/kg (n=20) twice daily. After 37 days of ruxolitinib therapy, blood was sampled via cheek bleed for hematological assessment. On Day 41, mice from each ruxolitinib dose were divided into two groups that received either TBS (vehicle) (n=10) or ActRIIA/B-mFc (7.5 mg/kg, n=10 per group), IP twice weekly for 14 days concomitant to continued ruxolitinib dosing. Hematological parameters were assessed in whole blood with the use of a Heska Element HT5 veterinary hematology analyzer. N=10. Data are presented as mean±SEM. Two-way ANOVA was used for statistical analysis. **=p<0.01 between Veh and Rux 90 mg/kg+ActRIIA/B-mFc at each time point. ***=p<0.005 between Veh and Rux 90 mg/kg+ActRIIA/B-mFc at each time point. ****=p<0.0001 between Veh and Rux 90 mg/kg+ActRIIA/B-mFc at each time point. #=p<0.05 between Veh and RUX 120 mg/kg+ActRIIA/B-mFc at each time point. ##=p<0.01 between Veh and RUX 120 mg/kg+ActRIIA/B-mFc at each time point.

FIG. 12 is a series of graphs showing that ruxolitinib administration resulted in reduced red blood cell volume, hemoglobin, and hematocrit. Data are represented as mean±SEM. Vehicle N=10, Rux 90 N=20, Rux 120 N=20. Statistical analysis was performed using a one-way ANOVA followed by Dunnett post hoc test. * p≤0.05; ** p≤0.01; *** p≤0.001; **** p≤0.0001.

FIG. 13 is a series of graphs showing that ActRIIA/B-mFc abrogated ruxolitinib-associated reductions red blood cell volume, hemoglobin, and hematocrit. Data are represented as mean±SEM.

All groups N=10. Statistical analysis was performed using a one-way ANOVA followed by Tukey post hoc test. ns—not significant; * p≤0.05; ** p≤0.01; *** p≤0.001; **** p≤0.0001.

FIGS. 14A-14B are a series of graphs showing the expression of TGF-β receptors and ligands in megakaryocyte precursor cells from untreated mice. Murine bone marrow megakaryocyte precursors expressed activins, GDFs, BMPs and TGF-β ligands (FIG. 14B) and their cognate receptors (FIG. 14A). Receptors and ligands directly related to ActRIIA/B-mFc are in bold. ND, not detected.

### DETAILED DESCRIPTION OF THE INVENTION

The invention features methods of co-administering an activin receptor type II (ActRII) signaling inhibitor and a cytopenia-associated myelofibrosis treatment. The ActRII signaling inhibitor can be an antibody that binds to an ActRII ligand, an anti-ActRII antibody, or an ActRII ligand trap, and exemplary cytopenia-associated myelofibrosis treatments include ruxolitinib (JAKAFI®/JAKAVI®), fedratinib (INREBIC®), pacritinib (VONJO™), and imetelstat. These methods can be used to treat myelofibrosis, such as intermediate or high-risk myelofibrosis, including primary myelofibrosis (PMF), post-polycythemia vera myelofibrosis (post-PV MF), and post-essential thrombocythemia myelofibrosis (post-ET MF). These methods can also reduce or ameliorate cytopenias (e.g., anemia, thrombocytopenia, and or neutropenia) due to the cytopenia-associated myelofibrosis treatment, and can thereby improve adherence to treatment, treatment duration, and maintenance of dose intensity for the cytopenia-associated myelofibrosis treatment, reduce treatment interruptions or discontinuations, and decrease episodes of cytopenia associated with the cytopenia-associated myelofibrosis treatment.

### ActRII Signaling

Activin type II receptors are single transmembrane domain receptors that modulate signals for ligands in the transforming growth factor β (TGF-β) superfamily. Ligands in the TGF-β superfamily are involved in a host of physiological processes, such as muscle growth, vascular growth, cell differentiation, homeostasis, and osteogenesis. Examples of ligands in the TGF-β superfamily include, e.g., activin A, activin B, inhibin, growth differentiation factors (GDFs) (e.g., GDF8, also known as myostatin, and GDF11), and bone morphogenetic proteins (BMPs) (e.g., BMP9).

TGF-β signaling pathways regulate hematopoiesis, with signaling pathways involving activins preventing the differentiation of red blood cell, platelet, and neutrophil progenitor cells in order to maintain progenitor cells in a quiescent state, and signaling pathways involving BMPs promoting differentiation of progenitor cells. Homeostasis of this process is essential to ensure that all cell types, including red cells, white cells, and platelets, are properly replenished in the blood. Relatedly, activin receptor ligand GDF11 has been found to be overexpressed in a mouse model of hemolytic anemia and associated with defects in red blood cell production. These data suggest that increased signaling through endogenous activin receptors, either due to increased expression of activin receptor ligands (e.g., activin A, activin B, myostatin) or increased expression of activin receptors themselves, could disrupt hematopoiesis. Methods that reduce or inhibit activin A, activin B, and/or myostatin signaling could, therefore, be used to promote hematopoiesis and treat diseases and conditions involving ineffective hematopoiesis, such as a cytopenia (e.g., anemia, thrombocytopenia, or neutropenia) associated with myelofibrosis.

The present invention is based, in part, on the discovery by the present inventors that an ActRIIA ligand trap including an ActRIIA variant increased platelets and megakaryocyte progenitors, promoted megakaryocyte maturation, improved TPO-induced anemia and reduced TPO-induced splenic extramedullary hematopoiesis and TPO-mediated increases in white blood cells and lymphocytes in the TPO^{high} model of myelofibrosis, and reversed ruxolitinib-associated reductions in RBC volume, hematocrit, and hemoglobin. The inventors also found that inhibition of activin A with an anti-activin A antibody increased platelets. These data suggest that co-administration of ActRII signaling inhibitors and cytopenia-associated myelofibrosis treatments may be able to improve or ameliorate cytopenias associated with the cytopenia-associated myelofibrosis treatments, which could address the adverse reactions associated with cytopenia-associated myelofibrosis treatments and improve adherence to treatment, treatment duration, and maintenance of dose intensity for the cytopenia-associated myelofibrosis treatment and reduce treatment interruptions or discontinuations and episodes of cytopenia associated with the cytopenia-associated myelofibrosis treatment.

### ActRII Signaling Inhibitors

ActRII signaling inhibitors are agents that reduce or prevent the interaction of ActRII ligands with ActRIIA and/or ActRIIB, by either binding to the ligand or to the receptor. ActRII signaling inhibitors for use in the methods described herein provided herein below.

In some embodiments, the ActRII signaling inhibitor is an activin A antibody or an antigen binding fragment thereof. In some embodiments, the activin A antibody is Garetosmab (also known as REGN-2477). Additional activin A antibodies that may be used in the methods described herein include those described in International Patent Application Publication Nos. WO2015017576, WO2013074557, and WO2008031061; US Patent Application No. US2015/0359850; and U.S. Pat. Nos. 9,718,881, 10,526,403, 8,309,082, 8,753,627, and 10,100,109, each of which is incorporated herein by reference.

In some embodiments, the activin A antibody or an antigen binding fragment thereof has a heavy chain variable region (HCVR) and a light chain variable region (LCVR) listed in Table 1 (e.g., an HCVR and an LCVR from the same row of Table 1). In some embodiments, the activin A antibody or antigen binding fragment thereof includes a HCVR sequence having at least 90% (e.g., at least 91%, 92%, 93%, 94%, 95%, 96%, 97%, 98%, 99%, or 100%) sequence identity to a HCVR sequence in Table 1, such as any one of SEQ ID NOs: 138, 140, 142, 143, 144, 146, 148, 150, 151, 172, and 174, and a LCVR sequence having at least 90% (e.g., at least 91%, 92%, 93%, 94%, 95%, 96%, 97%, 98%, 99%, or 100%) sequence identity to a LCVR sequence in Table 1, such as any one of SEQ ID NOs: 139, 141, 145, 147, 149, 173, and 175. In some embodiments, the activin A antibody or an antigen binding fragment thereof, apart from the light chain CDR1, CDR2, and CDR3 and the heavy chain CDR1, CDR2, and CDR3, has a HCVR and LCVR sequence having at least 90% sequence identity (e.g., 90%, 91%, 92%, 93%, 94%, 95%, 95%, 97%, 98%, 99% or more sequence identity) to a HCVR and LCVR sequence listed in Table 1. In some embodiments, the activin A antibody or an antigen binding fragment thereof has the light chain CDR1, CDR2, and CDR3 and the heavy chain CDR1, CDR2, and CDR3 sequences of an HCVR sequence and an LCVR sequence in Table 1. In some embodiments, the activin A antibody or antigen binding fragment thereof includes an HCVR sequence and an LCVR sequence from the same row of Table 1.

TABLE 1
Exemplary HCVR and LCVR sequences of activin A antibodies

| HCVR | LCVR |
|---|---|
| QVQLQESGPGLVKPSETLSLTCTVSGGSFSSHF | EIVLTQSPGTLSLSPGERATLSCRASQSVSSSYL |
| WSWIRQPPGKGLEWIGYILYTGGTSFNPSLKSR | AWYQQKPGQAPRLLIYGASSRATGIPDRFSGSG |
| VSMSVGTSKNQFSLKLSSVTAADTAVYYCARAR | SGTDFTLTISRLEPEDFAVYYCQQYGSSPWTFG |
| SGITFTGIIVPGSFDIWGQGTMVTVSS (SEQ ID | QGTKVEIK (SEQ ID NO: 139) |
| NO: 138) | |
| QVQLQQSGPGLLKPSQTLSLTCAISGDSVSSNS | DIVMTQSPDSLAVSLGERATINCKSSQSVLYSSN |
| AAWSWIRQSPSRGLEWLGRTYYRANWENDYA | NKNYLAWYQQKPGQPPTLLIYWASTRESGVPD |
| LSVKSRITINPVTSTNHFSLQLHSVTPEDTAVYY | RFSGSGSGTDFTLTISSLQAEDVAIYYCHQYFIT |
| CAREGALGYYFDSWGQGTLVTVSS (SEQ ID | PLTFGGGTKVEIK (SEQ ID NO: 141) |
| NO: 140) | |
| QVQLQESGPGLVKPSETLSITCTVSGGSFSSHF | EIVLTQSPGTLSLSPGERATLSCRASQSVSSSYL |
| WTWIRQPPGKGLEWIGYLHYSGGTSYNPSLKS | AWYQQKPGQAPRLLIYGASSRATGIPDRFSGSG |
| RVIISVDTSKNQFSLKLNSVTAADTAVYYCARAR | SGTDFTLTISRLEPEDFAVYYCQQYGSSPWTFG |
| SGITFGGLIVPGSFDIWGQGTMVTVSS | QGTKVEIK (SEQ ID NO: 139) |
| (SEQ ID NO: 142) | |
| QVQLQESGPGLVKPSETLSLTCTVSGGSFSSHF | EIVLTQSPGTLSLSPGERATLSCRASQSVSSSYL |
| WSWIRQPPGKGLEWIGYIYYSGGTHYNPSLESR | AWYQQKPGQAPRLLIYGASSRATGIPDRFSGSG |
| VTISVDTSKNQFSLKLNSVTAADTAVYYCARARS | SGTDFTLTISRLEPEDFAVYYCQQYGSSPWTFG |
| GITFGGLIVPGSFDIWGQGTMVTVSS | QGTKVEIK (SEQ ID NO: 139) |
| (SEQ ID NO: 143) | |
| EVQLVESGGGLVQPGRSLRLSCKASGFAFDDF | EIVLTQSPATLSLSPGERATLSCRASQTISTYLV |
| AMHWVRQAPGKGLEWVSGIVWNSGDIGYADS | WYRQRPGQAPSLLIYDASNRATDIPARFSGSGS |
| VKGRFTISRDNAKNSLYLQLNSLRTEDTALYFCV | GTDFTLTISSLEPEDFAVYYCQQRSNWPITFGQ |
| KDMVRGLMGFNYYGMDVWGQGTTVTVSS | GTRLEIK (SEQ ID NO: 145) |
| (SEQ ID NO: 144) | |
| QVQLVESGGGVVQPGRSLRLSCVASGFTVSSY | DIQMTQSPSSLSASVGDRVTITCRASQNINSFLS |
| GIHWVRQAPGKGLEWVSVIWYDGRNKDYADSV | WYQQKPGKAPKFLIYDASSIQSGAPSRFSGSGS |
| KGRFTISRDNSKNTVYLEMKGLRAEDTALYYCA | GTDFTLTISSLQPEDFATYYCQQSYSSPFTFGQ |
| RDKTGDFDSWGQGTLVTVSS (SEQ ID NO: 146) | GTKLEIK (SEQ ID NO: 147) |
| EVQLVESGGGLVQPGRSLTLSCAVSGFTFDDH | DIQMTQSPSSLSASVGDRVTITCRASQSISSYLN |
| AMHWVRQAPGKGLEWVSGISWNSVSIGYADSV | WYQQKPGKAPKLLIYAASSLQSGVPSRFSGSG |
| KGRFTISRDNAKTSLYLQMNSLRVDDTALYYCV | SGTDFTLTISSLQPEDFATYYCQQSYSTPPITFG |
| QDLNDILTGYPLFDFWGQGTLVTVSS (SEQ ID | QGTRLEIK (SEQ ID NO: 149) |
| NO: 148) | |
| QVQLVESGGGVVQPGRSLRLTCVASGFTVSSY | DIQMTQSPSSLSASVGDRVTITCRASQSISSYLN |
| GMHWVRQAPGKGLEWVAVMFYDESKKYYADS | WYQQKPGKAPKLLIYAASSLQSGVPSRFSGSG |
| VKGRFTISRDNSKNTLYLQMNSLRVEDTAVYYC | SGTDFTLTISSLQPEDFATYYCQQSYSTPPITFG |
| ARDEQLDFEYWGQGTLVTVSS (SEQ ID NO: | QGTRLEIK (SEQ ID NO: 149) |
| 150) | |
| EVQLVESGGGVVQPGGSLRLSCAASTFTFDDF | DIQMTQSPSSLSASVGDRVTITCRASQSISSYLN |
| AMHWVRQAPGKGLEWVSLITGDGGSTYYADPV | WYQQKPGKAPKLLIYAASSLQSGVPSRFSGSG |
| KGRFTISRDNSKNSLYLQMNSLRTEDTALYYCV | SGTDFTLTISSLQPEDFATYYCQQSYSTPPITFG |
| KDWWIAARPDYYYYGMDVWGQGTTVTVSS | QGTRLEIK (SEQ ID NO: 149) |
| (SEQ ID NO: 151) | |
| QVQLVQSGAEVKKPGASVKVSCKASGYTFTSY | SYEVTQAPSVSVSPGQTASITCSGDKLGDKYAC |
| GLSWVRQAPGQGLEWMGWIIPYNGNTNSAQKL | WYQQKPGQSPVLVIYQDSKRPSGIPERFSGSN |
| QGRVTMTTDTSTSTAYMELRSLRSDDTAVYFCA | SGNTATLTISGTQAMDEADYYCQAWDSSTAVF |
| RDRDYGVNYDAFDIWGQGTMVTVSS (SEQ ID | GGGTKLTVL (SEQ ID NO: 173) |
| NO: 172) | |
| EVQLVESGGGLVQPGGSLRLSCAASGFTFSSY | DIQMTQSPSSLSASVGDRVTITCRASQGIRNDL |
| WMSWVRQAPGKGLECVANIKQDGSEEYYVDS | GWYQQKPGKAPKRLIYAASSLQSGVPSRFSGS |
| VKGRFTISRDNAKNSLYLQMNSLRAEDTAVYYC | GSGTEFTLTISSLQPEDFATYYCRQQNTYPLTF |
| ARGSSSWYYYNYGMDVWGQGTTVTVSS (SEQ | GGGTKVEIK (SEQ ID NO: 175) |
| ID NO: 174) | |

In some embodiments, the activin A antibody or an antigen-binding fragment thereof, has the CDR sequences described in Table 2 (i.e., a light chain CDR1, CDR2, and CDR3 and a heavy chain CDR1, CDR2, and CDR3). In some embodiments, the activin A antibody or antigen binding fragment thereof includes a light chain variable CDR1 sequence having at least 90% (e.g., at least 91%, 92%, 93%, 94%, 95%, 96%, 97%, 98%, 99%, or 100%) sequence identity to a light chain variable CDR1 sequence in Table 2, such as any one of SEQ ID NOs: 155, 161, 179, and 185; a light chain variable CDR2 sequence having at least 90% (e.g., at least 91%, 92%, 93%, 94%, 95%, 96%, 97%, 98%, 99%, or 100%) sequence identity to a light chain variable CDR2 sequence in Table 2, such as any one of SEQ ID NOs: 156, 162, 180, and 186; a light chain variable CDR3 sequence having at least 90% (e.g., at least 91%, 92%, 93%, 94%, 95%, 96%, 97%, 98%, 99%, or 100%) sequence identity to a light chain variable CDR3 sequence in Table 2, such as any one of SEQ ID NOs: 157, 163, 181, and 187; a heavy chain variable CDR1 sequence having at least 90% (e.g., at least 91%, 92%, 93%, 94%, 95%, 96%, 97%, 98%, 99%, or 100%) sequence identity to a heavy chain variable CDR1 sequence in Table 2, such as any one of SEQ ID NOs: 152, 158, 176, and 182; a heavy chain variable CDR2 sequence having at least 90% (e.g., at least 91%, 92%, 93%, 94%, 95%, 96%, 97%, 98%, 99%, or more) sequence identity to a heavy chain variable CDR2 sequence in Table 2, such as any one of SEQ ID NOs: 153, 159, 177, and 183; and a heavy chain variable CDR3 sequence having at least 90% (e.g., at least 91%, 92%, 93%, 94%, 95%, 96%, 97%, 98%, 99%, or more) sequence identity to a heavy chain variable CDR3 sequence in Table 2, such as any one of SEQ ID NOs: 154, 160, 178, and 184. In some embodiments, the activin A antibody or antigen binding fragment thereof includes a light chain CDR1, CDR2, and CDR3 sequence and a heavy chain CDR1, CDR2, and CDR3 sequence from the same row of Table 2.

TABLE 2
Exemplary CDR sequences of activin A antibodies

| HCDR1 | HCDR2 | HCRD3 | LCDR1 | LCDR2 | LCDR3 |
|---|---|---|---|---|---|
| GGSFSSHF | ILYTGGT | ARARSGITFT | QSVSSSY | GAS (SEQ ID | QQYGSSPWT |
| (SEQ ID NO: | (SEQ ID NO: | GIIVPGSFDI | (SEQ ID NO: | NO: 156) | (SEQ ID NO: |
| 152) | 153) | (SEQ ID NO: | 155) | | 157) |
| | | 154) | | | |
| GFAFDDFA | IVWNSGDI | VKDMVRGLM | QTISTY (SEQ | DAS (SEQ ID | QQRSNWPIT |
| (SEQ ID NO: | (SEQ ID NO: | GFNYYGMDV | ID NO: 161) | NO: 162) | (SEQ ID NO: |
| 158) | 159) | (SEQ ID NO: | | | 163) |
| | | 160) | | | |
| GYTFTSYGLS | WIIPYNGNTN | DRDYGVNYD | SGDKLGDKY | QDSKRPS | QAWDSSTAV |
| (SEQ ID NO: | SAQKLQG | AFDI (SEQ ID | AC (SEQ ID | (SEQ ID NO: | (SEQ ID NO: |
| 176) | (SEQ ID NO: | NO: 178) | NO: 179) | 180) | 181) |
| | 177) | | | | |
| GFTFSSYWM | NIKQDGSEEY | GSSSWYYYN | RASQGIRNDL | AASSLQS | RQQNTYPLT |
| S (SEQ ID | YVDSVKG | YGMDV (SEQ | G (SEQ ID | (SEQ ID NO: | (SEQ ID NO: |
| NO: 182) | (SEQ ID NO: | ID NO: 184) | NO: 185) | 186) | 187) |
| | 183) | | | | |

In some embodiments, the ActRII signaling inhibitor is a myostatin antibody or an antigen binding fragment thereof. In some embodiments, the myostatin antibody is Domagrozumab (also known as PF-06252616), Landogrozumab (also known as LY2495655), Trevogrumab (also known as REGN-1033), or SRK-015. Additional myostatin antibodies that may be used in the methods described herein include those described in International Patent Application Publication Nos. WO2007047112, WO2007044411, WO2006116269, WO2012024242, WO2016073853, WO2013186719, WO2009058346, WO2011150008, WO2016168613, WO2007024535, and WO2016098357, US Patent Application Nos. US20070178095 and US20210246198; and U.S. Pat. Nos. 10,000,560, 10,738,111, 7,632,499, 8,066,995, 7,635,760, 7,745,583, 7,745,583, 7,807,159, 8,999,343, 10,307,480, 8,992,913, 9,751,937, 9,409,981, 9,850,301, 8,840,894, 9,890,212, 9,260,515, 10,934,349, 8,871,209, 10,400,036, 7,888,486, and 8,372,625, each of which is incorporated herein by reference.

In some embodiments, the myostatin antibody or an antigen binding fragment thereof has a HCVR and a LCVR listed in Table 3 (e.g., an HCVR and an LCVR from the same row of Table 3). In some embodiments, the myostatin antibody or antigen binding fragment thereof includes a HCVR sequence having at least 90% (e.g., at least 91%, 92%, 93%, 94%, 95%, 96%, 97%, 98%, 99%, or 100%) sequence identity to a HCVR sequence in Table 3, such as any one of SEQ ID NOs: 164, 188, 201, 204-210, 222-228, 234, 250, 252, 254, 256, 258, 260, 262, 264, 266, 268, 270, 272, 298, 306, 308, 310, 312, 314, 316, 318, 320, 356, 371, 373, 387, 389, 391, 405, 407, 409, 411, 413, 415, 417, 419, 421-423, 425, 427, 429, 431, 433, 435, 437, 439, 441, 444, 446, and 448-476, and a LCVR sequence having at least 90% (e.g., at least 91%, 92%, 93%, 94%, 95%, 96%, 97%, 98%, 99%, or 100%) sequence identity to a LCVR sequence in Table 3, such as any one of SEQ ID NOs: 165, 189, 202, 203, 221, 229-233, 251, 253, 255, 257, 259, 261, 263, 265, 267, 269, 271, 273, 299, 307, 309, 311, 313, 315, 317, 319, 321, 358, 372, 374, 388, 390, 392, 406, 408, 410, 412, 414, 416, 418, 420, 424, 426, 428, 430, 432, 434, 436, 438, 440, 442, 443, 445, 447, and 477-486. In some embodiments, the myostatin antibody or an antigen binding fragment thereof, apart from the light chain CDR1, CDR2, and CDR3 and the heavy chain CDR1, CDR2, and CDR3, has a HCVR and LCVR sequence having at least 90% sequence identity (e.g., 90%, 91%, 92%, 93%, 94%, 95%, 95%, 97%, 98%, 99% or more sequence identity) to a HCVR and LCVR sequence listed in Table 3. In some embodiments, the myostatin antibody or an antigen binding fragment thereof has the light chain CDR1, CDR2, and CDR3 and the heavy chain CDR1, CDR2, and CDR3 sequences of an HCVR sequence and an LCVR sequence in Table 3. In some embodiments, the myostatin antibody or antigen binding fragment thereof includes an HCVR sequence and an LCVR sequence from the same row of Table 3. In some embodiments, the myostatin antibody or antigen binding fragment thereof includes an HCVR sequence of any one of SEQ ID NOs: 448-476 and an LCVR sequence of any one of SEQ ID NOs: 477-486.

TABLE 3
Exemplary HCVR and LCVR sequences of myostatin antibodies

| HCVR | LCVR |
|---|---|
| EVQVLESGGDLVQPGGSLRLSCAASGFTFSAY | DIQMTQSPASLSASVGDRVTITCRASQDISDYLA |
| AMTWVRQAPGKGLEWVSAISGSGGSAYYADSV | WYQQKPGKIPRLLIYTTSTLQSGVPSRFRGSGS |
| KGRFTISRDNSKNTVYLQMNSLRAEDTAVYYCA | GTDFTLTISSLQPEDVATYYCQKYDSAPLTFGG |
| KDGAWKMSGLDVWGQGTTVIVSS (SEQ ID NO: | GTKVEIK (SEQ ID NO: 165) |
| 164) | |
| EVQLVESGGGLVQPGGSLRLSCAASGLTFSRY | EIVLTQSPGTLSLSPGERATLSCRASSSVSSSYL |
| PMSWVRQAPGKGLVWVSAITSSGGSTYYSDTV | HWYQQKPGQAPRLLIYSTSNLVAGIPDRFSGSG |
| KGRFTISRDNAKNTLYLQMNSLRAEDTAVYYCA | SGTDFTLTISRLEPEDFAVYYCQHHSGYHFTFG |
| RLPDYWGQGTLVTVSS (SEQ ID NO: 188) | GGTKVEIK (SEQ ID NO: 189) |
| QVTLRESGPALVKPTQTLTLTCTFSGFSLRKVG | DIQMTQSPSSLSASVGDRVTITCSASSSISYMH |
| SSVSWIRQPPGKALEWLAHIYWDDDKRYNPSL | WYQQKPGKAPKLLIYDTSKLAVGVPSRFSGSGS |
| RNRLTISKDTSKNQVVLTMTNMDPVDTATYYCA | GTDFTLTISSLQPEDFATYYCQQWYRNPLTFGG |
| RRAITTVIGGGTFDYWGQGTTVTVSS (SEQ ID | GTKVEIK (SEQ ID NO: 202) |
| NO: 201) | |
| QVTLRESGPALVKPTQTLTLTCTFSGFSLRKVG | DIQMTQSPSSLSASVGDRVTITCSASSSISYMH |
| SSVSWIRQPPGKALEWLAHIYWDDDKRYNPSL | WYQQKPGKAPKLLIYDTSKLARGVPSRFSGSGS |
| RNRLTISKDTSKNQVVLTMTNMDPVDTATYYCA | GTDFTLTISSLQPEDFATYYCQQWYRNPLTFGG |
| RRAITTVIGGGTMDYWGQGTTVTVSS (SEQ ID | GTKVEIK (SEQ ID NO: 203) |
| NO: 204) | |
| QVTLRESGPALVKPTQTLTLTCTFSGFSLRKVG | DIQMTQSPSSLSASVGDRVTITCSASSSISYMH |
| SSVSWIRQPPGKALEWLAHIYWDDDKRYNPSL | WYQQKPGKAPKLLIYDTSKLARGVPSRFSGSGS |
| RNRLTISKDTSKNQVVLTMTNMDPVDTATYYCA | GTDFTLTISSLQPEDFATYYCQQWYRNPLTFGG |
| RRAITTVIGGGTFDYWGQGTTVTVSS (SEQ ID | GTKVEIK (SEQ ID NO: 203) |
| NO: 201) | |
| QVTLRESGPALVKPTQTLTLTCTFSGFSLRKVG | DIQMTQSPSSLSASVGDRVTITCSASSSISYMH |
| RSVSWIRQPPGKALEWLAHIYWDDDKRYNPSL | WYQQKPGKAPKLLIYDTSKLARGVPSRFSGSGS |
| RNRLTISKDTSKNQVVLTMTNMDPVDTATYYCA | GTDFTLTISSLQPEDFATYYCQQWYRNPLTFGG |
| RRAITTVIGGGTFDYWGQGTTVVSS (SEQ ID | GTKVEIK (SEQ ID NO: 203) |
| NO: 205) | |
| QVTLRESGPALVKPTQTLTLTCTFSGFSWRKVG | DIQMTQSPSSLSASVGDRVTITCSASSSISYMH |
| SSVSWIRQPPGKALEWLAHIYWDDDKRYNPSL | WYQQKPGKAPKLLIYDTSKLARGVPSRFSGSGS |
| RNRLTISKDTSKNQVVLTMTNMDPVDTATYYCA | GTDFTLTISSLQPEDFATYYCQQWYRNPLTFGG |
| RRAITTVIGGGTFDYWGQGTTVTVSS (SEQ ID | GTKVEIK (SEQ ID NO: 203) |
| NO: 206) | |
| QVTLRESGPALVKPTQTLTLTCTFSGFSLRKVG | DIQMTQSPSSLSASVGDRVTITCSASSSISYMH |
| SSVSWIRQPPGKALEWLAHIYWDDDLRYNPSLR | WYQQKPGKAPKLLIYDTSKLARGVPSRFSGSGS |
| NRLTISKDTSKNQVVLTMTNMDPVDTATYYCAR | GTDFTLTISSLQPEDFATYYCQQWYRNPLTFGG |
| RAITTVIGGGTFDYWGQGTTVTVSS (SEQ ID | GTKVEIK (SEQ ID NO: 203) |
| NO: 207) | |
| QVTLRESGPALVKPTQTLTLTCTFSGFSMRKVG | DIQMTQSPSSLSASVGDRVTITCSASSSISYMH |
| SSVSWIRQPPGKALEWLAHIYWDDDKRYNPSL | WYQQKPGKAPKLLIYDTSKLARGVPSRFSGSGS |
| RNRLTISKDTSKNQVVLTMTNMDPVDTATYYCA | GTDFTLTISSLQPEDFATYYCQQWYRNPLTFGG |
| RRAITTVIGGGTFDYWGQGTTVTVSS (SEQ ID | GTKVEIK (SEQ ID NO: 203) |
| NO: 208) | |
| QVTLRESGPALVKPTQTLTLTCTFSGFSLRKVG | DIQMTQSPSSLSASVGDRVTITCSASSSISYMH |
| SSISWIRQPPGKALEWLAHIYWDDDKRYNPSLR | WYQQKPGKAPKLLIYDTSKLARGVPSRFSGSGS |
| NRLTISKDTSKNQVVLTMTNMDPVDTATYYCAR | GTDFTLTISSLQPEDFATYYCQQWYRNPLTFGG |
| RAITTVIGGGTFDYWGQGTTVTVSS (SEQ ID | GTKVEIK (SEQ ID NO: 203) |
| NO: 209) | |
| QVTLRESGPALVKPTQTLTLTCTFSGFSLRKVG | DIQMTQSPSSLSASVGDRVTITCSASSSISYMH |
| SSVSWIRQPPGKALEWLAHIYWDDDKRYNPSL | WYQQKPGKAPKLLIYDTSKLARGVPSRFSGSGS |
| RNRLTISKDTSKNQVVLTMTNMDPVDTATYYCA | GTDFTLTISSLQPEDFATYYCQQWYRNPLTFGG |
| RRKITTVIGGGTFDYWGQGTTVTVSS (SEQ ID | GTKVEIK (SEQ ID NO: 203) |
| NO: 210) | |
| QLQLQESGPGLVKPSETLSLTCTVSGFSLRRVG | DIQMTQSPSSLSASVGDRVTITCSASSSISYMH |
| SSVSWIRQPPGKGLEWIGHIYWDDDKRYNPSL | WYQQKPGKAPKLLIYDTSKLARGVPSRFSGSGS |
| RNRVTISVDTSKNQFSLKLSSVTAADTAVYYCA | GTDFTLTISSLQPEDFATYYCQQWYSNPLTFGG |
| RRAITTVIGGGTFDYWGQGTLVTVSS (SEQ ID | GTKVEIK (SEQ ID NO: 221) |
| NO: 222) | |
| QLQLQESGPGLVKPSETLSLTCTVSGFSLRMVG | DIQMTQSPSSLSASVGDRVTITCSASSSISYMH |
| SSVSWIRQPPGKGLEWIGHIYWDDDKRLNPSLR | WYQQKPGKAPKLLIYDTSKLARGVPSRFSGSGS |
| NRVTISVDTSKNQFSLKLSSVTAADTAVYYCAR | GTDFTLTISSLQPEDFATYYCQQWYSNPLTFGG |
| RAITTVIGGGTFDYWGQGTLVTVSS (SEQ ID | GTKVEIK (SEQ ID NO: 221) |
| NO: 223) | |
| QLQLQESGPGLVKPSETLSLTCTVSGFSLRKVG | DIQMTQSPSSLSASVGDRVTITCSASSSISYMH |
| SSVSWIRQPPGKGLEWIGHIYWDDDLRYNPSLR | WYQQKPGKAPKLLIYDTSKLARGVPSRFSGSGS |
| NRVTISVDTSKNQFSLKLSSVTAADTAVYYCAR | GTDFTLTISSLQPEDFATYYCQQWYSNPLTFGG |
| RAITTVIGGGTFDYWGQGTLVTVSS (SEQ ID | GTKVEIK (SEQ ID NO: 221) |
| NO: 224) | |
| QLQLQESGPGLVKPSETLSLTCTVSGFSLRKVG | DIQMTQSPSSLSASVGDRVTITCSASSSISYMH |
| SSVSWIRQPPGKGLEWIGHIYWDDDKRYNPSL | WYQQKPGKAPKLLIYDTSKLARGVPSRFSGSGS |
| RNRVTISVDTSKNQFSLKLSSVTAADTAVYYCA | GTDFTLTISSLQPEDFATYYCQQWYSNPLTFGG |
| RRAITTVIGGGTFDMWGQGTLVTVSS (SEQ ID | GTKVEIK (SEQ ID NO: 221) |
| NO: 225) | |
| QLQLQESGPGLVKPSETLSLTCTVSGFSLRKVG | DIQMTQSPSSLSASVGDRVTITCSASSSISYMH |
| SSVSWIRQPPGKGLEWIGHIYWDDDKRYNPSL | WYQQKPGKAPKLLIYDTSKLARGVPSRFSGSGS |
| RNRVTISVDTSKNQFSLKLSSVTAADTAVYYCA | GTDFTLTISSLQPEDFATYYCQQWYSNPLTFGG |
| RRAITTVIGGGTFDLWGQGTLVTVSS (SEQ ID | GTKVEIK (SEQ ID NO: 221) |
| NO: 226) | |
| QLQLQESGPGLVKPSETLSLTCTVSGFSLRKVG | DIQMTQSPSSLSASVGDRVTITCSASSSISYMH |
| SSVSWIRQPPGKGLEWIGHIYWDDDKRLNPSLR | WYQQKPGKAPKLLIYDTSKLARGVPSRFSGSGS |
| NRVTISVDTSKNQFSLKLSSVTAADTAVYYCAR | GTDFTLTISSLQPEDFATYYCQQWYSNPLTFGG |
| RAITTVIGGGTFDYWGQGTLVTVSS (SEQ ID | GTKVEIK (SEQ ID NO: 221) |
| NO: 227) | |
| QLQLQESGPGLVKPSETLSLTCTVSGFSLRKVG | DIQMTQSPSSLSASVGDRVTITCSASSSISYSHW |
| SSVSWIRQPPGKGLEWIGHIYWDDDKRYNPSL | YQQKPGKAPKLLIYDTSKLARGVPSRFSGSGSG |
| RNRVTISVDTSKNQFSLKLSSVTAADTAVYYCA | TDFTLTISSLQPEDFATYYCQQWYSNPLTFGGG |
| RRAITTVIGGGTFDYWGQGTLVTVSS (SEQ ID | TKVEIK (SEQ ID NO: 229) |
| NO: 228) | |
| QLQLQESGPGLVKPSETLSLTCTVSGFSLRKVG | DIQMTQSPSSLSASVGDRVTITCSASSSISYAHW |
| SSVSWIRQPPGKGLEWIGHIYWDDDKRYNPSL | YQQKPGKAPKLLIYDTSKLARGVPSRFSGSGSG |
| RNRVTISVDTSKNQFSLKLSSVTAADTAVYYCA | TDFTLTISSLQPEDFATYYCQQWYSNPLTFGGG |
| RRAITTVIGGGTFDYWGQGTLVTVSS (SEQ ID | TKVEIK (SEQ ID NO: 230) |
| NO: 228) | |
| QLQLQESGPGLVKPSETLSLTCTVSGFSLRKVG | DIQMTQSPSSLSASVGDRVTITCSASSSISYMH |
| SSVSWIRQPPGKGLEWIGHIYWDDDKRYNPSL | WYQQKPGKAPKLLIYDTSKLARGVPSRFSGSGS |
| RNRVTISVDTSKNQFSLKLSSVTAADTAVYYCA | GTDFTLTISSLQPEDFATYYCQQWYLNPLTFGG |
| RRAITTVIGGGTFDYWGQGTLVTVSS (SEQ ID | GTKVEIK (SEQ ID NO: 231) |
| NO: 228) | |
| QLQLQESGPGLVKPSETLSLTCTVSGFSLRKVG | DIQMTQSPSSLSASVGDRVTITCSASSSISYMH |
| SSVSWIRQPPGKGLEWIGHIYWDDDKRYNPSL | WYQQKPGKAPKLLIYDTSKLARGVPSRFSGSGS |
| RNRVTISVDTSKNQFSLKLSSVTAADTAVYYCA | GTDFTLTISSLQPEDFATYYCQQWYENPLTFGG |
| RRAITTVIGGGTFDYWGQGTLVTVSS (SEQ ID | GTKVEIK (SEQ ID NO: 232) |
| NO: 228) | |
| QLQLQESGPGLVKPSETLSLTCTVSGFSLRKVG | DIQMTQSPSSLSASVGDRVTITCSASSSISYMH |
| SSVSWIRQPPGKGLEWIGHIYWDDDKRYNPSL | WYQQKPGKAPKLLIYDTSKLARGVPSRFSGSGS |
| RNRVTISVDTSKNQFSLKLSSVTAADTAVYYCA | GTDFTLTISSLQPEDFATYYCQQWYFNPLTFGG |
| RRAITTVIGGGTFDYWGQGTLVTVSS (SEQ ID | GTKVEIK (SEQ ID NO: 233) |
| NO: 228) | |
| QLQLQESGPGLVKPSETLSLTCTVSGFSLRKVG | DIQMTQSPSSLSASVGDRVTITCSASSSISYMH |
| SSVSWIRQPPGKGLEWIGHIYWDDDKRLNPSLR | WYQQKPGKAPKLLIYDTSKLARGVPSRFSGSGS |
| NRVTISVDTSKNQFSLKLSSVTAADTAVYYCAR | GTDFTLTISSLQPEDFATYYCQQWYLNPLTFGG |
| RAITTVIGGGTFDLWGQGTLVTVSS (SEQ ID | GTKVEIK (SEQ ID NO: 231) |
| NO: 234) | |
| EVQLVESGGGLVKPGGSLRLSCAASGFTFSSY | DIQMTQSPSSLSASVGDRVTITCRASQGIRNAL |
| SMNWVRQAPGKGLEWVSSISSSSSYIYYADSV | GWYQQKPGKAPQRLIYAASSLQSGVPSRFSGS |
| KGRFTISRDNAKNSLYLQMNSLRAEDTAVYYCA | GSGTEFTLTISSLQPEDFATYYCLQHNSYPRTF |
| RDRGYSYGLDYWGQGTLVTVSS (SEQ ID NO: | GQGTKVEIK (SEQ ID NO: 251) |
| 250) | |
| QVQLVQSGAEVKKPGASVKVSCKASGYTFTGY | DIVMTQSPLSLPVTPGEPASISCRSSQSLLHSNG |
| YMHWVRQAPGQGLEWMGWINPNNGGTNYAQ | YNYLDWYLQKSGQSPQLLIHLGSNRASGVPDR |
| KFQGRVTMTRDTSISTAYMELSRLRSDDTAVYH | FSGSGSGTDFTLKISRVEAEDVGVYYCMQVLQT |
| CARNTVGSGYYYYGMDVWGQGTTVT | PLTFGGGTKVEIK (SEQ ID NO: 253) |
| VSS (SEQ ID NO: 252) | |
| EVQLVESGGGLVKPGGSLRLSCAASGFTFSSY | DIQMTQSPSSLSASVGDRVTITCRASQGIRNAL |
| SMNWVRQAPGKGLEWVSSISSSSSYIYYADSV | GWYQQKPGKAPKRLIYAASSLQSGVPSRFSGS |
| KGRFTISRDNAKNSLYLQMNSLRAEDTAVYYCA | GSGTEFTLTISSLQPEDFATYYCLQHNSYPRTF |
| RDRGYSYGIDYWGQGTLVTVSS (SEQ ID NO: | GQGTKVEIK (SEQ ID NO: 255) |
| 254) | |
| EVQLVESGGGLVKPGGSLRLSCAASGFTFSSY | DIQMTQSPSSLSASVGDRVTLTCRASQGIRNAL |
| SMNWVRQAPGKGLEWVSSISSSSSYIYYADSV | GWYQQKPGKAPKRLIYGASSLQSGVPSRFSGS |
| KGRFTISRDNAKNSLYLQMNSLRAEDTAVYYCA | GSGTEFTLTISSLQPEDFATYYCLQHNSYPFTFG |
| RDRGYYYGMDVWGQGTTVTVSS (SEQ ID NO: | PGTKVDIK (SEQ ID NO: 257) |
| 256) | |
| EVQLVESGGGLVKPGGSLRLSCAASGFTFSSY | DMQMTQSPSSLSASVGDRVTITCRASQGIRNDL |
| SMNWVRQAPGKGLEWVSSISSSSSYIYYADSV | GWYQQKPGKAPKRLIYGASSLQSGVPSRFSGS |
| KGRFTISRDNAKNSLYLQMNSLRAEDTAVYYCA | GSGTEFTLTISSLQPEDSATYYCLQHNSYPLTFG |
| RDRGSYFLFDYWGQGTLVTVSS (SEQ ID NO: | GGTKVEIK (SEQ ID NO: 259) |
| 258) | |
| EVQLVESGGGLVKPGGSLRLSCAASGFTFSSY | DIQMTQSPSSLSASVGDRVTITCRASQAIRNDLG |
| SMNWVRQAPGKGLEWVSSISSSSSYIYYADSV | WYQQKPGKAPKRLIYAASSLQSGVPSRFSGSG |
| KGRFTISRDNAKNSLYLQMNSLRAEDTTVYYCA | SGTEFTLTISSLQPEDFATYYCLQHNSYPRTFG |
| RDRGYLYGMDVWGQGTTVTVSS (SEQ ID NO: | QGTKVEIK (SEQ ID NO: 261) |
| 260) | |
| QVQLVQSGAEVKKPGASVKVSCKASGYTFTGY | DIVMTQSPLSLPVTPGEPASISCRSSQSLLHSNG |
| YMHWVRQAPGQGLEWMGWINPNSGGTNYAQ | YNYLDWYLQKPGQSPQVLIYLVSNRASGVPDRF |
| KFQGRVTMTRDTSISTAYLELSRLRSDDTAVYY | SGSGSGTDFTLKISRVEAEDVGVYYCMQALQTP |
| CARNTVGTGYYYYGMDVWGQGTTVT | PTFGGGTKVEIK (SEQ ID NO: 263) |
| VSS (SEQ ID NO: 262) | |
| EVQLVESGGGLVKPGGSLRLSCAASGFTFSSY | DIQMTQSPSSLSASVGDRVTITCRASQGIRNDL |
| SMNWVRQAPGKGLEWVSSISSSSSYIYYADSV | GWYQQKPGKAPKRLIYAASSLQSGVPSRFSGS |
| KGRFTISRDNAKNSLYLQMNSLRAEDTAVYYCA | GSGTEFTLTISSLQPEDFATYYCLQLNSYPFTFG |
| RDRGYSYGLDYWGQGTLVTVSS (SEQ ID NO: | PGTKVDIK (SEQ ID NO: 265) |
| 264) | |
| EVQLLESGGGLIQPGGSLRLSCAASGFTFSSFA | EIVMTQSPATLSVSPGERATLSCRASQSVSSNL |
| MSWVRQAPGKGLEWVSTISGSGGYTFYADSVK | AWYQQKPGQAPRLLIYGASTRATGFPARFSGS |
| GRFTISRDNSKNTLYLQMNSLRAEDTAVYYCAK | GSGTEFTLTISSLQSEDFAIYYCQQYNNWPLTF |
| DGRYNWNYGAFDIWGQGTMVTVSS (SEQ ID | GGGTKVEIK (SEQ ID NO: 267) |
| NO: 266) | |
| EVQLLESGGGLIQPGGSLRLSCAASGFTFSSFA | EIVMTQSPATLSVSPGERATLSCRASQSVRSNL |
| MSWVRQAPGKGLEWVSTISGSGGSTYYADFVK | AWFQQKPGQAPRLLIYGASTRATGIPARFSGSG |
| GRFTISRDNSKNTLYLQMNSLRAEDTAVYYCAK | SGTEFTLTISSLQSEDFAVYYCQQYNNWPLTFG |
| DGRYNWNYGAFDIWGQGTMVTVSS (SEQ ID | GGTKVEIK (SEQ ID NO: 269) |
| NO: 268) | |
| EVQLLESGGGLVQPGGSLRLSCAASGFTFSSY | EIVMTQSPATLSVSPGERATLSCRTSQSVSSNL |
| AMSWVRQAPGKGLEWVSAISGSGGSTFYADSV | AWYQQKPGQAPRLLIYGASTRATGIPARFSGSG |
| TGRFTISRDNSKNTLFLQLNSLRAEDTAVYHCAK | SGTEFTLTISSLQSEDFAVYYCQQYNNWPLTFG |
| DGRFNWNYGASDIWGQGTMVTVSS (SEQ ID | GGTKVEIK (SEQ ID NO: 271) |
| NO: 270) | |
| EVQLLESGGGLVQPGGSLRLSCAASGFTFSSFA | EIVMTQSPATLSVSPGERATLSCRASQSVSSNL |
| MSWVRQAPGKGLEWVSTISGSGGYTFYADSVK | AWYQQKPGQAPRLLIYGASTRATGIPARFSGSG |
| GRFTISRDNSKNTLYLQMNSLRAEDTAVYYCAK | SGTEFTLTISSLQSEDFAVYYCQQYNNWPLTFG |
| DGRYNWNYGAFDIWGQGTMVTVSS (SEQ ID | GGTKVEIK (SEQ ID NO: 273) |
| NO: 272) | |
| EVQLVESGGGLVQPGGSLRLSCAASGFTFSRY | DIQMTQSPSSLSASVGDRVTITCKASQDINKYVA |
| WMNWVRQAPGKGLEWVAQIRLKSDNYATHYA | WYQQKPGKAPKLLIYYTSWLQPGVPSRFSGSG |
| ESVKGRFTISRDNAKNSLYLQMNSLRAEDTAVY | SGTDFTFTISSLQPEDIATYYCLQYDNLLYTFGQ |
| YCTEGLDYWGQGTTVTVSS (SEQ ID NO: 298) | GTKLEIK (SEQ ID NO: 299) |
| QIQLVQSGGGVVQPGRSLRLSCAASGFTFSSY | QPVLTQPPSASGTPGQRVTISCSGSSSNIGSNT |
| GMHWVRQAPGKGLEWVAVISYDGSNKYYADS | VHWYQQLPGTAPKLLIYSDNQRPSGVPDRFSG |
| VKGRFTISRDNSKNTLYLQMNSLRAEDTAVYYC | SKSGTSASLVISGLQSDDEADYYCAAWDDSLN |
| ARDLLVRFLEWSHYYGMDVWGQGTTVTVSS | GVFGGGTKLTVL (SEQ ID NO: 307) |
| (SEQ ID NO: 306) | |
| QVQLVESGGGVVQPGRSLRLSCAASGFTFSSY | QSVLTQPPSASGTPGQRVTISCSGSSSNIGSNT |
| GMHWVRQAPGKGLEWVAVISYDGSNKYYADS | VHWYQQLPGTAPKLLIYSDNQRPSGVPDRFSG |
| VKGRFTISRDNSKNTLYLQMNSLRAEDTAVYYC | SKSGTSASLAISGLQSEDEADYYCAAWDDSLNG |
| ARDLLVRFLEWSHYYGMDVWGQGTTVTVSS | VFGGGTKLTVL (SEQ ID NO: 309) |
| (SEQ ID NO: 308) | |
| QVQLQQSGGGLVQPGGSLRLSCAASGFTFSSY | DIQMTQSPSFLSASVGDRVTITCRASQGISSYLA |
| AMTWVRQAPGKGLEWVSAISGGGGATYYADS | WYQQKPGKAPKLLIYAASSLQSGVPSRFSGSG |
| VEGRFTISRDNSKNTLYLQMNSLRAEDTAIYYCA | SGTDFTLTISSLQPEDFATYYCQQSYSTPLTFGG |
| SPAGHICSGGSCQYYYYGMDVWGQGTTVTVS | GTRVDMARTVAAPSVF (SEQ ID NO: 311) |
| SGS (SEQ ID NO: 310) | |
| QVQLVQSGAEVKKPGSSVKVSCKASGGTFSSY | EIVMTQSPGTLSLSPGERATLSCRASQSVSSGY |
| AISWVRQAPGQGLEWMGGIIPIFGTANYAQKFQ | LAWYQQKAGQAPRLLIYGGSARAPGIPARFSGS |
| GRVTITADESTSTAYMELSSLRSEDTAVYYCAR | GSGTDFTLTISSLEPEDFAVYYCQQRSIWPLTFG |
| DRHLYRRGYSSVDGMDVWGQGTTVTVSSGS | GGTKVEIKRTVAAPSVF (SEQ ID NO: 313) |
| (SEQ ID NO: 312) | |
| QVQLVQSGAEVKKPGESLKISCKGSGYSFTSY | DIVMTQSPVSLPVTLGQPASISCRSSQSVVYRD |
| WIGWVRQMPGKGLEWMGIIYPGDSDTRYSPSF | GNTYLSWFLQRPGQSPRRLIYKVSNRDSGVPD |
| QGQVTISADKSISTAYLQWSSLKASDTAMYYCA | RFSGSGSATDFTLKISRVEAEDVGVYYCMQGSS |
| RHPIGSGYDYWGQGTLVTVSSGS (SEQ ID NO: | WPYTFGQGTKLEIKRTVAAPSVF (SEQ ID NO: |
| 314) | 315) |
| QVQLVQSGGGVVRPGGSLRLSCAASGFTFDDY | QSALTQPASVSGSPGQSITISCTGTSSDVGGYN |
| GMSWVRQAPGKGLEWVSGISWNSGSIGYADS | YISWYQQYPGKAPKLMIYDVSKRPSGVSNRFSG |
| VKGRFTISRDNAKNSLYLQMNSLRAEDTALYYC | SKSGNTASLTISGLQAEDEADYYCNSYTSSRTV |
| AKEISDTAMGFDYWGQGTLVTVSSGS (SEQ ID | VFGGGTKLTVLGQPKAAPSVTLFPPSS (SEQ ID |
| NO: 316) | NO: 317) |
| QVQLVQSGGGLVQPGGSLRLSCAASGFTFSNY | QSALTQPASVSGSPGQSITISCTGTSSDVGGYN |
| WMYWVRQAPGKGLVWVSNINSDGSSTSYADS | YVSWYQQHPGKAPKLLIYDVTKRPSGVSNRFS |
| VKGRFTISRDNAKNSLYLQMNSLRDEDTAVYYC | GSKSGNTASLTISGLQAEDEADYYCSSYTSSST |
| AREGYNAHYFDYWGQGTLVTVSSGS (SEQ ID | VVFGGGTKLTVLGQPKAAPSVTLFPPSSKASGA |
| NO: 318) | (SEQ ID NO: 319) |
| QIQLVQSGGGVVQPGRSLRLSCAASGFTFSSY | DIVMTQSPDSLAVSLGERATINCKSSQSVLYSSD |
| GMHWVRQAPGKGLEWVAVIWYDGSNKYYADS | NKNYLAWYQQKPGQPPKLLIYWASTRESGVPD |
| VKGRFTISRDNSKNTLYLQMNSLRAEDTAVYYC | RFSGSGSGTDFTLTISSLQAEDVAVYYCQQYYS |
| AKGLGYHPLFDYWGQGTLVTVSSGS (SEQ ID | TPSFGPGTKVDIKRTVAAPSVFKASGA (SEQ ID |
| NO: 320) | NO: 321) |
| EVQLLESGGGLVQPGGSLRLSCAASGFTFSSY | DIQMTQSPSSLSASVGDRVTITCKASQDVSTAV |
| AMSWVRQAPGKGLEWVSTISSGGSYTSYPDSV | AWYQQKPGKAPKLLIYSASYRYTGVPSRFSGS |
| KGRFTISRDNSKNTLYLQMNSLRAEDTAVYYCA | GSGTDFTLTISSLQPEDFATYYCQQHYSTPWTF |
| KQDYAMNYWGQGTLVTVSS (SEQ ID NO: 356) | GGGTKVEIK (SEQ ID NO: 357) |
| EVQLLESGGGLVQPGGSLRLSCAASGFTFSSY | DIQMTQSPSSLSASVGDRVTITCKASQDVSTAV |
| AMSWVRQAPGKGLEWVSTISSGGSYTSYPDSV | AWYQQKPGKAPKLLIYSASYRYTGVPSRFSGS |
| KGRFTISRDNSKNTLYLQMNSLRAEDTAVYYCA | GSGTDFTLTISSLQPEDFATYYCQQHYSTPWTF |
| KQDYAMNYWGQGTLVTVSS (SEQ ID NO: 356) | GQGTKVEIK (SEQ ID NO: 358) |
| QVQLVQSGAEVKKPGASVKVSCKASGYTFTSY | DIVMTQSPDSLAVSLGERATINCKSSQSLLNSAN |
| WMQWVRQAPGQGLEWMGAIYPGDGDTRYTQ | QKNYLAWYQQKPGQPPKLLIYFASTRESGVPD |
| KFKGRVTMTRDTSTSTVYMELSSLRSEDTAVYY | RFSGSGSGTDFTLTISSLQAEDVAVYYCQQHYN |
| CARMGGYDRYYFDYWGQGTLVTVSS (SEQ ID | TPLTFGGGTKVEIK (SEQ ID NO: 372) |
| NO: 371) | |
| QVQLQQSGAELARPGASVKLSCKASGYTFTSY | DIVMTQSPSSLAMSVGQKVTMSCKSSQSLLNSA |
| WMQWVKQRPGQGLEWIGAIYPGDGDTRYTQK | NQKNYLAWYQQKPGQSPKLLVYFASTRESGVP |
| FKGKATLTADKSSSTAYMQLSSLASEDSAVYYC | DRFIGSGSGTDFTLTISSVQAEDLADYFCQQHY |
| ARMGGYDRYYFDYWGQGTTLTVSS (SEQ ID | NTPLTFGAGTKLELK (SEQ ID NO: 374) |
| NO: 373) | |
| QVQLVESGGGVVQPGRSLRLSCAASGFTFSRY | DIVMTQAAPSIPVIPGESVSMSCRSSKSLLYSNG |
| GIHWVRQAPGKGLEWVAVISYDGSDEYYVDSV | HTYVYWFVQRPGQSPQLLIYRMSNLASGVPDR |
| KGRFSISRDNSKNTLYLQMNSLRPADSAVYYCV | FSGSGSGTAFTLRISRVEAEDVGVYYCMQNLEF |
| KGDLELGFDYWGQGTLVTVSS (SEQ ID NO: | PLTFGAGTKLELK (SEQ ID NO: 388) |
| 387) | |
| EVQVLESGGDLVQPGGSLRLSCAASGFTFSAY | DIQMTQSPASLSASVGDRVTITCRASQDISDYLA |
| AMTWVRQAPGKGLEWVSAISGSGGSAYYADSV | WYQQKPGKIPRLLIYTTSTLQSGVPSRFRGSGS |
| KGRFTISRDNSKNTVYLQMNSLRAEDTAVYYCA | GTDFTLTISSLQPEDVATYYCQKYDSAPLTFGG |
| KDGAWKMSGLDVWGQGTTVIVSS (SEQ ID NO: | GTKVEIK (SEQ ID NO: 390) |
| 389) | |
| QVQLVESGGGVVQPGKSLRLSCAASGFTFSSF | DIQMTQSPSSVSASVGDRVTITCRASQGISNWL |
| GMHWVRQAPGKGLEWVAVIGYDGGNEYYADS | AWYQQKPGKAPKLLIFAASSLQSGVPSRFSGSA |
| VKGRFTISRDNSKNTLNLQMSSLRAEDTAVYYC | SGTDFTLTINSLQPEDFATYYCQQANSFPLTFG |
| STISHYDILSGMDVWGRGTTVTVSS (SEQ ID | GGTKVEIKR (SEQ ID NO: 392) |
| NO: 391) | |
| QVQLVQSGGGLVQPGGSLRLSCAASGFTFNTY | DIQMTQSPATLSVSPGERATLSCRASQSVSSNL |
| AISWVRQAPGKGLEWVSTITGSGYNTYYADSVK | AWYHQKPGQAPRLLIYGVSTRATGIPARFSGNG |
| GRFTISRDNSKNTLYLQMSSLRAEDTAVFYCAK | SGTEFTLTISSLQSEDFAVYYCQQHNNWPLTFG |
| DSRYNWNYGIFDYWGQGTTVTVSS (SEQ ID | GGTKVEIKR (SEQ ID NO: 406) |
| NO: 405) | |
| QVQLVQSGPGLVKPSETLSLTCTVSGGSISNSN | DIQLTQSPSSLSASVGDRVTITCRASQDIRNDLG |
| YYWGWIRQPPGKGLEWIGTTYYSGTTYYNPSL | WYQQKPGKAPKRLIYAASSLQSGVPSRFSGSG |
| KSRVTISVDTSKNQFSLKLSSVTAADTAVYYCAR | SGTEFSLTLSSLQPEDFATYFCLQHHIYPWTFG |
| DYYDSSGYYYNWFDPWGQGTTVTVSS (SEQ | QGTKLEIKR (SEQ ID NO: 408) |
| ID NO: 407) | |
| QVQLVESGPGLVKPSETLSLTCTVYGGSISSGN | AIQMTQSPSSLSASVGDRVTITCRASQDIRHDLG |
| YYWGWIRQPPGKGLEWIGTIYYSGSAYYNPSLK | WYQQKPGKAPKRLIYAASSLQSGVPSRFSGSG |
| SRVTMSVDTSKNQFSLKLSSVTAADTAVYYCVR | SGTEFTLTISSLQPEDFATYYCLQHNTYPWTFG |
| DYYDSSGHYYNWFDPWGQGTTVTVSS (SEQ | QGTKVEIKR (SEQ ID NO: 410) |
| ID NO: 409) | |
| QVQLVQSGGGLVKPGGSLRLSCAASGFTFSSY | AIQMTQSPSSLSASVGDRVTITCRASQGIRNDL |
| SMNWVRQAPGKGLEWVSSISSSSSYIYYADSV | GWYQQKPGKAPKRLIYAASSLQSGVPSRFSGS |
| KGRFTISRDNAKNSLYLQMNSLRAEDTAVYYCA | GSGTEFTLTISSLQPEDFATYYCLQHNSYPYTFG |
| RDRGYTFGVDYWGQGTTVTVSS (SEQ ID NO: | QGTKVEIKR (SEQ ID NO: 412) |
| 411) | |
| QVQLVQSGPGLVKPSETLSLTCTVSGGSIITYSY | AIQMTQSPSSLSASVGDRVTITCRASQGIRNDL |
| YWGWIRQPPGKGLEWIGTIHHSGSTYYNPSLKS | GWYQQKPGKAPKRLIYAASSLQSGVPSRFSGS |
| RVTISVDTSKNQFSLTLSSVTAADTAVYYCARDY | GSGTEFTLTISSLQPEDFATYYCLQHNSSPWTF |
| YDSSGYYYNWFDPWGQGTMVTVSS (SEQ ID | GQGTKVEIKR (SEQ ID NO: 414) |
| NO: 413) | |
| QVHLKESGPTLVKPTQTLTLTCTFSGFSLSTSGV | AIQMTQSPSSLSASVGDRVTITCRASQGISNYLA |
| GVGWIRQPPGKALEWLALIYWNDDKRYSPSLK | WFQQKPGKAPKSLIYAASSLQSGVPSKFSGSG |
| SRLTITKDTSKNQVVLTMTNMDPVDTATYYCTH | SGTDFTLTISSLQPEDFATYYCQQYNSYPLTFG |
| TSRYNWHYGFLDYWGQGTTVTVSS (SEQ ID | GGTKVEIKR (SEQ ID NO: 416) |
| NO: 415) | |
| EVQLVQLGPGLVKPSETLSLTCTVSGGSISNSN | AIQMTQSPSSLSASVGDRVTITCRASQDIRNDLG |
| YYWGWIRQPPGKGLEWIGTTYYSGTTYYNPSL | WYQQKPGKAPKRLIYAASSLQSGVPSRFSGSG |
| KSRVTISVDTSKNQFSLKLSSVTAADTAVYYCAR | SGTEFSLTLSSLQPEDFATYFCLQHHIYPWTFG |
| DYYDSSGYYYNWFDPWGQGTTVTVSS (SEQ | QGTKVEIKR (SEQ ID NO: 418) |
| ID NO: 417) | |
| QVQLVEAGGGVVQPGRSLRLSCAASGFTFSRY | AIRLTQSPSSVSASVGDRVTITCRASQDISIWLA |
| GIHWVRQAPGKGLEWVAVISYDGTDEYYADSV | WYQQSPGKAPKLLINVASRLQSGVPSRFSGSG |
| KGRFTISRDNSKNTLYLQMNSLRPADSAVYYCA | SGTDFTLTINGLQPEDFVTYYCQQANSFPITFGQ |
| KGDLELGFDYWGQGTLVTVSS (SEQ ID NO: | GTRLATK (SEQ ID NO: 420) |
| 419) | |
| QVQLVQSGGGLVKPGGSLRLSCAASGFIFNTYT | DIQMTQSPSSLSASVGDRVTITCRASQDIRNDL |
| MNWVRQAPGKGLEWVSSITSRGTYIFYSDSLK | GWYQQKPGKAPKGLIYAASSLQSGVPSRFSGS |
| GRFTISRDNANNSLFLQMNSLRVEDTAVYYCSR | GSGTEFTLTISSLQPEDFATYHCLHYDFHPRTFG |
| DRGYTFGPDYWGQGTLVTVSS (SEQ ID NO: | QGTKVEIK (SEQ ID NO: 422) |
| 421) | |
| QVQLVESGGGVVQPGRSLRLSCAASGFTFSSY | EIVMTQSPATLSVSPGERATLSCRASQTFSSNL |
| AMHWVRQAPGKGLEWVAVIWYDGTNYYYADS | AWYQQKPGQAPRLLIYGASTRATGIPARFSGSG |
| VKGRFTISRDNSKNTLYLQMNSLRAEDTAVYYC | SGTEFTLTISSLQSEDFAVYYCQQYNKWPLTFG |
| ARDPLYYDILTGYSPDYYYGMDVWGQGTTVTV | GGTKVEIK (SEQ ID NO: 424) |
| SS (SEQ ID NO: 423) | |
| EVQLLESGGGLVQPGESLRLSCAASGFTFSSYA | EIVMTQSPATLSVSPGERATLSCRASQNVSSNL |
| MNWVRQAPGKGLEWVSTISGSGGYIYYADSVK | AWNKQKPGQAPRLLIYATSTRATGVPARFSASG |
| GRFTISRDNSKNTLYLQMNSLRAEDTAVYFCAK | SGTDFALTINSLQSEDFAVYYCQQYNNWPLTFG |
| DSRYNWNYGNFDYWGQGTLVTVSS (SEQ ID | GGTKVEIK (SEQ ID NO: 426) |
| NO: 425) | |
| EVQLLESGGGLVQPGGSLRLSCVASRFTFSSNA | EIVMTQSPATLSVSPGERATLSCRASQSVSSNL |
| MSWVRQAPGTGLEWVSAITGSGSRTYYADSVK | AWYQQKPGQAPRLLIYGASTRATGLPARFSGS |
| GRFTISRDNSKNTVYLQMNSLRGEDTAVYYCAK | GSGTDFTLTISSLQSEDFAVYYCQQYNNWPLTF |
| DQGGTWNYGDFDYWGQGTLVTVSS (SEQ ID | GGGTKVEIK (SEQ ID NO: 428) |
| NO: 427) | |
| QVQLVESGGDVVQPGRSLRLSCTASGFTFSSY | EIMMTQSPATLSVSPGERGTLSCRASQSVSSNL |
| GMHWVRQAPGRGLEWVAVISFDGKNKYYADS | AWYQQKPGQAPRLLIYGASTRASGIPARFSGSG |
| VKGRFTVSRDNSKNTLFLQMNSLRAEDTALYYC | SGTEFTLTVSSLQSEDFAVYYCQQYNNWPLTF |
| AKRIAATGYYYFYGLDVWGQGTTVTVSS (SEQ | GGGTKVEIK (SEQ ID NO: 430) |
| ID NO: 429) | |
| EVQLLESGGGLVQPGGSLRLSCAASGFTSITYA | EIVMTQSPATLSVSPGERATLSCRASQSVDSNL |
| MSWVRQAPGKGLEWVSAISVSGTNTYYADSVK | VWYQQKPGQVPRLLIYGASTRATGIPARFSGSG |
| GRFTISRDKSKNMLYLQMNSLRAEDTAVYYCAK | SGTEFTLTISSLQSEDFAVYYCQQYNKWPLTFG |
| DLLHNWKYGTFDIWGQGTMVTVSS (SEQ ID | GGTKVEIK (SEQ ID NO: 432) |
| NO: 431) | |
| EVQLLESGGGLVQPGGSLRLSCAASGFTSITYA | EIVMTQSPATLSVSPGERATLSCRASQSVSSNL |
| MSWVRQAPGKGLEWVSAISVSGTNTYYADSVK | AWNQQKPGQAPRLLIYGASTRATGIPARFSGSG |
| GRFTISRDKSKNMLYLQMNSLRAEDTAVYYCAK | SGTEFTLTISSLQSEDFAVYYCQQYNNWPMYTF |
| DLLHNWKYGTFDIWGQGTMVTVSS (SEQ ID | GQGTKLEIK (SEQ ID NO: 434) |
| NO: 433) | |
| QVQLVESGGGVVQPGRSLRLSCEASGFTFSSS | DIVNTQSPLSLPVTPGEPASISCRSSQSLLYGNG |
| GMHWVRQAPGKGLQWVAVISYDGNNKFYEDS | YNYLDWYLQKPGHSPQLLIYLGSNRGSGVPDR |
| VKGRLTISRDNSNNTLWLQMNSLRVEDTAVYYC | FSGSGSGTDFTLKISRVEAEDVGIYYCMQTLQT |
| AKSGGRVGAAFAYWGQGTLVTVSS (SEQ ID | PFTFGPGTKMYIK (SEQ ID NO: 436) |
| NO: 435) | |
| EVQLLESGGGLVQPGGSLRLSCAGSGITFSSYA | EIVMTQSPATLSMSPGERATLSCRASQSVSSNL |
| MSWVRQAPGKGLEWVSAISGNGGTTNYADSV | AWYQQKPGQAPRLLIYGASTRATGIPARFSGSG |
| KGRFTISRDNSKNTLYLQMNSLRAEDTAVYYCA | SGTEFTLTISSLQSEDFAVYYCQQYNNWPLTFG |
| KERILTSSWTRYGIMDVWGQGTTVTVSS (SEQ | GGTKLEIK (SEQ ID NO: 438) |
| ID NO: 437) | |
| QVQLQQWGAGLLKPSETLSLTCAVYGGSFSGY | DIQLTQSPSFLSASVGDRVTITCWASQGISSYLA |
| YWSWIRQPPGKGLEWIGEINHSGNTNYNPSLKS | WYQQKPGKAPKLLIYAASTLQSGVPSRFSGSGS |
| RVTISVDTSKNQFSLKLNSVTAADTAVYYCARR | GTEFTLTISSLQPEDFATYYCQQLNSYPLTFGG |
| EATVTPYFDYWGQGTLVTVSS (SEQ ID NO: | GTKVEIK (SEQ ID NO: 440) |
| 439) | |
| QVQLVESGGGVVQPGRSLRLSCVVSGFNFSRN | DIVMTQSPLSLPVTPGEPASISCRSSQSLLHSNG |
| GIHWVRQAPGKGLEWVAVISYDGRNKFYVESV | YNYLDWYLQKPGQSPQLMIYLGSHRASGVPDR |
| KGRFTISRDNSKNTLYLQMNSLRVEDTAVYYCA | FSGSGSGTDFTLKISRVEAEDVGVYYCIQVQQT |
| KSSIGGFFEYWGQGTLVTVSS (SEQ ID NO: | PITFGQGTRLEIK (SEQ ID NO: 442) |
| 441) | |
| QVQLVESGGGVVQPGRSLRLSCAASGFTFSRY | DIVMTQTPLSSPVTLGQPASISCRSSQSLVHSD |
| GIHWVRQAPGKGLEWVAVISYDGSDEYYVDSV | GNTYLSWLQQRPGQPPRLLIYKISNRFSGVPDR |
| KGRFSISRDNSKNTLYLQMNSLRPADSAVYYCV | FSGSGAGTDFTLKISRVEAEDVGVYFCMQATQF |
| KGDLELGFDYWGQGTLVTVSS (SEQ ID NO: | PYTFGQGTKLEIK (SEQ ID NO: 443) |
| 387) | |
| EVQLLESGGGLVQPGGSLRLSCAASGFTFSSY | DIQMTQSPSSLSASVGDRVTITCKASQDVSTAV |
| AMSWVRQAPGKGLEWVSTISSGGSYTSYPDSV | AWYQQKPGKAPKLLIYSASYRYTGVPSRFSGS |
| KGRFTISRDNSKNTLYLQMNSLRAEDTAVYYCA | GSGTDFTLTISSLQPEDFATYYCQQHYSTPWTF |
| KQDYAMNYWGQGTMVTVSS (SEQ ID NO: 444) | GQGTKVEIK (SEQ ID NO: 445) |
| EVQLVESGGGLVKPGGSLKLSCAASGFTFSSYA | DIEMTQSHKFMSTSVGDRVSITCKASQDVSTAV |
| MSWVRQTPEKRLEWVATISSGGSYTSYPDSVK | AWYQQKPGQSPKLLLYSASYRYTGVPDRFTGS |
| GRFTISRDNAKNTLYLQMSSLRSEDTAMYYCAR | GSGTDFTFTISSVQAEDLAVYYCQQHYSTPWTF |
| QDYAMNYWGQGTSVTVSS (SEQ ID NO: 446) | GGGTKLEIK (SEQ ID NO: 447) |
| EVQLLESGGGLVQPGGSLRLSCAASGFTFSSY | DIEMTQSHKFMSTSVGDRVSITCKASQDVSTAV |
| AMSWVRQAPGKGLEWVSTISSGGSYTSYPDSV | AWYQQKPGQSPKLLLYSASYRYTGVPDRFTGS |
| KGRFTISRDNSKNTLYLQMNSLRAEDTAVYYCA | GSGTDFTFTISSVQAEDLAVYYCQQHYSTPWTF |
| KQDYAMNYWGQGTMVTVSS (SEQ ID NO: 444) | GGGTKLEIK (SEQ ID NO: 447) |
| EVQLVESGGGLVKPGGSLKLSCAASGFTFSSYA | DIQMTQSPSSLSASVGDRVTITCKASQDVSTAV |
| MSWVRQTPEKRLEWVATISSGGSYTSYPDSVK | AWYQQKPGKAPKLLIYSASYRYTGVPSRFSGS |
| GRFTISRDNAKNTLYLQMSSLRSEDTAMYYCAR | GSGTDFTLTISSLQPEDFATYYCQQHYSTPWTF |
| QDYAMNYWGQGTSVTVSS (SEQ ID NO: 446) | GQGTKVEIK (SEQ ID NO: 445) |
| QVTLKESGPVLVKPTETLTLTCTVSGIDLSHDDIS | DIVMTQSPATLSLSPGERATLSCTSSQSVYHAN |
| WVRQAPGKGLEWVGIISYAGSMYYASWAKGRL | WLSWFQQKPGQPPKLLIYWASTLAYGVPSRFS |
| TISKDTSKNQVVLTMTNMDPVDTATYYCARGVP | GSGSGTDFTLTISSLQPEDAATYYCAGGYGGG |
| AYSHGGDKWGQGTLVTVSS (SEQ ID NO: 468) | RYAFGQGTKVEIK (SEQ ID NO: 483) |
| QVTLKESGPVLVKPTETLTLTCTVSGIDLSHTDIS | DIVMTQSPATLSLSPGERATLSCTSSQSVYHAN |
| WVRQAPGKGLEWVGIISYAGKTYYASWAKKRL | WLSWFQQKPGQPPKLLIYWASTLAYGVPSRFS |
| TISKDTSKNQVVLTMTNMDPVDTATYYCARGVP | GSGSGTDFTLTISSLQPEDAATYYCAGGYGGG |
| AYSHGGDLWGQGTLVTVSS (SEQ ID NO: 469) | RYAFGQGTKVEIK (SEQ ID NO: 483) |
| QVTLKESGPVLVKPTETLTLTCTVSGIDLSHTDIS | DIVMTQSPATLSLSPGERATLSCTSSQSVYHAN |
| WVRQAPGKGLEWVGIISYAGKKYYASWAKGRL | WLSWFQQKPGQPPKLLIYWASTLAYGVPSRFS |
| TISKDTSKNQVVLTMTNMDPVDTATYYCARGVP | GSGSGTDFTLTISSLQPEDAATYYCAGGYGGG |
| AYSHGGDLWGQGTLVTVSS (SEQ ID NO: 470) | RYAFGQGTKVEIK (SEQ ID NO: 483) |
| QVTLKESGPVLVKPTETLTLTCTVSGIDLSHTDIS | DIVMTQSPATLSLSPGERATLSCTSSQSVYHAN |
| WVRQAPGKGLEWVGIISYAGSKYKASWAKGRL | WLSWFQQKPGQPPKLLIYWASTLAYGVPSRFS |
| TISKDTSKNQVVLTMTNMDPVDTATYYCARGVP | GSGSGTDFTLTISSLQPEDAATYYCAGGYGGG |
| AYSHGGDLWGQGTLVTVSS (SEQ ID NO: 471) | RYAFGQGTKVEIK (SEQ ID NO: 483) |
| QVTLKESGPVLVKPTETLTLTCTVSGIDLSHDDIS | DIVMTQSPATLSLSPGERATLSCTSSQSVYHAN |
| WVRQAPGKGLEWVGIISYAGKKYYASWAKGRL | WLSWFQQKPGQPPKLLIYWASTLAYGVPSRFS |
| TISKDTSKNQVVLTMTNMDPVDTATYYCARGVP | GSGSGTDFTLTISSLQPEDAATYYCAGGYGGG |
| AYSHGGDLWGQGTLVTVSS (SEQ ID NO: 472) | RYAFGQGTKVEIK (SEQ ID NO: 483) |
| QVTLKESGPVLVKPTETLTLTCKVSGIDLSHDDI | DIVMTQSPATLSLSPGERATLSCTTSQSVYHEN |
| SWVRQAPGKGLEWVSIISYAGSTYYASWAKGR | WLSWFQQKPGQPPKLLIYWASTLAYGVPSRFS |
| LTISKDTSKNQVVLTMTNMDPVDTATYYCARGV | GSGSGTDFTLTISSLQPEDAATYYCAGGYGGG |
| PAYSHGGDLWGQGTLVTVSS (SEQ ID NO: 474) | RYAFGQGTKVEIK (SEQ ID NO: 484) |
| QVTLKESGPVLVKPTETLTLTCKVSGIDLSHEDIS | DIVMTQSPATLSLSPGERATLSCTTSQSVYHEN |
| WVRQAPGKGLEWVSIISYAGSTYYASWAKGRL | WLSWFQQKPGQPPKLLIYWASTLAYGVPSRFS |
| TISKDTSKNQVVLTMTNMDPVDTATYYCARGVP | GSGSGTDFTLTISSLQPEDAATYYCAGGYGGG |
| AYSHGGDLWGQGTLVTVSS (SEQ ID NO: 475) | RYAFGQGTKVEIK (SEQ ID NO: 484) |
| QVTLKESGPVLVKPTETLTLTCTVSGIDLSHDDIS | DIVMTQSPATLSLSPGERATLSCTSSQSVFHEN |
| WVRQAPGKGLEWVGIISYAGSTYYASWAKGRL | WLSWFQQKPGQPPKLLIYWASTLAWGVPSRFS |
| TISKDTSKNQVVLTMTNMDPVDTATYYCARGVP | GSGSGTDFTLTISSLQPEDAATYYCAGGYGGG |
| AYSHGGDLWGQGTLVTVSS (SEQ ID NO: 467) | RYAFGQGTKVEIK (SEQ ID NO: 485) |
| QVQLVESGGGLVQPGGSLRLSCAVSGIDLSHE | DIVMTQSPATLSLSPGERATLSCTTSQSVYHEN |
| DISWVRQAPGKGLEWVSIISYAGSKYYASWAKG | WLSWFQQKPGQPPKLLIYWASTLAYGVPSRFS |
| RLTISKDTSKNQVVLTMTNMDPVDTATYYCARG | GSGSGTDFTLTISSLQPEDAATYYCAGGYGGG |
| VPAYSHGGDLWGQGTLVTVSS (SEQ ID NO: | RYAFGQGTKVEIK (SEQ ID NO: 484) |
| 476) | |
| QVTLKESGPVLVKPTETLTLTCTVSGIDLSHDDIS | DIVMTQSPATLSLSPGERATLSCTSSQSVYHAN |
| WVRQAPGKGLEWVGIISYAGSTYYASWAKGRL | WLSWFQQKPGQPPKLLIYWASTLAYGVPSRFS |
| TISKDTSKNQVVLTMTNMDPVDTATYYCARGVP | GSGSGTDFTLTISSLQPEDAATYYCAGGYGGG |
| AYSHGGDLWGQGTLVTVSS (SEQ ID NO: 467) | RYAFGQGTKVEIK (SEQ ID NO: 483) |
| QVQLVESGGGLVQPGGSLRLSCAVSGIDLSHD | DIVMTQSPATLSLSPGERATLSCTTSQSVYHEN |
| DISWVRQAPGKGLEWVSIISYAGSTYYASWAKG | WLSWFQQKPGQPPKLLIYWASTLAYGVPSRFS |
| RLTISKDTSKNQVVLTMTNMDPVDTATYYCARG | GSGSGTDFTLTISSLQPEDAATYYCAGGYGGG |
| VPAYSHGGDLWGQGTLVTVSS (SEQ ID NO: | RYAFGQGTKVEIK (SEQ ID NO: 484) |
| 473) | |
| QVTLKESGPVLVKPTETLTLTCTVSGIDLSSYDIS | DIVMTQSPATLSLSPGERATLSCQSSQSVYDNN |
| WVRQAPGKGLEWVGIISYAGSTYYASWAKGRL | WLSWFQQKPGQPPKLLIYWASTLASGVPSRFS |
| TISKDTSKNQVVLTMTNMDPVDTATYYCARGVP | GSGSGTDFTLTISSLQPEDAATYYCAGGYGGGL |
| AYSTGGDLWGQGTLVTVSS (SEQ ID NO: 448) | YAFGQGTKVEIK (SEQ ID NO: 477) |
| QVTLKESGPVLVKPTETLTLTCTVSGIDLSHYDIS | DIVMTQSPATLSLSPGERATLSCQSSQSVYHNN |
| WVRQAPGKGLEWVGIISYAGSTYYASWAKGRL | WLSWFQQKPGQPPKLLIYWASTLASGVPSRFS |
| TISKDTSKNQVVLTMTNMDPVDTATYYCARGVP | GSGSGTDFTLTISSLQPEDAATYYCAGGYGGGL |
| AYSTGGDLWGQGTLVTVSS (SEQ ID NO: 449) | YAFGQGTKVEIK (SEQ ID NO: 478) |
| QVTLKESGPVLVKPTETLTLTCTVSGIDLSSYDIS | DIVMTQSPATLSLSPGERATLSCTSSQSVYHNN |
| WVRQAPGKGLEWVGIISHAGSTYYASWAKGRL | WLSWFQQKPGQPPKLLIYWASTLAYGVPSRFS |
| TISKDTSKNQVVLTMTNMDPVDTATYYCARGVP | GSGSGTDFTLTISSLQPEDAATYYCAGGYGGG |
| AYSTGGDLWGQGTLVTVSS (SEQ ID NO: 450) | RYAFGQGTKVEIK (SEQ ID NO: 479) |
| QVTLKESGPVLVKPTETLTLTCTVSGIDLSSYDIS | DIVMTQSPATLSLSPGERATLSCQSSQSVYHNN |
| WVRQAPGKGLEWVGIISYAGSTYYASWAKGRL | WLSWFQQKPGQPPKLLIYWASTLAFGVPSRFS |
| TISKDTSKNQVVLTMTNMDPVDTATYYCARGVP | GSGSGTDFTLTISSLQPEDAATYYCAGGYGGGL |
| AHSTGGDLWGQGTLVTVSS (SEQ ID NO: 451) | YAFGQGTKVEIK (SEQ ID NO: 480) |
| QVTLKESGPVLVKPTETLTLTCTVSGIDLSSYDIS | DIVMTQSPATLSLSPGERATLSCQTSQSVYHDN |
| WVRQAPGKGLEWVGIISYAGSTYYASWAKGRL | WLSWFQQKPGQPPKLLIYWASTLASGVPSRFS |
| TISKDTSKNQVVLTMTNMDPVDTATYYCARGVP | GSGSGTDFTLTISSLQPEDAATYYCAGGYGGG |
| AYSHGGDLWGQGTLVTVSS (SEQ ID NO: 452) | RYAFGQGTKVEIK (SEQ ID NO: 481) |
| QVTLKESGPVLVKPTETLTLTCTVSGIDLSHYDIS | DIVMTQSPATLSLSPGERATLSCTSSQSVYHDN |
| WVRQAPGKGLEWVGIISHAGSTYYASWAKGRL | WLSWFQQKPGQPPKLLIYWASTLAYGVPSRFS |
| TISKDTSKNQVVLTMTNMDPVDTATYYCARGVP | GSGSGTDFTLTISSLQPEDAATYYCAGGYGGGL |
| AYSTGGDLWGQGTLVTVSS (SEQ ID NO: 453) | YAFGQGTKVEIK (SEQ ID NO: 482) |
| QVTLKESGPVLVKPTETLTLTCTVSGIDLSSYDIS | DIVMTQSPATLSLSPGERATLSCTTSQSVYHEN |
| WVRQAPGKGLEWVGIISHAGSTYYASWAKGRL | WLSWYQQKPGQPPKLLIYWASTLAYGVPSRFS |
| TISKDTSKNQVVLTMTNMDPVDTATYYCARGVP | GSGSGTDFTLTISSLQPEDVATYYCAGGYGGG |
| AHSTGGDLWGQGTLVTVSS (SEQ ID NO: 454) | RYAFGQGTKVEIK (SEQ ID NO: 486) |
| QVTLKESGPVLVKPTETLTLTCTVSGIDLSHYDIS | |
| WVRQAPGKGLEWVGIISYAGSTYYASWAKGRL | |
| TISKDTSKNQVVLTMTNMDPVDTATYYCARGVP | |
| AHSTGGDLWGQGTLVTVSS (SEQ ID NO: 455) | |
| QVTLKESGPVLVKPTETLTLTCTVSGIDLSHYDIS | |
| WVRQAPGKGLEWVGIISHAGSTYYASWAKGRL | |
| TISKDTSKNQVVLTMTNMDPVDTATYYCARGVP | |
| AHSTGGDLWGQGTLVTVSS (SEQ ID NO: 456) | |
| QVTLKESGPVLVKPTETLTLTCTVSGIDLSHYDIS | |
| WVRQAPGKGLEWVGIISYAGSTYYASWAKGRL | |
| TISKDTSKNQVVLTMTNMDPVDTATYYCARGVP | |
| AYSHGGDLWGQGTLVTVSS (SEQ ID NO: 457) | |
| QVTLKESGPVLVKPTETLTLTCTVSGIDLSSYDIS | |
| WVRQAPGKGLEWVGIISHAGSTYYASWAKGRL | |
| TISKDTSKNQVVLTMTNMDPVDTATYYCARGVP | |
| AYSHGGDLWGQGTLVTVSS (SEQ ID NO: 458) | |
| QVTLKESGPVLVKPTETLTLTCTVSGIDLSSYDIS | |
| WVRQAPGKGLEWVGIISYAGSTYYASWAKGRL | |
| TISKDTSKNQVVLTMTNMDPVDTATYYCARGVP | |
| AHSHGGDLWGQGTLVTVSS (SEQ ID NO: 459) | |
| QVTLKESGPVLVKPTETLTLTCTVSGIDLSHYDIS | |
| WVRQAPGKGLEWVGIISHAGSTYYASWAKGRL | |
| TISKDTSKNQVVLTMTNMDPVDTATYYCARGVP | |
| AYSHGGDLWGQGTLVTVSS (SEQ ID NO: 460) | |
| QVTLKESGPVLVKPTETLTLTCTVSGIDLSSYDIS | |
| WVRQAPGKGLEWVGIISHAGSTYYASWAKGRL | |
| TISKDTSKNQVVLTMTNMDPVDTATYYCARGVP | |
| AHSHGGDLWGQGTLVTVSS (SEQ ID NO: 461) | |
| QVTLKESGPVLVKPTETLTLTCTVSGIDLSHYDIS | |
| WVRQAPGKGLEWVGIISYAGSTYYASWAKGRL | |
| TISKDTSKNQVVLTMTNMDPVDTATYYCARGVP | |
| AHSHGGDLWGQGTLVTVSS (SEQ ID NO: 462) | |
| QVTLKESGPVLVKPTETLTLTCTVSGIDLSHYDIS | |
| WVRQAPGKGLEWVGIISHAGSTYYASWAKGRL | |
| TISKDTSKNQVVLTMTNMDPVDTATYYCARGVP | |
| AHSHGGDLWGQGTLVTVSS (SEQ ID NO: 463) | |
| QVTLKESGPVLVKPTETLTLTCTVSGIDLSHTDIS | |
| WVRQAPGKGLEWVGIISYAGSTYYASWAKGRL | |
| TISKDTSKNQVVLTMTNMDPVDTATYYCARGVP | |
| AYSHGGDLWGQGTLVTVSS (SEQ ID NO: 464) | |
| QVTLKESGPVLVKPTETLTLTCTVSGIDLSHTDIS | |
| WVRQAPGKGLEWVGIISYAGSTYYMSWAKGRL | |
| TISKDTSKNQVVLTMTNMDPVDTATYYCARGVP | |
| AYSHGGDLWGQGTLVTVSS (SEQ ID NO: 465) | |
| QVTLKESGPVLVKPTETLTLTCTVSGIDLSHYDIS | |
| WVRQAPGKGLEWVGIISYAGSTYYASWAKGRL | |
| TISKDTSKNQVVLTMTNMDPVDTATYYCARGVP | |
| AYSHGGDLWGQGTLVTVSS (SEQ ID NO: 466) | |

In some embodiments, the myostatin antibody or an antigen-binding fragment thereof, has the CDR sequences described in Table 4, 5, or 6 (i.e., a light chain CDR1, CDR2, and CDR3 and a heavy chain CDR1, CDR2, and CDR3). In some embodiments, the myostatin antibody or antigen binding fragment thereof includes a light chain variable CDR1 sequence having at least 90% (e.g., at least 91%, 92%, 93%, 94%, 95%, 96%, 97%, 98%, 99%, or 100%) sequence identity to a light chain variable CDR1 sequence in Table 4 or Table 6, such as any one of SEQ ID NOs: 169, 193, 198, 238, 241, 303, 325, 330, 362, 378, 384, 396, 402, 826, 490, 493, and 343-346; a light chain variable CDR2 sequence having at least 90% (e.g., at least 91%, 92%, 93%, 94%, 95%, 96%, 97%, 98%, 99%, or 100%) sequence identity to a light chain variable CDR2 sequence in Table 4 or Table 6, such as any one of SEQ ID NOs: 170, 194, 199, 239, 304, 326, 331, 363, 379, 385, 397, 403, 827, 491, and 347-349; a light chain variable CDR3 sequence having at least 90% (e.g., at least 91%, 92%, 93%, 94%, 95%, 96%, 97%, 98%, 99%, or 100%) sequence identity to a light chain variable CDR3 sequence in Table 4 or Table 6, such as any one of SEQ ID NOs: 171, 195, 200, 240, 245, 249, 305, 327, 364, 380, 386, 398, 404, 828, 492, and 350-355; a heavy chain variable CDR1 sequence having at least 90% (e.g., at least 91%, 92%, 93%, 94%, 95%, 96%, 97%, 98%, 99%, or 100%) sequence identity to a heavy chain variable CDR1 sequence in Table 4 or Table 5, such as any one of SEQ ID NOs: 166, 190 196, 235, 242, 246, 300, 322, 328, 359, 366, 375, 381, 393, 399, 823, 487, and 332-334; a heavy chain variable CDR2 sequence having at least 90% (e.g., at least 91%, 92%, 93%, 94%, 95%, 96%, 97%, 98%, 99%, or more) sequence identity to a heavy chain variable CDR2 sequence in Table 4 or Table 5, such as any one of SEQ ID NOs: 167, 191, 197, 236, 243, 247, 301, 323, 329, 360, 365, 376, 382, 394, 400, 824, 488, 335, and 336; and a heavy chain variable CDR3 sequence having at least 90% (e.g., at least 91%, 92%, 93%, 94%, 95%, 96%, 97%, 98%, 99%, or more) sequence identity to a heavy chain variable CDR3 sequence in Table 4 or Table 5, such as any one of SEQ ID NOs: 168, 192, 237, 244, 248, 302, 324, 361, 377, 383, 395, 401, 825, 489, and 337-342. In some embodiments, the myostatin antibody or antigen binding fragment thereof includes a light chain CDR1, CDR2, and CDR3 sequence and a heavy chain CDR1, CDR2, and CDR3 sequence from the same row of Table 4.

TABLE 4
Exemplary CDR sequences of myostatin antibodies

| HCDR1 | HCDR2 | HCRD3 | LCDR1 | LCDR2 | LCDR3 |
|---|---|---|---|---|---|
| GFTFSAYA | ISGSGGSA | AKDGAWKMS | QDISDY (SEQ | TTS (SEQ ID | QKYDSAPLT |
| (SEQ ID NO: | (SEQ ID NO: | GLDV (SEQ | ID NO: 169) | NO: 170) | (SEQ ID NO: |
| 166) | 167) | ID NO: 168) | | | 171) |
| GLXFSRYXM | XXXXXGXST | LPDY (SEQ ID | RAXXSVSSS | STSNLXX, in | QXXXGYXXT, |
| S, in which X | XYSDTVKG, | NO: 192) | YLH, in which | which X at | in which X at |
| at position 3 is | in which X at | | X at position 3 | position 6 is A, | position 2 is Q, |
| T or N and X | position 1 is A, | | is S or L and X | V, T, or M and | N, H, L, P or |
| at position 8 is | R or H, X at | | at position | X at position 7 | W, X at |
| G, T or P | position 2 is I | | 4 is | is S, A, T, N, | position 3 is Y |
| (SEQ ID NO: | or Q, X at | | S or Q (SEQ | W, D or Y | or H, X at |
| 190) | position 3 is | | ID NO: 193) | (SEQ ID NO: | position 4 is S |
| | N, T or K, X at | | | 194) | or L, X at |
| | position 4 is S | | | | position 7 is H, |
| | or A, X at | | | | F, Q or T, and |
| | position 5 is H, | | | | X at position 8 |
| | S, V, L, M, N, | | | | is F or W |
| | G, D, W, R, A, | | | | (SEQ ID NO: |
| | Y, K, P, T, I or | | | | 195) |
| | E, X at | | | | |
| | position 7 is G | | | | |
| | or S, and X at | | | | |
| | position 10 is | | | | |
| | Y or K (SEQ | | | | |
| | ID NO: 191) | | | | |
| GLTFSRYPM | AITSSGGSTY | LPDY (SEQ ID | RASSSVSSS | STSNLVA | QHHSGYHFT |
| S (SEQ ID | YSDTVKG | NO: 192) | YLH (SEQ ID | (SEQ ID NO: | (SEQ ID NO: |
| NO: 196) | (SEQ ID NO: | | NO: 198) | 199) | 200) |
| | 197) | | | | |
| GFSXRXVGX | HIYWDDDXR | RXITTVIGGG | SASSSISYMH | DTSKLAR | QQWYXNPLT, |
| SVS, in which | XNPSLRN, in | TXDX, in | (SEQ ID NO: | (SEQ ID NO: | in which X at |
| X at position 4 | which X at | which X at | 238) | 239) | position 5 is S, |
| is L, W, or M, | position 8 is K | position 2 is A | | | R, L, E, or F |
| X at position 6 | or L and X at | or K, X at | | | (SEQ ID NO: |
| is K, R, or M, | position 10 is | position 12 is | | | 240) |
| and X at | Y or L (SEQ ID | Mor F, and X | | | |
| position 9 is S | NO: 236) | at position 14 | | | |
| or R (SEQ ID | | is M or L (SEQ | | | |
| NO: 235) | | ID NO: 237) | | | |
| GFSXRXVGX | HIYWDDDXR | RXITTVIGGG | SASSSISYSH | DTSKLAR | QQWYXNPLT, |
| SVS, in which | XNPSLRN, in | TXDX, in | (SEQ ID NO: | (SEQ ID NO: | in which X at |
| X at position 4 | which X at | which X at | 241) | 239) | position 5 is S, |
| is L, W, or M, | position 8 is K | position 2 is A | | | R, L, E, or F |
| X at position 6 | or L and X at | or K, X at | | | (SEQ ID NO: |
| is K, R, or M, | position 10 is | position 12 is | | | 240 |
| and X at | Y or L (SEQ ID | Mor F, and X | | | |
| position 9 is S | NO: 236) | at position 14 | | | |
| or R (SEQ ID | | is M or L (SEQ | | | |
| NO: 235) | | ID NO: 237) | | | |
| GFSLRKVGR | HIYWDDDKR | RAITTVIGGG | SASSSISYMH | DTSKLAR | QQWYRNPLT |
| SVS (SEQ ID | YNPSLRN | TFDY (SEQ ID | (SEQ ID NO: | (SEQ ID NO: | (SEQ ID NO: |
| NO: 242) | (SEQ ID NO: | NO: 244) | 238) | 239) | 245) |
| | 243) | | | | |
| GFSLRKVGS | HIYWDDDKR | RAITTVIGGG | SASSSISYMH | DTSKLAR | QQWYLNPLT |
| SVS (SEQ ID | LNPSLRN | TFDL (SEQ ID | (SEQ ID NO: | (SEQ ID NO: | (SEQ ID NO: |
| NO: 246) | (SEQ ID NO: | NO: 248) | 238) | 239) | 249) |
| | 247) | | | | |
| RYWMN (SEQ | QIRLKSDNYA | GLDY (SEQ | KASQDINKYV | YTSWLQP | LQYDNLLYT |
| ID NO: 300) | THYAESVKG | ID NO: 302) | A (SEQ ID | (SEQ ID NO: | (SEQ ID NO: |
| | (SEQ ID NO: | | NO: 303) | 304) | 305) |
| | 301) | | | | |
| SSYGMH | VISYDGSNKY | DLLVRFLEWS | SGSSSNIGSN | SDNQRPS | AAWDDSLNG |
| (SEQ ID NO: | YADSVKG | HYYGMDV | TVH (SEQ ID | (SEQ ID NO: | V (SEQ ID |
| 322) | (SEQ ID NO: | (SEQ ID NO: | NO: 325) | 326) | NO: 327) |
| | 323) | 324) | | | |
| GFTFSSYGM | ISYDGSN | DLLVRFLEWS | SSNIGSNT | SDN (SEQ ID | AAWDDSLNG |
| H (SEQ ID | (SEQ ID NO: | HYYGMDV | (SEQ ID NO: | NO: 331) | V (SEQ ID |
| NO: 328) | 329) | (SEQ ID NO: | 330) | | NO: 327) |
| | | 324) | | | |
| SYAMS (SEQ | TISSGGSYTS | QDYAMNY | KASQDVSTA | SASYRYT | QQHYSTPWT |
| ID NO: 359) | YPDSVKG | (SEQ ID NO: | VA (SEQ ID | (SEQ ID NO: | (SEQ ID NO: |
| | (SEQ ID NO: | 361) | NO: 362) | 363) | 364) |
| | 360) | | | | |
| SYAMS (SEQ | TISSGGSYTS | QDYAMNY | KASQDVSTA | SASYRYT | QQHYSTPWT |
| ID NO: 359) | (SEQ ID NO: | (SEQ ID NO: | VA (SEQ ID | (SEQ ID NO: | (SEQ ID NO: |
| | 365) | 361) | NO: 362) | 363) | 364) |
| GFTFSSYAM | TISSGGSYTS | QDYAMNY | KASQDVSTA | SASYRYT | QQHYSTPWT |
| S (SEQ ID | YPDSVKG | (SEQ ID NO: | VA (SEQ ID | (SEQ ID NO: | (SEQ ID NO: |
| NO: 366) | (SEQ ID NO: | 361) | NO: 362) | 363) | 364) |
| | 360) | | | | |
| GFTFSSYAM | TISSGGSYTS | QDYAMNY | KASQDVSTA | SASYRYT | QQHYSTPWT |
| S (SEQ ID | (SEQ ID NO: | (SEQ ID NO: | VA (SEQ ID | (SEQ ID NO: | (SEQ ID NO: |
| NO: 366) | 365) | 361) | NO: 362) | 363) | 364) |
| SYWMQ (SEQ | AIYPGDGDTR | ARMGGYDRY | KSSQSLLNSA | FASTRES | QQHYNTPLT |
| ID NO: 375) | YTQKFKG | YFDY (SEQ ID | NQKNYLA | (SEQ ID NO: | (SEQ ID NO: |
| | (SEQ ID NO: | NO: 377) | (SEQ ID NO: | 379) | 380) |
| | 376) | | 378) | | |
| GYTFTSYWM | AIYPGDGDT | ARMGGYDRY | KSSQSLLNSA | FASTRES | QQHYNTPLT |
| Q (SEQ ID | (SEQ ID NO: | YFDY (SEQ ID | NQKNYLA | (SEQ ID NO: | (SEQ ID NO: |
| NO: 381) | 382) | NO: 383) | (SEQ ID NO: | 385) | 386) |
| | | | 384) | | |
| GFTFSAYA | ISGSGGSA | AKDGAWKMS | QDISDY (SEQ | TTS (SEQ ID | QKYDSAPLT |
| (SEQ ID NO: | (SEQ ID NO: | GLDV (SEQ | ID NO: 396) | NO: 397) | (SEQ ID NO: |
| 393) | 394) | ID NO: 395) | | | 398) |
| GFTFSSFG | IGYDGGNE | STISHYDILSG | QGISNW | AAS | QQANSFPLT |
| (SEQ ID NO: | (SEQ ID NO: | MDV (SEQ ID | (SEQ ID | (SEQ ID | (SEQ ID NO: |
| 399) | 400) | NO: 401) | NO: 402) | NO: 403) | 404) |
| X₁X₂DIS (SEQ | IISX₁AGX₂X₃Y | GVPAX₁SX₂G | X₁X₂SQX₃VX₄ | WAX₁TLAX₂ | AGGYGGGX₁ |
| ID NO: 823), in | X₄X₅X₆W | GDX₃ (SEQ ID | X₅X₆NWLS | (SEQ ID NO: | YA (SEQ ID |
| which X₁ is S | AKX₇ (SEQ ID | NO: 825), in | (SEQ ID NO: | 827), in which | NO: 828), in |
| or H, X₂ is Y, | NO: 824), in | which X₁ is Y | 826), in which | X₁ is S or E, | which X₁ is L |
| T, D or E | which X₁ is Y | or H, X₂ is T or | X₁ is Q or T, | X₂ is S, Y, F | or R |
| | or H, X₂ is S or | H, X₃ is L or K | X₂ is S or T, | or W | |
| | K, X₃ is T, M or | | X₃ is S or E, | | |
| | K, X₄ is Y or K, | | X₄ is Y or F, | | |
| | X₅ is A, M or E, | | X₅ is D or H, | | |
| | X₆ is S or E, | | X₆ is N, D, A | | |
| | Xis G or K | | or E | | |
| HDDIS (SEQ | IISYAGSTYYA | GVPAYSHGG | TSSQSVYHA | WASTLAY | AGGYGGGRY |
| ID NO: 487) | SWAKG (SEQ | DL (SEQ ID | NWLS (SEQ | (SEQ ID NO: | A (SEQ ID |
| | ID NO: 488) | NO: 489) | ID NO: 490) | 491) | NO: 492) |
| HDDIS (SEQ | IISYAGSTYYA | GVPAYSHGG | TTSQSVYHE | WASTLAY | AGGYGGGRY |
| ID NO: 487) | SWAKG (SEQ | DL (SEQ ID | NWLS (SEQ | (SEQ ID NO: | A (SEQ ID |
| | ID NO: 488) | NO: 489) | ID NO: 493) | 491) | NO: 492) |

TABLE 5
Exemplary heavy chain CDR sequences
of myostatin antibodies

| HCDR1 | HCDR2 | HCDR3 |
|---|---|---|
| SSYG | VISY | DLLV |
| MH | DGSN | RFLE |
| (SEQ ID | KYYA | WSHY |
| NO: 322) | DSVK | YGMD |
| | G | V |
| | (SEQ ID | (SEQ ID |
| | NO: 323) | NO: 324) |
| GFTF | ISYD | ASPA |
| SSYG | GSN | GHIC |
| MH | (SEQ ID | SGGS |
| (SEQ ID | NO: 329) | CQYY |
| NO: 328) | | YYGM |
| | | DV |
| | | (SEQ ID |
| | | NO: 337) |
| GFTF | ISGG | ARDR |
| SSYA | GGAT | HLYR |
| (SEQ ID | (SEQ ID | RGYS |
| NO: 332) | NO: 335) | SVDG |
| | | MDV |
| | | (SEQ ID |
| | | NO: 338) |
| GFTF | INSD | ARHP |
| DDYG | GSST | IGSG |
| (SEQ ID | (SEQ ID | YDY |
| NO: 333) | NO: 336) | (SEQ ID |
| | | NO: 339) |
| GFTF | | AKEI |
| SNYW | | SDTA |
| (SEQ ID | | MGFD |
| NO: 334) | | Y |
| | | (SEQ ID |
| | | NO: 340) |
| | | AREG |
| | | YNAH |
| | | YFDY |
| | | (SEQ ID |
| | | NO: 341) |
| | | AKGL |
| | | GYHP |
| | | LFDY |
| | | (SEQ ID |
| | | NO: 342) |

TABLE 6
Exemplary light chain CDR sequences
of myostatin antibodies

| LCDR1 | LCDR2 | LCDR3 |
|---|---|---|
| SGSS | SDNQ | AAWD |
| SNIG | RPS | DSLN |
| SNTV | (SEQ ID | GV |
| H | NO: 326) | (SEQ ID |
| (SEQ ID | | NO: 327) |
| NO: 325) | | |
| SSNI | SDN | QQSY |
| GSNT | (SEQ ID | STPL |
| (SEQ ID | NO: 331) | T |
| NO: 330) | | (SEQ ID |
| | | NO: 350) |
| QGIS | AAS | QQRS |
| SY | (SEQ ID | IWPL |
| (SEQ ID | NO: 347) | T |
| NO: 343) | | (SEQ ID |
| | | NO: 351) |
| QSVS | GGS | MQGS |
| SGY | (SEQ ID | SWPY |
| (SEQ ID | NO: 348) | T |
| NO: 344) | | (SEQ ID |
| | | NO: 352) |
| QSVV | DVT | NSYT |
| YRDG | (SEQ ID | SSRT |
| NTY | NO: 349) | VV |
| (SEQ ID | | (SEQ ID |
| NO: 345) | | NO: 353) |
| QSVL | | SSYT |
| YSSD | | SSST |
| NKNY | | VV |
| (SEQ ID | | (SEQ ID |
| NO: 346) | | NO: 354) |
| | | QQYY |
| | | STPS |
| | | (SEQ ID |
| | | NO: 355) |

In some embodiments, the myostatin antibody or an antigen-binding fragment thereof, has a heavy chain and light chain sequence having at least 90% sequence identity (e.g., 90%, 91%, 92%, 93%, 94%, 95%, 95%, 97%, 98%, 99% or 100% sequence identity) to a heavy chain and light chain sequence provided in Table 7. In some embodiments, the myostatin antibody or an antigen binding fragment thereof, has a heavy chain and light chain sequence from the same row of Table 7. In some embodiments, the heavy chain and light chain have the sequence of SEQ ID NOs: 274 and 275; 276 and 277; 278 and 279; 280 and 281; 282 and 283; 284 and 285; 286 and 287; 288 and 289; 290 and 291; 292 and 293; 294 and 295; 296 and 297; 367 and 368; or 369 and 370 (e.g., the heavy chain has at least 90% sequence identity (e.g., 90%, 91%, 92%, 93%, 94%, 95%, 95%, 97%, 98%, 99% or 100% sequence identity) to the sequence of the first SEQ ID NO: in each pair and the light chain has at least 90% sequence identity (e.g., 90%, 91%, 92%, 93%, 94%, 95%, 95%, 97%, 98%, 99% or 100% sequence identity) to the sequence of the second SEQ ID NO: in each pair).

TABLE 7
Exemplary heavy and light chain
sequences of myostatin antibodies

| Heavy chain | Light chain |
|---|---|
| EVQLVESGGGLVKPGGSLRL | DIQMTQSPSSLSASVGDRVT |
| SCAASGFTFSSYSMNWVRQA | ITCRASQGIRNALGWYQQKP |
| PGKGLEWVSSISSSSSYIYY | GKAPQRLIYAASSLQSGVPS |
| ADSVKGRFTISRDNAKNSLY | RFSGSGSGTEFTLTISSLQP |
| LQMNSLRAEDTAVYYCARDR | EDFATYYCLQHNSYPRTFGQ |
| GYSYGLDYWGQGTLVTVSSA | GTKVEIKRTVAAPSVFIFPP |
| STKGPSVFPLAPCSRSTSES | SDEQLKSGTASVVCLLNNFY |
| TAALGCLVKDYFPEPVTVSW | PREAKVQWKVDNALQSGNSQ |
| NSGALTSGVHTFPAVLQSSG | ESVTEQDSKDSTYSLSSTLT |
| LYSLSSVVTVPSSNFGTQTY | LSKADYEKHKVYACEVTHQG |
| TCNVDHKPSNTKVDKTVERK | LSSPVTKSFNRGEC |
| CCVECPPCPAPPVAGPSVFL | (SEQ ID NO: 275) |
| FPPKPKDTLMISRTPEVTCV | |
| VVDVSHEDPEVQFNWYVDGV | |
| EVHNAKTKPREEQFNSTFRV | |
| VSVLTVVHQDWLNGKEYKCK | |
| VSNKGLPAPIEKTISKTKGQ | |
| PREPQVYTLPPSREEMTKNQ | |
| VSLTCLVKGFYPSDIAVEWE | |
| SNGQPENNYKTTPPMLDSDG | |
| SFFLYSKLTVDKSRWQQGNV | |
| FSCSVMHEALHNHYTQKSLS | |
| LSPGK | |
| (SEQ ID NO: 274) | |
| QVQLVQSGAEVKKPGASVKV | DIVMTQSPLSLPVTPGEPAS |
| SCKASGYTFTGYYMHWVRQA | ISCRSSQSLLHSNGYNYLDW |
| PGQGLEWMGWINPNNGGTNY | YLQKSGQSPQLLIHLGSNRA |
| AQKFQGRVTMTRDTSISTAY | SGVPDRFSGSGSGTDFTLKI |
| MELSRLRSDDTAVYHCARNT | SRVEAEDVGVYYCMQVLQTP |
| VGSGYYYYGMDVWGQGTTVT | LTFGGGTKVEIKRTVAAPSV |
| VSSASTKGPSVFPLAPCSRS | FIFPPSDEQLKSGTASVVCL |
| TSESTAALGCLVKDYFPEPV | LNNFYPREAKVQWKVDNALQ |
| TVSWNSGALTSGVHTFPAVL | SGNSQESVTEQDSKDSTYSL |
| QSSGLYSLSSVVTVPSSNFG | SSTLTLSKADYEKHKVYACE |
| TQTYTCNVDHKPSNTKVDKT | VTHQGLSSPVTKSFNRGEC |
| VERKCCVECPPCPAPPVAGP | (SEQ ID NO: 277) |
| SVFLFPPKPKDTLMISRTPE | |
| VTCVVVDVSHEDPEVQFNWY | |
| VDGVEVHNAKTKPREEQFNS | |
| TFRVVSVLTVVHQDWLNGKE | |
| YKCKVSNKGLPAPIEKTISK | |
| TKGQPREPQVYTLPPSREEM | |
| TKNQVSLTCLVKGFYPSDIA | |
| VEWESNGQPENNYKTTPPML | |
| DSDGSFFLYSKLTVDKSRWQ | |
| QGNVFSCSVMHEALHNHYTQ | |
| KSLSLSPGK | |
| (SEQ ID NO: 276) | |
| EVQLVESGGGLVKPGGSLRL | DIQMTQSPSSLSASVGDRVT |
| SCAASGFTFSSYSMNWVRQA | ITCRASQGIRNALGWYQQKP |
| PGKGLEWVSSISSSSSYIYY | GKAPKRLIYAASSLQSGVPS |
| ADSVKGRFTISRDNAKNSLY | RFSGSGSGTEFTLTISSLQP |
| LQMNSLRAEDTAVYYCARDR | EDFATYYCLQHNSYPRTFGQ |
| GYSYGIDYWGQGTLVTVSSA | GTKVEIKRTVAAPSVFIFPP |
| STKGPSVFPLAPCSRSTSES | SDEQLKSGTASVVCLLNNFY |
| TAALGCLVKDYFPEPVTVSW | PREAKVQWKVDNALQSGNSQ |
| NSGALTSGVHTFPAVLQSSG | ESVTEQDSKDSTYSLSSTLT |
| LYSLSSVVTVPSSNFGTQTY | LSKADYEKHKVYACEVTHQG |
| TCNVDHKPSNTKVDKTVERK | LSSPVTKSFNRGEC |
| CCVECPPCPAPPVAGPSVFL | (SEQ ID NO: 279) |
| FPPKPKDTLMISRTPEVTCV | |
| VVDVSHEDPEVQFNWYVDGV | |
| EVHNAKTKPREEQFNSTFRV | |
| VSVLTVVHQDWLNGKEYKCK | |
| VSNKGLPAPIEKTISKTKGQ | |
| PREPQVYTLPPSREEMTKNQ | |
| VSLTCLVKGFYPSDIAVEWE | |
| SNGQPENNYKTTPPMLDSDG | |
| SFFLYSKLTVDKSRWQQGNV | |
| FSCSVMHEALHNHYTQKSLS | |
| LSPGK | |
| (SEQ ID NO: 278) | |
| EVQLVESGGGLVKPGGSLRL | DIQMTQSPSSLSASVGDRVT |
| SCAASGFTFSSYSMNWVRQA | LTCRASQGIRNALGWYQQKP |
| PGKGLEWVSSISSSSSYIYY | GKAPKRLIYGASSLQSGVPS |
| ADSVKGRFTISRDNAKNSLY | RFSGSGSGTEFTLTISSLQP |
| LQMNSLRAEDTAVYYCARDR | EDFATYYCLQHNSYPFTFGP |
| GYYYGMDVWGQGTTVTVSSA | GTKVDIKRTVAAPSVFIFPP |
| STKGPSVFPLAPCSRSTSES | SDEQLKSGTASVVCLLNNFY |
| TAALGCLVKDYFPEPVTVSW | PREAKVQWKVDNALQSGNSQ |
| NSGALTSGVHTFPAVLQSSG | ESVTEQDSKDSTYSLSSTLT |
| LYSLSSVVTVPSSNFGTQTY | LSKADYEKHKVYACEVTHQG |
| TCNVDHKPSNTKVDKTVERK | LSSPVTKSFNRGEC |
| CCVECPPCPAPPVAGPSVFL | (SEQ ID NO: 281) |
| FPPKPKDTLMISRTPEVTCV | |
| VVDVSHEDPEVQFNWYVDGV | |
| EVHNAKTKPREEQFNSTFRV | |
| VSVLTVVHQDWLNGKEYKCK | |
| VSNKGLPAPIEKTISKTKGQ | |
| PREPQVYTLPPSREEMTKNQ | |
| VSLTCLVKGFYPSDIAVEWE | |
| SNGQPENNYKTTPPMLDSDG | |
| SFFLYSKLTVDKSRWQQGNV | |
| FSCSVMHEALHNHYTQKSLS | |
| LSPGK | |
| (SEQ ID NO: 280) | |
| EVQLVESGGGLVKPGGSLRL | DIQMTQSPSSLSASVGDRVT |
| SCAASGFTFSSYSMNWVRQA | ITCRASQAIRNDLGWYQQKP |
| PGKGLEWVSSISSSSSYIYY | GKAPKRLIYAASSLQSGVPS |
| ADSVKGRFTISRDNAKNSLY | RFSGSGSGTEFTLTISSLQP |
| LQMNSLRAEDTTVYYCARDR | EDFATYYCLQHNSYPRTFGQ |
| GYLYGMDVWGQGTTVTVSSA | GTKVEIKRTVAAPSVFIFPP |
| STKGPSVFPLAPCSRSTSES | SDEQLKSGTASVVCLLNNFY |
| TAALGCLVKDYFPEPVTVSW | PREAKVQWKVDNALQSGNSQ |
| NSGALTSGVHTFPAVLQSSG | ESVTEQDSKDSTYSLSSTLT |
| LYSLSSVVTVPSSNFGTQTY | LSKADYEKHKVYACEVTHQG |
| TCNVDHKPSNTKVDKTVERK | LSSPVTKSFNRGEC |
| CCVECPPCPAPPVAGPSVFL | (SEQ ID NO: 283) |
| FPPKPKDTLMISRTPEVTCV | |
| VVDVSHEDPEVQFNWYVDGV | |
| EVHNAKTKPREEQFNSTFRV | |
| VSVLTVVHQDWLNGKEYKCK | |
| VSNKGLPAPIEKTISKTKGQ | |
| PREPQVYTLPPSREEMTKNQ | |
| VSLTCLVKGFYPSDIAVEWE | |
| SNGQPENNYKTTPPMLDSDG | |
| SFFLYSKLTVDKSRWQQGNV | |
| FSCSVMHEALHNHYTQKSLS | |
| LSPGK | |
| (SEQ ID NO: 282) | |
| QVQLVQSGAEVKKPGASVKV | DIVMTQSPLSLPVTPGEPAS |
| SCKASGYTFTGYYMHWVRQA | ISCRSSQSLLHSNGYNYLDW |
| PGQGLEWMGWINPNSGGTNY | YLQKPGQSPQVLIYLVSNRA |
| AQKFQGRVTMTRDTSISTAY | SGVPDRFSGSGSGTDFTLKI |
| LELSRLRSDDTAVYYCARNT | SRVEAEDVGVYYCMQALQTP |
| VGTGYYYYGMDVWGQGTTVT | PTFGGGTKVEIKRTVAAPSV |
| VSSASTKGPSVFPLAPCSRS | FIFPPSDEQLKSGTASVVCL |
| TSESTAALGCLVKDYFPEPV | LNNFYPREAKVQWKVDNALQ |
| TVSWNSGALTSGVHTFPAVL | SGNSQESVTEQDSKDSTYSL |
| QSSGLYSLSSVVTVPSSNFG | SSTLTLSKADYEKHKVYACE |
| TQTYTCNVDHKPSNTKVDKT | VTHQGLSSPVTKSFNRGEC |
| VERKCCVECPPCPAPPVAGP | (SEQ ID NO: 285) |
| SVFLFPPKPKDTLMISRTPE | |
| VTCVVVDVSHEDPEVQFNWY | |
| VDGVEVHNAKTKPREEQFNS | |
| TFRVVSVLTVVHQDWLNGKE | |
| YKCKVSNKGLPAPIEKTISK | |
| TKGQPREPQVYTLPPSREEM | |
| TKNQVSLTCLVKGFYPSDIA | |
| VEWESNGQPENNYKTTPPML | |
| DSDGSFFLYSKLTVDKSRWQ | |
| QGNVFSCSVMHEALHNHYTQ | |
| KSLSLSPGK | |
| (SEQ ID NO: 284) | |
| EVQLLESGGGLIQPGGSLRL | EIVMTQSPATLSVSPGERAT |
| SCAASGFTFSSFAMSWVRQA | LSCRASQSVSSNLAWYQQKP |
| PGKGLEWVSTISGSGGYTFY | GQAPRLLIYGASTRATGFPA |
| ADSVKGRFTISRDNSKNTLY | RFSGSGSGTEFTLTISSLQS |
| LQMNSLRAEDTAVYYCAKDG | EDFAIYYCQQYNNWPLTFGG |
| RYNWNYGAFDIWGQGTMVTV | GTKVEIKRTVAAPSVFIFPP |
| SSASTKGPSVFPLAPCSRST | SDEQLKSGTASVVCLLNNFY |
| SESTAALGCLVKDYFPEPVT | PREAKVQWKVDNALQSGNSQ |
| VSWNSGALTSGVHTFPAVLQ | ESVTEQDSKDSTYSLSSTLT |
| SSGLYSLSSVVTVPSSSLGT | LSKADYEKHKVYACEVTHQG |
| KTYTCNVDHKPSNTKVDKRV | LSSPVTKSFNRGEC |
| ESKYGPPCPSCPAPEFLGGP | (SEQ ID NO: 287) |
| SVFLFPPKPKDTLMISRTPE | |
| VTCVVVDVSQEDPEVQFNWY | |
| VDGVEVHNAKTKPREEQFNS | |
| TYRVVSVLTVLHQDWLNGKE | |
| YKCKVSNKGLPSSIEKTISK | |
| AKGQPREPQVYTLPPSQEEM | |
| TKNQVSLTCLVKGFYPSDIA | |
| VEWESNGQPENNYKTTPPVL | |
| DSDGSFFLYSRLTVDKSRWQ | |
| EGNVFSCSVMHEALHNHYTQ | |
| KSLSLSLGK | |
| (SEQ ID NO: 286) | |
| EVQLVESGGGLVKPGGSLRL | DMQMTQSPSSLSASVGDRVT |
| SCAASGFTFSSYSMNWVRQA | ITCRASQGIRNDLGWYQQKP |
| PGKGLEWVSSISSSSSYIYY | GKAPKRLIYGASSLQSGVPS |
| ADSVKGRFTISRDNAKNSLY | RFSGSGSGTEFTLTISSLQP |
| LQMNSLRAEDTAVYYCARDR | EDSATYYCLQHNSYPLTFGG |
| GSYFLFDYWGQGTLVTVSSA | GTKVEIKRTVAAPSVFIFPP |
| STKGPSVFPLAPCSRSTSES | SDEQLKSGTASVVCLLNNFY |
| TAALGCLVKDYFPEPVTVSW | PREAKVQWKVDNALQSGNSQ |
| NSGALTSGVHTFPAVLQSSG | ESVTEQDSKDSTYSLSSTLT |
| LYSLSSVVTVPSSNFGTQTY | LSKADYEKHKVYACEVTHQG |
| TCNVDHKPSNTKVDKTVERK | LSSPVTKSFNRGEC |
| CCVECPPCPAPPVAGPSVFL | (SEQ ID NO: 289) |
| FPPKPKDTLMISRTPEVTCV | |
| VVDVSHEDPEVQFNWYVDGV | |
| EVHNAKTKPREEQFNSTFRV | |
| VSVLTVVHQDWLNGKEYKCK | |
| VSNKGLPAPIEKTISKTKGQ | |
| PREPQVYTLPPSREEMTKNQ | |
| VSLTCLVKGFYPSDIAVEWE | |
| SNGQPENNYKTTPPMLDSDG | |
| SFFLYSKLTVDKSRWQQGNV | |
| FSCSVMHEALHNHYTQKSLS | |
| LSPGK | |
| (SEQ ID NO: 288) | |
| EVQLVESGGGLVKPGGSLRL | DIQMTQSPSSLSASVGDRVT |
| SCAASGFTFSSYSMNWVRQA | ITCRASQGIRNDLGWYQQKP |
| PGKGLEWVSSISSSSSYIYY | GKAPKRLIYAASSLQSGVPS |
| ADSVKGRFTISRDNAKNSLY | RFSGSGSGTEFTLTISSLQP |
| LQMNSLRAEDTAVYYCARDR | EDFATYYCLQLNSYPFTFGP |
| GYSYGLDYWGQGTLVTVSSA | GTKVDIKRTVAAPSVFIFPP |
| STKGPSVFPLAPCSRSTSES | SDEQLKSGTASVVCLLNNFY |
| TAALGCLVKDYFPEPVTVSW | PREAKVQWKVDNALQSGNSQ |
| NSGALTSGVHTFPAVLQSSG | ESVTEQDSKDSTYSLSSTLT |
| LYSLSSVVTVPSSNFGTQTY | LSKADYEKHKVYACEVTHQG |
| TCNVDHKPSNTKVDKTVERK | LSSPVTKSFNRGEC |
| CCVECPPCPAPPVAGPSVFL | (SEQ ID NO: 291) |
| FPPKPKDTLMISRTPEVTCV | |
| VVDVSHEDPEVQFNWYVDGV | |
| EVHNAKTKPREEQFNSTFRV | |
| VSVLTVVHQDWLNGKEYKCK | |
| VSNKGLPAPIEKTISKTKGQ | |
| PREPQVYTLPPSREEMTKNQ | |
| VSLTCLVKGFYPSDIAVEWE | |
| SNGQPENNYKTTPPMLDSDG | |
| SFFLYSKLTVDKSRWQQGNV | |
| FSCSVMHEALHNHYTQKSLS | |
| LSPGK | |
| (SEQ ID NO: 290) | |
| EVQLLESGGGLIQPGGSLRL | EIVMTQSPATLSVSPGERAT |
| SCAASGFTFSSFAMSWVRQA | LSCRASQSVRSNLAWFQQKP |
| PGKGLEWVSTISGSGGSTYY | GQAPRLLIYGASTRATGIPA |
| ADFVKGRFTISRDNSKNTLY | RFSGSGSGTEFTLTISSLQS |
| LQMNSLRAEDTAVYYCAKDG | EDFAVYYCQQYNNWPLTFGG |
| RYNWNYGAFDIWGQGTMVTV | GTKVEIKRTVAAPSVFIFPP |
| SSASTKGPSVFPLAPCSRST | SDEQLKSGTASVVCLLNNFY |
| SESTAALGCLVKDYFPEPVT | PREAKVQWKVDNALQSGNSQ |
| VSWNSGALTSGVHTFPAVLQ | ESVTEQDSKDSTYSLSSTLT |
| SSGLYSLSSVVTVPSSSLGT | LSKADYEKHKVYACEVTHQG |
| KTYTCNVDHKPSNTKVDKRV | LSSPVTKSFNRGEC |
| ESKYGPPCPSCPAPEFLGGP | (SEQ ID NO: 293) |
| SVFLFPPKPKDTLMISRTPE | |
| VTCVVVDVSQEDPEVQFNWY | |
| VDGVEVHNAKTKPREEQFNS | |
| TYRVVSVLTVLHQDWLNGKE | |
| YKCKVSNKGLPSSIEKTISK | |
| AKGQPREPQVYTLPPSQEEM | |
| TKNQVSLTCLVKGFYPSDIA | |
| VEWESNGQPENNYKTTPPVL | |
| DSDGSFFLYSRLTVDKSRWQ | |
| EGNVFSCSVMHEALHNHYTQ | |
| KSLSLSLGK | |
| (SEQ ID NO: 292) | |
| EVQLLESGGGLVQPGGSLRL | EIVMTQSPATLSVSPGERAT |
| SCAASGFTFSSYAMSWVRQA | LSCRTSQSVSSNLAWYQQKP |
| PGKGLEWVSAISGSGGSTFY | GQAPRLLIYGASTRATGIPA |
| ADSVTGRFTISRDNSKNTLF | RFSGSGSGTEFTLTISSLQS |
| LQLNSLRAEDTAVYHCAKDG | EDFAVYYCQQYNNWPLTFGG |
| RFNWNYGASDIWGQGTMVTV | GTKVEIKRTVAAPSVFIFPP |
| SSASTKGPSVFPLAPCSRST | SDEQLKSGTASVVCLLNNFY |
| SESTAALGCLVKDYFPEPVT | PREAKVQWKVDNALQSGNSQ |
| VSWNSGALTSGVHTFPAVLQ | ESVTEQDSKDSTYSLSSTLT |
| SSGLYSLSSVVTVPSSSLGT | LSKADYEKHKVYACEVTHQG |
| KTYTCNVDHKPSNTKVDKRV | LSSPVTKSFNRGEC |
| ESKYGPPCPSCPAPEFLGGP | (SEQ ID NO: 295) |
| SVFLFPPKPKDTLMISRTPE | |
| VTCVVVDVSQEDPEVQFNWY | |
| VDGVEVHNAKTKPREEQFNS | |
| TYRVVSVLTVLHQDWLNGKE | |
| YKCKVSNKGLPSSIEKTISK | |
| AKGQPREPQVYTLPPSQEEM | |
| TKNQVSLTCLVKGFYPSDIA | |
| VEWESNGQPENNYKTTPPVL | |
| DSDGSFFLYSRLTVDKSRWQ | |
| EGNVFSCSVMHEALHNHYTQ | |
| KSLSLSLGK | |
| (SEQ ID NO: 294) | |
| EVQLLESGGGLVQPGGSLRL | EIVMTQSPATLSVSPGERAT |
| SCAASGFTFSSFAMSWVRQA | LSCRASQSVSSNLAWYQQKP |
| PGKGLEWVSTISGSGGYTFY | GQAPRLLIYGASTRATGIPA |
| ADSVKGRFTISRDNSKNTLY | RFSGSGSGTEFTLTISSLQS |
| LQMNSLRAEDTAVYYCAKDG | EDFAVYYCQQYNNWPLTFGG |
| RYNWNYGAFDIWGQGTMVTV | GTKVEIKRTVAAPSVFIFPP |
| SSASTKGPSVFPLAPCSRST | SDEQLKSGTASVVCLLNNFY |
| SESTAALGCLVKDYFPEPVT | PREAKVQWKVDNALQSGNSQ |
| VSWNSGALTSGVHTFPAVLQ | ESVTEQDSKDSTYSLSSTLT |
| SSGLYSLSSVVTVPSSNFGT | LSKADYEKHKVYACEVTHQG |
| QTYTCNVDHKPSNTKVDKTV | LSSPVTKSFNRGEC |
| ERKCCVECPPCPAPPVAGPS | (SEQ ID NO: 297) |
| VFLFPPKPKDTLMISRTPEV | |
| TCVVVDVSHEDPEVQFNWYV | |
| DGVEVHNAKTKPREEQFNST | |
| FRVVSVLTVVHQDWLNGKEY | |
| KCKVSNKGLPAPIEKTISKT | |
| KGQPREPQVYTLPPSREEMT | |
| KNQVSLTCLVKGFYPSDIAV | |
| EWESNGQPENNYKTTPPMLD | |
| SDGSFFLYSKLTVDKSRWQQ | |
| GNVFSCSVMHEALHNHYTQK | |
| SLSLSPGK | |
| (SEQ ID NO: 296) | |
| ASTKGPSVFPLAPSSKSTSG | RTVAAPSVFIFPPSDEQLKS |
| GTAALGCLVKDYFPEPVTVS | GTASVVCLLNNFYPREAKVQ |
| WNSGALTSGVHTFPAVLQSS | WKVDNALQSGNSQESVTEQD |
| GLYSLSSVVTVPSSSLGTQT | SKDSTYSLSSTLTLSKADYE |
| YICNVNHKPSNTKVDKKVEP | KHKVYACEVTHQGLSSPVTK |
| KSCDKTHTCPPCPAPEAAGA | SFNRGEC |
| PSVFLFPPKPKDTLMISRTP | (SEQ ID NO: 368) |
| EVTCVVVDVSHEDPEVKFNW | |
| YVDGVEVHNAKTKPREEQYN | |
| STYRVVSVLTVLHQDWLNGK | |
| EYKCKVSNKALPAPIEKTIS | |
| KAKGQPREPQVYTLPPSREE | |
| MTKNQVSLTCLVKGFYPSDI | |
| AVEWESNGQPENNYKTTPPV | |
| LDSDGSFFLYSKLTVDKSRW | |
| QQGNVFSCSVMHEALHNHYT | |
| QKSLSLSPGK | |
| (SEQ ID NO: 367) | |
| EVQLLESGGGLVQPGGSLRL | DIQMTQSPSSLSASVGDRVT |
| SCAASGFTFSSYAMSWVRQA | ITCKASQDVSTAVAWYQQKP |
| PGKGLEWVSTISSGGSYTSY | GKAPKLLIYSASYRYTGVPS |
| PDSVKGRFTISRDNSKNTLY | RFSGSGSGTDFTLTISSLQP |
| LQMNSLRAEDTAVYYCAKQD | EDFATYYCQQHYSTPWTFGG |
| YAMNYWGQGTLVTVSSASTK | GTKVEIKRTVAAPSVFIFPP |
| GPSVFPLAPSSKSTSGGTAA | SDEQLKSGTASVVCLLNNFY |
| LGCLVKDYFPEPVTVSWNSG | PREAKVQWKVDNALQSGNSQ |
| ALTSGVHTFPAVLQSSGLYS | ESVTEQDSKDSTYSLSSTLT |
| LSSVVTVPSSSLGTQTYICN | LSKADYEKHKVYACEVTHQG |
| VNHKPSNTKVDKKVEPKSCD | LSSPVTKSFNRGEC |
| KTHTCPPCPAPEAAGAPSVF | (SEQ ID NO: 370) |
| LFPPKPKDTLMISRTPEVTC | |
| VVVDVSHEDPEVKFNWYVDG | |
| VEVHNAKTKPREEQYNSTYR | |
| VVSVLTVLHQDWLNGKEYKC | |
| KVSNKALPAPIEKTISKAKG | |
| QPREPQVYTLPPSREEMTKN | |
| QVSLTCLVKGFYPSDIAVEW | |
| ESNGQPENNYKTTPPVLDSD | |
| GSFFLYSKLTVDKSRWQQGN | |
| VFSCSVMHEALHNHYTQKSL | |
| SLSPGK | |
| (SEQ ID NO: 369) | |

In some embodiments, the myostatin antibody is a bi-specific antibody that also binds to activin A. Exemplary bi-specific myostatin antibodies that may be used in the methods described herein include those described in U.S. Pat. Nos. 9,718,881, 10,526,403, 10,400,036 and 8,871,209, the disclosures of which are incorporated herein by reference. In some embodiments, the bi-specific antibody includes an activin A HCVR and LCVR from Table 1 (e.g., a HCVR sequence having at least 90% (e.g., at least 91%, 92%, 93%, 94%, 95%, 96%, 97%, 98%, 99%, or 100%) sequence identity to a HCVR sequence in Table 1, such as any one of SEQ ID NOs: 138, 140, 142, 143, 144, 146, 148, 150, 151, 172, and 174, and a LCVR sequence having at least 90% (e.g., at least 91%, 92%, 93%, 94%, 95%, 96%, 97%, 98%, 99%, or 100%) sequence identity to a LCVR sequence in Table 1, such as any one of SEQ ID NOs: 139, 141, 145, 147, 149, 173, and 175) and a myostatin HCVR and LCVR from Table 3 (e.g., a HCVR sequence having at least 90% (e.g., at least 91%, 92%, 93%, 94%, 95%, 96%, 97%, 98%, 99%, or 100%) sequence identity to a HCVR sequence in Table 3, such as any one of SEQ ID NOs: 164, 188, 201, 204-210, 222-228, 234, 250, 252, 254, 256, 258, 260, 262, 264, 266, 268, 270, 272, 298, 306, 308, 310, 312, 314, 316, 318, 320, 356, 371, 373, 387, 389, 391, 405, 407, 409, 411, 413, 415, 417, 419, 421-423, 425, 427, 429, 431, 433, 435, 437, 439, 441, 444, 446, and 448-476, and a LCVR sequence having at least 90% (e.g., at least 91%, 92%, 93%, 94%, 95%, 96%, 97%, 98%, 99%, or 100%) sequence identity to a LCVR sequence in Table 3, such as any one of SEQ ID NOs: 165, 189, 202, 203, 221, 229-233, 251, 253, 255, 257, 259, 261, 263, 265, 267, 269, 271, 273, 299, 307, 309, 311, 313, 315, 317, 319, 321, 358, 372, 374, 388, 390, 392, 406, 408, 410, 412, 414, 416, 418, 420, 424, 426, 428, 430, 432, 434, 436, 438, 440, 442, 443, 445, 447, and 477-486). In some embodiments, the bi-specific antibody includes an activin A heavy chain CDR1, CDR2, and CDR3 and a light chain CDR1, CDR2, and CDR3 from Table 2 (e.g., an activin A heavy chain CDR1, CDR2, and CDR3 and a light chain CDR1, CDR2, and CDR3 from the same row of Table 2) and a myostatin heavy chain CDR1, CDR2, and CDR3 and a light chain CDR1, CDR2, and CDR3 from Table 4 (e.g., a myostatin heavy chain CDR1, CDR2, and CDR3 and a light chain CDR1, CDR2, and CDR3 from the same row of Table 4). In some embodiments, the bi-specific antibody includes an activin A HCVR of SEQ ID NO: 138 and LCVR of SEQ ID NO: 139 and a myostatin HCVR of SEQ ID NO: 164 and LCVR of SEQ ID NO: 165. In some embodiments, the bi-specific antibody includes an activin A HCVR of SEQ ID NO: 138 and LCVR of SEQ ID NO: 139 and a myostatin HCVR of SEQ ID NO: 387 and LCVR of SEQ ID NO: 388. In some embodiments, the bi-specific antibody includes an activin A HCVR of SEQ ID NO: 138 and LCVR of SEQ ID NO: 139 and a myostatin HCVR of SEQ ID NO: 391 and LCVR of SEQ ID NO: 392. In some embodiments, the bi-specific antibody includes an activin A HCVR of SEQ ID NO: 144 and LCVR of SEQ ID NO: 145 and a myostatin HCVR of SEQ ID NO: 164 and LCVR of SEQ ID NO: 165. In some embodiments, the bi-specific antibody includes an activin A HCVR of SEQ ID NO: 144 and LCVR of SEQ ID NO: 145 and a myostatin HCVR of SEQ ID NO: 387 and LCVR of SEQ ID NO: 388. In some embodiments, the bi-specific antibody includes an activin A HCVR of SEQ ID NO: 144 and LCVR of SEQ ID NO: 145 and a myostatin HCVR of SEQ ID NO: 391 and LCVR of SEQ ID NO: 392. In some embodiments, the bi-specific antibody includes an activin A heavy chain CDR1, CDR2, and CDR3 and a light chain CDR1, CDR2, and CDR3 of SEQ ID NOs: 152-157 and a myostatin heavy chain CDR1, CDR2, and CDR3 and a light chain CDR1, CDR2, and CDR3 of SEQ ID NOs: 166-171. In some embodiments, the bi-specific antibody includes an activin A heavy chain CDR1, CDR2, and CDR3 and a light chain CDR1, CDR2, and CDR3 of SEQ ID NOs: 158-163 and a myostatin heavy chain CDR1, CDR2, and CDR3 and a light chain CDR1, CDR2, and CDR3 of SEQ ID NOs: 166-171.

In some embodiments, the ActRII signaling inhibitor is an activin B antibody or an antigen binding fragment thereof. Activin B antibodies that may be used in the methods described herein include those described in U.S. Pat. No. 8,383,351, which is incorporated herein by reference. In some embodiments, the activin B antibody or an antigen binding fragment thereof has a HCVR including three CDRs from the HCVR sequence of SEQ ID NO: 494 and a LCVR including three CDRs from the LCVR sequence of SEQ ID NO: 495. In some embodiments, the activin B antibody or an antigen binding fragment thereof has a HCVR having at least 90% (e.g., at least 91%, 92%, 93%, 94%, 95%, 96%, 97%, 98%, 99%, or 100%) sequence identity to SEQ ID NO: 494. In some embodiments, the activin B antibody or an antigen binding fragment thereof has a LCVR having at least 90% (e.g., at least 91%, 92%, 93%, 94%, 95%, 96%, 97%, 98%, 99%, or 100%) sequence identity to SEQ ID NO: 495.

(SEQ ID NO: 494)
MKCSWIMFFLVATATGVHSQVQLQQPGAELVKPGASVKLSCKASG
YTFTNYWMYWVKQRPGQGLEWIGMIHPNSGSTNYNGKFKTGATLT
VDKSSSTVYMQLSSLTSEDSAVYYCARWGYGGNYDYAMDYWGQGT
SVTVSSAKTTPPSVYPLAPGSL
(SEQ ID NO: 495)
MDFQVQIFSFLLISASVIMSRGQIVLTQSPAIMSASLGERVTMTC
TASSSVSSSYFHWYQQKPGSSPKLWIYSTSNLASGVPARFSGSGS
GTSYSLTISTMEAEDAVTYYCHQYHRSPWTFGGGTKLEIKRADAA
PTVSIFPPSSEQLTSGGASVVCFLNNFYPK

In some embodiments, the ActRII signaling inhibitor is a GDF-11 antibody or an antigen binding fragment thereof.

In some embodiments, the ActRII signaling inhibitor is ActRII antibody or an antigen binding fragment thereof. There exist two types of activin type II receptors: ActRIIA and ActRIIB. In some embodiments, the ActRII antibody is an ActRIIA antibody or an antigen binding fragment thereof. In some embodiments, the ActRII antibody is an ActRIIB antibody or an antigen binding fragment thereof. In some embodiments, the ActRII antibody or an antigen binding fragment thereof binds to both ActRIIA and ActRIIB. In some embodiments, the ActRII antibody is Bimagrumab (also known as BYM338), CSJ089, CQ1876, or CDD861 (described in Morvan et al., PNAS 114:12448-12453 (2017)). Additional ActRII antibodies that may be used in the methods described herein include those described in International Patent Application Publication Nos. WO2010125003, WO2012064771, WO2017156488, WO2013063536, WO2018175460, WO2021044287, WO2013188448, and WO2020243448; US Patent Application No. US20180066061, US20180230221, US20180111991, US20200181271, US20210309749, and US20160200818; and U.S. Pat. Nos. 9,453,080, 10,266,598, 10,981,999, 10,266,598, 10,981,999, 10,307,455, 11,000,565, 10,982,000, 9,969,806, 9,365,651, 8,388,968, 8,551,482, 9,493,556, 8,765,385, and 9,624,301, each of which is incorporated herein by reference.

In some embodiments, the ActRII antibody or an antigen binding fragment thereof has a HCVR and a LCVR listed in Table 8 (e.g., an HCVR and an LCVR from the same row of Table 8). In some embodiments, the ActRII antibody or antigen binding fragment thereof includes a HCVR sequence having at least 90% (e.g., at least 91%, 92%, 93%, 94%, 95%, 96%, 97%, 98%, 99%, or 100%) sequence identity to a HCVR sequence in Table 8, such as any one of SEQ ID NOs: 512, 514, 516, 518, 520, 522, 524, 526, 528, 530, 532, 534, 536, 538, 583, 591, 593, 595-598, 600, 602, 603, 605, 606, 608, 610-614, 687, 689, 692, 695, and 697, and a LCVR sequence having at least 90% (e.g., at least 91%, 92%, 93%, 94%, 95%, 96%, 97%, 98%, 99%, or 100%) sequence identity to a LCVR sequence in Table 8, such as any one of SEQ ID NOs: 513, 515, 517, 519, 521, 523, 525, 527, 529, 531, 533, 535, 537, 539, 584, 592, 594, 601, 604, 607, 609, 615, 688, 690, 691, 693, 694, 696, and 698. In some embodiments, the ActRII antibody or an antigen binding fragment thereof, apart from the light chain CDR1, CDR2, and CDR3 and the heavy chain CDR1, CDR2, and CDR3, has a HCVR and LCVR sequence having at least 90% sequence identity (e.g., 90%, 91%, 92%, 93%, 94%, 95%, 95%, 97%, 98%, 99% or more sequence identity) to a HCVR and LCVR sequence listed in Table 8. In some embodiments, the ActRII antibody or an antigen binding fragment thereof has the light chain CDR1, CDR2, and CDR3 and the heavy chain CDR1, CDR2, and CDR3 sequences of an HCVR sequence and an LCVR sequence in Table 8. In some embodiments, the ActRII antibody or antigen binding fragment thereof includes an HCVR sequence and an LCVR sequence from the same row of Table 8.

TABLE 8
Exemplary HCVR and LCVR sequences of ActRII antibodies

| HCVR | LCVR |
|---|---|
| QVQLVQSGAEVKKPGASVKVSCKASGYTFTSS | DIALTQPASVSGSPGQSITISCTGTSSDVGSYNY |
| YINWVRQAPGQGLEWMGTINPVSGSTSYAQKF | VNWYQQHPGKAPKLMIYGVSKRPSGVSNRFSG |
| QGRVTMTRDTSISTAYMELSSLRSEDTAVYYCA | SKSGNTASLTISGLQAEDEADYYCGTFAGGSYY |
| RGGWFDYWGQGTLVTVSS (SEQ ID NO: 512) | GVFGGGTKLTVLGQ (SEQ ID NO: 513) |
| QVQLVQSGAEVKKPGASVKVSCKASGYTFTSS | DIALTQPASVSGSPGQSITISCTGTSSDVGSYNY |
| YINWVRQAPGQGLEWMGTINPVSGNTSYAQKF | VNWYQQHPGKAPKLMIYGVSKRPSGVSNRFSG |
| QGRVTMTRDTSISTAYMELSSLRSEDTAVYYCA | SKSGNTASLTISGLQAEDEADYYCQAWTSKMA |
| RGGWFDYWGQGTLVTVSS (SEQ ID NO: 514) | GVFGGGTKLTVLGQ (SEQ ID NO: 515) |
| QVQLVQSGAEVKKPGASVKVSCKASGYTFTSS | DIALTQPASVSGSPGQSITISCTGTSSDVGSYNY |
| YINWVRQAPGQGLEWMGTINPVSGNTSYAQKF | VNWYQQHPGKAPKLMIYGVSKRPSGVSNRFSG |
| QGRVTMTRDTSISTAYMELSSLRSEDTAVYYCA | SKSGNTASLTISGLQAEDEADYYCSSYTRMGHP |
| RGGWFDYWGQGTLVTVSS (SEQ ID NO: 516) | VFGGGTKLTVLGQ (SEQ ID NO: 517) |
| QVQLVQSGAEVKKPGASVKVSCKASGYTFTSS | DIALTQPASVSGSPGQSITISCTGTSSDVGSYNY |
| YINWVRQAPGQGLEWMGTINPVSGNTSYAQKF | VNWYQQHPGKAPKLMIYGVSKRPSGVSNRFSG |
| QGRVTMTRDTSISTAYMELSSLRSEDTAVYYCA | SKSGNTASLTISGLQAEDEADYYCATYGKGVTP |
| RGGWFDYWGQGTLVTVSS (SEQ ID NO: 518) | PVFGGGTKLTVLGQ (SEQ ID NO: 519) |
| QVQLVQSGAEVKKPGASVKVSCKASGYTFTSS | DIALTQPASVSGSPGQSITISCTGTSSDVGSYNY |
| YINWVRQAPGQGLEWMGTINPVSGNTSYAQKF | VNWYQQHPGKAPKLMIYGVSKRPSGVSNRFSG |
| QGRVTMTRDTSISTAYMELSSLRSEDTAVYYCA | SKSGNTASLTISGLQAEDEADYYCGTFAGGSYY |
| RGGWFDYWGQGTLVTVSS (SEQ ID NO: 520) | GVFGGGTKLTVLGQ (SEQ ID NO: 521) |
| QVQLVQSGAEVKKPGASVKVSCKASGYTFTSS | DIALTQPASVSGSPGQSITISCTGTSSDVGSYNY |
| YINWVRQAPGQGLEWMGMINAPIGTTRYAQKF | VNWYQQHPGKAPKLMIYGVSKRPSGVSNRFSG |
| QGRVTMTRDTSISTAYMELSSLRSEDTAVYYCA | SKSGNTASLTISGLQAEDEADYYCQAWTSKMA |
| RGGWFDYWGQGTLVTVSS (SEQ ID NO: 522) | GVFGGGTKLTVLGQ (SEQ ID NO: 523) |
| QVQLVQSGAEVKKPGASVKVSCKASGYTFTSS | DIALTQPASVSGSPGQSITISCTGTSSDVGSYNY |
| YINWVRQAPGQGLEWMGQINAASGMTRYAQK | VNWYQQHPGKAPKLMIYGVSKRPSGVSNRFSG |
| FQGRVTMTRDTSISTAYMELSSLRSEDTAVYYC | SKSGNTASLTISGLQAEDEADYYCQAWTSKMA |
| ARGGWFDYWGQGTLVTVSS (SEQ ID NO: 524) | GVFGGGTKLTVLGQ (SEQ ID NO: 525) |
| QVQLVQSGAEVKKPGASVKVSCKASGYTFTSS | DIALTQPASVSGSPGQSITISCTGTSSDVGSYNY |
| YINWVRQAPGQGLEWMGMINAPIGTTRYAQKF | VNWYQQHPGKAPKLMIYGVSKRPSGVSNRFSG |
| QGRVTMTRDTSISTAYMELSSLRSEDTAVYYCA | SKSGNTASLTISGLQAEDEADYYCGTFAGGSYY |
| RGGWFDYWGQGTLVTVSS (SEQ ID NO: 526) | GVFGGGTKLTVLGQ (SEQ ID NO: 527) |
| QVQLVQSGAEVKKPGASVKVSCKASGYTFTSS | DIALTQPASVSGSPGQSITISCTGTSSDVGSYNY |
| YINWVRQAPGQGLEWMGTINPVSGNTRYAQKF | VNWYQQHPGKAPKLMIYGVSKRPSGVSNRFSG |
| QGRVTMTRDTSISTAYMELSSLRSEDTAVYYCA | SKSGNTASLTISGLQAEDEADYYCGTFAGGSYY |
| RGGWFDYWGQGTLVTVSS (SEQ ID NO: 528) | GVFGGGTKLTVLGQ (SEQ ID NO: 529) |
| QVQLVQSGAEVKKPGASVKVSCKASGYTFTSS | DIALTQPASVSGSPGQSITISCTGTSSDVGSYNY |
| YINWVRQAPGQGLEWMGQINAASGMTRYAQK | VNWYQQHPGKAPKLMIYGVSKRPSGVSNRFSG |
| FQGRVTMTRDTSISTAYMELSSLRSEDTAVYYC | SKSGNTASLTISGLQAEDEADYYCGTFAGGSYY |
| ARGGWFDYWGQGTLVTVSS (SEQ ID NO: 530) | GVFGGGTKLTVLGQ (SEQ ID NO: 531) |
| QVQLVQSGAEVKKPGASVKVSCKASGYTFTSS | DIALTQPASVSGSPGQSITISCTGTSSDVGSYNY |
| YINWVRQAPGQGLEWMGNINAAAGITLYAQKF | VNWYQQHPGKAPKLMIYGVSKRPSGVSNRFSG |
| QGRVTMTRDTSISTAYMELSSLRSEDTAVYYCA | SKSGNTASLTISGLQAEDEADYYCGTFAGGSYY |
| RGGWFDYWGQGTLVTVSS (SEQ ID NO: 532) | GVFGGGTKLTVLGQ (SEQ ID NO: 533) |
| QVQLVQSGAEVKKPGASVKVSCKASGYTFTSS | DIALTQPASVSGSPGQSITISCTGTSSDVGSYNY |
| YINWVRQAPGQGLEWMGTINPPTGGTYYAQKF | VNWYQQHPGKAPKLMIYGVSKRPSGVSNRFSG |
| QGRVTMTRDTSISTAYMELSSLRSEDTAVYYCA | SKSGNTASLTISGLQAEDEADYYCGTFAGGSYY |
| RGGWFDYWGQGTLVTVSS (SEQ ID NO: 534) | GVFGGGTKLTVLGQ (SEQ ID NO: 535) |
| QVQLVQSGAEVKKPGASVKVSCKASGYTFTSS | DIALTQPASVSGSPGQSITISCTGTSSDVGSYNY |
| YINWVRQAPGQGLEWMGGINPPAGTTSYAQKF | VNWYQQHPGKAPKLMIYGVSKRPSGVSNRFSG |
| QGRVTMTRDTSISTAYMELSSLRSEDTAVYYCA | SKSGNTASLTISGLQAEDEADYYCGTFAGGSYY |
| RGGWFDYWGQGTLVTVSS (SEQ ID NO: 536) | GVFGGGTKLTVLGQ (SEQ ID NO: 537) |
| QVQLVQSGAEVKKPGASVKVSCKASGYTFTSS | DIALTQPASVSGSPGQSITISCTGTSSDVGSYNY |
| YINWVRQAPGQGLEWMGNINPATGHADYAQKF | VNWYQQHPGKAPKLMIYGVSKRPSGVSNRFSG |
| QGRVTMTRDTSISTAYMELSSLRSEDTAVYYCA | SKSGNTASLTISGLQAEDEADYYCGTFAGGSYY |
| RGGWFDYWGQGTLVTVSS (SEQ ID NO: 538) | GVFGGGTKLTVLGQ (SEQ ID NO: 539) |
| DVQLQESGPGLVKPSQSLSLTCSVTGYSITSGY | DIQMTQTTSSLSASLGDRVTISCRASQDISNFLN |
| YWNWIRQFPGNKLEWMGYISYDGSNNYNPSLI | WYQQKPDGTVKLLIYFTSRLHSGVPSRFSGSGS |
| NRISITRDTSKNQFFLKLNSVTTEDTATYFCASY | GTDYSLTITNLEQEDIATYFCQQGNTLPWTFGG |
| AYRNDVRFAYWGQGTLVTVSA (SEQ ID NO: | GTKLEIK (SEQ ID NO: 584) |
| 583) | |
| QVQLVQSGAEVKKPGASVKVSCKASGYTFTSY | DIQMTQSPSSVSASVGDRVTITCRASQGISRWL |
| RMHWVRQAPGQGLEWMGFIVPSGGSTGYAQK | AWYQQKPGKAPKLLIYAASSLQSGVPSRFSGS |
| FQGRVTMTRDTSTSTVYMELSSLRSEDTAVYYC | GSGTDFTLTISSLQPEDFATYYCQQAFSHPWTF |
| ARVSRYAPEPMDVWGQGTTVTVSS (SEQ ID | GGGTKVEIK (SEQ ID NO: 592) |
| NO: 591) | |
| QLQLQESGPGLVKPSETLSLTCTVSGGSISSSS | EIVLTQSPATLSVSPGERATLSCRASQSVGSNL |
| YAWGWIRQPPGKGLEWIGSIYYSGSTYYNPSLK | AWYQQKPGQAPRLLIYGASTRATGIPARFSGSG |
| SRVTISVDTSKNQFSLKLSSVTAADTAVYYCARD | SGTEFTLTISSLQSEDFAVYYCQQYFHFPLTFGG |
| SGIGYSYASSHGYYYYMDVWGKGTTVTVSS | GTKVEIK (SEQ ID NO: 594) |
| (SEQ ID NO: 593) | |
| QVQLQESGPGLVKPSQTLSLTCTVSGGSIGSGG | EIVLTQSPATLSVSPGERATLSCRASQSVGSNL |
| YYWSWIRQHPGKGLEWIGGIYGSGSTYYNPSL | AWYQQKPGQAPRLLIYGASTRATGIPARFSGSG |
| KSRVTISVDTSKNQFSLKLSSVTAADTAVYYCAR | SGTEFTLTISSLQSEDFAVYYCQQYFHFPLTFGG |
| DSGIGYSYASSHGYYYYMDVWGKGTTVTVSS | GTKVEIK (SEQ ID NO: 594) |
| (SEQ ID NO: 595) | |
| QVQLQESGPGLVKPSQTLSLTCTVSGGSIKSGG | EIVLTQSPATLSVSPGERATLSCRASQSVGSNL |
| YYWSWIRQHPGKGLEWIGGIYPSGSTYYNPSLK | AWYQQKPGQAPRLLIYGASTRATGIPARFSGSG |
| SRVTISVDTSKNQFSLKLSSVTAADTAVYYCARD | SGTEFTLTISSLQSEDFAVYYCQQYFHFPLTFGG |
| SGIGYSYASSHGYYYYMDVWGKGTTVTVSS | GTKVEIK (SEQ ID NO: 594) |
| (SEQ ID NO: 596) | |
| QVQLQESGPGLVKPSQTLSLTCTVSGGSILSGG | EIVLTQSPATLSVSPGERATLSCRASQSVGSNL |
| YYWSWIRQHPGKGLEWIGGIYYSGKTYYNPSLK | AWYQQKPGQAPRLLIYGASTRATGIPARFSGSG |
| SRVTISVDTSKNQFSLKLSSVTAADTAVYYCARD | SGTEFTLTISSLQSEDFAVYYCQQYFHFPLTFGG |
| SGIGYSYASSHGYYYYMDVWGKGTTVTVSS | GTKVEIK (SEQ ID NO: 594) |
| (SEQ ID NO: 597) | |
| QVQLQESGPGLVKPSQTLSLTCTVSGGSISSGG | EIVLTQSPATLSVSPGERATLSCRASQSVGSNL |
| YFWSWIRQHPGKGLEWIGGIYYSGRTYYNPSLK | AWYQQKPGQAPRLLIYGASTRATGIPARFSGSG |
| SRVTISVDTSKNQFSLKLSSVTAADTAVYYCARD | SGTEFTLTISSLQSEDFAVYYCQQYFHFPLTFGG |
| SGIGYSYASSHGYYYYMDVWGKGTTVTVSS | GTKVEIK (SEQ ID NO: 594) |
| (SEQ ID NO: 598) | |
| QVQLQESGPGLVKPSQTLSLTCTVSGGSIESGG | EIVLTQSPATLSVSPGERATLSCRASQSVGSNL |
| YYWSWIRQHPGKGLEWIGGIYGSGSTYYNPSL | AWYQQKPGQAPRLLIYGASTRATGIPARFSGSG |
| KSRVTISVDTSKNQFSLKLSSVTAADTAVYYCAR | SGTEFTLTISSLQSEDFAVYYCQQYFHFPLTFGG |
| DSGIGYSYASSHGYYYYMDVWGKGTTVTVSS | GTKVEIK (SEQ ID NO: 594) |
| (SEQ ID NO: 599) | |
| QLQLQESGPGLVKPSETLSLTCTVSGGSISSSS | DIQMTQSPSSVSASVGDRVTITCRASQGISSWL |
| YAWGWIRQPPGKGLEWIGSIYYSGSTYYNPSLK | AWYQQKPGKAPKLLIYAASNLQSGVPSRFSGS |
| SRVTISVDTSKNQFSLKLSSVTAADTAVYYCARA | GSGTDFTLTISSLQPEDFATYYCQQAPDLPITFG |
| GKYRWHGMDVWGQGTTVTVSS (SEQ ID NO: | GGTKVEIK (SEQ ID NO: 601) |
| 600) | |
| QVQLQESGPGLVKPSETLSLTCAVSGYSISSGV | DIQMTQSPSSVSASVGDRVTITCRASQGISSWL |
| YWMWIRQPPGKGLEWIGSIVHSGHTYYNPSLK | AWYQQKPGKAPKLLIYAASNLQSGVPSRFSGS |
| SRVTISVDTSKNQFSLKLSSVTAADTAVYYCARA | GSGTDFTLTISSLQPEDFATYYCQQAPDLPITFG |
| GKYRWHGMDVWGQGTTVTVSS (SEQ ID NO: | GGTKVEIK (SEQ ID NO: 601) |
| 602) | |
| EVQLLESGGGLVQPGGSLRLSCAASGFTFSSY | DIQMTQSPSTLSASVGDRVTITCRASQSISSWLA |
| AMSWVRQAPGKGLEWVSGISGSGGSTYYADS | WYQQKPGKAPKLLIYDASSLESGVPSRFSGSGS |
| VKGRFTISRDNSKNTLYLQMNSLRAEDTAVYYC | GTEFTLTISSLQPDDFATYYCQQYNRHSPTFGG |
| AKDPLSLLLGYFDYWGQGALVTVSS (SEQ ID | GTKVEIK (SEQ ID NO: 604) |
| NO: 603) | |
| EVQLLESGGGLVQPGGSLRLSCAASGFTFSRY | DIQMTQSPSTLSASVGDRVTITCRASQSISSWLA |
| AMSWVRQAPGKGLEWVSGISGSGGATYYADS | WYQQKPGKAPKLLIYDASSLESGVPSRFSGSGS |
| VKGRFTISRDNSKNTLYLQMNSLRAEDTAVYYC | GTEFTLTISSLQPDDFATYYCQQYNRHSPTFGG |
| AKDPLSLLLGYFDYWGQGALVTVSS (SEQ ID | GTKVEIK (SEQ ID NO: 604) |
| NO: 605) | |
| EVQLLESGGGLVQPGGSLRLSCAASGFTFGSY | DIQMTQSPSSVSASVGDRVTITCRASQGISSWL |
| GMTWVRQAPGKGLEWVSVISGSGGGTYYADS | AWYQQKPGKAPKLLIYAASSLQSGVPSRFSGS |
| VKGRFTISRDNSKNTLYLQMNSLRAEDTAVYYC | GSGTDFTLTISSLQPEDFATYYCQQVFSYPLTFG |
| AKGPRIVGMDVWGQGTTVTVS (SEQ ID NO: | GGTKVEIK (SEQ ID NO: 607) |
| 606) | |
| QVQLVQSGAEVKKPGASVKVSCKASGYTFTSY | DIQMTQSPSSVSASVGDRVTITCRASQGISRWL |
| GISWVRQAPGQGLEWMGWISPYNGNTNYAQK | AWYQQKPGKAPKLLIYAASSLQSGVPSRFSGS |
| LQGRVTMTTDTSTSTAYMELRSLRSDDTAVYYC | GSGTDFTLTISSLQPEDFATYYCQQAFSHPWTF |
| ARVSMYAPEPMDVWGQGTTVTVSS (SEQ ID | GGGTKVEIK (SEQ ID NO: 609) |
| NO: 608) | |
| QVQLVQSGAEVKKPGASVKVSCKASGYTFTGH | DIQMTQSPSSVSASVGDRVTITCRASQGISRWL |
| KMHWVRQAPGQGLEWMGWINPASGWTNYAQ | AWYQQKPGKAPKLLIYAASSLQSGVPSRFSGS |
| KFQGRVTMTRDTSISTAYMELSRLRSDDTAVYY | GSGTDFTLTISSLQPEDFATYYCQQAFSHPWTF |
| CARVSMYAPEPMDVWGQGTTVTVSS (SEQ ID | GGGTKVEIK (SEQ ID NO: 609) |
| NO: 610) | |
| QVQLVQSGAEVKKPGASVKVSCKASGYTFTSY | DIQMTQSPSSVSASVGDRVTITCRASQGISRWL |
| NMAWVRQAPGQGLEWMGIIRPSVGSTSYAQKF | AWYQQKPGKAPKLLIYAASSLQSGVPSRFSGS |
| QGRVTMTRDTSTSTVYMELSSLRSEDTAVYYC | GSGTDFTLTISSLQPEDFATYYCQQAFSHPWTF |
| ARVSMYAPEPMDVWGQGTTVTVSS (SEQ ID | GGGTKVEIK (SEQ ID NO: 609) |
| NO: 611) | |
| QVQLVQSGAEVKKPGASVKVSCKASGYTFTSY | DIQMTQSPSSVSASVGDRVTITCRASQGISRWL |
| RMHWVRQAPGQGLEWMGFIVPSGGSTSYAQK | AWYQQKPGKAPKLLIYAASSLQSGVPSRFSGS |
| FQGRVTMTRDTSTSTVYMELSSLRSEDTAVYYC | GSGTDFTLTISSLQPEDFATYYCQQAFSHPWTF |
| ARVSMYAPEPMDVWGQGTTVTVSS (SEQ ID | GGGTKVEIK (SEQ ID NO: 609) |
| NO: 612) | |
| QVQLVQSGAEVKKPGASVKVSCKASGYTFTSY | DIQMTQSPSSVSASVGDRVTITCRASQGISRWL |
| RMHWVRQAPGQGLEWMGFIVPSGGSTGYAQK | AWYQQKPGKAPKLLIYAASSLQSGVPSRFSGS |
| FQGRVTMTRDTSTSTVYMELSSLRSEDTAVYYC | GSGTDFTLTISSLQPEDFATYYCQQAFSHPWTF |
| ARVSRYAPEPMDVWGQGTTVTVSS (SEQ ID | GGGTKVEIK (SEQ ID NO: 609) |
| NO: 613) | |
| QVQLQESGPGLVKPSQTLSLTCTVSGGSISSGS | EIVLTQSPATLSLSPGERATLSCRASQSVSSYLA |
| YYWSWIRQHPGKGLEWIGYIYYSGSTYYNPSLK | WYQQKPGQAPRLLIYDASNRATGIPARFSGSGS |
| SRVTISVDTSKNQFSLKLSSVTAADTAVYYCAR | GTDFTLTISSLEPEDFAVYYCQQYFHWPPTFGG |
| GLGMYYHVPFDIWGQGTMVTVSS (SEQ ID NO: | GTKVEIK (SEQ ID NO: 615) |
| 614) | |
| QVQLVQSGAEVKKPGASVKVSCKASGYTFTSS | QSALTQPASVSGSPGQSITISCTGTSSDVGSYN |
| YINWVRQAPGQGLEWMGTINPVSGSTSYAQKF | YVNWYQQHPGKAPKLMIYGVSKRPSGVSNRFS |
| QGRVTMTRDTSISTAYMELSRLRSDDTAVYYCA | GSKSGNTASLTISGLQAEDEADYYCGTFAGGSY |
| RGGWFDYWGQGTLVTVSS (SEQ ID NO: 687) | YGVFGGGTKLTVL (SEQ ID NO: 688) |
| QLQLQESGPGLVKPSETLSLTCTVSGGSISSSS | DIVMTQSPDSLAVSLGERATINCKSSQSVLYSSN |
| YYWGWIRQPPGKGLEWIGSISYSGSTYYNPSLK | NKNYLAWYQQKPGQPPKLLIYWASTRESGVPD |
| SRVTISVDTSKNQFSLKLSSVTAADTAVYYCARD | RFSGSGSGTDFTLTISSLQAEDVAVYYCQQYAL |
| SLRYGMDVWGQGTTVTVSS (SEQ ID NO: 689) | APPRTFGGGTKVEIK (SEQ ID NO: 690) |
| QLQLQESGPGLVKPSETLSLTCTVSGGSISSSS | EIVMTQSPATLSVSPGERATLSCRASQSVSSNL |
| YYWGWIRQPPGKGLEWIGSISYSGSTYYNPSLK | AWYQQKPGQAPRLLIYGASTRATGIPARFSGSG |
| SRVTISVDTSKNQFSLKLSSVTAADTAVYYCARD | SGTEFTLTISSLQSEDFAVYYCQVYNVWPRTFG |
| SLRYGMDVWGQGTTVTVSS (SEQ ID NO: 689) | GGTKVEIK (SEQ ID NO: 691) |
| EVQLLESGGGLVQPGGSLRLSCAASGFTFGSY | DIQMTQSPSSVSASVGDRVTITCRASQGISSWL |
| GMTWVRQAPGKGLEWVSVISGSGGGTYYADS | AWYQQKPGKAPKLLIYAASSLQSGVPSRFSGS |
| VKGRFTISRDNSKNTLYLQMNSLRAEDTAVYYC | GSGTDYTLTISSLQPEDFATYYCQQVFSYPLTF |
| AKGPRVVGMDVWGQGTTVTVSS (SEQ ID NO: | GGGTKVEIK (SEQ ID NO: 693) |
| 692) | |
| QLQLQESGPGLVKPSETLSLTCTVSGGSISSSS | DIQMTQSPSTLSASVGDRVTITCRASQSIGSWL |
| YYWGWIRQPPGKGLEWIGSISYSGSTYYNPSLK | AWYQQKPGKAPKLLIYKASSLESGVPSRFSGSG |
| SRVTISVDTSKNQFSLKLSSVTAADTAVYYCARD | SGTEFTLTISSLQPDDFATYYCQVYGSYSPRTF |
| SLRYGMDVWGQGTTVTVSS (SEQ ID NO: 689) | GGGTKVEIK (SEQ ID NO: 694) |
| EVQLLESGGGLVQPGGSLRLSCAASGFTFSSY | DIQMTQSPSSVSASVGDRVTITCRASQGISSWL |
| AMSWVRQAPGKGLEWVSAISGSGGSTSYADSV | AWYQQKPGKAPKLLIYAASSLQSGVPSRFSGS |
| KGRFTISRDNSKNTLYLQMNSLRAEDTAVYYCA | GSGTDFTLTISSLQPEDFATYYCQQGHSFPLTF |
| RLPQYSRPFDYWGQGTLVTVSS (SEQ ID NO: | GGGTKVEIK (SEQ ID NO: 696) |
| 695) | |
| QVQLVQSGAEVKKPGASVKVSCKASGYTFTSY | DIQMTQSPSSVSASVGDRVTITCRASQGISRWL |
| RMHWVRQAPGQGLEWMGFIVPSGGSTSYAQK | AWYQQKPGKAPKLLIYAASSLQSGVPSRFSGS |
| FQGRVTMTRDTSTSTVYMELSSLRSEDTAVYYC | GSGTDFTLTISSLQPEDFATYYCQQAFSHPWTF |
| ARVSRYAPEPMDVWGQGTTVTVSS (SEQ ID | GGGTKVEIK (SEQ ID NO: 698) |
| NO: 697) | |

In some embodiments, the ActRII antibody or an antigen-binding fragment thereof, has the CDR sequences described in Table 9 (i.e., a light chain CDR1, CDR2, and CDR3 and a heavy chain CDR1, CDR2, and CDR3). In some embodiments, the ActRII antibody or antigen binding fragment thereof includes a light chain variable CDR1 sequence having at least 90% (e.g., at least 91%, 92%, 93%, 94%, 95%, 96%, 97%, 98%, 99%, or 100%) sequence identity to a light chain variable CDR1 sequence in Table 9, such as any one of SEQ ID NOs: 499, 505, 543, 580, 588, 619, 625, 633, 640, 648, 654, 663, 684, 702, 705, 711, 714, 720, and 726; a light chain variable CDR2 sequence having at least 90% (e.g., at least 91%, 92%, 93%, 94%, 95%, 96%, 97%, 98%, 99%, or 100%) sequence identity to a light chain variable CDR2 sequence in Table 9, such as any one of SEQ ID NOs: 500, 506, 544, 581, 589, 620, 626, 634, 641, 649, 655, 664, 685, 703, 706, 712, 715, 721, and 727; a light chain variable CDR3 sequence having at least 90% (e.g., at least 91%, 92%, 93%, 94%, 95%, 96%, 97%, 98%, 99%, or 100%) sequence identity to a light chain variable CDR3 sequence in Table 9, such as any one of SEQ ID NOs: 501, 507, 545, 547, 548, 549, 582, 590, 621, 627, 635, 635, 642, 650, 656, 665, 686, 704, 707, 713, 716, 722, and 728; a heavy chain variable CDR1 sequence having at least 90% (e.g., at least 91%, 92%, 93%, 94%, 95%, 96%, 97%, 98%, 99%, or 100%) sequence identity to a heavy chain variable CDR1 sequence in Table 9, such as any one of SEQ ID NOs: 496, 502, 540, 577, 585, 616, 622, 629, 630, 638, 644, 651, 659, 660, 669-672, 679-681, 699, 708, 717, and 723; a heavy chain variable CDR2 sequence having at least 90% (e.g., at least 91%, 92%, 93%, 94%, 95%, 96%, 97%, 98%, 99%, or more) sequence identity to a heavy chain variable CDR2 sequence in Table 9, such as any one of SEQ ID NOs: 497, 503, 541, 546, 550-556, 578, 586, 617, 623, 628, 631, 637, 643, 646, 652, 658, 661, 666, 667, 668, 676, 677, 678, 682, 700, 709, 718, and 724; and a heavy chain variable CDR3 sequence having at least 90% (e.g., at least 91%, 92%, 93%, 94%, 95%, 96%, 97%, 98%, 99%, or more) sequence identity to a heavy chain variable CDR3 sequence in Table 9, such as any one of SEQ ID NOs: 498, 504, 542, 579, 587, 618, 624, 632, 636, 639, 647, 653, 657, 662, 673, 674, 675, 683, 701, 710, 719, and 725. In some embodiments, the ActRII antibody or antigen binding fragment thereof includes a light chain CDR1, CDR2, and CDR3 sequence and a heavy chain CDR1, CDR2, and CDR3 sequence from the same row of Table 9.

TABLE 9
Exemplary CDR sequences of ActRII antibodes

| HCDR1 | HCDR2 | HCRD3 | LCDR1 | LCDR2 | LCDR3 |
|---|---|---|---|---|---|
| NSAMS (SEQ | GISVTGGSTF | VYYYSFFDY | GFNSGSVST | NTNTRSS | VLWMGSGIW |
| ID NO: 496) | YTDSVKGR | (SEQ ID NO: | SYWPS (SEQ | (SEQ ID NO: | V (SEQ ID |
| | (SEQ ID NO: | 498) | ID NO: 499) | 500) | NO: 501) |
| | 497) | | | | |
| NYAMS (SEQ | GISVTGGSTY | VYYSSFFDY | GFNSGSVST | NTNTRSS | VLYMGSGIW |
| ID NO: 502) | YTDSVKGR | (SEQ ID NO: | SYYPS (SEQ | (SEQ ID NO: | V (SEQ ID |
| | (SEQ ID NO: | 504) | ID NO: 505) | 506) | NO: 507) |
| | 503) | | | | |
| GYTFTSSYIN | TINPVSGSTS | GGWFDY | TGTSSDVGS | LMIYGVSKRP | GTFAGGSYY |
| (SEQ ID NO: | YAQKFQG | (SEQ ID NO: | YNYVN (SEQ | S (SEQ ID | G (SEQ ID |
| 540) | (SEQ ID NO: | 542) | ID NO: 543) | NO: 544) | NO: 545) |
| | 541) | | | | |
| GYTFTSSYIN | TINPVSGNTS | GGWFDY | TGTSSDVGS | LMIYGVSKRP | QAWTSKMAG |
| (SEQ ID NO: | YAQKFQG | (SEQ ID NO: | YNYVN (SEQ | S (SEQ ID | (SEQ ID NO: |
| 540) | (SEQ ID NO: | 542) | ID NO: 543) | NO: 544) | 547) |
| | 546) | | | | |
| GYTFTSSYIN | TINPVSGNTS | GGWFDY | TGTSSDVGS | LMIYGVSKRP | SSYTRMGHP |
| (SEQ ID NO: | YAQKFQG | (SEQ ID NO: | YNYVN (SEQ | S (SEQ ID | (SEQ ID NO: |
| 540) | (SEQ ID NO: | 542) | ID NO: 543) | NO: 544) | 548) |
| | 546) | | | | |
| GYTFTSSYIN | TINPVSGNTS | GGWFDY | TGTSSDVGS | LMIYGVSKRP | ATYGKGVTP |
| (SEQ ID NO: | YAQKFQG | (SEQ ID NO: | YNYVN (SEQ | S (SEQ ID | P (SEQ ID |
| 540) | (SEQ ID NO: | 542) | ID NO: 543) | NO: 544) | NO: 549) |
| | 546) | | | | |
| GYTFTSSYIN | TINPVSGNTS | GGWFDY | TGTSSDVGS | LMIYGVSKRP | GTFAGGSYY |
| (SEQ ID NO: | YAQKFQG | (SEQ ID NO: | YNYVN (SEQ | S (SEQ ID | G (SEQ ID |
| 540) | (SEQ ID NO: | 542) | ID NO: 543) | NO: 544) | NO: 545) |
| | 546) | | | | |
| GYTFTSSYIN | MINAPIGTTR | GGWFDY | TGTSSDVGS | LMIYGVSKRP | QAWTSKMAG |
| (SEQ ID NO: | YAQKFQG | (SEQ ID NO: | YNYVN (SEQ | S (SEQ ID | (SEQ ID NO: |
| 540) | (SEQ ID NO: | 542) | ID NO: 543) | NO: 544) | 547) |
| | 550) | | | | |
| GYTFTSSYIN | QINAASGMT | GGWFDY | TGTSSDVGS | LMIYGVSKRP | QAWTSKMAG |
| (SEQ ID NO: | RYAQKFQG | (SEQ ID NO: | YNYVN (SEQ | S (SEQ ID | (SEQ ID NO: |
| 540) | (SEQ ID NO: | 542) | ID NO: 543) | NO: 544) | 547) |
| | 551) | | | | |
| GYTFTSSYIN | MINAPIGTTR | GGWFDY | TGTSSDVGS | LMIYGVSKRP | GTFAGGSYY |
| (SEQ ID NO: | YAQKFQG | (SEQ ID NO: | YNYVN (SEQ | S (SEQ ID | G (SEQ ID |
| 540) | (SEQ ID NO: | 542) | ID NO: 543) | NO: 544) | NO: 545) |
| | 550) | | | | |
| GYTFTSSYIN | TINPVSGNTR | GGWFDY | TGTSSDVGS | LMIYGVSKRP | GTFAGGSYY |
| (SEQ ID NO: | YAQKFQG | (SEQ ID NO: | YNYVN (SEQ | S (SEQ ID | G (SEQ ID |
| 540) | (SEQ ID NO: | 542) | ID NO: 543) | NO: 544) | NO: 545) |
| | 552) | | | | |
| GYTFTSSYIN | QINAASGMT | GGWFDY | TGTSSDVGS | LMIYGVSKRP | GTFAGGSYY |
| (SEQ ID NO: | RYAQKFQG | (SEQ ID NO: | YNYVN (SEQ | S (SEQ ID | G (SEQ ID |
| 540) | (SEQ ID NO: | 542) | ID NO: 543) | NO: 544) | NO: 545) |
| | 551) | | | | |
| GYTFTSSYIN | NINAAAGITLY | GGWFDY | TGTSSDVGS | LMIYGVSKRP | GTFAGGSYY |
| (SEQ ID NO: | AQKFQG | (SEQ ID NO: | YNYVN (SEQ | S (SEQ ID | G (SEQ ID |
| 540) | (SEQ ID NO: | 542) | ID NO: 543) | NO: 544) | NO: 545) |
| | 553) | | | | |
| GYTFTSSYIN | TINPPTGGTY | GGWFDY | TGTSSDVGS | LMIYGVSKRP | GTFAGGSYY |
| (SEQ ID NO: | YAQKFQG | (SEQ ID NO: | YNYVN (SEQ | S (SEQ ID | G (SEQ ID |
| 540) | (SEQ ID NO: | 542) | ID NO: 543) | NO: 544) | NO: 545) |
| | 554) | | | | |
| GYTFTSSYIN | GINPPAGTTS | GGWFDY | TGTSSDVGS | LMIYGVSKRP | GTFAGGSYY |
| (SEQ ID NO: | YAQKFQG | (SEQ ID NO: | YNYVN (SEQ | S (SEQ ID | G (SEQ ID |
| 540) | (SEQ ID NO: | 542) | ID NO: 543) | NO: 544) | NO: 545) |
| | 555) | | | | |
| GYTFTSSYIN | NINPATGHAD | GGWFDY | TGTSSDVGS | LMIYGVSKRP | GTFAGGSYY |
| (SEQ ID NO: | YAQKFQG | (SEQ ID NO: | YNYVN (SEQ | S (SEQ ID | G (SEQ ID |
| 540) | (SEQ ID NO: | 542) | ID NO: 543) | NO: 544) | NO: 545) |
| | 556) | | | | |
| NYWMG (SEQ | YIRSSGGLTH | GLYSFDY | RASQGVNNF | AASTLQS | QQSYSTPRG |
| ID NO: 577) | YADSVKG | (SEQ ID NO: | LA (SEQ ID | (SEQ ID NO: | (SEQ ID NO: |
| | (SEQ ID NO: | 579) | NO: 580) | 581) | 582) |
| | 578) | | | | |
| YSITSGYYWN | YISYDGSNNY | YAYRNDVRF | RASQDISNFL | FTSRLHS | QQGNTLPWT |
| (SEQ ID NO: | NPSLIN (SEQ | AY (SEQ ID | N (SEQ ID | (SEQ ID NO: | (SEQ ID NO: |
| 585) | ID NO: 586) | NO: 587) | NO: 588) | 589) | 590) |
| SYRMH (SEQ | FIVPSGGSTG | VSRYAPEPM | RASQGISRW | AASSLQS | QQAFSHPWT |
| ID NO: 616) | YAQKFQG | DV (SEQ ID | LA (SEQ ID | (SEQ ID NO: | (SEQ ID NO: |
| | (SEQ ID NO: | NO: 618) | NO: 619) | 620) | 621) |
| | 617) | | | | |
| GGSISSSSY | YYSGS (SEQ | DSGIGYSYAS | RASQSVGSN | GASTRAT | QQYFHFPLT |
| (SEQ ID NO: | ID NO: 623) | SHGYYYYMD | LA (SEQ ID | (SEQ ID NO: | (SEQ ID NO: |
| 622) | | V (SEQ ID | NO: 625) | 626) | 627) |
| | | NO: 624) | | | |
| GGSISSSSY | YGSG (SEQ | DSGIGYSYAS | RASQSVGSN | GASTRAT | QQYFHFPLT |
| (SEQ ID NO: | ID NO: 628) | SHGYYYYMD | LA (SEQ ID | (SEQ ID NO: | (SEQ ID NO: |
| 622) | | V (SEQ ID | NO: 625) | 626) | 627) |
| | | NO: 624) | | | |
| GGSIGSGGY | YYSGS (SEQ | DSGIGYSYAS | RASQSVGSN | GASTRAT | QQYFHFPLT |
| (SEQ ID NO: | ID NO: 623) | SHGYYYYMD | LA (SEQ ID | (SEQ ID NO: | (SEQ ID NO: |
| 629) | | V (SEQ ID | NO: 625) | 626) | 627) |
| | | NO: 624) | | | |
| GGSIGSGGY | YGSG (SEQ | DSGIGYSYAS | RASQSVGSN | GASTRAT | QQYFHFPLT |
| (SEQ ID NO: | ID NO: 628) | SHGYYYYMD | LA (SEQ ID | (SEQ ID NO: | (SEQ ID NO: |
| 629) | | V (SEQ ID | NO: 625) | 626) | 627) |
| | | NO: 624) | | | |
| GFTFSSY | SGSGGS | DPLSLLLGYF | RASQSISSWL | DASSLES | QQYNRHSPT |
| (SEQ ID NO: | (SEQ ID NO: | DY (SEQ ID | A (SEQ ID | (SEQ ID NO: | (SEQ ID NO: |
| 630) | 631) | NO: 632) | NO: 633) | 634) | 635) |
| GFTFSSY | SGSGGS | DPLSLLLGYF | RASQSISSWL | DASSLES | QQYNRHSPT |
| (SEQ ID NO: | (SEQ ID NO: | DY (SEQ ID | A (SEQ ID | (SEQ ID NO: | (SEQ ID NO: |
| 630) | 631) | NO: 636) | NO: 633) | 634) | 635) |
| GFTFSSY | SGSGGA | DPLSLLLGYF | RASQSISSWL | DASSLES | QQYNRHSPT |
| (SEQ ID NO: | (SEQ ID NO: | DY (SEQ ID | A (SEQ ID | (SEQ ID NO: | (SEQ ID NO: |
| 630) | 637) | NO: 632) | NO: 633) | 634) | 635) |
| GFTFSSY | SGSGGA | DPLSLLLGYF | RASQSISSWL | DASSLES | QQYNRHSPT |
| (SEQ ID NO: | (SEQ ID NO: | DY (SEQ ID | A (SEQ ID | (SEQ ID NO: | (SEQ ID NO: |
| 630) | 637) | NO: 636) | NO: 633) | 634) | 635) |
| GFTFSRY | SGSGGS | DPLSLLLGYF | RASQSISSWL | DASSLES | QQYNRHSPT |
| (SEQ ID NO: | (SEQ ID NO: | DY (SEQ ID | A (SEQ ID | (SEQ ID NO: | (SEQ ID NO: |
| 638) | 631) | NO: 632) | NO: 633) | 634) | 635) |
| GFTFSRY | SGSGGS | DPLSLLLGYF | RASQSISSWL | DASSLES | QQYNRHSPT |
| (SEQ ID NO: | (SEQ ID NO: | DY (SEQ ID | A (SEQ ID | (SEQ ID NO: | (SEQ ID NO: |
| 638) | 631) | NO: 636) | NO: 633) | 634) | 635) |
| GFTFSRY | SGSGGA | DPLSLLLGYF | RASQSISSWL | DASSLES | QQYNRHSPT |
| (SEQ ID NO: | (SEQ ID NO: | DY (SEQ ID | A (SEQ ID | (SEQ ID NO: | (SEQ ID NO: |
| 638) | 637) | NO: 632) | NO: 633) | 634) | 635) |
| GFTFSRY | SGSGGA | DPLSLLLGYF | RASQSISSWL | DASSLES | QQYNRHSPT |
| (SEQ ID NO: | (SEQ ID NO: | DY (SEQ ID | A (SEQ ID | (SEQ ID NO: | (SEQ ID NO: |
| 638) | 637) | NO: 636) | NO: 633) | 634) | 635) |
| GGSISSSSY | YYSGS (SEQ | AGKYRWHG | RASQGISSW | AASNLQS | QQAPDLPIT |
| (SEQ ID NO: | ID NO: 623) | MDV (SEQ ID | LA (SEQ ID | (SEQ ID NO: | (SEQ ID NO: |
| 622) | | NO: 639) | NO: 640) | 641) | 642) |
| GGSISSSSY | VHSGH (SEQ | AGKYRWHG | RASQGISSW | AASNLQS | QQAPDLPIT |
| (SEQ ID NO: | ID NO: 643) | MDV (SEQ ID | LA (SEQ ID | (SEQ ID NO: | (SEQ ID NO: |
| 622) | | NO: 639) | NO: 640) | 641) | 642) |
| GYSISSGV | YYSGS (SEQ | AGKYRWHG | RASQGISSW | AASNLQS | QQAPDLPIT |
| (SEQ ID NO: | ID NO: 623) | MDV (SEQ ID | LA (SEQ ID | (SEQ ID NO: | (SEQ ID NO: |
| 644) | | NO: 639) | NO: 640) | 641) | 642) |
| GYSISSGV | VHSGH (SEQ | AGKYRWHG | RASQGISSW | AASNLQS | QQAPDLPIT |
| (SEQ ID NO: | ID NO: 643) | MDV (SEQ ID | LA (SEQ ID | (SEQ ID NO: | (SEQ ID NO: |
| 644) | | NO: 639) | NO: 640) | 641) | 642) |
| SYGMT (SEQ | VISGSGGGTY | GPRIVGMDV | RASQGISSW | AASSLQS | QQVFSYPLT |
| ID NO: 645) | YADSVKG | (SEQ ID NO: | LA (SEQ ID | (SEQ ID NO: | (SEQ ID NO: |
| | (SEQ ID NO: | 647) | NO: 648) | 649) | 650) |
| | 646) | | | | |
| GYTFTSY | SPYNGN | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 652) | NO: 653) | NO: 654) | 655) | 656) |
| GYTFTSY | SPYNGN | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 652) | NO: 657) | NO: 654) | 655) | 656) |
| GYTFTSY | NPASGW | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 658) | NO: 653) | NO: 654) | 655) | 656) |
| GYTFTSY | NPASGW | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 658) | NO: 657) | NO: 654) | 655) | 656) |
| GYTFTGHKM | SPYNGN | VSMYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO: 659) | 652) | NO: 653) | NO: 654) | 655) | 656) |
| GYTFTGHKM | SPYNGN | VSMYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO: 659) | 652) | NO: 657) | NO: 654) | 655) | 656) |
| GYTFTGHKM | NPASGW | VSMYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO: 659) | 658) | NO: 653) | NO: 654) | 655) | 656) |
| GYTFTGHKM | NPASGW | VSMYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO: 659) | 658) | NO: 657) | NO: 654) | 655) | 656) |
| GGSISSGSY | YYSGS (SEQ | GLGMYYHVP | RASQSVSSY | DASNRAT | QQYFHWPPT |
| (SEQ ID NO: | ID NO: 661) | FDI (SEQ ID | LA (SEQ ID | (SEQ ID NO: | (SEQ ID NO: |
| 660) | | NO: 662) | NO: 663) | 664) | 665) |
| GGSISSSSY | WIGGIYPSGS | DSGIGYSYAS | RASQSVGSN | GASTRAT | QQYFHFPLT |
| (SEQ ID NO: | TYY (SEQ ID | SHGYYYYMD | LA (SEQ ID | (SEQ ID NO: | (SEQ ID NO: |
| 622) | NO: 666) | V (SEQ ID | NO: 625) | 626) | 627) |
| | | NO: 624) | | | |
| GGSISSSSY | YYSGK (SEQ | DSGIGYSYAS | RASQSVGSN | GASTRAT | QQYFHFPLT |
| (SEQ ID NO: | ID NO: 667) | SHGYYYYMD | LA (SEQ ID | (SEQ ID NO: | (SEQ ID NO: |
| 622) | | V (SEQ ID | NO: 625) | 626) | 627) |
| | | NO: 624) | | | |
| GGSIGSGGY | WIGGIYPSGS | DSGIGYSYAS | RASQSVGSN | GASTRAT | QQYFHFPLT |
| (SEQ ID NO: | TYY (SEQ ID | SHGYYYYMD | LA (SEQ ID | (SEQ ID NO: | (SEQ ID NO: |
| 629) | NO: 666) | V (SEQ ID | NO: 625) | 626) | 627) |
| | | NO: 624) | | | |
| GGSIGSGGY | YYSGK (SEQ | DSGIGYSYAS | RASQSVGSN | GASTRAT | QQYFHFPLT |
| (SEQ ID NO: | ID NO: 667) | SHGYYYYMD | LA (SEQ ID | (SEQ ID NO: | (SEQ ID NO: |
| 629) | | V (SEQ ID | NO: 625) | 626) | 627) |
| | | NO: 624) | | | |
| GGSISSSSY | YYSGRT | DSGIGYSYAS | RASQSVGSN | GASTRAT | QQYFHFPLT |
| (SEQ ID NO: | (SEQ ID NO: | SHGYYYYMD | LA (SEQ ID | (SEQ ID NO: | (SEQ ID NO: |
| 622) | 668) | V (SEQ ID | NO: 625) | 626) | 627) |
| | | NO: 624) | | | |
| GGSIGSGGY | YYSGRT | DSGIGYSYAS | RASQSVGSN | GASTRAT | QQYFHFPLT |
| (SEQ ID NO: | (SEQ ID NO: | SHGYYYYMD | LA (SEQ ID | (SEQ ID NO: | (SEQ ID NO: |
| 629) | 668) | V (SEQ ID | NO: 625) | 626) | 627) |
| | | NO: 624) | | | |
| GGSIKSGGY | YYSGS (SEQ | DSGIGYSYAS | RASQSVGSN | GASTRAT | QQYFHFPLT |
| (SEQ ID NO: | ID NO: 623) | SHGYYYYMD | LA (SEQ ID | (SEQ ID NO: | (SEQ ID NO: |
| 669) | | V (SEQ ID | NO: 625) | 626) | 627) |
| | | NO: 624) | | | |
| GGSIKSGGY | YGSG (SEQ | DSGIGYSYAS | RASQSVGSN | GASTRAT | QQYFHFPLT |
| (SEQ ID NO: | ID NO: 628) | SHGYYYYMD | LA (SEQ ID | (SEQ ID NO: | (SEQ ID NO: |
| 669) | | V (SEQ ID | NO: 625) | 626) | 627) |
| | | NO: 624) | | | |
| GGSILSGGY | YYSGS (SEQ | DSGIGYSYAS | RASQSVGSN | GASTRAT | QQYFHFPLT |
| (SEQ ID NO: | ID NO: 623) | SHGYYYYMD | LA (SEQ ID | (SEQ ID NO: | |
| 670) | | V (SEQ ID | NO: 625) | 626) | (SEQ ID NO: |
| | | NO: 624) | | | 627) |
| GGSILSGGY | YGSG (SEQ | DSGIGYSYAS | RASQSVGSN | GASTRAT | QQYFHFPLT |
| (SEQ ID NO: | ID NO: 628) | SHGYYYYMD | LA (SEQ ID | (SEQ ID NO: | (SEQ ID NO: |
| 670) | | V (SEQ ID | NO: 625) | 626) | 627) |
| | | NO: 624) | | | |
| GGSIKSGGY | WIGGIYPSGS | DSGIGYSYAS | RASQSVGSN | GASTRAT | QQYFHFPLT |
| (SEQ ID NO: | TYY (SEQ ID | SHGYYYYMD | LA (SEQ ID | (SEQ ID NO: | (SEQ ID NO: |
| 669) | NO: 666) | V (SEQ ID | NO: 625) | 626) | 627) |
| | | NO: 624) | | | |
| GGSIKSGGY | YYSGK (SEQ | DSGIGYSYAS | RASQSVGSN | GASTRAT | QQYFHFPLT |
| (SEQ ID NO: | ID NO: 667) | SHGYYYYMD | LA (SEQ ID | (SEQ ID NO: | (SEQ ID NO: |
| 669) | | V (SEQ ID | NO: 625) | 626) | 627) |
| | | NO: 624) | | | |
| GGSILSGGY | WIGGIYPSGS | DSGIGYSYAS | RASQSVGSN | GASTRAT | QQYFHFPLT |
| (SEQ ID NO: | TYY (SEQ ID | SHGYYYYMD | LA (SEQ ID | (SEQ ID NO: | (SEQ ID NO: |
| 670) | NO: 666) | V (SEQ ID | NO: 625) | 626) | 627) |
| | | NO: 624) | | | |
| GGSILSGGY | YYSGK (SEQ | DSGIGYSYAS | RASQSVGSN | GASTRAT | QQYFHFPLT |
| (SEQ ID NO: | ID NO: 667) | SHGYYYYMD | LA (SEQ ID | (SEQ ID NO: | (SEQ ID NO: |
| 670) | | V (SEQ ID | NO: 625) | 626) | 627) |
| | | NO: 624) | | | |
| GGSIKSGGY | YYSGRT | DSGIGYSYAS | RASQSVGSN | GASTRAT | QQYFHFPLT |
| (SEQ ID NO: | (SEQ ID NO: | SHGYYYYMD | LA (SEQ ID | (SEQ ID NO: | (SEQ ID NO: |
| 669) | 668) | V (SEQ ID | NO: 625) | 626) | 627) |
| | | NO: 624) | | | |
| GGSIKSGGY | YYSGRT | DSGIGYSYAS | RASQSVGSN | GASTRAT | QQYFHFPLT |
| (SEQ ID NO: | (SEQ ID NO: | SHGYYYYMD | LA (SEQ ID | (SEQ ID NO: | (SEQ ID NO: |
| 669) | 668) | V (SEQ ID | NO: 625) | 626) | 627) |
| | | NO: 624) | | | |
| GGSISSGGY | YYSGS (SEQ | DSGIGYSYAS | RASQSVGSN | GASTRAT | QQYFHFPLT |
| (SEQ ID NO: | ID NO: 623) | SHGYYYYMD | LA (SEQ ID | (SEQ ID NO: | (SEQ ID NO: |
| 671) | | V (SEQ ID | NO: 625) | 626) | 627) |
| | | NO: 624) | | | |
| GGSISSGGY | YGSG (SEQ | DSGIGYSYAS | RASQSVGSN | GASTRAT | QQYFHFPLT |
| (SEQ ID NO: | ID NO: 628) | SHGYYYYMD | LA (SEQ ID | (SEQ ID NO: | (SEQ ID NO: |
| 671) | | V (SEQ ID | NO: 625) | 626) | 627) |
| | | NO: 624) | | | |
| GGSIESGGY | YYSGS (SEQ | DSGIGYSYAS | RASQSVGSN | GASTRAT | QQYFHFPLT |
| (SEQ ID NO: | ID NO: 623) | SHGYYYYMD | LA (SEQ ID | (SEQ ID NO: | (SEQ ID NO: |
| 672) | | V (SEQ ID | NO: 625) | 626) | 627) |
| | | NO: 624) | | | |
| GGSIESGGY | YGSG (SEQ | DSGIGYSYAS | RASQSVGSN | GASTRAT | QQYFHFPLT |
| (SEQ ID NO: | ID NO: 628) | SHGYYYYMD | LA (SEQ ID | (SEQ ID NO: | (SEQ ID NO: |
| 672) | | V (SEQ ID | NO: 625) | 626) | 627) |
| | | NO: 624) | | | |
| GGSISSGGY | WIGGIYPSGS | DSGIGYSYAS | RASQSVGSN | GASTRAT | QQYFHFPLT |
| (SEQ ID NO: | TYY (SEQ ID | SHGYYYYMD | LA (SEQ ID | (SEQ ID NO: | (SEQ ID NO: |
| 671) | NO: 666) | V (SEQ ID | NO: 625) | 626) | 627) |
| | | NO: 624) | | | |
| GGSISSGGY | YYSGK (SEQ | DSGIGYSYAS | RASQSVGSN | GASTRAT | QQYFHFPLT |
| (SEQ ID NO: | ID NO: 667) | SHGYYYYMD | LA (SEQ ID | (SEQ ID NO: | (SEQ ID NO: |
| 671) | | V (SEQ ID | NO: 625) | 626) | 627) |
| | | NO: 624) | | | |
| GGSIESGGY | WIGGIYPSGS | DSGIGYSYAS | RASQSVGSN | GASTRAT | QQYFHFPLT |
| (SEQ ID NO: | TYY (SEQ ID | SHGYYYYMD | LA (SEQ ID | (SEQ ID NO: | (SEQ ID NO: |
| 672) | NO: 666) | V (SEQ ID | NO: 625) | 626) | 627) |
| | | NO: 624) | | | |
| GGSIESGGY | YYSGK (SEQ | DSGIGYSYAS | RASQSVGSN | GASTRAT | QQYFHFPLT |
| (SEQ ID NO: | ID NO: 667) | SHGYYYYMD | LA (SEQ ID | (SEQ ID NO: | (SEQ ID NO: |
| 672) | | V (SEQ ID | NO: 625) | 626) | 627) |
| | | NO: 624) | | | |
| GGSISSGGY | YYSGRT | DSGIGYSYAS | RASQSVGSN | GASTRAT | QQYFHFPLT |
| (SEQ ID NO: | (SEQ ID NO: | SHGYYYYMD | LA (SEQ ID | (SEQ ID NO: | (SEQ ID NO: |
| 671) | 668) | V (SEQ ID | NO: 625) | 626) | 627) |
| | | NO: 624) | | | |
| GGSIESGGY | YYSGRT | DSGIGYSYAS | RASQSVGSN | GASTRAT | QQYFHFPLT |
| (SEQ ID NO: | (SEQ ID NO: | SHGYYYYMD | LA (SEQ ID | (SEQ ID NO: | (SEQ ID NO: |
| 672) | 668) | V (SEQ ID | NO: 625) | 626) | 627) |
| | | NO: 624) | | | |
| GYTFTSY | SPYNGN | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 652) | NO: 673) | NO: 654) | 655) | 656) |
| GYTFTSY | SPYNGN | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 652) | NO: 674) | NO: 654) | 655) | 656) |
| GYTFTSY | NPASGW | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 658) | NO: 673) | NO: 654) | 655) | 656) |
| GYTFTSY | NPASGW | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 658) | NO: 674) | NO: 654) | 655) | 656) |
| GYTFTGHKM | SPYNGN | VSMYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO: 659) | 652) | NO: 673) | NO: 654) | 655) | 656) |
| GYTFTGHKM | SPYNGN | VSMYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO: 659) | 652) | NO: 674) | NO: 654) | 655) | 656) |
| GYTFTGHKM | NPASGW | VSMYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO: 659) | 658) | NO: 673) | NO: 654) | 655) | 656) |
| GYTFTGHKM | NPASGW | VSMYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO: 659) | 658) | NO: 674) | NO: 654) | 655) | 656) |
| GYTFTSY | SPYNGN | VSRYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 652) | NO: 675) | NO: 654) | 655) | 656) |
| GYTFTSY | NPASGW | VSRYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 658) | NO: 675) | NO: 654) | 655) | 656) |
| GYTFTGHKM | SPYNGN | VSRYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO: 659) | 652) | NO: 675) | NO: 654) | 655) | 656) |
| GYTFTGHKM | NPASGW | VSRYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO: 659) | 658) | NO: 675) | NO: 654) | 655) | 656) |
| GYTFTSY | RPSVGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 676) | NO: 653) | NO: 654) | 655) | 656) |
| GYTFTSY | RPSVGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 676) | NO: 657) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 677) | NO: 653) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 677) | NO: 657) | NO: 654) | 655) | 656) |
| GYTFTGHKM | RPSVGS | VSMYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO: 659) | 676) | NO: 653) | NO: 654) | 655) | 656) |
| GYTFTGHKM | RPSVGS | VSMYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO: 659) | 676) | NO: 657) | NO: 654) | 655) | 656) |
| GYTFTGHKM | VPSGGS | VSMYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO: 659) | 677) | NO: 653) | NO: 654) | 655) | 656) |
| GYTFTGHKM | VPSGGS | VSMYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO: 659) | 677) | NO: 657) | NO: 654) | 655) | 656) |
| GYTFTSY | RPSVGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 676) | NO: 673) | NO: 654) | 655) | 656) |
| GYTFTSY | RPSVGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 676) | NO: 674) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 677) | NO: 673) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 677) | NO: 674) | NO: 654) | 655) | 656) |
| GYTFTGHKM | RPSVGS | VSMYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO: 659) | 676) | NO: 673) | NO: 654) | 655) | 656) |
| GYTFTGHKM | RPSVGS | VSMYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO: 659) | 676) | NO: 674) | NO: 654) | 655) | 656) |
| GYTFTGHKM | VPSGGS | VSMYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO: 659) | 677) | NO: 673) | NO: 654) | 655) | 656) |
| GYTFTGHKM | VPSGGS | VSMYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO: 659) | 677) | NO: 674) | NO: 654) | 655) | 656) |
| GYTFTSY | RPSVGS | VSRYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 676) | NO: 675) | NO: 654) | 655) | 656) |
| GYTFTSY | VPSGGS | VSRYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 677) | NO: 675) | NO: 654) | 655) | 656) |
| GYTFTGHKM | RPSVGS | VSRYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO: 659) | 676) | NO: 675) | NO: 654) | 655) | 656) |
| GYTFTGHKM | VPSGGS | VSRYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO: 659) | 677) | NO: 675) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 678) | NO: 653) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 678) | NO: 657) | NO: 654) | 655) | 656) |
| GYTFTGHKM | VPSGGS | VSMYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO: 659) | 678) | NO: 653) | NO: 654) | 655) | 656) |
| GYTFTGHKM | VPSGGS | VSMYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO: 659) | 678) | NO: 657) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 678) | NO: 673) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 678) | NO: 674) | NO: 654) | 655) | 656) |
| GYTFTGHKM | VPSGGS | VSMYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO: 659) | 678) | NO: 673) | NO: 654) | 655) | 656) |
| GYTFTGHKM | VPSGGS | VSMYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO: 659) | 678) | NO: 674) | NO: 654) | 655) | 656) |
| GYTFTSY | VPSGGS | VSRYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 678) | NO: 675) | NO: 654) | 655) | 656) |
| GYTFTGHKM | VPSGGS | VSRYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO: 659) | 678) | NO: 675) | NO: 654) | 655) | 656) |
| GYTFTSY | SPYNGN | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 652) | NO: 653) | NO: 654) | 655) | 656) |
| GYTFTSY | SPYNGN | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 652) | NO: 657) | NO: 654) | 655) | 656) |
| GYTFTSY | NPASGW | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 658) | NO: 653) | NO: 654) | 655) | 656) |
| GYTFTSY | NPASGW | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 658) | NO: 657) | NO: 654) | 655) | 656) |
| GYTFTSY | SPYNGN | VSMYAPEPM | RASQGISRW | ASSLOS | QQAFSHPWT |
| (SEQ ID NO: | (SEQ ID NO: | DV (SEQ ID | LA (SEQ ID | (SEQ ID NO: | (SEQ ID NO: |
| 680) | 652) | NO: 653) | NO: 654) | 655) | 656) |
| GYTFTSY | SPYNGN | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 652) | NO: 657) | NO: 654) | 655) | 656) |
| GYTFTSY | NPASGW | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 658) | NO: 653) | NO: 654) | 655) | 656) |
| GYTFTSY | NPASGW | VSMYAPEPM | RASQGISRW | ASSLOQS | QQAFSHPWT |
| (SEQ ID NO: | (SEQ ID NO: | DV (SEQ ID | LA (SEQ ID | (SEQ ID NO: | (SEQ ID NO: |
| 680) | 658) | NO: 657) | NO: 654) | 655) | 656) |
| GYTFTSY | SPYNGN | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 652) | NO: 673) | NO: 654) | 655) | 656) |
| GYTFTSY | SPYNGN | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 652) | NO: 674) | NO: 654) | 655) | 656) |
| GYTFTSY | NPASGW | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 658) | NO: 673) | NO: 654) | 655) | 656) |
| GYTFTSY | NPASGW | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 658) | NO: 674) | NO: 654) | 655) | 656) |
| GYTFTSY | SPYNGN | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 652) | NO: 673) | NO: 654) | 655) | 656) |
| GYTFTSY | SPYNGN | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 652) | NO: 674) | NO: 654) | 655) | 656) |
| GYTFTSY | NPASGW | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 658) | NO: 673) | NO: 654) | 655) | 656) |
| GYTFTSY | NPASGW | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 658) | NO: 674) | NO: 654) | 655) | 656) |
| GYTFTSY | SPYNGN | VSRYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 652) | NO: 675) | NO: 654) | 655) | 656) |
| GYTFTSY | NPASGW | VSRYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 658) | NO: 675) | NO: 654) | 655) | 656) |
| GYTFTSY | SPYNGN | VSRYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 652) | NO: 675) | NO: 654) | 655) | 656) |
| GYTFTSY | NPASGW | VSRYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 658) | NO: 675) | NO: 654) | 655) | 656) |
| GYTFTSY | RPSVGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 676) | NO: 653) | NO: 654) | 655) | 656) |
| GYTFTSY | RPSVGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 676) | NO: 657) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 677) | NO: 653) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 677) | NO: 657) | NO: 654) | 655) | 656) |
| GYTFTSY | RPSVGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 676) | NO: 653) | NO: 654) | 655) | 656) |
| GYTFTSY | RPSVGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 676) | NO: 657) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 677) | NO: 653) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 677) | NO: 657) | NO: 654) | 655) | 656) |
| GYTFTSY | RPSVGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 676) | NO: 673) | NO: 654) | 655) | 656) |
| GYTFTSY | RPSVGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 676) | NO: 674) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 677) | NO: 673) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 677) | NO: 674) | NO: 654) | 655) | 656) |
| GYTFTSY | RPSVGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 676) | NO: 673) | NO: 654) | 655) | 656) |
| GYTFTSY | RPSVGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 676) | NO: 674) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 677) | NO: 673) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 677) | NO: 674) | NO: 654) | 655) | 656) |
| GYTFTSY | RPSVGS | VSRYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 676) | NO: 675) | NO: 654) | 655) | 656) |
| GYTFTSY | VPSGGS | VSRYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 677) | NO: 675) | NO: 654) | 655) | 656) |
| GYTFTSY | RPSVGS | VSRYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 676) | NO: 675) | NO: 654) | 655) | 656) |
| GYTFTSY | VPSGGS | VSRYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 677) | NO: 675) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 678) | NO: 653) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 678) | NO: 657) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 678) | NO: 653) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 678) | NO: 657) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 678) | NO: 673) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 678) | NO: 674) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 678) | NO: 673) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 678) | NO: 674) | NO: 654) | 655) | 656) |
| GYTFTSY | VPSGGS | VSRYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 678) | NO: 675) | NO: 654) | 655) | 656) |
| GYTFTSY | VPSGGS | VSRYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 678) | NO: 675) | NO: 654) | 655) | 656) |
| NYWMG (SEQ | YIRSSGGLTH | GLYSFDY | RASQGVNNF | AASTLQS | QQSYSTPRG |
| ID NO: 681) | YADSVKG | (SEQ ID NO: | LA (SEQ ID | (SEQ ID NO: | (SEQ ID NO: |
| | (SEQ ID NO: | 683) | NO: 684) | 685) | 686) |
| | 682) | | | | |
| GSISSSSYYW | SISYSGSTYY | ARDSLRYGM | KSSQSVLYSS | WASTRES | QQYALAPPR |
| G (SEQ ID | NPSLKS (SEQ | DV (SEQ ID | NNKNYLA | (SEQ ID NO: | T (SEQ ID NO: |
| NO: 699) | ID NO: 700) | NO: 701) | (SEQ ID NO: | 703) | 704) |
| | | | 702) | | |
| GSISSSSYYW | SISYSGSTYY | ARDSLRYGM | RASQSVSSN | GASTRAT | QVYNVWPRT |
| G (SEQ ID | NPSLKS (SEQ | DV (SEQ ID | LA (SEQ ID | (SEQ ID NO: | (SEQ ID NO: |
| NO: 699) | ID NO: 700) | NO: 701) | NO: 705) | 706) | 707) |
| SYGMT (SEQ | VISGSGGGTY | AKGPRVVGM | RASQGISSW | AASSLQS | QQVFSYPLT |
| ID NO: 708) | YADSVKG | DV (SEQ ID | LA (SEQ ID | (SEQ ID NO: | (SEQ ID NO: |
| | (SEQ ID NO: | NO: 710) | NO: 711) | 712) | 713) |
| | 709) | | | | |
| GSISSSSYYW | SISYSGSTYY | ARDSLRYGM | RASQSIGSW | KASSLES | QVYGSYSPR |
| G (SEQ ID | NPSLKS (SEQ | DV (SEQ ID | LA (SEQ ID | (SEQ ID NO: | T (SEQ ID NO: |
| NO: 699) | ID NO: 700) | NO: 701) | NO: 714) | 715) | 716) |
| GFTFSSY | AISGSGGSTS | ARLPQYSRP | RASQGISSW | AASSLQS | QQGHSFPLT |
| (SEQ ID NO: | YADSVKG | FDY (SEQ ID | LA (SEQ ID | (SEQ ID NO: | (SEQ ID NO: |
| 717) | (SEQ ID NO: | NO: 719) | NO: 720) | 721) | 722) |
| | 718) | | | | |
| SYRMH (SEQ | FIVPSGGSTS | VSRYAPEPM | RASQGISRW | AASSLQS | QQAFSHPWT |
| ID NO: 723) | YAQKFQG | DV (SEQ ID | LA (SEQ ID | (SEQ ID NO: | (SEQ ID NO: |
| | (SEQ ID NO: | NO: 725) | NO: 726) | 727) | 728) |
| | 724) | | | | |

In some embodiments, the ActRII antibody or an antigen-binding fragment thereof, has a heavy chain and light chain sequence having at least 90% sequence identity (e.g., 90%, 91%, 92%, 93%, 94%, 95%, 95%, 97%, 98%, 99% or 100% sequence identity) to a heavy chain and light chain sequence provided in Table 10. In some embodiments, the ActRII antibody or an antigen binding fragment thereof, has a heavy chain and light chain sequence from the same row of Table 10. In some embodiments, the heavy chain and light chain have the sequence of SEQ ID NOs: 508 and 509; 510 and 511; 557 and 558; 559 and 560; 561 and 562; 563 and 564; 565 and 566; 567 and 568; 569 and 570; 571 and 572; 573 and 574; or 575 and 576 (e.g., the heavy chain has at least 90% sequence identity (e.g., 90%, 91%, 92%, 93%, 94%, 95%, 95%, 97%, 98%, 99% or 100% sequence identity) to the sequence of the first SEQ ID NO: in each pair and the light chain has at least 90% sequence identity (e.g., 90%, 91%, 92%, 93%, 94%, 95%, 95%, 97%, 98%, 99% or 100% sequence identity) to the sequence of the second SEQ ID NO: in each pair).

TABLE 10
Exemplary heavy and light chain sequences of ActRII antibodies

| Heavy chain | Light chain |
|---|---|
| EVQLLESGGGLVQPGGSLRLSCAASGFTFRNS | QTVVTQEPSFSVSPGGTVTLTCGFNSGSVSTSY |
| AMSWVRQAPGKGLEWVSGISVTGGSTFYTDSV | WPSWYQQTPGQAPRTLIYNTNTRSSGVPDRFS |
| KGRFTVSRDNSRNTLYLQMNSLRAEDTAVYYC | GSILGNKAALTITGAQADDESDYYCVLWMGSGI |
| AKVYYYSFFDYWGQGTLVTVSSASTKGPSVFPL | WVFGGGTKLTVLGQPKANPTVTLFPPSSEELQA |
| APCSRSTSESTAALGCLVKDYFPEPVTVSWNS | NKATLVCLISDFYPGAVTVAWKADGSPVKAGVE |
| GALTSGVHTFPAVLQSSGLYSLSSVVTVPSSNF | TTKPSKQSNNKYAASSYLSLTPEQWKSHRSYS |
| GTQTYTCNVDHKPSNTKVDKTVERKCCVECPP | CQVTHEGSTVEKTVAPTECS (SEQ ID NO: 509) |
| CPAPPVAGPSVFLFPPKPKDTLMISRTPEVTCVV | |
| VDVSHEDPEVQFNWYVDGVEVHNAKTKPREEQ | |
| FNSTFRVVSVLTVVHQDWLNGKEYKCKVSNKG | |
| LPAPIEKTISKTKGQPREPQVYTLPPSREEMTKN | |
| QVSLTCLVKGFYPSDIAVEWESNGQPENNYKTT | |
| PPMLDSDGSFFLYSKLTVDKSRWQQGNVFSCS | |
| VMHEALHNHYTQKSLSLSPGK (SEQ ID NO: | |
| 508) | |
| EVQLLESGGGLVQPGGSLRLSCAASGFTFRND | QTVVTQEPSFSVSPGGTVTLTCGFNSGSVSTSY |
| AMSWVRQAPGKGLEWVSGISVTGGSTFYTDSV | YPSWYQQTPGQAPRTLIYNTNTRSSGVPDRFS |
| KGRFTVSRDNSRNTLYLQMNSLRAEDTAVYYC | GSILGNKAALTITGAQADDESDYYCVLYMGSGI |
| AKVYYTSFFDYWGQGTLVTVSSASTKGPSVFPL | WVFGGGTKLTVLGQPKANPTVTLFPPSSEELQA |
| APCSRSTSESTAALGCLVKDYFPEPVTVSWNS | NKATLVCLISDFYPGAVTVAWKADGSPVKAGVE |
| GALTSGVHTFPAVLQSSGLYSLSSVVTVPSSNF | TTKPSKQSNNKYAASSYLSLTPEQWKSHRSYS |
| GTQTYTCNVDHKPSNTKVDKTVERKCCVECPP | CQVTHEGSTVEKTVAPTECS (SEQ ID NO: 511) |
| CPAPPVAGPSVFLFPPKPKDTLMISRTPEVTCVV | |
| VDVSHEDPEVQFNWYVDGVEVHNAKTKPREEQ | |
| FNSTFRVVSVLTVVHQDWLNGKEYKCKVSNKG | |
| LPAPIEKTISKTKGQPREPQVYTLPPSREEMTKN | |
| QVSLTCLVKGFYPSDIAVEWESNGQPENNYKTT | |
| PPMLDSDGSFFLYSKLTVDKSRWQQGNVFSCS | |
| VMHEALHNHYTQKSLSLSPGK (SEQ ID NO: | |
| 510) | |
| QVQLVQSGAEVKKPGASVKVSCKASGYTFTSS | QSALTQPASVSGSPGQSITISCTGTSSDVGSYN |
| YINWVRQAPGQGLEWMGTINPVSGSTSYAQKF | YVNWYQQHPGKAPKLMIYGVSKRPSGVSNRFS |
| QGRVTMTRDTSISTAYMELSRLRSDDTAVYYCA | GSKSGNTASLTISGLQAEDEADYYCGTFAGGSY |
| RGGWFDYWGQGTLVTVSSASTKGPSVFPLAPS | YGVFGGGTKLTVLGQPKAAPSVTLFPPSSEELQ |
| SKSTSGGTAALGCLVKDYFPEPVTVSWNSGALT | ANKATLVCLISDFYPGAVTVAWKADSSPVKAGV |
| SGVHTFPAVLQSSGLYSLSSVVTVPSSSLGTQT | ETTTPSKQSNNKYAASSYLSLTPEQWKSHRSYS |
| YICNVNHKPSNTKVDKRVEPKSCDKTHTCPPCP | CQVTHEGSTVEKTVAPTECS (SEQ ID NO: 558) |
| APEAAGGPSVFLFPPKPKDTLMISRTPEVTCVV | |
| VDVSHEDPEVKFNWYVDGVEVHNAKTKPREEQ | |
| YNSTYRVVSVLTVLHQDWLNGKEYKCKVSNKA | |
| LPAPIEKTISKAKGQPREPQVYTLPPSREEMTKN | |
| QVSLTCLVKGFYPSDIAVEWESNGQPENNYKTT | |
| PPVLDSDGSFFLYSKLTVDKSRWQQGNVFSCS | |
| VMHEALHNHYTQKSLSLSPGK (SEQ ID NO: | |
| 557) | |
| QVQLVQSGAEVKKPGASVKVSCKASGYTFTSS | QSALTQPASVSGSPGQSITISCTGTSSDVGSYN |
| YINWVRQAPGQGLEWMGQINAASGMTRYAQK | YVNWYQQHPGKAPKLMIYGVSKRPSGVSNRFS |
| FQGRVTMTRDTSISTAYMELSRLRSDDTAVYYC | GSKSGNTASLTISGLQAEDEADYYCGTFAGGSY |
| ARGGWFDYWGQGTLVTVSSASTKGPSVFPLAP | YGVFGGGTKLTVLGQPKAAPSVTLFPPSSEELQ |
| SSKSTSGGTAALGCLVKDYFPEPVTVSWNSGA | ANKATLVCLISDFYPGAVTVAWKADSSPVKAGV |
| LTSGVHTFPAVLQSSGLYSLSSVVTVPSSSLGT | ETTTPSKQSNNKYAASSYLSLTPEQWKSHRSYS |
| QTYICNVNHKPSNTKVDKRVEPKSCDKTHTCPP | CQVTHEGSTVEKTVAPTECS (SEQ ID NO: 560) |
| CPAPEAAGGPSVFLFPPKPKDTLMISRTPEVTC | |
| VVVDVSHEDPEVKFNWYVDGVEVHNAKTKPRE | |
| EQYNSTYRVVSVLTVLHQDWLNGKEYKCKVSN | |
| KALPAPIEKTISKAKGQPREPQVYTLPPSREEMT | |
| KNQVSLTCLVKGFYPSDIAVEWESNGQPENNY | |
| KTTPPVLDSDGSFFLYSKLTVDKSRWQQGNV | |
| FSCSVMHEALHNHYTQKSLSLSPGK (SEQ ID | |
| NO: 559) | |
| QVQLVQSGAEVKKPGASVKVSCKASGYTFTSS | QSALTQPASVSGSPGQSITISCTGTSSDVGSYN |
| YINWVRQAPGQGLEWMGNINAAAGITLYAQKF | YVNWYQQHPGKAPKLMIYGVSKRPSGVSNRFS |
| QGRVTMTRDTSISTAYMELSRLRSDDTAVYYCA | GSKSGNTASLTISGLQAEDEADYYCGTFAGGSY |
| RGGWFDYWGQGTLVTVSSASTKGPSVFPLAPS | YGVFGGGTKLTVLGQPKAAPSVTLFPPSSEELQ |
| SKSTSGGTAALGCLVKDYFPEPVTVSWNSGALT | ANKATLVCLISDFYPGAVTVAWKADSSPVKAGV |
| SGVHTFPAVLQSSGLYSLSSVVTVPSSSLGTQT | ETTTPSKQSNNKYAASSYLSLTPEQWKSHRSYS |
| YICNVNHKPSNTKVDKRVEPKSCDKTHTCPPCP | CQVTHEGSTVEKTVAPTECS (SEQ ID NO: 562) |
| APEAAGGPSVFLFPPKPKDTLMISRTPEVTCVV | |
| VDVSHEDPEVKFNWYVDGVEVHNAKTKPREEQ | |
| YNSTYRVVSVLTVLHQDWLNGKEYKCKVSNKA | |
| LPAPIEKTISKAKGQPREPQVYTLPPSREEMTKN | |
| QVSLTCLVKGFYPSDIAVEWESNGQPENNYKTT | |
| PPVLDSDGSFFLYSKLTVDKSRWQQGNVFSCS | |
| VMHEALHNHYTQKSLSLSPGK (SEQ ID NO: | |
| 561) | |
| QVQLVQSGAEVKKPGASVKVSCKASGYTFTSS | QSALTQPASVSGSPGQSITISCTGTSSDVGSYN |
| YINWVRQAPGQGLEWMGGINPPAGTTSYAQKF | YVNWYQQHPGKAPKLMIYGVSKRPSGVSNRFS |
| QGRVTMTRDTSISTAYMELSRLRSDDTAVYYCA | GSKSGNTASLTISGLQAEDEADYYCGTFAGGSY |
| RGGWFDYWGQGTLVTVSSASTKGPSVFPLAPS | YGVFGGGTKLTVLGQPKAAPSVTLFPPSSEELQ |
| SKSTSGGTAALGCLVKDYFPEPVTVSWNSGALT | ANKATLVCLISDFYPGAVTVAWKADSSPVKAGV |
| SGVHTFPAVLQSSGLYSLSSVVTVPSSSLGTQT | ETTTPSKQSNNKYAASSYLSLTPEQWKSHRSYS |
| YICNVNHKPSNTKVDKRVEPKSCDKTHTCPPCP | CQVTHEGSTVEKTVAPTECS (SEQ ID NO: 564) |
| APEAAGGPSVFLFPPKPKDTLMISRTPEVTCVV | |
| VDVSHEDPEVKFNWYVDGVEVHNAKTKPREEQ | |
| YNSTYRVVSVLTVLHQDWLNGKEYKCKVSNKA | |
| LPAPIEKTISKAKGQPREPQVYTLPPSREEMTKN | |
| QVSLTCLVKGFYPSDIAVEWESNGQPENNYKTT | |
| PPVLDSDGSFFLYSKLTVDKSRWQQGNVFSCS | |
| VMHEALHNHYTQKSLSLSPGK (SEQ ID NO: | |
| 563) | |
| QVQLVQSGAEVKKPGASVKVSCKASGYTFTSS | QSALTQPASVSGSPGQSITISCTGTSSDVGSYN |
| YINWVRQAPGQGLEWMGNINPATGHADYAQKF | YVNWYQQHPGKAPKLMIYGVSKRPSGVSNRFS |
| QGRVTMTRDTSISTAYMELSRLRSDDTAVYYCA | GSKSGNTASLTISGLQAEDEADYYCGTFAGGSY |
| RGGWFDYWGQGTLVTVSSASTKGPSVFPLAPS | YGVFGGGTKLTVLGQPKAAPSVTLFPPSSEELQ |
| SKSTSGGTAALGCLVKDYFPEPVTVSWNSGALT | ANKATLVCLISDFYPGAVTVAWKADSSPVKAGV |
| SGVHTFPAVLQSSGLYSLSSVVTVPSSSLGTQT | ETTTPSKQSNNKYAASSYLSLTPEQWKSHRSYS |
| YICNVNHKPSNTKVDKRVEPKSCDKTHTCPPCP | CQVTHEGSTVEKTVAPTECS (SEQ ID NO: 566) |
| APEAAGGPSVFLFPPKPKDTLMISRTPEVTCVV | |
| VDVSHEDPEVKFNWYVDGVEVHNAKTKPREEQ | |
| YNSTYRVVSVLTVLHQDWLNGKEYKCKVSNKA | |
| LPAPIEKTISKAKGQPREPQVYTLPPSREEMTKN | |
| QVSLTCLVKGFYPSDIAVEWESNGQPENNYKTT | |
| PPVLDSDGSFFLYSKLTVDKSRWQQGNVFSCS | |
| VMHEALHNHYTQKSLSLSPGK (SEQ ID NO: | |
| 565) | |
| QVQLVQSGAEVKKPGASVKVSCKASGYTFTSS | QSALTQPASVSGSPGQSITISCTGTSSDVGSYN |
| YINWVRQAPGQGLEWMGTINPVSGSTSYAQKF | YVNWYQQHPGKAPKLMIYGVSKRPSGVSNRFS |
| QGRVTMTRDTSISTAYMELSRLRSDDTAVYYCA | GSKSGNTASLTISGLQAEDEADYYCGTFAGGSY |
| RGGWFDYWGQGTLVTVSSASTKGPSVFPLAPC | YGVFGGGTKLTVLGQPKAAPSVTLFPPSSEELQ |
| SRSTSESTAALGCLVKDYFPEPVTVSWNSGALT | ANKATLVCLISDFYPGAVTVAWKADSSPVKAGV |
| SGVHTFPAVLQSSGLYSLSSVVTVPSSNFGTQT | ETTTPSKQSNNKYAASSYLSLTPEQWKSHRSYS |
| YTCNVDHKPSNTKVDKTVERKCCVECPPCPAP | CQVTHEGSTVEKTVAPTECS (SEQ ID NO: 568) |
| PVAGPSVFLFPPKPKDTLMISRTPEVTCVVVDVS | |
| HEDPEVQFNWYVDGVEVHNAKTKPREEQFNST | |
| FRVVSVLTVVHQDWLNGKEYKCKVSNKGLPAPI | |
| EKTISKTKGQPREPQVYTLPPSREEMTKNQVSL | |
| TCLVKGFYPSDIAVEWESNGQPENNYKTTPPML | |
| DSDGSFFLYSKLTVDKSRWQQGNVFSCSVMHE | |
| ALHNHYTQKSLSLSPGK (SEQ ID NO: 567) | |
| QVQLVQSGAEVKKPGASVKVSCKASGYTFTSS | QSALTQPASVSGSPGQSITISCTGTSSDVGSYN |
| YINWVRQAPGQGLEWMGQINAASGMTRYAQK | YVNWYQQHPGKAPKLMIYGVSKRPSGVSNRFS |
| FQGRVTMTRDTSISTAYMELSRLRSDDTAVYYC | GSKSGNTASLTISGLQAEDEADYYCGTFAGGSY |
| ARGGWFDYWGQGTLVTVSSASTKGPSVFPLAP | YGVFGGGTKLTVLGQPKAAPSVTLFPPSSEELQ |
| CSRSTSESTAALGCLVKDYFPEPVTVSWNSGAL | ANKATLVCLISDFYPGAVTVAWKADSSPVKAGV |
| TSGVHTFPAVLQSSGLYSLSSVVTVPSSNFGTQ | ETTTPSKQSNNKYAASSYLSLTPEQWKSHRSYS |
| TYTCNVDHKPSNTKVDKTVERKCCVECPPCPA | CQVTHEGSTVEKTVAPTECS (SEQ ID NO: 570) |
| PPVAGPSVFLFPPKPKDTLMISRTPEVTCVVVDV | |
| SHEDPEVQFNWYVDGVEVHNAKTKPREEQFNS | |
| TFRVVSVLTVVHQDWLNGKEYKCKVSNKGLPA | |
| PIEKTISKTKGQPREPQVYTLPPSREEMTKNQV | |
| SLTCLVKGFYPSDIAVEWESNGQPENNYKTTPP | |
| MLDSDGSFFLYSKLTVDKSRWQQGNVFSCSVM | |
| HEALHNHYTQKSLSLSPGK (SEQ ID NO: 569) | |
| QVQLVQSGAEVKKPGASVKVSCKASGYTFTSS | QSALTQPASVSGSPGQSITISCTGTSSDVGSYN |
| YINWVRQAPGQGLEWMGNINAAAGITLYAQKF | YVNWYQQHPGKAPKLMIYGVSKRPSGVSNRFS |
| QGRVTMTRDTSISTAYMELSRLRSDDTAVYYCA | GSKSGNTASLTISGLQAEDEADYYCGTFAGGSY |
| RGGWFDYWGQGTLVTVSSASTKGPSVFPLAPC | YGVFGGGTKLTVLGQPKAAPSVTLFPPSSEELQ |
| SRSTSESTAALGCLVKDYFPEPVTVSWNSGALT | ANKATLVCLISDFYPGAVTVAWKADSSPVKAGV |
| SGVHTFPAVLQSSGLYSLSSVVTVPSSNFGTQT | ETTTPSKQSNNKYAASSYLSLTPEQWKSHRSYS |
| YTCNVDHKPSNTKVDKTVERKCCVECPPCPAP | CQVTHEGSTVEKTVAPTECS (SEQ ID NO: 572) |
| PVAGPSVFLFPPKPKDTLMISRTPEVTCVVVDVS | |
| HEDPEVQFNWYVDGVEVHNAKTKPREEQFNST | |
| FRVVSVLTVVHQDWLNGKEYKCKVSNKGLPAPI | |
| EKTISKTKGQPREPQVYTLPPSREEMTKNQVSL | |
| TCLVKGFYPSDIAVEWESNGQPENNYKTTPPML | |
| DSDGSFFLYSKLTVDKSRWQQGNVFSCSVMHE | |
| ALHNHYTQKSLSLSPGK (SEQ ID NO: 571) | |
| QVQLVQSGAEVKKPGASVKVSCKASGYTFTSS | QSALTQPASVSGSPGQSITISCTGTSSDVGSYN |
| YINWVRQAPGQGLEWMGGINPPAGTTSYAQKF | YVNWYQQHPGKAPKLMIYGVSKRPSGVSNRFS |
| QGRVTMTRDTSISTAYMELSRLRSDDTAVYYCA | GSKSGNTASLTISGLQAEDEADYYCGTFAGGSY |
| RGGWFDYWGQGTLVTVSSASTKGPSVFPLAPC | YGVFGGGTKLTVLGQPKAAPSVTLFPPSSEELQ |
| SRSTSESTAALGCLVKDYFPEPVTVSWNSGALT | ANKATLVCLISDFYPGAVTVAWKADSSPVKAGV |
| SGVHTFPAVLQSSGLYSLSSVVTVPSSNFGTQT | ETTTPSKQSNNKYAASSYLSLTPEQWKSHRSYS |
| YTCNVDHKPSNTKVDKTVERKCCVECPPCPAP | CQVTHEGSTVEKTVAPTECS (SEQ ID NO: 574) |
| PVAGPSVFLFPPKPKDTLMISRTPEVTCVVVDVS | |
| HEDPEVQFNWYVDGVEVHNAKTKPREEQFNST | |
| FRVVSVLTVVHQDWLNGKEYKCKVSNKGLPAPI | |
| EKTISKTKGQPREPQVYTLPPSREEMTKNQVSL | |
| TCLVKGFYPSDIAVEWESNGQPENNYKTTPPML | |
| DSDGSFFLYSKLTVDKSRWQQGNVFSCSVMHE | |
| ALHNHYTQKSLSLSPGK (SEQ ID NO: 573) | |
| QVQLVQSGAEVKKPGASVKVSCKASGYTFTSS | QSALTQPASVSGSPGQSITISCTGTSSDVGSYN |
| YINWVRQAPGQGLEWMGNINPATGHADYAQKF | YVNWYQQHPGKAPKLMIYGVSKRPSGVSNRFS |
| QGRVTMTRDTSISTAYMELSRLRSDDTAVYYCA | GSKSGNTASLTISGLQAEDEADYYCGTFAGGSY |
| RGGWFDYWGQGTLVTVSSASTKGPSVFPLAPC | YGVFGGGTKLTVLGQPKAAPSVTLFPPSSEELQ |
| SRSTSESTAALGCLVKDYFPEPVTVSWNSGALT | ANKATLVCLISDFYPGAVTVAWKADSSPVKAGV |
| SGVHTFPAVLQSSGLYSLSSVVTVPSSNFGTQT | ETTTPSKQSNNKYAASSYLSLTPEQWKSHRSYS |
| YTCNVDHKPSNTKVDKTVERKCCVECPPCPAP | CQVTHEGSTVEKTVAPTECS (SEQ ID NO: 576) |
| PVAGPSVFLFPPKPKDTLMISRTPEVTCVVVDVS | |
| HEDPEVQFNWYVDGVEVHNAKTKPREEQFNST | |
| FRVVSVLTVVHQDWLNGKEYKCKVSNKGLPAPI | |
| EKTISKTKGQPREPQVYTLPPSREEMTKNQVSL | |
| TCLVKGFYPSDIAVEWESNGQPENNYKTTPPML | |
| DSDGSFFLYSKLTVDKSRWQQGNVFSCSVMHE | |
| ALHNHYTQKSLSLSPGK (SEQ ID NO: 575) | |

In some embodiments, the ActRII signaling inhibitor is an ActRII ligand trap. ActRII ligand traps are polypeptides that contain an extracellular portion of ActRIIA and/or ActRIIB that are capable of binding to one or more ActRII ligands (e.g., activin A, activin B, myostatin, or GDF11). The extracellular portion of ActRIIA and/or ActRIIB may be fused to a moiety (e.g., an Fc domain, an Fc domain monomer, an albumin-binding peptide, a fibronectin domain, or a human serum albumin) by way of a linker. ActRII ligand traps can reduce or inhibit the binding of ActRII ligands to endogenous activin type II receptors, thereby reducing ActRII signaling. As the ActRII ligand traps contain the extracellular portion of the receptor, they will be soluble and able to bind to and sequester ligands (e.g., activins A and B, myostatin, GDF11) without activating intracellular signaling pathways.

In some embodiments, the ActRII ligand trap is an ActRIIA ligand trap. The ActRIIA ligand trap may contain an extracellular portion of wild-type ActRIIA (e.g., human or murine ActRIIA) or may contain an extracellular portion of wild-type ActRIIA that contains one or more amino acid substitutions relative to the wild-type human extracellular ActRIIA. The wild-type amino acid sequence of the extracellular portion of human ActRIIA is shown below.

Human ActRIIA, extracellular portion (SEQ ID NO: 73):
GAILGRSETQECLFFNANWEKDRTNQTGVEPCYGDKDKRRHCFATWKNISGSIEIVKQGC
WLDDINCYDRTDCVEKKDSPEVYFCCCEGNMCNEKFSYFPEMEVTQPTS

An ActRIIA ligand trap may contain the sequence of SEQ ID NO: 73 or a variant thereof that contains one or more amino acid substitutions. In some embodiments, the ActRIIA ligand trap contains a portion of SEQ ID NO: 73 (e.g., a contiguous portion that is shortened by the removal of amino acids from the N-terminus, C-terminus, or both) or a variant thereof that contains one or more amino acid substitutions. In some embodiments, the ActRIIA ligand trap contains the sequence of SEQ ID NO: 73 or a portion thereof with additional amino acids at the C-terminus from the wild-type sequence of ActRIIA (SEQ ID NO: 75). An exemplary sequence of a portion of wild-type ActRIIA protein that is shortened at the N-terminus and includes additional amino acids from SEQ ID NO: 75 at the C-terminus that can be included in an ActRIIA ligand trap is provided below:

(SEQ ID NO: 729)
ILGRSETQECLFFNANWEKDRTNQTGVEPCYGDKDKRRHCFATWKNISG
SIEIVKQGCWLDDINCYDRTDCVEKKDSPEVYFCCCEGNMCNEKFSYFP
EMEVTQPTSNPVTPKPP

Studies have shown that BMP9 binds ActRIIB with about 300-fold higher binding affinity than ActRIIA (see, e.g., Townson et al., J. Biol. Chem. 287:27313, 2012). ActRIIA-Fc is known to have a longer half-life compared to ActRIIB-Fc. Described herein below are ActRIIA ligand traps containing extracellular ActRIIA variants that are constructed by introducing amino acid residues of ActRIIB to ActRIIA, with the goal of imparting physiological properties conferred by ActRIIB, while also maintaining beneficial physiological and pharmacokinetic properties of ActRIIA. The optimum peptides promote hematopoiesis (e.g., increase red blood cell count, hemoglobin levels, hematocrit, reticulocytes, platelet levels (e.g., platelet count), and/or neutrophil levels (e.g., neutrophil count)), while retaining low binding-affinity to BMP9 and longer serum half-life as an Fc fusion protein, for example. The preferred ActRIIA variants also exhibit similar or improved binding to activins and/or myostatin compared to wild-type ActRIIA, which allows them to compete with endogenous activin receptors for ligand binding and reduce or inhibit endogenous activin receptor signaling. These variants can be used to treat a cytopenia (e.g., anemia, thrombocytopenia, and/or neutropenia) associated with myelofibrosis or myelofibrosis treatment by increasing hemoglobin levels, hematocrit, red blood cell count (e.g., increasing red blood cell production and/or red cell mass or volume), erythroid progenitor maturation and/or differentiation (e.g., the maturation and/or differentiation of early-stage or late- (e.g., terminal) stage erythroid progenitors into proerythroblasts, reticulocytes, or red blood cells), reducing the accumulation of red blood cell progenitor cells (e.g., by stimulating progenitor cells to progress to maturation), increasing late-stage precursor (erythroid precursor) maturation (e.g., terminal maturation, such as the maturation of reticulocytes into red blood cells, or the maturation of erythroblasts into reticulocytes and/or red blood cells), recruiting early-stage progenitors into the erythroid lineage, increasing the number of early-stage erythroid precursors and/or progenitors, promoting the progression of erythroid precursors and/or progenitors through erythropoiesis (e.g., progression through the erythropoiesis pathway), increasing proerythroblasts, increasing reticulocytes, increasing platelet levels (e.g., increasing platelet count, megakaryocyte differentiation and/or maturation, megakaryocyte progenitor renewal, and/or platelet production), increasing megakaryocyte progenitors, reducing the accumulation of platelet progenitor cells (e.g., by stimulating progenitor cells to progress to maturation), increasing neutrophil levels (e.g., increasing neutrophil count, e.g., increasing neutrophil production), and/or increasing the differentiation and/or maturation of progenitor cells (e.g., myeloid progenitors, myeloblasts, or myelocytes) into neutrophils. In some embodiments, amino acid substitutions may be introduced to an extracellular ActRIIA variant to reduce or remove the binding affinity of the variant to BMP9.

ActRIIA ligand traps described herein can include an extracellular ActRIIA variant having at least one amino acid substitution relative to the wild-type extracellular ActRIIA having the sequence of SEQ ID NO: 73. Possible amino acid substitutions at 27 different positions may be introduced to an extracellular ActRIIA variant (Table 11). In some embodiments, an extracellular ActRIIA variant may have at least 85% (e.g., at least 85%, 87%, 90%, 92%, 95%, 97%, or greater) amino acid sequence identity to the sequence of a wild-type extracellular ActRIIA (SEQ ID NO: 73). An extracellular ActRIIA variant may have one or more (e.g., 1-27, 1-25, 1-23, 1-21, 1-19, 1-17, 1-15, 1-13, 1-11, 1-9, 1-7, 1-5, 1-3, or 1-2; e.g., 1, 2, 3, 4, 5, 6, 7, 8, 9, 10, 11, 12, 13, 14, 15, 16, 17, 18, 19, 20, 21, 22, 23, 24, 25, 26, or 27) amino acid substitutions relative the sequence of a wild-type extracellular ActRIIA (SEQ ID NO: 73). In some embodiments, an extracellular ActRIIA variant (e.g., an extracellular ActRIIA variant having a sequence of SEQ ID NO: 1) may include amino acid substitutions at all of the 27 positions as listed in Table 11. In some embodiments, an extracellular ActRIIA variant may include amino acid substitutions at a number of positions, e.g., at 2, 4, 6, 8, 10, 12, 14, 16, 18, 20, 22, 24, or 26 out of the 27 positions, as listed in Table 11.

Amino acid substitutions can worsen or improve the activity and/or binding affinity of the ActRIIA variants of the invention. To maintain polypeptide function, it is important that the lysine (K) at position X17 in the sequences shown in Tables 11 and 12 (SEQ ID NOs: 1-72 (e.g., SEQ ID NOs: 6-72)) be retained. Substitutions at that position can lead to a loss of activity. For example, an ActRIIA variant having the sequence GAILGRSETQECLFYNANWELERTNQTGVERCEGEKDKRLHCYATWRNISGSIEIVAKGCWLDDENCYD RTDCVETEENPQVYFCCCEGNMCNEKFSYFPEMEVTQPTS (SEQ ID NO: 85) has reduced activity in vivo, indicating that the substitution of alanine (A) for lysine (K) at X17 is not tolerated. ActRIIA variants of the invention, including variants in Tables 11 and 12 (e.g., SEQ ID NOs: 1-72 (e.g., SEQ ID NOs: 6-72), therefore, retain amino acid K at position X₁₇.

The ActRIIA variants of the invention preferably have reduced, weak, or no substantial binding to BMP9. BMP9 binding is reduced in ActRIIA variants (e.g., reduced compared to wild-type ActRIIA) containing the amino acid sequence TEEN (SEQ ID NO: 76) at positions X₂₃, X₂₄, X₂₅, and X₂₆, as well as in variants that maintain the amino acid K at position X₂₄ and have the amino acid sequence TKEN (SEQ ID NO: 77) at positions X₂₃, X₂₄, X₂₅, and X₂₆. The sequences TEEN (SEQ ID NO: 76) and TKEN (SEQ ID NO: 77) can be employed interchangeably in the ActRIIA variants (e.g., the variants in Tables 11 and 12, e.g., SEQ ID NOs: 1-72 (e.g., SEQ ID NOs: 6-72)) of the invention to provide reduced BMP9 binding.

TABLE 11
Amino acid substitutions in an extracellular ActRIIA variant having
a sequence of any one of SEQ ID NOs: 1-5

GAILGRSETQECLX₁X₂NANWX₃X₄X₅X₆TNQTGVEX₇CX₈GX₉X₁₀X₁₁X₁₂X₁₃X₁₄HCX₁₅ATWX₁₆NISGSIEIV
X₁₇X₁₈GCX₁₉X₂₀X₂₁DX₂₂NCYDRTDCVEX₂₃X₂₄X₂₅X₂₆PX₂₇VYFCCCEGNMCNEKFSYFPEMEVTQPTS
(SEQ ID NO: 1)
GAILGRSETQECLFX₂NANWX₃X₄X₅X₆TNQTGVEX₇CX₈GX₉KX₁₁X₁₂X₁₃X₁₄HCX₁₅ATWX₁₆NISGSIEIVX₁
₇X₁₈GCX₁₉X₂₀X₂₁DX₂₂NCYDRTDCVEX₂₃X₂₄X₂₅X₂₆PX₂₇VYFCCCEGNMCNEKFSYFPEMEVTQPTS
(SEQ ID NO: 2)
GAILGRSETQECLFX₂NANWEX₄X₅RTNQTGVEX₇CX₈GX₉KDKRX₁₄HCX₁₅ATWX₁₆NISGSIEIVKX₁₈GC
WLDDX₂₂NCYDRTDCVEX₂₃X₂₄X₂₅X₂₆PX₂₇VYFCCCEGNMCNEKFSYFPEMEVTQPTS (SEQ ID NO: 3)
GAILGRSETQECLFX₂NANWEX₄DRTNQTGVEX₇CX₈GX₉KDKRX₁₄HCX₁₅ATWX₁₆NISGSIEIVKX₁₈GC
WLDDX₂₂NCYDRTDCVEX₂₃KX₂₅X₂₆PX₂₇VYFCCCEGNMCNEKFSYFPEMEVTQPTS (SEQ ID NO: 4)
GAILGRSETQECLFX₂NANWEX₄DRTNQTGVEPCX₈GX₉KDKRX₁₄HCFATWKNISGSIEIVKX₁₈GCWLD
DINCYDRTDCVEX₂₃KX₂₅X₂₆PX₂₇VYFCCCEGNMCNEKFSYFPEMEVTQPTS (SEQ ID NO: 5)

| | | | |
|---|---|---|---|
| X₁ | F or Y | X₁₅ | F or Y |
| X₂ | F or Y | X₁₆ | K, R, or A |
| X₃ | E or A | X₁₇ | K, A, Y, F, or I |
| X₄ | K or L | X₁₈ | Q or K |
| X₅ | D or E | X₁₉ | W or A |
| X₆ | R or A | X₂₀ | L or A |
| X₇ | P or R | X₂₁ | D, K, R, A, F, G, M, N, or I |
| X₈ | Y or E | X₂₂ | I, F, or A |
| X₉ | D or E | X₂₃ | K or T |
| X₁₀ | K or Q | X₂₄ | K or E |
| X₁₁ | D or A | X₂₅ | D or E |
| X₁₂ | K or A | X₂₆ | S or N |
| X₁₃ | R or A | X₂₇ | E or Q |
| X₁₄ | R or L | | |

The ActRIIA variants of the invention may further include a C-terminal extension (e.g., additional amino acids at the C-terminus). The C-terminal extension can add one or more additional amino acids at the C-terminus (e.g., 1, 2, 3, 4, 5, 6 or more additional amino acids) to any of the variants shown in Tables 11 and 12 (e.g., SEQ ID NOs: 1-70 (e.g., SEQ ID NOs: 6-70)). The C-terminal extension may correspond to sequence from the same position in wild-type ActRIIA. One potential C-terminal extension that can be included in the ActRIIA variants of the invention is amino acid sequence NP. For example, a sequence including the C-terminal extension NP is SEQ ID NO: 71 (e.g., SEQ ID NO: 69 with a C-terminal extension of NP). Another exemplary C-terminal extension that can be included in the ActRIIA variants of the invention is amino acid sequence NPVTPK (SEQ ID NO: 78). For example, a sequence including the C-terminal extension NPVTPK (SEQ ID NO: 78) is SEQ ID NO: 72 (e.g., SEQ ID NO: 69 with a C-terminal extension of NPVTPK (SEQ ID NO: 78)).

In some embodiments of the extracellular ActRIIA variant having the sequence of SEQ ID NO: 1 or 2, X₃ is E, X₆ is R, X₁₁ is D, X₁₂ is K, X₁₃ is R, X₁₆ is Kor R, X₁₇ is K, X₁₉ is W, X₂₀ is L, X₂₁ is D, and X₂₂ is I or F. In some embodiments of the extracellular ActRIIA variant having the sequence of SEQ ID NO: 1, X₂ is Y; X₄ is L; X₈ is E; X₉ is E; X₁₄ is L; X₁₈ is K; X₂₃ is T; X₂₅ is E; X₂₆ is N; and X₂₇ is Q. These substitutions in SEQ ID NO: 1 can also be made in SEQ ID NOs: 2-5. In some embodiments of the extracellular ActRIIA variant having the sequence of SEQ ID NO: 1, X₁ is F or Y; X₂ is Y; X₄ is L; X₅ is D or E; X₇ is P or R; X₈ is E; X₉ is E; X₁₀ is K or Q; X₁₄ is L; X₁₅ is F or Y; X₁₆ is K or R; X₁₈ is K; X₂₂ is I or F; X₂₃ is T; X₂₄ is K or E; X₂₅ is E; X₂₆ is N; and X₂₇ is Q. In some embodiments of the extracellular ActRIIA variant having the sequence of SEQ ID NO: 1, X₁ is F or Y; X₂ is Y; X₃ is E; X₄ is L; X₅ is D or E; X₆ is R; X₇ is P or R; X₈ is E; X₉ is E; X₁₀ is K or Q; X₁₁ is D; X₁₂ is K; X₁₃ is R; X₁₄ is L; X₁₅ is F or Y; X₁₆ is K or R; X₁₇ is K; X₁₈ is K; X₁₉ is W; X₂₀ is L; X₂₁ is D; X₂₂ is I or F; X₂₃ is T; X₂₄ is K or E; X₂₅ is E; X₂₆ is N; and X₂₇ is Q. In some embodiments of the extracellular ActRIIA variant having the sequence of SEQ ID NO: 1 or 2, X₁₇ is K. In some embodiments of the extracellular ActRIIA variant having the sequence of SEQ ID NOs: 1-3, X₁₇ is K, X₂₃ is T, X₂₄ is E, X₂₅ is E, and X₂₆ is N. In some embodiments of the extracellular ActRIIA variant having the sequence of any one of SEQ ID NOs: 1-5, X₁₇ is K, X₂₃ is T, X₂₄ is K, X₂₅ is E, and X₂₆ is N.

In some embodiments, an ActRIIA ligand trap described herein includes an extracellular ActRIIA variant having a sequence of any one of SEQ ID NOs: 6-72 (Table 12).

TABLE 12
Extracellular ActRIIA variants having the sequences of SEQ ID NOs: 6-72

| SEQ ID NO | Amino Acid Sequence |
|---|---|
| 6 | GAILGRSETQECLFYNANWELDRTNQTGVEPCEGEKDKRLHCFATWKNISGSIEIV |
| | KKGCWLDDINCYDRTDCVETKENPQVYFCCCEGNMCNEKFSYFPEMEVTQPTS |
| 7 | GAILGRSETQECLFYNANWELERTNQTGVEPCEGEKDKRLHCFATWKNISGSIEIV |
| | KKGCWLDDINCYDRTDCVETKENPQVYFCCCEGNMCNEKFSYFPEMEVTQPTS |
| 8 | GAILGRSETQECLFYNANWELDRTNQTGVERCEGEKDKRLHCFATWKNISGSIEIV |
| | KKGCWLDDINCYDRTDCVETKENPQVYFCCCEGNMCNEKFSYFPEMEVTQPTS |
| 9 | GAILGRSETQECLFYNANWELDRTNQTGVEPCEGEKDKRLHCYATWKNISGSIEIV |
| | KKGCWLDDINCYDRTDCVETKENPQVYFCCCEGNMCNEKFSYFPEMEVTQPTS |
| 10 | GAILGRSETQECLFYNANWELDRTNQTGVEPCEGEKDKRLHCFATWRNISGSIEIV |
| | KKGCWLDDINCYDRTDCVETKENPQVYFCCCEGNMCNEKFSYFPEMEVTQPTS |
| 11 | GAILGRSETQECLFYNANWELDRTNQTGVEPCEGEKDKRLHCFATWKNISGSIEIV |
| | KKGCWLDDFNCYDRTDCVETKENPQVYFCCCEGNMCNEKFSYFPEMEVTQPTS |
| 12 | GAILGRSETQECLFYNANWELDRTNQTGVEPCEGEKDKRLHCFATWKNISGSIEIV |
| | KKGCWLDDINCYDRTDCVETEENPQVYFCCCEGNMCNEKFSYFPEMEVTQPTS |
| 13 | GAILGRSETQECLFYNANWELERTNQTGVERCEGEKDKRLHCFATWKNISGSIEIV |
| | KKGCWLDDINCYDRTDCVETKENPQVYFCCCEGNMCNEKFSYFPEMEVTQPTS |
| 14 | GAILGRSETQECLFYNANWELERTNQTGVEPCEGEKDKRLHCYATWKNISGSIEIV |
| | KKGCWLDDINCYDRTDCVETKENPQVYFCCCEGNMCNEKFSYFPEMEVTQPTS |
| 15 | GAILGRSETQECLFYNANWELERTNQTGVEPCEGEKDKRLHCFATWRNISGSIEIV |
| | KKGCWLDDINCYDRTDCVETKENPQVYFCCCEGNMCNEKFSYFPEMEVTQPTS |
| 16 | GAILGRSETQECLFYNANWELERTNQTGVEPCEGEKDKRLHCFATWKNISGSIEIV |
| | KKGCWLDDFNCYDRTDCVETKENPQVYFCCCEGNMCNEKFSYFPEMEVTQPTS |
| 17 | GAILGRSETQECLFYNANWELERTNQTGVEPCEGEKDKRLHCFATWKNISGSIEIV |
| | KKGCWLDDINCYDRTDCVETEENPQVYFCCCEGNMCNEKFSYFPEMEVTQPTS |
| 18 | GAILGRSETQECLFYNANWELDRTNQTGVERCEGEKDKRLHCYATWKNISGSIEIV |
| | KKGCWLDDINCYDRTDCVETKENPQVYFCCCEGNMCNEKFSYFPEMEVTQPTS |
| 19 | GAILGRSETQECLFYNANWELDRTNQTGVERCEGEKDKRLHCFATWRNISGSIEIV |
| | KKGCWLDDINCYDRTDCVETKENPQVYFCCCEGNMCNEKFSYFPEMEVTQPTS |
| 20 | GAILGRSETQECLFYNANWELDRTNQTGVERCEGEKDKRLHCFATWKNISGSIEIV |
| | KKGCWLDDFNCYDRTDCVETKENPQVYFCCCEGNMCNEKFSYFPEMEVTQPTS |
| 21 | GAILGRSETQECLFYNANWELDRTNQTGVERCEGEKDKRLHCFATWKNISGSIEIV |
| | KKGCWLDDINCYDRTDCVETEENPQVYFCCCEGNMCNEKFSYFPEMEVTQPTS |
| 22 | GAILGRSETQECLFYNANWELDRTNQTGVEPCEGEKDKRLHCYATWRNISGSIEIV |
| | KKGCWLDDINCYDRTDCVETKENPQVYFCCCEGNMCNEKFSYFPEMEVTQPTS |
| 23 | GAILGRSETQECLFYNANWELDRTNQTGVEPCEGEKDKRLHCYATWKNISGSIEIV |
| | KKGCWLDDFNCYDRTDCVETKENPQVYFCCCEGNMCNEKFSYFPEMEVTQPTS |
| 24 | GAILGRSETQECLFYNANWELDRTNQTGVEPCEGEKDKRLHCYATWKNISGSIEIV |
| | KKGCWLDDINCYDRTDCVETEENPQVYFCCCEGNMCNEKFSYFPEMEVTQPTS |
| 25 | GAILGRSETQECLFYNANWELDRTNQTGVEPCEGEKDKRLHCFATWRNISGSIEIV |
| | KKGCWLDDFNCYDRTDCVETKENPQVYFCCCEGNMCNEKFSYFPEMEVTQPTS |
| 26 | GAILGRSETQECLFYNANWELDRTNQTGVEPCEGEKDKRLHCFATWRNISGSIEIV |
| | KKGCWLDDINCYDRTDCVETEENPQVYFCCCEGNMCNEKFSYFPEMEVTQPTS |
| 27 | GAILGRSETQECLFYNANWELDRTNQTGVEPCEGEKDKRLHCFATWKNISGSIEIV |
| | KKGCWLDDFNCYDRTDCVETEENPQVYFCCCEGNMCNEKFSYFPEMEVTQPTS |
| 28 | GAILGRSETQECLFYNANWELERTNQTGVERCEGEKDKRLHCYATWKNISGSIEIV |
| | KKGCWLDDINCYDRTDCVETKENPQVYFCCCEGNMCNEKFSYFPEMEVTQPTS |
| 29 | GAILGRSETQECLFYNANWELERTNQTGVERCEGEKDKRLHCFATWRNISGSIEIV |
| | KKGCWLDDINCYDRTDCVETKENPQVYFCCCEGNMCNEKFSYFPEMEVTQPTS |
| 30 | GAILGRSETQECLFYNANWELERTNQTGVERCEGEKDKRLHCFATWKNISGSIEIV |
| | KKGCWLDDFNCYDRTDCVETKENPQVYFCCCEGNMCNEKFSYFPEMEVTQPTS |
| 31 | GAILGRSETQECLFYNANWELERTNQTGVERCEGEKDKRLHCFATWKNISGSIEIV |
| | KKGCWLDDINCYDRTDCVETEENPQVYFCCCEGNMCNEKFSYFPEMEVTQPTS |
| 32 | GAILGRSETQECLFYNANWELERTNQTGVEPCEGEKDKRLHCYATWRNISGSIEIV |
| | KKGCWLDDINCYDRTDCVETKENPQVYFCCCEGNMCNEKFSYFPEMEVTQPTS |
| 33 | GAILGRSETQECLFYNANWELERTNQTGVEPCEGEKDKRLHCYATWKNISGSIEIV |
| | KKGCWLDDFNCYDRTDCVETKENPQVYFCCCEGNMCNEKFSYFPEMEVTQPTS |
| 34 | GAILGRSETQECLFYNANWELERTNQTGVEPCEGEKDKRLHCYATWKNISGSIEIV |
| | KKGCWLDDINCYDRTDCVETEENPQVYFCCCEGNMCNEKFSYFPEMEVTQPTS |
| 35 | GAILGRSETQECLFYNANWELERTNQTGVEPCEGEKDKRLHCFATWRNISGSIEIV |
| | KKGCWLDDFNCYDRTDCVETKENPQVYFCCCEGNMCNEKFSYFPEMEVTQPTS |
| 36 | GAILGRSETQECLFYNANWELERTNQTGVEPCEGEKDKRLHCFATWRNISGSIEIV |
| | KKGCWLDDINCYDRTDCVETEENPQVYFCCCEGNMCNEKFSYFPEMEVTQPTS |
| 37 | GAILGRSETQECLFYNANWELERTNQTGVEPCEGEKDKRLHCFATWKNISGSIEIV |
| | KKGCWLDDFNCYDRTDCVETEENPQVYFCCCEGNMCNEKFSYFPEMEVTQPTS |
| 38 | GAILGRSETQECLFYNANWELDRTNQTGVERCEGEKDKRLHCYATWRNISGSIEIV |
| | KKGCWLDDINCYDRTDCVETKENPQVYFCCCEGNMCNEKFSYFPEMEVTQPTS |
| 39 | GAILGRSETQECLFYNANWELDRTNQTGVERCEGEKDKRLHCYATWKNISGSIEIV |
| | KKGCWLDDFNCYDRTDCVETKENPQVYFCCCEGNMCNEKFSYFPEMEVTQPTS |
| 40 | GAILGRSETQECLFYNANWELDRTNQTGVERCEGEKDKRLHCYATWKNISGSIEIV |
| | KKGCWLDDINCYDRTDCVETEENPQVYFCCCEGNMCNEKFSYFPEMEVTQPTS |
| 41 | GAILGRSETQECLFYNANWELDRTNQTGVERCEGEKDKRLHCFATWRNISGSIEIV |
| | KKGCWLDDFNCYDRTDCVETKENPQVYFCCCEGNMCNEKFSYFPEMEVTQPTS |
| 42 | GAILGRSETQECLFYNANWELDRTNQTGVERCEGEKDKRLHCFATWRNISGSIEIV |
| | KKGCWLDDINCYDRTDCVETEENPQVYFCCCEGNMCNEKFSYFPEMEVTQPTS |
| 43 | GAILGRSETQECLFYNANWELDRTNQTGVERCEGEKDKRLHCFATWKNISGSIEIV |
| | KKGCWLDDFNCYDRTDCVETEENPQVYFCCCEGNMCNEKFSYFPEMEVTQPTS |
| 44 | GAILGRSETQECLFYNANWELDRTNQTGVEPCEGEKDKRLHCYATWRNISGSIEIV |
| | KKGCWLDDFNCYDRTDCVETKENPQVYFCCCEGNMCNEKFSYFPEMEVTQPTS |
| 45 | GAILGRSETQECLFYNANWELDRTNQTGVEPCEGEKDKRLHCYATWRNISGSIEIV |
| | KKGCWLDDINCYDRTDCVETEENPQVYFCCCEGNMCNEKFSYFPEMEVTQPTS |
| 46 | GAILGRSETQECLFYNANWELDRTNQTGVEPCEGEKDKRLHCYATWKNISGSIEIV |
| | KKGCWLDDFNCYDRTDCVETEENPQVYFCCCEGNMCNEKFSYFPEMEVTQPTS |
| 47 | GAILGRSETQECLFYNANWELDRTNQTGVEPCEGEKDKRLHCFATWRNISGSIEIV |
| | KKGCWLDDFNCYDRTDCVETEENPQVYFCCCEGNMCNEKFSYFPEMEVTQPTS |
| 48 | GAILGRSETQECLFYNANWELERTNQTGVERCEGEKDKRLHCYATWRNISGSIEIV |
| | KKGCWLDDINCYDRTDCVETKENPQVYFCCCEGNMCNEKFSYFPEMEVTQPTS |
| 49 | GAILGRSETQECLFYNANWELERTNQTGVERCEGEKDKRLHCYATWKNISGSIEIV |
| | KKGCWLDDFNCYDRTDCVETKENPQVYFCCCEGNMCNEKFSYFPEMEVTQPTS |
| 50 | GAILGRSETQECLFYNANWELERTNQTGVERCEGEKDKRLHCYATWKNISGSIEIV |
| | KKGCWLDDINCYDRTDCVETEENPQVYFCCCEGNMCNEKFSYFPEMEVTQPTS |
| 51 | GAILGRSETQECLFYNANWELERTNQTGVERCEGEKDKRLHCFATWRNISGSIEIV |
| | KKGCWLDDFNCYDRTDCVETKENPQVYFCCCEGNMCNEKFSYFPEMEVTQPTS |
| 52 | GAILGRSETQECLFYNANWELERTNQTGVERCEGEKDKRLHCFATWRNISGSIEIV |
| | KKGCWLDDINCYDRTDCVETEENPQVYFCCCEGNMCNEKFSYFPEMEVTQPTS |
| 53 | GAILGRSETQECLFYNANWELERTNQTGVERCEGEKDKRLHCFATWKNISGSIEIV |
| | KKGCWLDDFNCYDRTDCVETEENPQVYFCCCEGNMCNEKFSYFPEMEVTQPTS |
| 54 | GAILGRSETQECLFYNANWELERTNQTGVEPCEGEKDKRLHCYATWRNISGSIEIV |
| | KKGCWLDDFNCYDRTDCVETKENPQVYFCCCEGNMCNEKFSYFPEMEVTQPTS |
| 55 | GAILGRSETQECLFYNANWELERTNQTGVEPCEGEKDKRLHCYATWRNISGSIEIV |
| | KKGCWLDDINCYDRTDCVETEENPQVYFCCCEGNMCNEKFSYFPEMEVTQPTS |
| 56 | GAILGRSETQECLFYNANWELERTNQTGVEPCEGEKDKRLHCYATWKNISGSIEIV |
| | KKGCWLDDFNCYDRTDCVETEENPQVYFCCCEGNMCNEKFSYFPEMEVTQPTS |
| 57 | GAILGRSETQECLFYNANWELERTNQTGVEPCEGEKDKRLHCFATWRNISGSIEIV |
| | KKGCWLDDFNCYDRTDCVETEENPQVYFCCCEGNMCNEKFSYFPEMEVTQPTS |
| 58 | GAILGRSETQECLFYNANWELDRTNQTGVERCEGEKDKRLHCYATWRNISGSIEIV |
| | KKGCWLDDFNCYDRTDCVETKENPQVYFCCCEGNMCNEKFSYFPEMEVTQPTS |
| 59 | GAILGRSETQECLFYNANWELDRTNQTGVERCEGEKDKRLHCYATWRNISGSIEIV |
| | KKGCWLDDINCYDRTDCVETEENPQVYFCCCEGNMCNEKFSYFPEMEVTQPTS |
| 60 | GAILGRSETQECLFYNANWELDRTNQTGVERCEGEKDKRLHCYATWKNISGSIEIV |
| | KKGCWLDDFNCYDRTDCVETEENPQVYFCCCEGNMCNEKFSYFPEMEVTQPTS |
| 61 | GAILGRSETQECLFYNANWELDRTNQTGVERCEGEKDKRLHCFATWRNISGSIEIV |
| | KKGCWLDDFNCYDRTDCVETEENPQVYFCCCEGNMCNEKFSYFPEMEVTQPTS |
| 62 | GAILGRSETQECLFYNANWELDRTNQTGVEPCEGEKDKRLHCYATWRNISGSIEIV |
| | KKGCWLDDFNCYDRTDCVETEENPQVYFCCCEGNMCNEKFSYFPEMEVTQPTS |
| 63 | GAILGRSETQECLFYNANWELERTNQTGVERCEGEKDKRLHCYATWRNISGSIEIV |
| | KKGCWLDDFNCYDRTDCVETKENPQVYFCCCEGNMCNEKFSYFPEMEVTQPTS |
| 64 | GAILGRSETQECLFYNANWELERTNQTGVERCEGEKDKRLHCYATWRNISGSIEIV |
| | KKGCWLDDINCYDRTDCVETEENPQVYFCCCEGNMCNEKFSYFPEMEVTQPTS |
| 65 | GAILGRSETQECLFYNANWELERTNQTGVERCEGEKDKRLHCYATWKNISGSIEIV |
| | KKGCWLDDFNCYDRTDCVETEENPQVYFCCCEGNMCNEKFSYFPEMEVTQPTS |
| 66 | GAILGRSETQECLFYNANWELERTNQTGVERCEGEKDKRLHCFATWRNISGSIEIV |
| | KKGCWLDDFNCYDRTDCVETEENPQVYFCCCEGNMCNEKFSYFPEMEVTQPTS |
| 67 | GAILGRSETQECLFYNANWELDRTNQTGVERCEGEKDKRLHCYATWRNISGSIEIV |
| | KKGCWLDDFNCYDRTDCVETEENPQVYFCCCEGNMCNEKFSYFPEMEVTQPTS |
| 68 | GAILGRSETQECLFYNANWELERTNQTGVEPCEGEKDKRLHCYATWRNISGSIEIV |
| | KKGCWLDDFNCYDRTDCVETEENPQVYFCCCEGNMCNEKFSYFPEMEVTQPTS |
| 69 | GAILGRSETQECLFYNANWELERTNQTGVERCEGEKDKRLHCYATWRNISGSIEIV |
| | KKGCWLDDFNCYDRTDCVETEENPQVYFCCCEGNMCNEKFSYFPEMEVTQPTS |
| 70 | GAILGRSETQECLYYNANWELERTNQTGVERCEGEQDKRLHCYATWRNISGSIEIV |
| | KKGCWLDDFNCYDRTDCVETEENPQVYFCCCEGNMCNEKFSYFPEMEVTQPTS |
| 71 | GAILGRSETQECLFYNANWELERTNQTGVERCEGEKDKRLHCYATWRNISGSIEIV |
| | KKGCWLDDFNCYDRTDCVETEENPQVYFCCCEGNMCNEKFSYFPEMEVTQPTSN |
| | P |
| 72 | GAILGRSETQECLFYNANWELERTNQTGVERCEGEKDKRLHCYATWRNISGSIEIV |
| | KKGCWLDDFNCYDRTDCVETEENPQVYFCCCEGNMCNEKFSYFPEMEVTQPTSN |
| | PVTPK |

In some embodiments, an ActRIIA ligand trap including an extracellular ActRIIA variant (e.g., any one of SEQ ID NOs: 1-72 (e.g., SEQ ID NOs: 6-72)) has amino acid K at position X₁₇. Altering the amino acid at position X₁₇ can result in reduced activity. For example, an ActRIIA variant having the sequence GAILGRSETQECLFYNANWELERTNQTGVERCEGEKDKRLHCYATWRNISGSIEIVAKGCWLDDENCYD RTDCVETEENPQVYFCCCEGNMCNEKFSYFPEMEVTQPTS (SEQ ID NO: 85) has reduced activity in vivo, indicating that the substitution of A for K at X₁₇ is not tolerated.

In some embodiments, an ActRIIA ligand trap including an extracellular ActRIIA variant (e.g., any one of SEQ ID NOs: 1-72 (e.g., SEQ ID NOs: 6-72)) with the sequence TEEN (SEQ ID NO: 76) at positions X₂₃, X₂₄, X₂₅, and X₂₆ can have a substitution of the amino acid K for the amino acid E at position X₂₄. In some embodiments, an ActRIIA ligand trap including an extracellular ActRIIA variant (e.g., any one of SEQ ID NOs: 1-72 (e.g., SEQ ID NOs: 6-72) with the sequence TKEN (SEQ ID NO: 77) at positions X₂₃, X₂₄, X₂₅, and X₂₆ can have a substitution of the amino acid E for the amino acid K at position X₂₄. ActRIIA variants having the sequence TEEN (SEQ ID NO: 76) or TKEN (SEQ ID NO: 77) at positions X₂₃, X₂₄, X₂₅, and X₂₆ have reduced or weak binding to BMP9 (e.g., reduced binding to BMP9 compared to BMP9 binding of wild-type ActRIIA).

In some embodiments, an ActRIIA ligand trap including an extracellular ActRIIA variant (e.g., any one of SEQ ID NOs: 1-70 (e.g., SEQ ID NOs: 6-70)) may further include a C-terminal extension (e.g., one more additional amino acids at the C-terminus of the ActRIIA variant). The C-terminal extension may correspond to sequence from the same position in wild-type ActRIIA. In some embodiments, the C-terminal extension is amino acid sequence NP. For example, a sequence including the C-terminal extension NP is SEQ ID NO: 71 (e.g., SEQ ID NO: 69 with a C-terminal extension of NP). In some embodiments, the C-terminal extension is amino acid sequence NPVTPK (SEQ ID NO: 78). For example, a sequence including the C-terminal extension NPVTPK (SEQ ID NO: 78) is SEQ ID NO: 72 (e.g., SEQ ID NO: 69 with a C-terminal extension of NPVTPK (SEQ ID NO: 78)). The C-terminal extension can add one or more amino acids at the C-terminus of the ActRIIA variant (e.g., 1, 2, 3, 4, 5, 6 or more additional amino acids).

In some embodiments, an ActRIIA ligand trap including an extracellular ActRIIA variant may further include a moiety (e.g., Fc domain monomer, an Fc domain, an albumin-binding peptide, a fibronectin domain, or a human serum albumin), which may be fused to the N- or C-terminus (e.g., C-terminus) of the extracellular ActRIIA variant by way of a linker or other covalent bonds. A polypeptide including an extracellular ActRIIA variant fused to an Fc domain monomer may form a dimer (e.g., homodimer or heterodimer) through the interaction between two Fc domain monomers, which combine to form an Fc domain in the dimer.

Furthermore, in some embodiments, an ActRIIA ligand trap described herein (e.g., an ActRIIA variant-Fc fusion protein) has a serum half-life of at least 7 days in humans. The ActRIIA ligand trap may bind to activin A with a K_{D} of 10 pM or higher. In some embodiments, the ActRIIA ligand trap does not bind to BMP9 or activin A. In some embodiments, the ActRIIA ligand trap binds to activin A, activin B, and/or myostatin and exhibits reduced (e.g., weak) binding to BMP9 (e.g., reduced BMP9 binding compared to BMP9 binding of wild-type ActRIIA). In some embodiments, the ActRIIA ligand trap that has reduced or weak binding to BMP9 has the sequence TEEN (SEQ ID NO: 76) or TKEN (SEQ ID NO: 77) at positions X₂₃, X₂₄, X₂₅, and X₂₆. In some embodiments, the ActRIIA ligand trap does not substantially bind to human BMP9.

In some embodiments, the ActRIIA ligand trap may bind to human activin A with a K_{D} of about 800 pM or less (e.g., a K_{D} of about 800, 700, 600, 500, 400, 300, 200, 100, 90, 80, 70, 60, 50, 40, 30, 20, 10, 9, 8, 7, 6, 5, 4, 3, 2, or 1 pM or less, e.g., a K_{D} of between about 800 pM and about 200 pM). In some embodiments, the ActRIIA ligand trap may bind to human activin B with a K_{D} of 800 pM or less (e.g., a K_{D} of about 800, 700, 600, 500, 400, 300, 200, 100, 90, 80, 70, 60, 50, 40, 30, 20, 10, 9, 8, 7, 6, 5, 4, 3, 2, or 1 pM or less, e.g., a K_{D} of between about 800 pM and about 200 pM) The ActRIIA ligand trap may also bind to growth and differentiation factor 11 (GDF-11) with a K_{D} of approximately 5 pM or higher (e.g., a K_{D} of about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 pM or higher).

In some embodiments, the ActRIIA ligand trap is sotatercept (also known as ACE-011). Additional ActRIIA ligand traps that may be used in the methods described herein include those described in International Patent Application Publication No. WO2007062188 and U.S. Pat. Nos. 7,709,605, 9,138,459, 7,612,041, 8,067,360, 8,629,109, 9,572,865, 9,163,075, 10,071,135, and 7,951,771, each of which is incorporated herein by reference.

In some embodiments, the ActRII ligand trap is an ActRIIB ligand trap. The ActRIIB ligand trap may contain an extracellular portion of wild-type ActRIIB (e.g., human or murine ActRIIB) or may contain an extracellular portion of wild-type ActRIIB that contains one or more amino acid substitutions relative to the wild-type human extracellular ActRIIB. The wild-type amino acid sequence of the extracellular portion of human ActRIIB is shown below.

Human ActRIIB, extracellular portion (SEQ ID
NO: 74):
GRGEAETRECIYYNANWELERTNQSGLERCEGEQDKRLHCYASWRNSSG
TIELVKKGCWLDDFNCYDRQECVATEENPQVYFCCCEGNFCNERFTHLP
EAGGPEVTYEPPPTAPT

An ActRIIB ligand trap may contain the sequence of SEQ ID NO: 74 or a variant thereof that contains one or more amino acid substitutions. In some embodiments, the ActRIIB ligand trap contains a portion of SEQ ID NO: 74 (e.g., a contiguous portion that is shortened by the removal of amino acids from the N-terminus, C-terminus, or both) or a variant thereof that contains one or more amino acid substitutions. For example, the ActRIIB ligand trap can include the sequence of SEQ ID NO: 74 with an L60D substitution. In another example, the ActRIIB ligand trap can include the sequence of SEQ ID NO: 74 with a substitution at position E9 (e.g., an E9W, E9A, E9F, E9Q, E9V, E91, E9L, E9M, E9K, E9H, or E9Y substitution), an S25T substitution, and/or an R45A substitution. In some embodiments, the ActRIIB ligand trap is BIIB110 (previously known as ALG-801), ALG-802, luspatercept (REBLOZYL®, also known as ACE-536), Ramatercept (also known as ACE-031), or ACE-2494. Additional ActRIIB ligand traps that may be used in the methods described herein include those described in International Patent Application Publication Nos. WO2010/062383, WO2015/192127, WO2019140283, and WO2021189010; US Patent Application Publication Nos. US20110250198 and US20200407415; and U.S. Pat. Nos. 10,913,782, 8,058,229, 8,216,997, 8,703,927, 9,439,945, 9,932,379, 10,131,700, 10,689,427, 10,889,626, 10,829,532, 10,829,533, 8,361,957, 9,505,813, 10,377,996, 9,617,319, 8,710,016, 7,709,605, 8,252,900, 7,842,663, 8,343,933, 9,399,669, 10,259,861, 8,138,142, 8,178,488, 8,293,881, 9,181,533, 9,745,559, 10,358,633, 11,066,654, 9,610,327, 9,284,364, 8,067,562, 8,614,292, 7,947,646, 8,716,459, 8,501,678, 8,999,917, 9,447,165, 9,809,638, 10,407,487, 8,410,043, 9,273,114, and 10,308,704, each of which is incorporated herein by reference.

In some embodiments, the ActRIIB ligand trap contains an ActRIIB variant having the sequence of SEQ ID NO: 730 shown in Table 13.

TABLE 13
Amino acid substitutions in an extracellular ActRIIB variant having
a sequence of SEQ ID NO: 730

GRGEAETRECX₁X₂YNANWEX₃X₄RTNQX₅GX₆EX₇CX₈GX₉X₁₀DKRX₁₁HCX₁₂ASWX₁₃NX₁₄X₁₅GX₁₆X₁₇E
X₁₈VKX₁₉GCWLDDX₂₀NCYDRX₂₁X₂₂CVAX₂₃X₂₄X₂₅X₂₆PX₂₇VYFCCCEGNX₂₈CNERFTHLPEAGGPEVT
YEPPPTAPT (SEQ ID NO: 730)

| | | | |
|---|---|---|---|
| X₁ | I or L | X₁₅ | S or T |
| X₂ | F or Y | X₁₆ | S or T |
| X₃ | L or K | X₁₇ | I or L |
| X₄ | D or E | X₁₈ | I or L |
| X₅ | T or S | X₁₉ | K or Q |
| X₆ | L or V | X₂₀ | F or I |
| X₇ | P or R | X₂₁ | Q, T, or D |
| X₈ | Y or E | X₂₂ | E, D, or T |
| X₉ | D or E | X₂₃ | K or T |
| X₁₀ | K or Q | X₂₄ | K or E |
| X₁₁ | R or L | X₂₅ | D or E |
| X₁₂ | Y or F | X₂₆ | S or N |
| X₁₃ | R or K | X₂₇ | E or Q |
| X₁₄ | S or I | X₂₈ | F or M |

In some embodiments, the ActRIIB variant has the sequence of any one of SEQ ID NOs: 731-744 (Table 14).

TABLE 14
Extracellular ActRIIB variants having the sequences of SEQ ID NOs: 731-744

| SEQ ID NO | Amino Acid Sequence |
|---|---|
| 731 | GRGEAETRECIFYNANWEKDRTNQSGLEPCYGDQDKRRHCFASWKNSSGTIELVKQGC |
| | WLDDINCYDRQECVAKKDSPEVYFCCCEGNFCNERFTHLPEAGGPEVTYEPPPTAPT |
| 732 | GRGEAETRECIYYNANWELDRTNQSGLERCEGEQDKRLHCYASWRNSSGTIELVKKGC |
| | WLDDINCYDRQECVATKENPQVYFCCCEGNFCNERFTHLPEAGGPEVTYEPPPTAPT |
| 733 | GRGEAETRECIYYNANWELERTNQSGLERCEGEQDKRLHCYASWRNSSGTIELVKKGC |
| | WLDDFNCYDRQECVAKKDSPEVYFCCCEGNFCNERFTHLPEAGGPEVTYEPPPTAPT |
| 734 | GRGEAETRECIYYNANWELERTNQTGLERCEGEQDKRLHCYASWRNISGTIELVKKGC |
| | WLDDFNCYDRQECVAKKDSPEVYFCCCEGNFCNERFTHLPEAGGPEVTYEPPPTAPT |
| 735 | GRGEAETRECIYYNANWELERTNQTGLERCEGEQDKRLHCYASWRNITGTIELVKKGC |
| | WLDDFNCYDRQECVAKKDSPEVYFCCCEGNFCNERFTHLPEAGGPEVTYEPPPTAPT |
| 736 | GRGEAETRECIYYNANWELERTNQSGLEPCEGEQDKRLHCYASWRNSSGTIELVKKGC |
| | WLDDFNCYDRQECVAKKDSPEVYFCCCEGNFCNERFTHLPEAGGPEVTYEPPPTAPT |
| 737 | GRGEAETRECIYYNANWELERTNQSGLERCYGDKDKRLHCYASWRNSSGTIELVKKGC |
| | WLDDFNCYDRQECVAKKDSPEVYFCCCEGNFCNERFTHLPEAGGPEVTYEPPPTAPT |
| 738 | GRGEAETRECIYYNANWELERTNQSGLERCEGEQDKRLHCFASWKNSSGTIELVKQGC |
| | WLDDFNCYDRQECVAKKDSPEVYFCCCEGNFCNERFTHLPEAGGPEVTYEPPPTAPT |
| 739 | GRGEAETRECIFYNANWEKDRTNQSGLERCEGEQDKRLHCYASWRNSSGTIELVKKGC |
| | WLDDFNCYDRQECVAKKDSPEVYFCCCEGNFCNERFTHLPEAGGPEVTYEPPPTAPT |
| 740 | GRGEAETRECIYYNANWELERTNQSGLERCYGDQDKRRHCYASWRNSSGTIELVKKGC |
| | WLDDFNCYDRQECVAKKDSPEVYFCCCEGNFCNERFTHLPEAGGPEVTYEPPPTAPT |
| 741 | GRGEAETRECLYYNANWELERTNQSGVERCEGEKDKRLHCYASWRNSSGSLEIVKKGC |
| | WLDDFNCYDRTDCVATEENPQVYFCCCEGNMCNERFTHLPEAGGPEVTYEPPPTAPT |
| 742 | GRGEAETRECLYYNANWELERTNQSGVERCEGEKDKRLHCYASWRNSSGSLEIVKKGC |
| | WLDDFNCYDRDTCVATEENPQVYFCCCEGNMCNERFTHLPEAGGPEVTYEPPPTAPT |
| 743 | GRGEAETRECLYYNANWELERTNQSGVERCEGEKDKRLHCYASWRNSSGSLEIVKKGC |
| | WLDDFNCYDRTDCVATKENPQVYFCCCEGNMCNERFTHLPEAGGPEVTYEPPPTAPT |
| 744 | GRGEAETRECLYYNANWELERTNQSGVERCEGEKDKRLHCYASWRNSSGSLEIVKKGC |
| | WLDDFNCYDRDTCVATKENPQVYFCCCEGNMCNERFTHLPEAGGPEVTYEPPPTAPT |

In some embodiments, the extracellular ActRIIB variant has an N-terminal truncation of 1-7 amino acids (e.g., 1, 2, 3, 4, 5, 6, or 7 amino acids). An N-terminal truncation can be produced by removing 1-7 amino acids from the N-terminus of an of an ActRIIB variant shown in Tables 13 and 14. The N-terminal truncation can remove amino acids up two to amino acids before the first cysteine (e.g., the two amino acids before the first cysteine (RE) are retained in the N-terminally truncated ActRIIB variants). Additional ActRIIB variants having an N-terminal truncation are provided below:

(SEQ ID NO: 745)
ETRECIYYNANWELERTNQSGLERCYGDKDKRRHCYASWRNSSGTIELV
KKGCWLDDFNCYDRQECVATEENPQVYFCCCEGNFCNERFTHLPEAGGP
EVTYEPPPTAPT
(SEQ ID NO: 746)
ETRECIYYNANWELERTNQSGLERCEGDQDKRLHCYASWRNSSGTIELV
KKGCWLDDINCYDRQECVATKENPQVYFCCCEGNFCNERFTHLPEAGGP
EVTYEPPPTAPT
(SEQ ID NO: 747)
ETRECIYYNANWELERTNQSGLERCEGEQDKRLHCYASWRNSSGTIELV
KKGCWDDDFNCYDRQECVATEENPQVYFCCCEGNFCNERFTHLPEAGGP
EVTYEPPPT
(SEQ ID NO: 748)
ETRWCIYYNANWELERTNQSGLERCEGEQDKRLHCYASWRNSSGTIELV
KKGCWLDDFNCYDRQECVATEENPQVYFCCCEGNFCNERFTHLPEAGGP
EVTYEPPPTAPT
(SEQ ID NO: 749)
ETRWCIYYNANWELERTNQTGLERCEGEQDKRLHCYASWRNSSGTIELV
KKGCWLDDFNCYDRQECVATEENPQVYFCCCEGNFCNERFTHLPEAGGP
EVTYEPPPTAPT
(SEQ ID NO: 750)
ETRYCIYYNANWELERTNQTGLERCEGEQDKRLHCYASWRNSSGTIELV
KKGCWLDDFNCYDRQECVATEENPQVYFCCCEGNFCNERFTHLPEAGGP
EVTYEPPPTAPT

In some embodiments, an ActRIIB ligand trap including an ActRIIB variant may further include a moiety (e.g., Fc domain monomer, an Fc domain, an albumin-binding peptide, a fibronectin domain, or a human serum albumin), which may be fused to the N- or C-terminus (e.g., C-terminus) of the extracellular ActRIIB variant by way of a linker or other covalent bonds. An ActRIIB ligand trap including an extracellular ActRIIB variant fused to an Fc domain monomer may form a dimer (e.g., homodimer or heterodimer) through the interaction between two Fc domain monomers, which combine to form an Fc domain in the dimer.

In some embodiments, the ActRII ligand trap is an ActRII chimera ligand trap. The ActRII chimera ligand traps contain portions of extracellular ActRIIA (e.g., human ActRIIA) and extracellular ActRIIB (e.g., human ActRIIB). In some embodiments, the ActRII chimera ligand traps contain an N-terminal portion of extracellular ActRIIB (SEQ ID NO: 74 shown above) joined to a C-terminal portion of extracellular ActRIIA (SEQ ID NO: 73 shown above) such that the sequences are contiguous (e.g., the ActRIIA sequence continues where the ActRIIB sequence left off, starting with the next the amino acid located in the corresponding position of ActRIIA). In some embodiments, the N-terminus of the ActRII chimera included in the ActRII chimera ligand trap includes the six amino acids found at the N-terminus of extracellular ActRIIA joined to the fifth amino acid of extracellular ActRIIB. In some embodiments, the N-terminus of the ActRII chimera included in the ActRII chimera ligand trap begins with the first amino acid located at the N-terminus of extracellular ActRIIB. In some embodiments, the N-terminus of the ActRII chimera included in the ActRII chimera ligand trap includes the first ten amino acids found at the N-terminus of extracellular ActRIIA joined to the ninth amino acid of extracellular ActRIIB. The extracellular ActRII chimera included in the ActRII chimera ligand trap may also include one or more amino acid substitutions in the portion of the chimera that corresponds to the sequence of ActRIIB compared to wild-type extracellular ActRIIB (e.g., SEQ ID NO: 74 shown above), and one or more amino acid substitutions in the portion of the chimera that corresponds to the sequence of ActRIIA compared to wild-type extracellular ActRIIA (e.g., SEQ ID NO: 73 shown above). Amino acid substitutions at 9 different positions may be introduced into an extracellular ActRII chimera (Table 15). An extracellular ActRII chimera may have one or more (e.g., 1, 2, 3, 4, 5, 6, 7, 8, or 9) amino acid substitutions relative the sequence of a wild-type sequence (e.g., relative to the sequence of wild-type extracellular ActRIIB (SEQ ID NO: 74) if the portion of the chimera corresponds to a region of wild-type extracellular ActRIIB, or relative to the sequence of wild-type extracellular ActRIIA (SEQ ID NO: 73) if the portion of the chimera corresponds to a region of wild-type extracellular ActRIIA). The positions at which amino acid substitutions may be made, as well as the amino acids that may be substituted at these positions, are listed in Table 15. ActRII chimera ligand traps that may be used in the methods described herein include those described in International Patent Application Publication No. WO2021189019A1, the disclosure of which is incorporated herein by reference.

TABLE 15
Amino acid substitutions in an extracellular ActRII chimera having a sequence
of any one of SEQ ID NOs: 751-771

GAILGRAETRECIYYNANWELERTNQSGLERCEGEQX₁KRRHCFATWKNISGSIEIVKQGCWLDDX₂X₃
CYDRTDCVX₄X₅X₆X₇X₈PX₉VYFCCCEGNMCNEKFSYFPEMEVTQPTS (SEQ ID NO: 751)
GAILGRAETRECIYYNANWELERTNQSGLERCEGEQX₁KRLHCFATWKNISGSIEIVKQGCWLDDX₂X₃
CYDRTDCVX₄X₅X₆X₇X₈PX₉VYFCCCEGNMCNEKFSYFPEMEVTQPTS (SEQ ID NO: 752)
GAILGRAETRECIYYNANWELERTNQSGLERCEGEQX₁KRLHCYASWKNISGSIEIVKQGCWLDDX₂X₃
CYDRTDCVX₄X₅X₆X₇X₈PX₉VYFCCCEGNMCNEKFSYFPEMEVTQPTS (SEQ ID NO: 753)
GAILGRAETRECIYYNANWELERTNQSGLERCEGEQX₁KRLHCYASWRNSSGSIEIVKQGCWLDDX₂
X₃CYDRTDCVX₄X₅X₆X₇X₈PX₉VYFCCCEGNMCNEKFSYFPEMEVTQPTS (SEQ ID NO: 754)
GAILGRAETRECIYYNANWELERTNQSGLERCEGEQX₁KRLHCYASWRNSSGTIEIVKQGCWLDDX₂X₃
CYDRTDCVX₄X₅X₆X₇X₈PX₉VYFCCCEGNMCNEKFSYFPEMEVTQPTS (SEQ ID NO: 755)
GAILGRAETRECIYYNANWELERTNQSGLERCEGEQX₁KRLHCYASWRNSSGTIELVKKGCWLDDX₂
X₃CYDRTDCVX₄X₅X₆X₇X₈PX₉VYFCCCEGNMCNEKFSYFPEMEVTQPTS (SEQ ID NO: 756)
GAILGRAETRECIYYNANWELERTNQSGLERCEGEQX₁KRLHCYASWRNSSGTIELVKKGCWLDDX₂
X₃CYDRQECVX₄X₅X₆X₇X₈PX₉VYFCCCEGNMCNEKFSYFPEMEVTQPTS (SEQ ID NO: 757)
GRGEAETRECIYYNANWELERTNQSGLERCEGEQX₁KRRHCFATWKNISGSIEIVKQGCWLDDX₂X₃C
YDRTDCVX₄X₅X₆X₇X₈PX₉VYFCCCEGNMCNEKFSYFPEMEVTQPTS (SEQ ID NO: 758)
GRGEAETRECIYYNANWELERTNQSGLERCEGEQX₁KRLHCFATWKNISGSIEIVKQGCWLDDX₂X₃C
YDRTDCVX₄X₅X₆X₇X₈PX₉VYFCCCEGNMCNEKFSYFPEMEVTQPTS (SEQ ID NO: 759)
GRGEAETRECIYYNANWELERTNQSGLERCEGEQX₁KRLHCYASWKNISGSIEIVKQGCWLDDX₂X₃C
YDRTDCVX₄X₅X₆X₇X₈PX₉VYFCCCEGNMCNEKFSYFPEMEVTQPTS (SEQ ID NO: 760)
GRGEAETRECIYYNANWELERTNQSGLERCEGEQX₁KRLHCYASWRNSSGSIEIVKQGCWLDDX₂X₃C
YDRTDCVX₄X₅X₆X₇X₈PX₉VYFCCCEGNMCNEKFSYFPEMEVTQPTS (SEQ ID NO: 761)
GRGEAETRECIYYNANWELERTNQSGLERCEGEQX₁KRLHCYASWRNSSGTIEIVKQGCWLDDX₂X₃C
YDRTDCVX₄X₅X₆X₇X₈PX₉VYFCCCEGNMCNEKFSYFPEMEVTQPTS (SEQ ID NO: 762)
GRGEAETRECIYYNANWELERTNQSGLERCEGEQX₁KRLHCYASWRNSSGTIELVKKGCWLDDX₂X₃
CYDRTDCVX₄X₅X₆X₇X₈PX₉VYFCCCEGNMCNEKFSYFPEMEVTQPTS (SEQ ID NO: 763)
GRGEAETRECIYYNANWELERTNQSGLERCEGEQX₁KRLHCYASWRNSSGTIELVKKGCWLDDX₂X₃
CYDRQECVX₄X₅X₆X₇X₈PX₉VYFCCCEGNMCNEKFSYFPEMEVTQPTS (SEQ ID NO: 764)
GAILGRSETQECIYYNANWELERTNQSGLERCEGEQX₁KRRHCFATWKNISGSIEIVKQGCWLDDX₂X₃
CYDRTDCVX₄X₅X₆X₇X₈PX₉VYFCCCEGNMCNEKFSYFPEMEVTQPTS (SEQ ID NO: 765)
GAILGRSETQECIYYNANWELERTNQSGLERCEGEQX₁KRLHCFATWKNISGSIEIVKQGCWLDDX₂X₃
CYDRTDCVX₄X₅X₆X₇X₈PX₉VYFCCCEGNMCNEKFSYFPEMEVTQPTS (SEQ ID NO: 766)
GAILGRSETQECIYYNANWELERTNQSGLERCEGEQX₁KRLHCYASWKNISGSIEIVKQGCWLDDX₂X₃
CYDRTDCVX₄X₅X₆X₇X₈PX₉VYFCCCEGNMCNEKFSYFPEMEVTQPTS (SEQ ID NO: 767)
GAILGRSETQECIYYNANWELERTNQSGLERCEGEQX₁KRLHCYASWRNSSGSIEIVKQGCWLDDX₂
X₃CYDRTDCVX₄X₅X₆X₇X₈PX₉VYFCCCEGNMCNEKFSYFPEMEVTQPTS (SEQ ID NO: 768)
GAILGRSETQECIYYNANWELERTNQSGLERCEGEQX₁KRLHCYASWRNSSGTIEIVKQGCWLDDX₂
X₃CYDRTDCVX₄X₅X₆X₇X₈PX₉VYFCCCEGNMCNEKFSYFPEMEVTQPTS (SEQ ID NO: 769)
GAILGRSETQECIYYNANWELERTNQSGLERCEGEQX₁KRLHCYASWRNSSGTIELVKKGCWLDDX₂
X₃CYDRTDCVX₄X₅X₆X₇X₈PX₉VYFCCCEGNMCNEKFSYFPEMEVTQPTS (SEQ ID NO: 770)
GAILGRSETQECIYYNANWELERTNQSGLERCEGEQX₁KRLHCYASWRNSSGTIELVKKGCWLDDX₂
X₃CYDRQECVX₄X₅X₆X₇X₈PX₉VYFCCCEGNMCNEKFSYFPEMEVTQPTS (SEQ ID NO: 771)

| | | | |
|---|---|---|---|
| X₁ | D or R | X₆ | E or K |
| X₂ | I, F, E, D, Y, S, N, Q, or | X₇ | E or D |
| | T | | |
| X₃ | N or T | X₈ | N or S |
| X₄ | A or E | X₉ | Q, E, K, R, D, or N |
| X₅ | T or K | | |

In some embodiments, in ActRII chimeras of SEQ ID NOs: 751-771 (shown in Table 15), X₁ is D, X₂ is I, F, or E, X₃ is N or T, X₄ is A or E, X₅ is T or K, X₆ is E or K, X₇ is E or D, X₈ is N or S, and X₉ is E or Q. In some embodiments, in the extracellular ActRII chimeras of SEQ ID NOs: 174-216, X₁ is D, X₂ is I or F, X₃ is N, X₄ is A or E, X₅ is T or K, X₆ is E or K, X₇ is E or D, X₈ is N or S, and Xe is E or Q.

In some embodiments, ActRII chimera ligand trap contains the sequence of any one of SEQ ID NOs: 772-793 (Table 16).

TABLE 16
Extracellular ActRII chimeras having the sequences of SEQ ID NOs: 772-793

| SEQ ID NO: | Amino Acid Sequence |
|---|---|
| 772 | GAILGRAETRECIYYNANWELERTNQSGLERCEGEQDKRLHCYASWRNSSGTIEIV |
| | KQGCWLDDINCYDRTDCVATEENPQVYFCCCEGNMCNEKFSYFPEMEVTQPTS |
| 773 | GRGEAETRECIYYNANWELERTNQSGLERCEGEQDKRLHCYASWRNSSGTIEIVK |
| | QGCWLDDINCYDRTDCVEKKDSPEVYFCCCEGNMCNEKFSYFPEMEVTQPTS |
| 774 | GRGEAETRECIYYNANWELERTNQSGLERCEGEQDKRLHCYASWRNSSGTIEIVK |
| | QGCWLDDINCYDRTDCVEKKDSPQVYFCCCEGNMCNEKFSYFPEMEVTQPTS |
| 775 | GRGEAETRECIYYNANWELERTNQSGLERCEGEQDKRLHCYASWRNSSGTIEIVK |
| | QGCWLDDFNCYDRTDCVEKKDSPQVYFCCCEGNMCNEKFSYFPEMEVTQPTS |
| 776 | GAILGRAETRECIYYNANWELERTNQSGLERCEGEQDKRLHCYASWRNSSGTIEIV |
| | KQGCWLDDFNCYDRTDCVATEENPQVYFCCCEGNMCNEKFSYFPEMEVTQPTS |
| 777 | GRGEAETRECIYYNANWELERTNQSGLERCEGEQDKRLHCYASWRNSSGTIEIVK |
| | QGCWLDDFNCYDRTDCVEKKDSPEVYFCCCEGNMCNEKFSYFPEMEVTQPTS |
| 778 | GRGEAETRECIYYNANWELERTNQSGLERCEGEQDKRRHCFATWKNISGSIEIVKQ |
| | GCWLDDFNCYDRTDCVEKKDSPQVYFCCCEGNMCNEKFSYFPEMEVTQPTS |
| 779 | GRGEAETRECIYYNANWELERTNQSGLERCEGEQDKRLHCFATWKNISGSIEIVKQ |
| | GCWLDDFNCYDRTDCVEKKDSPQVYFCCCEGNMCNEKFSYFPEMEVTQPTS |
| 780 | GRGEAETRECIYYNANWELERTNQSGLERCEGEQDKRLHCYASWKNISGSIEIVKQ |
| | GCWLDDFNCYDRTDCVEKKDSPQVYFCCCEGNMCNEKFSYFPEMEVTQPTS |
| 781 | GRGEAETRECIYYNANWELERTNQSGLERCEGEQDKRLHCYASWRNSSGSIEIVK |
| | QGCWLDDFNCYDRTDCVEKKDSPQVYFCCCEGNMCNEKFSYFPEMEVTQPTS |
| 782 | GRGEAETRECIYYNANWELERTNQSGLERCEGEQDKRLHCYASWRNSSGTIELVK |
| | KGCWLDDFNCYDRTDCVEKKDSPQVYFCCCEGNMCNEKFSYFPEMEVTQPTS |
| 783 | GRGEAETRECIYYNANWELERTNQSGLERCEGEQDKRLHCYASWRNSSGTIELVK |
| | KGCWLDDFNCYDRQECVATKDSPQVYFCCCEGNMCNEKFSYFPEMEVTQPTS |
| 784 | GRGEAETRECIYYNANWELERTNQSGLERCEGEQDKRLHCYASWRNSSGTIELVK |
| | KGCWLDDFNCYDRQECVATEENPQVYFCCCEGNMCNEKFSYFPEMEVTQPTS |
| 785 | GRGEAETRECIYYNANWELERTNQSGLERCEGEQDKRLHCYASWRNSSGTIEIVK |
| | QGCWLDDNNCYDRTDCVEKKDSPQVYFCCCEGNMCNEKFSYFPEMEVTQPTS |
| 786 | GRGEAETRECIYYNANWELERTNQSGLERCEGEQDKRLHCYASWRNSSGTIEIVK |
| | QGCWLDDTNCYDRTDCVEKKDSPQVYFCCCEGNMCNEKFSYFPEMEVTQPTS |
| 787 | GRGEAETRECIYYNANWELERTNQSGLERCEGEQDKRLHCYASWRNSSGTIEIVK |
| | QGCWLDDETCYDRTDCVEKKDSPQVYFCCCEGNMCNEKFSYFPEMEVTQPTS |
| 788 | GRGEAETRECIYYNANWELERTNQSGLERCEGEQRKRLHCYASWRNSSGTIEIVK |
| | QGCWLDDFNCYDRTDCVEKKDSPQVYFCCCEGNMCNEKFSYFPEMEVTQPTS |
| 789 | GRGEAETRECIYYNANWELERTNQSGLERCEGEQDKRLHCYASWRNSSGTIEIVK |
| | QGCWLDDFNCYDRTDCVETKDSPQVYFCCCEGNMCNEKFSYFPEMEVTQPTS |
| 790 | GAILGRAETRECIYYNANWELERTNQSGLERCEGEQDKRLHCYASWRNSSGTIEIV |
| | KQGCWLDDFNCYDRTDCVEKKDSPQVYFCCCEGNMCNEKFSYFPEMEVTQPTS |
| 791 | GAILGRSETQECIYYNANWELERTNQSGLERCEGEQDKRLHCYASWRNSSGTIEIV |
| | KQGCWLDDFNCYDRTDCVEKKDSPQVYFCCCEGNMCNEKFSYFPEMEVTQPTS |
| 792 | GRGEAETRECIYYNANWELERTNQSGLERCEGEQDKRLHCYASWRNSSGTIELVK |
| | KGCWLDDFNCYDRQECVATKDSPEVYFCCCEGNMCNEKFSYFPEMEVTQPTS |
| 793 | GRGEAETRECIYYNANWELERTNQSGLERCEGEQDKRLHCYASWRNSSGTIELVK |
| | KGCWLDDFNCYDRQECVATKENPQVYFCCCEGNMCNEKFSYFPEMEVTQPTS |

In some embodiments, the ActRII chimera included in the ActRII chimera ligand trap results from the substitution of one or more amino acid sequence corresponding to a B-sheet and, optionally, one or more intervening sequence (e.g., a sequence between the β-sheets), from one ActRII protein (e.g., ActRIIB) into the corresponding position of the other ActRII protein (e.g., ActRIIA). For example, an ActRII chimera may be produced by replacing one or more amino acid sequence corresponding to a β-sheet and, optionally, one or more or an intervening sequence, in ActRIIB with an amino acid sequence corresponding to the β-sheet and, optionally, the intervening sequence, from ActRIIA. An ActRII chimera may also be produced by replacing one or more amino acid sequence corresponding to a β-sheet and, optionally, one or more intervening sequence, in ActRIIA with an amino acid sequence corresponding to the β-sheet and, optionally, the intervening sequence, from ActRIIB. In the ActRII chimeras, a β-sheet and, optionally, an intervening sequence from one protein is replaced with the corresponding β-sheet and, optionally, the corresponding intervening sequence from the other protein (e.g., the 5^{th} β-sheet from ActRIIA (β_{5A}) can be replaced with the 5^{th} β-sheet from ActRIIB (β_{5B})). Each ActRII protein has seven β-sheets (β₁-β₇) and eight intervening sequences (X₁-X₈). The ActRII chimeras include at least one of β₁ₐ, β₂ₐ, β₃ₐ, β₄ₐ, β₅ₐ, or β₇ₐ and at least one of β_{1b}, β_{2b}, β_{3b}, β_{4b}, β_{5b}, or β_{7b}. Accordingly, an ActRII chimera included in the ActRII chimera ligand trap may have one to five β-sheet substitutions (e.g., 1, 2, 3, 4, or 5 of β₁, β₂, β₃, β₄, β₅, and β₇ from one ActRII protein may be substituted with the corresponding β-sheet sequence from the other ActRII protein). The ActRII chimera may also have one to seven intervening sequence substitutions (e.g., 1, 2, 3, 4, 5, 6, or 7 of X₁, X₂, X₃, X₅, X₆, X₇, and X₈ from one ActRII protein may be substituted with the corresponding intervening sequence from the other ActRII protein). In some embodiments, the β-sheet sequence that is substituted is a minimal β-sheet sequence (e.g., at least HCFATWK (SEQ ID NO: 805), which is a portion of RHCFATWKNI (β₃ₐ) (SEQ ID NO: 804); at least HCYASWR (SEQ ID NO: 807), which is a portion of LHCYASWRNS (β_{3b}) (SEQ ID NO: 806); at least EIVKQGCW (SEQ ID NO: 809), which is a portion of SIEIVKQGCW (β₄ₐ) (SEQ ID NO: 808); at least ELVKKGCW (SEQ ID NO: 811), which is a portion of TIELVKKGCW (β_{4b}) (SEQ ID NO: 810); at least VE, which is a portion of VEK (β₅ₐ); at least V, which is a portion of VAT (β_{5b}); at least SYF, which is a portion of KFSYF (β₇ₐ) (SEQ ID NO: 819); or at least T, which is a portion of RFTHL (β_{7b}) (SEQ ID NO: 820)). The extracellular ActRII chimeras are the same length (e.g., have the same number of amino acids) as wild-type extracellular ActRIIA and ActRIIB, therefore, in embodiments in which minimal β-sheet sequences are substituted, contiguous amino acids from ActRIIA or ActRIIB are used to connect the minimal β-sheet to the neighboring intervening sequences to maintain the length (e.g., the number of amino acids) of the ActRII chimeras (e.g., to prevent the extracellular ActRII chimeras from having fewer amino acids than the corresponding regions of extracellular ActRIIA and ActRIIB). Exemplary ActRII chimera sequences that can be included in an ActRII chimera ligand trap are provided in Table 17. ActRII chimera ligand traps that may be used in the methods described herein include those described in International Patent Application No. PCT/US2022/027399, the disclosure of which is incorporated herein by reference.

TABLE 17
Extracellular ActRII chimera sequences
For the ActRII chimera having the sequence
X₁β1X₂β2X₃β3X₄β4X₅β5X₆β6X₇β7X₈:

| | |
|---|---|
| X₁ | GAILGRSETQ (SEQ ID NO: 794) |
| | or |
| | GRGEAETR (SEQ ID NO: 795) |
| β₁ | ECLFFN (β₁ₐ) (SEQ ID NO: 796) |
| | or |
| | ECIYYN (ß_{1b}) (SEQ ID NO: 797) |
| X₂ | ANWEKDRTN (SEQ ID NO: 798) |
| | or |
| | ANWELERTN (SEQ ID NO: 799) |
| β₂ | QTGVEPC (β₂ₐ) (SEQ ID NO: 800) |
| | or |
| | QSGLERC (β_{2b}) (SEQ ID NO: 801) |
| X₃ | YGDKDKR (SEQ ID NO: 802) |
| | or |
| | EGEQDKR (SEQ ID NO: 803) |
| β₃ | RHCFATWKNI (β₃ₐ) (SEQ ID |
| | NO: 804) or a portion thereof |
| | that comprises HCFATWK (SEQ ID |
| | NO: 805) |
| | or |
| | LHCYASWRNS (ß_{3b}) (SEQ ID |
| | NO: 806) or a portion thereof |
| | that comprises HCYASWR (SEQ ID |
| | NO: 807) |
| X₄ | SG |
| β₄ | SIEIVKQGCW (β₄ₐ) (SEQ ID NO: |
| | 808) or a portion thereof that |
| | comprises EIVKQGCW (SEQ ID |
| | NO: 809) |
| | or |
| | TIELVKKGCW (β_{4b}) (SEQ ID |
| | NO: 810) or a portion thereof |
| | that comprises ELVKKGCW (SEQ ID |
| | NO: 811) |
| X₅ | LDDINCYDRTDC (SEQ ID NO: 812) |
| | or |
| | LDDFNCYDRQEC (SEQ ID NO: 813) |
| β₅ | VEK (β₅ₐ) or a portion thereof that |
| | comprises VE |
| | or |
| | VAT (β_{5b}) or a portion thereof that |
| | comprises V |
| X₆ | KDSPEV (SEQ ID NO: 814) |
| | or |
| | EENPQV (SEQ ID NO: 815) |
| β₆ | YFCCCE (SEQ ID NO: 816) |
| X₇ | GNMCNE (SEQ ID NO: 817) |
| | or |
| | GNFCNE (SEQ ID NO: 818) |
| β₇ | KFSYF (β₇ₐ) (SEQ ID NO: 819) or a |
| | portion thereof that comprises SYF |
| | or |
| | RFTHL (β_{7b}) (SEQ ID NO: 820) or a |
| | portion thereof that comprises T |
| X₈ | PEMEVTQPTS (SEQ ID NO: 821) |
| | or |
| | PEAGGPEVTYEPPPTAPT (SEQ ID NO: 822) |

In some embodiments, the extracellular ActRII chimeras have an N-terminal truncation of 1-9 amino acids (e.g., 1, 2, 3, 4, 5, 6, 7, 8, or 9 amino acids). The N-terminal truncation can involve the removal of 1-9 amino acids from the N-terminus of any of the chimeras shown in Tables 15-17. The N-terminal truncation can remove amino acids up two to amino acids before the first cysteine (e.g., the two amino acids before the first cysteine (RE or QE) are retained in the N-terminally truncated ActRII chimera ligand traps).

The extracellular ActRII chimera ligand traps may further include a C-terminal extension (e.g., additional amino acids at the C-terminus). The C-terminal extension can add one or more additional amino acids at the C-terminus (e.g., 1, 2, 3, 4, 5, 6 or more additional amino acids) to any of the chimeras shown in Tables 15-17. The C-terminal extension may correspond to sequence from the same position in wild-type ActRIIA or ActRIIB. For example, C-terminal extensions that can be included in the extracellular ActRII chimera ligand traps of the invention are the amino acid sequence NP and the amino acid sequence NPVTPK (SEQ ID NO: 78), which correspond to sequence found in the same position in wild-type ActRIIA.

In some embodiments, an extracellular ActRII chimera ligand trap may further include a moiety (e.g., Fc domain monomer, an Fc domain, an albumin-binding peptide, a fibronectin domain, or a human serum albumin), which may be fused to the N- or C-terminus (e.g., C-terminus) of the extracellular ActRII chimera by way of a linker or other covalent bonds. An ActRII chimera ligand trap including an extracellular ActRII chimera fused to an Fc domain monomer may form a dimer (e.g., homodimer or heterodimer) through the interaction between two Fc domain monomers, which combine to form an Fc domain in the dimer.

### Fc Domains

In some embodiments, an ActRII ligand trap described herein may include an extracellular portion of ActRIIA, ActRIIB, a variant thereof, or a chimera thereof fused to an Fc domain monomer of an immunoglobulin or a fragment of an Fc domain to increase the serum half-life of the polypeptide. An ActRII ligand trap including an extracellular portion of ActRIIA, ActRIIB, a variant thereof, or a chimera thereof fused to an Fc domain monomer may form a dimer (e.g., homodimer or heterodimer) through the interaction between two Fc domain monomers, which form an Fc domain in the dimer. As conventionally known in the art, an Fc domain is the protein structure that is found at the C-terminus of an immunoglobulin. An Fc domain includes two Fc domain monomers that are dimerized by the interaction between the C_{H}3 antibody constant domains. An Fc domain forms the minimum structure that binds to an Fc receptor, e.g., FcγRI, FcγRIIa, FcγRIIb, FcγRIIIa, FcγRIIIb, FcγRIV. In some embodiments, an Fc domain may be mutated to lack effector functions, typical of a “dead” Fc domain. For example, an Fc domain may include specific amino acid substitutions that are known to minimize the interaction between the Fc domain and an Fcγ receptor. In some embodiments, an Fc domain is from an IgG1 antibody and includes amino acid substitutions L234A, L235A, and G237A. In some embodiments, an Fc domain is from an IgG1 antibody and includes amino acid substitutions D265A, K322A, and N434A. The aforementioned amino acid positions are defined according to Kabat (Sequences of Proteins of Immunological Interest, 5th Ed. Public Health Service, National Institutes of Health, Bethesda, MD. (1991)). The Kabat numbering of amino acid residues may be determined for a given antibody by alignment at regions of homology of the sequence of the antibody with a “standard” Kabat numbered sequence. Furthermore, in some embodiments, an Fc domain does not induce any immune system-related response. For example, the Fc domain in a dimer of an ActRII ligand trap including an extracellular portion of ActRIIA, ActRIIB, a variant thereof, or a chimera thereof fused to an Fc domain monomer may be modified to reduce the interaction or binding between the Fc domain and an Fcγ receptor. The sequence of an Fc domain monomer that may be fused to an extracellular ActRIIA variant is shown below (SEQ ID NO: 97):

THTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYK
CKVSNKALPVPIEKTISKAKGQPREPQVYTLPPSREEMTKNQVSLTCLV
KGFYPSDIAVEWESNGQPENNYKTTPPVLDSDGPFFLYSKLTVDKSRWQ
QGNVFSCSVMHEALHNHYTQKSLSLSPGK

In some embodiments, an Fc domain is from an IgG1 antibody and includes amino acid substitutions L12A, L13A, and G15A, relative to the sequence of SEQ ID NO: 97. In some embodiments, an Fc domain is from an IgG1 antibody and includes amino acid substitutions D43A, K100A, and N212A, relative to the sequence of SEQ ID NO: 97. In some embodiments, the terminal lysine is absent from the Fc domain monomer having the sequence of SEQ ID NO: 97. In some embodiments, an extracellular portion of ActRIIA, ActRIIB, a variant thereof, or a chimera thereof described herein (e.g., an extracellular ActRIIA variant having the sequence of any one of SEQ ID NOs: 1-72 (e.g., SEQ ID NOs: 6-72)) may be fused to the N- or C-terminus of an Fc domain monomer (e.g., SEQ ID NO: 97) through conventional genetic or chemical means, e.g., chemical conjugation. If desired, a linker (e.g., a spacer) can be inserted between an extracellular portion of ActRIIA, ActRIIB, a variant thereof, or a chimera thereof and the Fc domain monomer. The Fc domain monomer can be fused to the N- or C-terminus (e.g., C-terminus) of an extracellular portion of ActRIIA, ActRIIB, a variant thereof, or a chimera thereof.

In some embodiments, an ActRII ligand trap described herein may include an extracellular portion of ActRIIA, ActRIIB, a variant thereof, or a chimera thereof fused to an Fc domain. In some embodiments, the Fc domain contains one or more amino acid substitutions that reduce or inhibit Fc domain dimerization. In some embodiments, the Fc domain contains a hinge domain. The Fc domain can be of immunoglobulin antibody isotype IgG, IgE, IgM, IgA, or IgD. Additionally, the Fc domain can be an IgG subtype (e.g., IgG1, IgG2a, IgG2b, IgG3, or IgG4). The Fc domain can also be a non-naturally occurring Fc domain, e.g., a recombinant Fc domain.

Methods of engineering Fc domains that have reduced dimerization are known in the art. In some embodiments, one or more amino acids with large side-chains (e.g., tyrosine or tryptophan) may be introduced to the C_{H}3-C_{H}3 dimer interface to hinder dimer formation due to steric clash. In other embodiments, one or more amino acids with small side-chains (e.g., alanine, valine, or threonine) may be introduced to the C_{H}3-C_{H}3 dimer interface to remove favorable interactions. Methods of introducing amino acids with large or small side-chains in the C_{H}3 domain are described in, e.g., Ying et al. (J Biol Chem. 287:19399-19408, 2012), U.S. Patent Publication No. 2006/0074225, U.S. Pat. Nos. 8,216,805 and 5,731,168, Ridgway et al. (Protein Eng. 9:617-612, 1996), Atwell et al. (J Mol Biol. 270:26-35, 1997), and Merchant et al. (Nat Biotechnol. 16:677-681, 1998), all of which are incorporated herein by reference in their entireties.

In yet other embodiments, one or more amino acid residues in the C_{H}3 domain that make up the C_{H}3-C_{H}3 interface between two Fc domains are replaced with positively charged amino acid residues (e.g., lysine, arginine, or histidine) or negatively charged amino acid residues (e.g., aspartic acid or glutamic acid) such that the interaction becomes electrostatically unfavorable depending on the specific charged amino acids introduced. Methods of introducing charged amino acids in the C_{H}3 domain to disfavor or prevent dimer formation are described in, e.g., Ying et al. (J Biol Chem. 287:19399-19408, 2012), U.S. Patent Publication Nos. 2006/0074225, 2012/0244578, and 2014/0024111, all of which are incorporated herein by reference in their entireties.

In some embodiments of the invention, an Fc domain includes one or more of the following amino acid substitutions: T366W, T366Y, T394W, F405W, Y349T, Y349E, Y349V, L351T, L351H, L351N, L352K, P353S, S354D, D356K, D356R, D356S, E357K, E357R, E357Q, S364A, T366E, L368T, L368Y, L368E, K370E, K370D, K370Q, K392E, K392D, T394N, P395N, P396T, V397T, V397Q, L398T, D399K, D399R, D399N, F405T, F405H, F405R, Y407T, Y407H, Y4071, K409E, K409D, K409T, and K4091, relative to the sequence of human IgG1. In some embodiments, the terminal lysine is absent from the Fc domain amino acid sequence. In one particular embodiment, an Fc domain includes the amino acid substitution T366W, relative to the sequence of human IgG1. The sequence of an Fc domain (a wild-type Fc domain) is shown below in SEQ ID NO: 84:

DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSR
WQQGNVFSCSVMHEALHNHYTQKSLSLSPGK.

An exemplary sequence for a wild-type Fc domain lacking the terminal lysine is provided below (SEQ ID NO: 79):

DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSR
WQQGNVFSCSVMHEALHNHYTQKSLSLSPG.

### Albumin-Binding Peptide

In some embodiments, an ActRII ligand trap described herein may include an extracellular portion of ActRIIA, ActRIIB, a variant thereof, or a chimera thereof fused to a serum protein-binding peptide. Binding to serum protein peptides can improve the pharmacokinetics of protein pharmaceuticals.

As one example, albumin-binding peptides that can be used in the methods and compositions described here are generally known in the art. In one embodiment, the albumin binding peptide includes the sequence DICLPRWGCLW (SEQ ID NO: 83).

In the present invention, albumin-binding peptides may be joined to the N- or C-terminus (e.g., C-terminus) of an extracellular portion of ActRIIA, ActRIIB, a variant thereof, or a chimera thereof described herein (e.g., an extracellular ActRIIA variant having the sequence of any one of SEQ ID NOs: 1-72 (e.g., SEQ ID NOs: 6-72)) to increase the serum half-life of the extracellular portion of ActRIIA, ActRIIB, a variant thereof, or a chimera thereof. In some embodiments, an albumin-binding peptide is joined, either directly or through a linker, to the N- or C-terminus of an extracellular portion of ActRIIA, ActRIIB, a variant thereof, or a chimera thereof.

In some embodiments, an extracellular portion of ActRIIA, ActRIIB, a variant thereof, or a chimera thereof described herein (e.g., an extracellular ActRIIA variant having the sequence of any one of SEQ ID NOs: 1-72 (e.g., SEQ ID NOs: 6-72)) may be fused to the N- or C-terminus of albumin-binding peptide (e.g., SEQ ID NO: 83) through conventional genetic or chemical means, e.g., chemical conjugation. If desired, a linker (e.g., a spacer) can be inserted between the extracellular portion of ActRIIA, ActRIIB, a variant thereof, or a chimera thereof and the albumin-binding peptide. Without being bound to a theory, it is expected that inclusion of an albumin-binding peptide in an extracellular portion of ActRIIA, ActRIIB, a variant thereof, or a chimera thereof described herein may lead to prolonged retention of the therapeutic protein through its binding to serum albumin.

### Fibronectin Domain

In some embodiments, an ActRII ligand trap described herein may include an extracellular portion of ActRIIA, ActRIIB, a variant thereof, or a chimera thereof fused to fibronectin domains. Binding to fibronectin domains can improve the pharmacokinetics of protein pharmaceuticals.

Fibronectin domain is a high molecular weight glycoprotein of the extracellular matrix, or a fragment thereof, that binds to, e.g., membrane-spanning receptor proteins such as integrins and extracellular matrix components such as collagens and fibrins. In some embodiments of the present invention, a fibronectin domain is joined to the N- or C-terminus (e.g., C-terminus) of an extracellular portion of ActRIIA, ActRIIB, a variant thereof, or a chimera thereof described herein (e.g., an extracellular ActRIIA variant having the sequence of any one of SEQ ID NOs: 1-72 (e.g., SEQ ID NOs: 6-72)) to increase the serum half-life of the extracellular portion of ActRIIA, ActRIIB, a variant thereof, or a chimera thereof. A fibronectin domain can be joined, either directly or through a linker, to the N- or C-terminus of an extracellular portion of ActRIIA, ActRIIB, a variant thereof, or a chimera thereof.

As one example, fibronectin domains that can be used in the methods and compositions described here are generally known in the art. In one embodiment, the fibronectin domain is a fibronectin type III domain (SEQ ID NO: 82, below) having amino acids 610-702 of the sequence of UniProt ID NO: P02751.

GPVEVFITETPSQPNSHPIQWNAPQPSHISKYILRWRPKNSVGRWKEAT
IPGHLNSYTIKGLKPGVVYEGQLISIQQYGHQEVTRFDFTTTST

In another embodiment, the fibronectin domain is an adnectin protein.

In some embodiments, an extracellular portion of ActRIIA, ActRIIB, a variant thereof, or a chimera thereof described herein (e.g., an extracellular ActRIIA variant having the sequence of any one of SEQ ID NOs: 1-72 (e.g., SEQ ID NOs: 6-72)) may be fused to the N- or C-terminus of a fibronectin domain (e.g., SEQ ID NO: 82) through conventional genetic or chemical means, e.g., chemical conjugation. If desired, a linker (e.g., a spacer) can be inserted between the extracellular portion of ActRIIA, ActRIIB, a variant thereof, or a chimera thereof and the fibronectin domain. Without being bound to a theory, it is expected that inclusion of a fibronectin domain in an extracellular portion of ActRIIA, ActRIIB, a variant thereof, or a chimera thereof described herein may lead to prolonged retention of the therapeutic protein through its binding to integrins and extracellular matrix components such as collagens and fibrins.

### Serum Albumin

In some embodiments, an ActRII ligand trap described herein may include an extracellular portion of ActRIIA, ActRIIB, a variant thereof, or a chimera thereof fused to serum albumin. Binding to serum albumins can improve the pharmacokinetics of protein pharmaceuticals.

Serum albumin is a globular protein that is the most abundant blood protein in mammals. Serum albumin is produced in the liver and constitutes about half of the blood serum proteins. It is monomeric and soluble in the blood. Some of the most crucial functions of serum albumin include transporting hormones, fatty acids, and other proteins in the body, buffering pH, and maintaining osmotic pressure needed for proper distribution of bodily fluids between blood vessels and body tissues. In preferred embodiments, serum albumin is human serum albumin. In some embodiments of the present invention, a human serum albumin is joined to the N- or C-terminus (e.g., C-terminus) of an extracellular portion of ActRIIA, ActRIIB, a variant thereof, or a chimera thereof described herein (e.g., an extracellular ActRIIA variant having the sequence of any one of SEQ ID NOs: 1-72 (e.g., SEQ ID NOs: 6-72)) to increase the serum half-life of the extracellular portion of ActRIIA, ActRIIB, a variant thereof, or a chimera thereof. A human serum albumin can be joined, either directly or through a linker, to the N- or C-terminus of an extracellular portion of ActRIIA, ActRIIB, a variant thereof, or a chimera thereof.

As one example, serum albumins that can be used in the methods and compositions described herein are generally known in the art. In one embodiment, the serum albumin includes the sequence of UniProt ID NO: P02768 (SEQ ID NO: 81, below).

MKWVTFISLLFLFSSAYSRGVFRRDAHKSEVAHRFKDLGEENFKALVLI
AFAQYLQQCPFEDHVKLVNEVTEFAKTCVADESAENCDKSLHTLFGDKL
CTVATLRETYGEMADCCAKQEPERNECFLQHKDDNPNLPRLVRPEVDVM
CTAFHDNEETFLKKYLYEIARRHPYFYAPELLFFAKRYKAAFTECCQAA
DKAACLLPKLDELRDEGKASSAKQRLKCASLQKFGERAFKAWAVARLSQ
RFPKAEFAEVSKLVTDLTKVHTECCHGDLLECADDRADLAKYICENQDS
ISSKLKECCEKPLLEKSHCIAEVENDEMPADLPSLAADFVESKDVCKNY
AEAKDVFLGMFLYEYARRHPDYSVVLLLRLAKTYETTLEKCCAAADPHE
CYAKVFDEFKPLVEEPQNLIKQNCELFEQLGEYKFQNALLVRYTKKVPQ
VSTPTLVEVSRNLGKVGSKCCKHPEAKRMPCAEDYLSVVLNQLCVLHEK
TPVSDRVTKCCTESLVNRRPCFSALEVDETYVPKEFNAETFTFHADICT
LSEKERQIKKQTALVELVKHKPKATKEQLKAVMDDFAAFVEKCCKADDK
ETCFAEEGKKLVAASQAALGL

In some embodiments, an extracellular portion of ActRIIA, ActRIIB, a variant thereof, or a chimera thereof described herein (e.g., an extracellular ActRIIA variant having the sequence of any one of SEQ ID NOs: 1-72 (e.g., SEQ ID NOs: 6-72) may be fused to the N- or C-terminus of a human serum albumin (e.g., SEQ ID NO: 81) through conventional genetic or chemical means, e.g., chemical conjugation. If desired, a linker (e.g., a spacer) can be inserted between the extracellular portion of ActRIIA, ActRIIB, a variant thereof, or a chimera thereof and the human serum albumin. Without being bound to a theory, it is expected that inclusion of a human serum albumin in an extracellular portion of ActRIIA, ActRIIB, a variant thereof, or a chimera thereof described herein may lead to prolonged retention of the therapeutic protein.

### Linkers

An ActRII ligand trap described herein may include an extracellular portion of ActRIIA, ActRIIB, a variant thereof, or a chimera thereof (e.g., an extracellular ActRIIA variant having a sequence of any one of SEQ ID NOs: 1-72 (e.g., SEQ ID NOs: 6-72) fused to a moiety by way of a linker. In some embodiments, the moiety increases stability of the polypeptide. Exemplary moieties include an Fc domain monomer, an Fc domain, an albumin-binding peptide, a fibronectin domain, or a human serum albumin. In the present invention, a linker between a moiety (e.g., an Fc domain monomer (e.g., the sequence of SEQ ID NO: 97), an Fc domain (e.g., SEQ ID NO: 84 or SEQ ID NO: 79), an albumin-binding peptide (e.g., SEQ ID NO: 83), a fibronectin domain (e.g., SEQ ID NO: 82), or a human serum albumin (e.g., SEQ ID NO: 81)) and an extracellular portion of ActRIIA, ActRIIB, a variant thereof, or a chimera thereof (e.g., an extracellular ActRIIA variant having the sequence of any one of SEQ ID NOs: 1-72 (e.g., SEQ ID NOs: 6-72)), can be an amino acid spacer including 1-200 amino acids. Suitable peptide spacers are known in the art, and include, for example, peptide linkers containing flexible amino acid residues such as glycine, alanine, and serine. In some embodiments, a spacer can contain motifs, e.g., multiple or repeating motifs, of GA, GS, GG, GGA, GGS, GGG, GGGA (SEQ ID NO: 98), GGGS (SEQ ID NO: 99), GGGG (SEQ ID NO: 100), GGGGA (SEQ ID NO: 101), GGGGS (SEQ ID NO: 102), GGGGG (SEQ ID NO: 103), GGAG (SEQ ID NO: 104), GGSG (SEQ ID NO: 105), AGGG (SEQ ID NO: 106), or SGGG (SEQ ID NO: 107). In some embodiments, a spacer can contain 2 to 12 amino acids including motifs of GA or GS, e.g., GA, GS, GAGA (SEQ ID NO: 108), GSGS (SEQ ID NO: 109), GAGAGA (SEQ ID NO: 110), GSGSGS (SEQ ID NO: 111), GAGAGAGA (SEQ ID NO: 112), GSGSGSGS (SEQ ID NO: 113), GAGAGAGAGA (SEQ ID NO: 114), GSGSGSGSGS (SEQ ID NO: 115), GAGAGAGAGAGA (SEQ ID NO: 116), and GSGSGSGSGSGS (SEQ ID NO: 117). In some embodiments, a spacer can contain 3 to 12 amino acids including motifs of GGA or GGS, e.g., GGA, GGS, GGAGGA (SEQ ID NO: 118), GGSGGS (SEQ ID NO: 119), GGAGGAGGA (SEQ ID NO: 120), GGSGGSGGS (SEQ ID NO: 121), GGAGGAGGAGGA (SEQ ID NO: 122), and GGSGGSGGSGGS (SEQ ID NO: 123). In yet some embodiments, a spacer can contain 4 to 12 amino acids including motifs of GGAG (SEQ ID NO: 104), GGSG (SEQ ID NO: 105), e.g., GGAG (SEQ ID NO: 104), GGSG (SEQ ID NO: 105), GGAGGGAG (SEQ ID NO: 124), GGSGGGSG (SEQ ID NO: 125), GGAGGGAGGGAG (SEQ ID NO: 126), and GGSGGGSGGGSG (SEQ ID NO: 127). In some embodiments, a spacer can contain motifs of GGGGA (SEQ ID NO: 101) or GGGGS (SEQ ID NO: 102), e.g., GGGGAGGGGAGGGGA (SEQ ID NO: 128) and GGGGSGGGGSGGGGS (SEQ ID NO: 129). In some embodiments of the invention, an amino acid spacer between a moiety (e.g., an Fc domain monomer, an Fc domain, an albumin-binding peptide, a fibronectin domain, or a human serum albumin) and an extracellular portion of ActRIIA, ActRIIB, a variant thereof, or a chimera thereof (e.g., an extracellular ActRIIA variant having the sequence of any one of SEQ ID NOs: 1-72 (e.g., SEQ ID NOs: 6-72)) may be GGG, GGGA (SEQ ID NO: 98), GGGG (SEQ ID NO: 100), GGGAG (SEQ ID NO: 130), GGGAGG (SEQ ID NO: 131), or GGGAGGG (SEQ ID NO: 132).

In some embodiments, a spacer can also contain amino acids other than glycine, alanine, and serine, e.g., AAAL (SEQ ID NO: 133), AAAK (SEQ ID NO: 134), AAAR (SEQ ID NO: 135), EGKSSGSGSESKST (SEQ ID NO: 136), GSAGSAAGSGEF (SEQ ID NO: 137), AEAAAKEAAAKA (SEQ ID NO: 96), KESGSVSSEQLAQFRSLD (SEQ ID NO: 95), GENLYFQSGG (SEQ ID NO: 94), SACYCELS (SEQ ID NO: 93), RSIAT (SEQ ID NO: 92), RPACKIPNDLKQKVMNH (SEQ ID NO: 91), GGSAGGSGSGSSGGSSGASGTGTAGGTGSGSGTGSG (SEQ ID NO: 90), AAANSSIDLISVPVDSR (SEQ ID NO: 89), or GGSGGGSEGGGSEGGGSEGGGSEGGGSEGGGSGGGS (SEQ ID NO: 88). In some embodiments, a spacer can contain motifs, e.g., multiple or repeating motifs, of EAAAK (SEQ ID NO: 87). In some embodiments, a spacer can contain motifs, e.g., multiple or repeating motifs, of proline-rich sequences such as (XP)ₙ, in which X may be any amino acid (e.g., A, K, or E) and n is from 1-5, and PAPAP (SEQ ID NO: 86).

The length of the peptide spacer and the amino acids used can be adjusted depending on the two proteins involved and the degree of flexibility desired in the final protein fusion polypeptide. The length of the spacer can be adjusted to ensure proper protein folding and avoid aggregate formation.

In some embodiments, the linker between a moiety (e.g., an Fc domain monomer (e.g., the sequence of SEQ ID NO: 97), an Fc domain (e.g., SEQ ID NO: 84 or SEQ ID NO: 79), an albumin-binding peptide (e.g., SEQ ID NO: 83), a fibronectin domain (e.g., SEQ ID NO: 82), or a human serum albumin (e.g., SEQ ID NO: 81)) and an extracellular portion of ActRIIA, ActRIIB, a variant thereof, or a chimera thereof described herein (e.g., an extracellular ActRIIA variant having the sequence of any one of SEQ ID NOs: 1-72 (e.g., SEQ ID NOs: 6-72)), is an amino acid spacer having the sequence GGG. For example, an ActRIIA ligand trap of the invention can contain an extracellular ActRIIA variant (e.g., any one of SEQ ID NOs: 6-72) fused to an Fc domain (e.g., SEQ ID NO: 79) by a GGG linker. An exemplary polypeptide containing an ActRIIA variant of SEQ ID NO: 69, a GGG linker, and an Fc domain lacking a terminal lysine (SEQ ID NO: 79) is provided below (SEQ ID NO: 80):

GAILGRSETQECLFYNANWELERTNQTGVERCEGEKDKRLHCYATWRNI
SGSIEIVKKGCWLDDFNCYDRTDCVETEENPQVYFCCCEGNMCNEKFSY
FPEMEVTQPTSGGGDKTHTCPPCPAPELLGGPSVFLFPPKPKDTLMISR
TPEVTCVVVDVSHEDPEVKFNWYVDGVEVHNAKTKPREEQYNSTYRVVS
VLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDG
SFFLYSKLTVDKSRWQQGNVFSCSVMHEALHNHYTQKSLSLSPG

### Vectors, Host Cells, and Protein Production

The ActRII signaling inhibitors of the invention can be produced from a host cell. A host cell refers to a vehicle that includes the necessary cellular components, e.g., organelles, needed to express the polypeptides and fusion polypeptides described herein from their corresponding nucleic acids. The nucleic acids may be included in nucleic acid vectors that can be introduced into the host cell by conventional techniques known in the art (e.g., transformation, transfection, electroporation, calcium phosphate precipitation, direct microinjection, infection, or the like). The choice of nucleic acid vectors depends in part on the host cells to be used. Generally, preferred host cells are of either eukaryotic (e.g., mammalian) or prokaryotic (e.g., bacterial) origin.

### Nucleic Acid Vector Construction and Host Cells

A nucleic acid sequence encoding the amino acid sequence of a polypeptide of the invention (i.e., an ActRII signaling inhibitor) may be prepared by a variety of methods known in the art. These methods include, but are not limited to, oligonucleotide-mediated (or site-directed) mutagenesis and PCR mutagenesis. A nucleic acid molecule encoding a polypeptide of the invention may be obtained using standard techniques, e.g., gene synthesis. Alternatively, for the production of ActRII ligand traps, a nucleic acid molecule encoding a wild-type portion of extracellular ActRIIA or ActRIIB may be mutated to include specific amino acid substitutions using standard techniques in the art, e.g., QuikChange™ mutagenesis. Nucleic acid molecules can be synthesized using a nucleotide synthesizer or PCR techniques.

A nucleic acid sequence encoding a polypeptide of the invention may be inserted into a vector capable of replicating and expressing the nucleic acid molecule in prokaryotic or eukaryotic host cells. Many vectors are available in the art and can be used for the purpose of the invention. Each vector may include various components that may be adjusted and optimized for compatibility with the particular host cell. For example, the vector components may include, but are not limited to, an origin of replication, a selection marker gene, a promoter, a ribosome binding site, a signal sequence, the nucleic acid sequence encoding protein of interest, and a transcription termination sequence.

In some embodiments, mammalian cells may be used as host cells for the invention. Examples of mammalian cell types include, but are not limited to, human embryonic kidney (HEK) (e.g., HEK293, HEK 293F), Chinese hamster ovary (CHO), HeLa, COS, PC3, Vero, MC3T3, NS0, Sp2/0, VERY, BHK, MDCK, W138, BT483, Hs578T, HTB2, BT20, T47D, NS0 (a murine myeloma cell line that does not endogenously produce any immunoglobulin chains), CRL7030, and HsS78Bst cells. In some embodiments, E. coli cells may also be used as host cells for the invention. Examples of E. coli strains include, but are not limited to, E. coli 294 (ATCC® 31,446), E. coli λ 1776 (ATCC®31,537, E. coli BL21 (DE3) (ATCC® BAA-1025), and E. coli RV308 (ATCC® 31,608). Different host cells have characteristic and specific mechanisms for the posttranslational processing and modification of protein products (e.g., glycosylation). Appropriate cell lines or host systems may be chosen to ensure the correct modification and processing of the polypeptide expressed. The above-described expression vectors may be introduced into appropriate host cells using conventional techniques in the art, e.g., transformation, transfection, electroporation, calcium phosphate precipitation, and direct microinjection. Once the vectors are introduced into host cells for protein production, host cells are cultured in conventional nutrient media modified as appropriate for inducing promoters, selecting transformants, or amplifying the genes encoding the desired sequences. Methods for expression of therapeutic proteins are known in the art, see, for example, Paulina Balbas, Argelia Lorence (eds.) Recombinant Gene Expression: Reviews and Protocols (Methods in Molecular Biology), Humana Press; 2nd ed. 2004 and Vladimir Voynov and Justin A. Caravella (eds.) Therapeutic Proteins: Methods and Protocols (Methods in Molecular Biology) Humana Press; 2nd ed. 2012.

### Protein Production, Recovery, and Purification

Host cells used to produce the polypeptides of the invention may be grown in media known in the art and suitable for culturing of the selected host cells. Examples of suitable media for mammalian host cells include Minimal Essential Medium (MEM), Dulbecco's Modified Eagle's Medium (DMEM), Expi293™ Expression Medium, DMEM with supplemented fetal bovine serum (FBS), and RPMI-1640. Examples of suitable media for bacterial host cells include Luria broth (LB) plus necessary supplements, such as a selection agent, e.g., ampicillin. Host cells are cultured at suitable temperatures, such as from about 20° C. to about 39° C., e.g., from 25° C. to about 37° C., preferably 37° C., and CO₂ levels, such as 5 to 10%. The pH of the medium is generally from about 6.8 to 7.4, e.g., 7.0, depending mainly on the host organism. If an inducible promoter is used in the expression vector of the invention, protein expression is induced under conditions suitable for the activation of the promoter.

In some embodiments, depending on the expression vector and the host cells used, the expressed protein may be secreted from the host cells (e.g., mammalian host cells) into the cell culture media. Protein recovery may involve filtering the cell culture media to remove cell debris. The proteins may be further purified. A polypeptide of the invention may be purified by any method known in the art of protein purification, for example, by chromatography (e.g., ion exchange, affinity, and size-exclusion column chromatography), centrifugation, differential solubility, or by any other standard technique for the purification of proteins. For example, the protein can be isolated and purified by appropriately selecting and combining affinity columns such as Protein A column (e.g., POROS Protein A chromatography) with chromatography columns (e.g., POROS HS-50 cation exchange chromatography), filtration, ultra-filtration, salting-out and dialysis procedures.

In other embodiments, host cells may be disrupted, e.g., by osmotic shock, sonication, or lysis, to recover the expressed protein. Once the cells are disrupted, cell debris may be removed by centrifugation or filtration. In some instances, a polypeptide can be conjugated to marker sequences, such as a peptide to facilitate purification. An example of a marker amino acid sequence is a hexa-histidine peptide (His-tag), which binds to nickel-functionalized agarose affinity column with micromolar affinity. Other peptide tags useful for purification include, but are not limited to, the hemagglutinin “HA” tag, which corresponds to an epitope derived from influenza hemagglutinin protein (Wilson et al., Cell 37:767, 1984).

Alternatively, the polypeptides of the invention can be produced by the cells of a subject (e.g., a human), e.g., in the context of gene therapy, by administrating a vector (such as a viral vector (e.g., a retroviral vector, adenoviral vector, poxviral vector (e.g., vaccinia viral vector, such as Modified Vaccinia Ankara (MVA)), adeno-associated viral vector, and alphaviral vector)) containing a nucleic acid molecule encoding the polypeptide of the invention. The vector, once inside a cell of the subject (e.g., by transformation, transfection, electroporation, calcium phosphate precipitation, direct microinjection, infection, etc.) will promote expression of the polypeptide, which is then secreted from the cell. If treatment of a disease or disorder is the desired outcome, no further action may be required. If collection of the protein is desired, blood may be collected from the subject and the protein purified from the blood by methods known in the art.

### Pharmaceutical Compositions and Preparations

The invention features pharmaceutical compositions that include the polypeptides described herein (e.g., an ActRII signaling inhibitor, such as an ActRII ligand trap including an extracellular portion of ActRIIA, ActRIIB, a variant thereof, or a chimera thereof (e.g., an extracellular ActRIIA variant having the sequence of any one of SEQ ID NOs: 1-72 (e.g., SEQ ID NOs: 6-72)). In some embodiments, a pharmaceutical composition of the invention includes an ActRII ligand trap including an extracellular ActRIIA variant (e.g., an extracellular ActRIIA variant having the sequence of any one of SEQ ID NOs: 1-70 (e.g., SEQ ID NOs: 6-70)) with a C-terminal extension (e.g., 1, 2, 3, 4, 5, 6 or more additional amino acids) as the therapeutic protein. In some embodiments, a pharmaceutical composition of the invention includes an ActRII ligand trap including an extracellular portion of ActRIIA, ActRIIB, a variant thereof, or a chimera thereof (e.g., an extracellular ActRIIA variant having the sequence of any one of SEQ ID NOs: 1-72 (e.g., SEQ ID NOs: 6-72)) fused to a moiety (e.g., Fc domain monomer, or a dimer thereof, an Fc domain, an albumin-binding peptide, a fibronectin domain, or a human serum albumin) as the therapeutic protein. In some embodiments, a pharmaceutical composition of the invention including a polypeptide of the invention may be used in combination with other agents (e.g., therapeutic biologics and/or small molecules) or compositions in a therapy. In addition to a therapeutically effective amount of the polypeptide, the pharmaceutical composition may include one or more pharmaceutically acceptable carriers or excipients, which can be formulated by methods known to those skilled in the art. In some embodiments, a pharmaceutical composition of the invention includes a nucleic acid molecule (DNA or RNA, e.g., mRNA) encoding a polypeptide of the invention, or a vector containing such a nucleic acid molecule.

Acceptable carriers and excipients in the pharmaceutical compositions are nontoxic to recipients at the dosages and concentrations employed. Acceptable carriers and excipients may include buffers such as phosphate, citrate, HEPES, and TAE, antioxidants such as ascorbic acid and methionine, preservatives such as hexamethonium chloride, octadecyldimethylbenzyl ammonium chloride, resorcinol, and benzalkonium chloride, proteins such as human serum albumin, gelatin, dextran, and immunoglobulins, hydrophilic polymers such as polyvinylpyrrolidone, amino acids such as glycine, glutamine, histidine, arginine, and lysine, and carbohydrates such as glucose, mannose, sucrose, and sorbitol. Pharmaceutical compositions of the invention can be administered parenterally in the form of an injectable formulation. Pharmaceutical compositions for injection can be formulated using a sterile solution or any pharmaceutically acceptable liquid as a vehicle. Pharmaceutically acceptable vehicles include, but are not limited to, sterile water, physiological saline, and cell culture media (e.g., Dulbecco's Modified Eagle Medium (DMEM), α-Modified Eagles Medium (α-MEM), F-12 medium). Formulation methods are known in the art, see e.g., Banga (ed.) Therapeutic Peptides and Proteins: Formulation, Processing and Delivery Systems (3rd ed.) Taylor & Francis Group, CRC Press (2015).

The pharmaceutical compositions of the invention may be prepared in microcapsules, such as hydroxylmethylcellulose or gelatin-microcapsule and poly-(methylmethacrylate) microcapsule. The pharmaceutical compositions of the invention may also be prepared in other drug delivery systems such as liposomes, albumin microspheres, microemulsions, nanoparticles, and nanocapsules. Such techniques are described in Remington: The Science and Practice of Pharmacy 22^{nd} edition (2012). The pharmaceutical compositions to be used for in vivo administration must be sterile. This is readily accomplished by filtration through sterile filtration membranes.

The pharmaceutical compositions of the invention may also be prepared as a sustained-release formulation. Suitable examples of sustained-release preparations include semipermeable matrices of solid hydrophobic polymers containing the polypeptides of the invention. Examples of sustained release matrices include polyesters, hydrogels, polylactides, copolymers of L-glutamic acid and γ ethyl-L-glutamate, non-degradable ethylene-vinyl acetate, degradable lactic acid-glycolic acid copolymers such as LUPRON DEPOT™, and poly-D-(−)-3-hydroxybutyric acid. Some sustained-release formulations enable release of molecules over a few months, e.g., one to six months, while other formulations release pharmaceutical compositions of the invention for shorter time periods, e.g., days to weeks.

The pharmaceutical composition may be formed in a unit dose form as needed. The amount of active component, e.g., a polypeptide of the invention, included in the pharmaceutical preparations is such that a suitable dose within the designated range is provided (e.g., a dose within the range of 0.01-100 mg/kg of body weight).

The pharmaceutical composition for gene therapy can be in an acceptable diluent or can include a slow-release matrix in which the gene delivery vehicle is imbedded. If hydrodynamic injection is used as the delivery method, the pharmaceutical composition containing a nucleic acid molecule encoding a polypeptide described herein or a vector (e.g., a viral vector) containing the nucleic acid molecule is delivered rapidly in a large fluid volume intravenously. Vectors that may be used as in vivo gene delivery vehicle include, but are not limited to, retroviral vectors, adenoviral vectors, poxviral vectors (e.g., vaccinia viral vectors, such as Modified Vaccinia Ankara), adeno-associated viral vectors, and alphaviral vectors.

### Routes, Dosage, and Administration

Pharmaceutical compositions that include the polypeptides of the invention as the therapeutic proteins may be formulated for, e.g., intravenous administration, parenteral administration, subcutaneous administration, intramuscular administration, intra-arterial administration, intrathecal administration, or intraperitoneal administration. The pharmaceutical composition may also be formulated for, or administered via, oral, nasal, spray, aerosol, rectal, or vaginal administration. For injectable formulations, various effective pharmaceutical carriers are known in the art. See, e.g., ASHP Handbook on Injectable Drugs, Toissel, 18th ed. (2014).

In some embodiments, a pharmaceutical composition that includes a nucleic acid molecule encoding a polypeptide of the invention or a vector containing such nucleic acid molecule may be administered by way of gene delivery. Methods of gene delivery are well-known to one of skill in the art. Vectors that may be used for in vivo gene delivery and expression include, but are not limited to, retroviral vectors, adenoviral vectors, poxviral vectors (e.g., vaccinia viral vectors, such as Modified Vaccinia Ankara (MVA)), adeno-associated viral vectors, and alphaviral vectors. In some embodiments, mRNA molecules encoding polypeptides of the invention may be administered directly to a subject.

In some embodiments of the present invention, nucleic acid molecules encoding a polypeptide described herein or vectors containing such nucleic acid molecules may be administered using a hydrodynamic injection platform. In the hydrodynamic injection method, a nucleic acid molecule encoding a polypeptide described herein is put under the control of a strong promoter in an engineered plasmid (e.g., a viral plasmid). The plasmid is often delivered rapidly in a large fluid volume intravenously. Hydrodynamic injection uses controlled hydrodynamic pressure in veins to enhance cell permeability such that the elevated pressure from the rapid injection of the large fluid volume results in fluid and plasmid extravasation from the vein. The expression of the nucleic acid molecule is driven primarily by the liver. In mice, hydrodynamic injection is often performed by injection of the plasmid into the tail vein. In certain embodiments, mRNA molecules encoding a polypeptide described herein may be administered using hydrodynamic injection.

The dosage of the pharmaceutical compositions of the invention depends on factors including the route of administration, the disease to be treated, and physical characteristics, e.g., age, weight, general health, of the subject. A pharmaceutical composition of the invention may include a dosage of an ActRII signaling inhibitor of the invention ranging from 0.01 to 500 mg/kg (e.g., 0.01, 0.1, 0.2, 0.3, 0.325, 0.35, 0.375, 0.4, 0.5, 0.75, 1, 1.25, 1.5, 1.75, 2, 2.25, 2.5, 2.75, 3, 3.25, 3.5, 3.75, 4, 4.25, 4.5, 4.75, 5, 10, 15, 20, 25, 30, 35, 40, 45, 50, 100, 150, 200, 250, 300, 350, 400, 450, or 500 mg/kg) and, in a more specific embodiment, about 0.1 to about 30 mg/kg and, in a more specific embodiment, about 0.3 to about 30 mg/kg. The dosage may be adapted by the physician in accordance with conventional factors such as the extent of the disease and different parameters of the subject.

The pharmaceutical compositions are administered in a manner compatible with the dosage formulation and in such amount as is therapeutically effective to result in an improvement or remediation of the symptoms. The pharmaceutical compositions are administered in a variety of dosage forms, e.g., intravenous dosage forms, subcutaneous dosage forms, and oral dosage forms (e.g., ingestible solutions, drug release capsules). Generally, therapeutic proteins are dosed at 0.1-100 mg/kg, e.g., 0.5-50 mg/kg. Pharmaceutical compositions that include a polypeptide of the invention may be administered to a subject in need thereof, for example, one or more times (e.g., 1-10 times or more) daily, weekly, biweekly, every four weeks, monthly, bimonthly, quarterly, biannually, annually, or as medically necessary. In some embodiments, pharmaceutical compositions that include a polypeptide of the invention may be administered to a subject in need thereof weekly, biweekly, every four weeks, monthly, bimonthly, or quarterly. Dosages may be provided in either a single or multiple dosage regimens. The timing between administrations may decrease as the medical condition improves or increase as the health of the patient declines.

### Methods of Treatment

The ActRII signaling inhibitors described herein (e.g., an activin A antibody, a myostatin antibody, an activin B antibody, a GDF-11 antibody, an ActRII antibody, or an ActRII ligand trap) can be used to treat a subject receiving treatment with a cytopenia-associated myelofibrosis treatment. In some embodiments, the subject has a cytopenia (e.g., anemia, thrombocytopenia, and/or neutropenia) (e.g., the subject has already developed a cytopenia or is identified as having a cytopenia prior to treatment with an ActRII signaling inhibitor described herein). In some embodiments, the subject has not yet developed a cytopenia or is not identified as having a cytopenia when treatment with the ActRII signaling inhibitor is initiated. In some embodiments, the subject is receiving treatment with a cytopenia-associated myelofibrosis treatment for myelofibrosis, such as PMF, post-ET MF, or post-PV MF (e.g., diagnosed according to the 2017 World Health Organization criteria). In some embodiments, the myelofibrosis is intermediate or high-risk myelofibrosis. In some embodiments, the subject has an Eastern Cooperative Oncology Group (ECOG) performance score of less than or equal to two. In some embodiments, the subject is receiving treatment with a cytopenia-associated myelofibrosis treatment for polycythemia vera. In some embodiments, the subject is receiving treatment with a cytopenia-associated myelofibrosis treatment for steroid-refractory acute graft-versus-host disease. In some embodiments, the subject receiving treatment with a cytopenia-associated myelofibrosis treatment has anemia. Anemia is defined as hemoglobin ≤10 g/dL during screening, or receiving RBC transfusions. In some embodiments, the subject receiving treatment with a cytopenia-associated myelofibrosis treatment has thrombocytopenia. In some embodiments, the subject receiving treatment with a cytopenia-associated myelofibrosis treatment has both anemia and thrombocytopenia. In some embodiments, the subject receiving treatment with a cytopenia-associated myelofibrosis treatment has neutropenia. In some embodiments, the subject receiving treatment with a cytopenia-associated myelofibrosis treatment has anemia and neutropenia. In some embodiments, the subject receiving treatment with a cytopenia-associated myelofibrosis treatment has thrombocytopenia and neutropenia. In some embodiments, the subject receiving treatment with a cytopenia-associated myelofibrosis treatment has anemia, thrombocytopenia, and neutropenia. In some embodiments, the subject has been receiving treatment with the cytopenia-associated myelofibrosis treatment for at least eight weeks (e.g., 8 weeks or longer, such as 8, 9, 10, 11, 12, 13, 14, 15, 16, 17, 18, 19, 20, 21, 22, 23, or more weeks, or 6, 7, 8, 9, 10, 11, 12, 13, 14, 15, 16, 17, 18, 19, 20, 21, 22, 23, 24 or more months) and has been on a stable dose of the cytopenia-associated myelofibrosis treatment for at least 4 weeks (e.g., 4 weeks or longer, such as 4, 5, 6, 7, 8, 9, 10, 11, 12, 13, 14, 15, 16, 17, 18, 19, 20, 21, 22, 23, or more weeks, or 6, 7, 8, 9, 10, 11, 12, 13, 14, 15, 16, 17, 18, 19, 20, 21, 22, 23, 24 or more months) prior to co-administration of an ActRII signaling inhibitor described herein. In some embodiments, the subject has been receiving treatment with the cytopenia-associated myelofibrosis treatment for at least eight weeks and less than six months (e.g., 8, 9, 10, 11, 12, 13, 14, 15, 16, 17, 18, 19, 20, 21, 22, 23 or 24 weeks) and has been on a stable dose of the cytopenia-associated myelofibrosis treatment for at least 4 weeks (e.g., 4, 5, 6, 7, 8, 9, 10, 11, 12, 13, 14, 15, 16, 17, 18, 19, 20, 21, 22, 23, or 24 weeks) prior to co-administration of an ActRII signaling inhibitor described herein. In some embodiments, the subject has been receiving treatment with the cytopenia-associated myelofibrosis treatment for less than eight weeks (e.g., 7, 6, 5, 4, 3, 2, 1 weeks or shorter) prior to co-administration of an ActRII signaling inhibitor described herein. In some embodiments, treatment with the cytopenia-associated myelofibrosis treatment and the ActRII signaling inhibitor is started concurrently (e.g., the subject begins treatment with both agents at approximately the same time, e.g., begins treatment with both agents during the same day, week, or month). In some embodiments, the subject is identified as having a cytopenia (e.g., anemia, thrombocytopenia, or neutropenia) associated with a cytopenia-associated myelofibrosis treatment prior to co-administration of an ActRII signaling inhibitor described herein. In some embodiments, the method includes a step of identifying the subject as having a cytopenia (e.g., anemia, thrombocytopenia, or neutropenia) associated with a cytopenia-associated myelofibrosis treatment (e.g., by evaluating red blood cell, hemoglobin, hematocrit, platelet, and/or neutrophil levels) prior to co-administration of an ActRII signaling inhibitor described herein. The method can further include evaluating red blood cell, hemoglobin, hematocrit, reticulocyte, platelet, and/or neutrophil levels after administration of an ActRII signaling inhibitor described herein (e.g., 12 hours, 24 hours, 1, 2, 3, 4, 5, 6, or 7 days, 1, 2, 3, 4, 5, 6, 7, 8, 9, 10, 11, or 12 weeks, or 1, 2, 3, 4, 5, 6, 8, 10, 12, 18, or 24 months or more after the start of treatment with an ActRII signaling inhibitor described herein, such as by taking a CBC). In some embodiments, the subject does not receive concurrent treatment with an erythropoiesis stimulating agent (ESA), granulocyte colony-stimulating factor (G-CSF), granulocyte-macrophage colony-stimulating factor (GM-CSF), a thrombopoietin agonist (TPO), an immunomodulator imide drug (IMiD; e.g., thalidomide, pomalidomide, lenalidomide), interferon, hydroxyurea, danazol, or a steroid (other than prednisone of less than or equal to 10 mg/day or corticosteroid equivalent). In some embodiments, the subject has not previously been treated with luspatercept, sotatercept, or other TGF-β inhibitors.

In some embodiments, the methods described herein increase hemoglobin levels, increase hematocrit, increase red blood cell count, increase red blood cell volume, increase red cell mass, increase reticulocytes, increase proerythroblasts, increase or induce red blood cell formation or production, increase the maturation and/or differentiation of erythroid progenitors (early or late- (e.g., terminal) stage progenitors, e.g., early-stage erythroid progenitors, such burst forming unit-erythroid cells (BFU-Es) and/or colony forming unit-erythroid cells (CFU-Es), e.g., increase the maturation and/or differentiation of BFU-Es and/or CFU-Es into proerythroblasts, reticulocytes, or red blood cells, e.g., increase proerythroblast and/or reticulocyte numbers), increase late-stage erythroid precursor maturation (e.g., terminal maturation, such as the maturation of reticulocytes into red blood cells, or the maturation of erythroblasts into reticulocytes and/or red blood cells), recruit early-stage progenitors into the erythroid lineage, increase the number of early-stage erythroid precursors and/or progenitors (e.g., expand the early-stage precursor population to provide a continuous supply of precursors to replenish polychromatic erythroblasts and allow for a continuous supply of maturing reticulocytes), promote the progression of erythroid precursors and/or progenitors through erythropoiesis, reduce the accumulation of red blood cell progenitor cells (e.g., by stimulating progenitor cells to progress to maturation), increase platelet levels (e.g., increase platelet count), increase or induce megakaryocyte differentiation and/or maturation (e.g., to produce platelets, e.g., terminal maturation of pro-platelets to platelets), reduce platelet progenitor accumulation (e.g., by stimulating progenitor cells to progress to maturation), increase megakaryocyte progenitors (e.g., increase megakaryocyte progenitor renewal), increase pro-platelets, promote or increase platelet formation or production, increase neutrophil levels (e.g., increase neutrophil count), increase or induce the differentiation and/or maturation of progenitor cells (e.g., myeloid progenitors, myeloblasts, or myelocytes) into neutrophils, and/or induce or increase neutrophil formation or production in the subject. In some embodiments, the methods described herein increase the rate of recovery from thrombocytopenia. These changes may be observed in a subject treated with an ActRII signaling inhibitor described herein compared to measurements obtained prior to treatment or compared to measurements obtained from subjects treated only with a cytopenia-associated myelofibrosis treatment. In some embodiments, the methods described herein improve or restore hematopoiesis in the bone marrow, reduce or reverse reticulin and/or collagen deposition, or reverse bony changes associated with myelofibrosis. In some embodiments, the methods described herein reduce or ameliorate megakaryocyte dysfunction (e.g., megakaryocyte dysfunction in the bone marrow), which may prevent or reduce inflammation/fibrosis, restore hematopoiesis in the bone marrow, and treat cytopenias due to myelofibrosis and those that result from JAK inhibitor treatment. In some embodiments, the methods described herein reduce or resolve hepatosplenomegaly or splenomegaly (e.g., reduce spleen volume and/or splenic extra medullary hematopoiesis) and its symptoms. In some embodiments, the methods described herein reduce bone marrow fibrosis and alleviate the symptoms caused by the loss of bone marrow function. In some embodiments, the methods described herein slow or reduce the progression of bone marrow fibrosis. In some embodiments, the methods described herein improve or ameliorate the attenuated bone resorption and osteosclerosis in patients with myelofibrosis. In some embodiments, the methods described herein improve fibrosis, bone histomorphology, spleen size (e.g., reduce spleen size), myelofibrosis symptoms, bone marrow fibrosis, and/or osteosclerotic dysplasia. In some embodiments, the methods described herein increase body weight. In some embodiments, the methods described treat or reduce cachexia. In some embodiments, the methods described treat or reverse a cytopenia (e.g., anemia, thrombocytopenia, and/or neutropenia) caused by a cytopenia-associated myelofibrosis treatment and reverse reductions in red blood cells, platelets, and/or neutrophils induced by the cytopenia-associated myelofibrosis treatment. In some embodiments, the methods described herein reduce bleeding events. In some embodiments, the methods described herein decrease infections.

In some embodiments, treatment according to methods described herein leads to a mean hemoglobin increase of greater than or equal to 1.5 g/dL or 2.0 g/dL from baseline or pretreatment measurements over a period of 12 consecutive weeks or more, such as 12 weeks, 14 weeks, 16 weeks, 18 weeks, 20 weeks, 22 weeks, 24 weeks, 26 weeks, 1 year, 2 years or more, during treatment with an ActRII signaling inhibitor described herein, for example during the first 24 weeks or 52 weeks of treatment of a transfusion-independent subject according to the methods described herein. In some embodiments, treatment according to methods described herein leads to a decrease of one or more in the brief fatigue inventory score from baseline within the first 24 weeks or 52 weeks of treatment of a transfusion-independent subject according to the methods described herein. In some embodiments, the methods described herein reduce the need of a subject, such as a subject with anemia requiring RBC transfusions, for a blood transfusion (e.g., reduce transfusion burden, for example, the subject no longer needs blood transfusions, or the subject needs less frequent blood transfusion than before treatment with the compositions and methods described herein). In some embodiments, treatment according to the methods described herein reduces the number of RBC transfusions from baseline pre-treatment measurements (e.g., measurements taken over 12 weeks directly preceding treatment initiation with an ActRII signaling inhibitor described herein) for a period of 12 consecutive weeks of more, such as 12 weeks, 14 weeks, 16 weeks, 18 weeks, 20 weeks, 22 weeks, 24 weeks, 26 weeks, 1 year, 2 years or more, during treatment with an ActRII signaling inhibitor described herein, for example during the first 24 weeks or 52 weeks of treatment according to the methods described herein). In some embodiments, the compositions and methods described herein promote transfusion independence (e.g., a subject who required 1 or more (e.g., 2, 3, 4, 5, 6, 7, 8, 9, 10, or more) RBC units over 12 weeks directly preceding treatment initiation with an ActRII signaling inhibitor described herein does not require a transfusion for 12 consecutive weeks of more, such as 12 weeks, 14 weeks, 16 weeks, 18 weeks, 20 weeks, 22 weeks, 24 weeks, 26 weeks, 1 year, 2 years or more, during treatment with an directly preceding treatment initiation with an ActRII signaling inhibitor described herein, for example during the first 24 weeks or 52 weeks of treatment according to the methods described herein). Concurrent treatment for anemia with RBC transfusions is recommended when hemoglobin is <8.0 g/dL, and may be recommended if Hgb is ≥8.0 g/dL and associated with symptom(s) of anemia (e.g., hemodynamic or pulmonary compromise requiring treatment) or comorbidity justifying a threshold ≥8.0 g/dL Hgb. A complete blood count (CBC) can be taken to assess the response of a subject to treatment with a composition described herein, and hemoglobin levels can be reviewed to determine whether the subject has a stable hemoglobin level above the transfusion threshold. In subjects who achieve transfusion independence, both hemoglobin levels and absolute reticulocyte counts may increase. In some embodiments, treatment according to the methods described herein leads to an improvement in the Myelofibrosis Symptom Assessment Form Total Symptom Score (MF-SAF-TSS) of greater than or equal to 50% from baseline (e.g., at least 50%, 55%, 60%, 65%, 70%, 75%, 80%, 85%, 90% or more from baseline), such as by 24 weeks or 52 weeks of treatment according to the methods described herein. In some embodiments, treatment according to the methods described herein leads to a decrease in spleen volume of greater than or equal to 35% from baseline (e.g., at least 35%, 40%, 45%, 50%, 55%, 60%, 65%, 70%, 75% or more from baseline) as measured by computed tomography, such as by 24 weeks or 52 weeks of treatment according to the methods described herein. In some embodiments, the compositions and methods described herein slow or inhibit the progression to acute myeloid leukemia (AML) (bone marrow blasts >20%) and/or accelerated MF (bone marrow blasts ≥10%), such as by 24 weeks or 52 weeks of treatment according to the methods described herein. In some embodiments, treatment according to the methods described herein leads to a mean platelet increase from baseline of greater than 30×10⁹/L for 12 weeks or more, such as 12 weeks, 14 weeks, 16 weeks, 18 weeks, 20 weeks, 22 weeks, 24 weeks, 26 weeks, 1 year, 2 years or more, during treatment with an ActRII signaling inhibitor described herein (in the absence of platelet transfusions), for example by 24 weeks or 52 weeks of treatment according to the methods described herein. In some embodiments, treatment according to the methods described herein reduces episodes of anemia, neutropenia, and thrombocytopenia of ≥Grade 1. In some embodiments, treatment according to the methods described herein allows subjects to maintain the dose intensity of the cytopenia-associated myelofibrosis treatment. In some embodiments, treatment according to the methods described herein improves tolerability or adherence to the cytopenia-associated myelofibrosis treatment, for example, the subject can remain on the cytopenia-associated myelofibrosis treatment for 12 weeks, 24 weeks, 52 weeks, or longer with concomitant treatment with an ActRII signaling inhibitor described herein. In some embodiments, treatment according to the methods described herein reduces osteosclerosis from baseline as assessed using CT, such as by 24 weeks or 52 weeks of treatment as described herein. In some embodiments, treatment according to the methods described herein leads to a decrease in Patient Reported Outcomes Measurement Information System (PROMIS) score or BFI score from baseline, such as by 24 weeks or 52 weeks of treatment as described herein. In some embodiments, treatment according to the methods described herein slows or reduces the progression of bone marrow fibrosis or improves (e.g., reverses) bone marrow fibrosis. For example, treatment according to the methods described herein may lead to an improvement in bone marrow fibrosis grade from baseline or may prevent bone marrow fibrosis grade from worsening, such as by 24 weeks or 52 weeks of treatment as described herein. Treatment according to the methods described herein may also increase red cell parameters, such as reticulocyte count, mean corpuscular volume (MCV), mean corpuscular hemoglobin (MCH), and reticulocyte cell hemoglobin, and/or biomarkers of blood cell production, such as erythropoietin (EPO) and thrombopoietin (TPO) levels. In some embodiments, treatment according to the methods described herein increases biomarkers of bone metabolism compared to baseline, such as bone specific alkaline phosphatase (BSAP) and serum C-telopeptide of type I collagen (CTX). In some embodiments, treatment according to the methods described herein reduces the development of myelofibrosis-associated molecular and cytogenic abnormalities over the duration of treatment. Treatment according to the methods described herein may also lead to changes in biomarkers of iron metabolism (e.g., serum iron, ferritin, transferrin, transferrin saturation, total iron binding capacity, soluble transferrin receptor level, and hepcidin), dose of iron chelators, and cytokine levels compared to baseline, such as by 24 weeks or 52 weeks of treatment as described herein.

In some embodiments, the methods described herein do not cause any vascular complications in the subject, such as increased vascular permeability or leakage.

In some embodiments the ActRII signaling inhibitor that is co-administered with a cytopenia-associated myelofibrosis treatment is an ActRIIA ligand trap including an extracellular ActRIIA variant (e.g., an extracellular ActRIIA variant having the sequence of any one of SEQ ID NOs: 1-72 (e.g., SEQ ID NOs: 6-72). In some embodiments, the ActRIIA ligand trap including an extracellular ActRIIA variant is administered at a dosage ranging from 0.01 to 500 mg/kg (e.g., 0.01, 0.1, 0.2, 0.3, 0.325, 0.35, 0.375, 0.4, 0.5, 0.75, 1, 1.25, 1.5, 1.75, 2, 2.25, 2.5, 2.75, 3, 3.25, 3.5, 3.75, 4, 4.25, 4.5, 4.75, 5, 10, 15, 20, 25, 30, 35, 40, 45, 50, 100, 150, 200, 250, 300, 350, 400, 450, or 500 mg/kg) and, in a more specific embodiment, about 0.1 to about 30 mg/kg and, in a more specific embodiment, about 0.3 to about 30 mg/kg. In any of the methods described herein, an ActRIIA ligand trap including an extracellular ActRIIA variant (e.g., an extracellular ActRIIA variant having the sequence of any one of SEQ ID NOs: 1-71 (e.g., SEQ ID NOs: 6-71)) that further includes a C-terminal extension of one or more amino acids (e.g., 1, 2, 3, 4, 5, 6 or more amino acids) may be used as the therapeutic protein. In any of the methods described herein, a dimer (e.g., homodimer or heterodimer) formed by the interaction of two Fc domain monomers that are each fused to a polypeptide including an extracellular ActRIIA variant (e.g., an extracellular ActRIIA variant having the sequence of any one of SEQ ID NOs: 1-72 (e.g., SEQ ID NOs: 6-72)) may be used as the therapeutic protein. In any of the methods described herein, an ActRIIA ligand trap including an extracellular ActRIIA variant (e.g., an extracellular ActRIIA variant having the sequence of any one of SEQ ID NOs: 1-72 (e.g., SEQ ID NOs: 6-72)) fused to a moiety (e.g., an Fc domain monomer, an Fc domain, an albumin-binding peptide, a fibronectin domain, or a human serum albumin) may be used as the therapeutic protein. Nucleic acids encoding the polypeptides described herein, or vectors containing said nucleic acids can also be administered according to any of the methods described herein. In any of the methods described herein, the polypeptide, nucleic acid, or vector can be administered as part of a pharmaceutical composition.

Compositions that can be administered to a subject according to the methods described herein are provided in Tables 18-21, below.

### Combination Therapy

An ActRII signaling inhibitor described herein is to be administered to the subject in combination with a cytopenia-associated myelofibrosis treatment. The cytopenia-associated myelofibrosis treatment may be, e.g., ruxolitinib (JAKAFI®/JAKAVI®), fedratinib (INREBIC®), pacritinib (VONJO™), or imetelstat. The cytopenia-associated myelofibrosis treatment may be administered at the same time (e.g., administration of all agents occurs within 15 minutes, 10 minutes, 5 minutes, 2 minutes or less) as the ActRII signaling inhibitor. The agents can also be administered simultaneously via co-formulation. The ActRII signaling inhibitor and the cytopenia-associated myelofibrosis treatment can also be administered sequentially, such that the action of the two overlaps and their combined effect is such that the reduction in a symptom, or other parameter related to the disorder is greater than what would be observed with one agent or treatment delivered alone or in the absence of the other. The effect of the ActRII signaling inhibitor and the cytopenia-associated myelofibrosis treatment can be partially additive, wholly additive, or greater than additive (e.g., synergistic). Sequential or substantially simultaneous administration of each of the ActRII signaling inhibitor and the cytopenia-associated myelofibrosis treatment can be performed by any appropriate route including, but not limited to, oral routes, intravenous routes, intramuscular routes, local routes, and direct absorption through mucous membrane tissues. The ActRII signaling inhibitor and the cytopenia-associated myelofibrosis treatment can be administered by the same route or by different routes. For example, an ActRII signaling inhibitor may be administered by subcutaneous (e.g., for an ActRII ligand trap) or intravenous (e.g., for an Activin A, Activin B, myostatin, GDF-11, or ActRII antibody) injection or infusion while the cytopenia-associated myelofibrosis treatment can be administered orally (for ruxolitinib, fedratinib, or pacritinib) or by intravenous injection or infusion (for imetelstat). The ActRII signaling inhibitor may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours, up to 24 hours or up to 1-7, 1-14, 1-21 or 1-30 days before or after the cytopenia-associated myelofibrosis treatment. In some embodiments, the ActRII signaling inhibitor and the cytopenia-associated myelofibrosis treatment are administered at different frequencies. For example, the ActRII signaling inhibitor can be administered once a week, once every two weeks, once every four weeks, once a month, once bimonthly, once every three months, once every four months, or once every six months and the cytopenia-associated myelofibrosis treatment can be administered once or twice daily (e.g., for ruxolitinib, fedratinib, or pacritinib). In some embodiments, the ActRII signaling inhibitor and the cytopenia-associated myelofibrosis treatment are administered at the same or at similar frequencies. For example, both the ActRII signaling inhibitor and the cytopenia-associated myelofibrosis treatment can be administered once a week, once every two weeks, once every four weeks, once a month, once bimonthly, once every three months, once every four months, or once every six months (e.g., when the cytopenia-associated myelofibrosis treatment is imetelstat).

In some embodiments, the cytopenia-associated myelofibrosis treatment is administered as indicated on the label. For example, ruxolitinib can be administered at a starting dose of 20 mg orally twice daily for subjects with myelofibrosis and greater than 200×10⁹/L platelets at baseline, 15 mg orally twice daily for subjects with myelofibrosis and 100×10⁹/L to 200×10⁹/L platelets at baseline, and 5 mg orally twice daily for subjects with myelofibrosis and 50×10⁹/L to less than 100×10⁹/L platelets at baseline, 10 mg orally twice daily for polycythemia vera, or 5 mg orally twice daily for acute graft-versus-host disease, which can be increased to 10 mg twice daily after at least three days of treatment. In some embodiments, ruxolitinib is administered at a dose of 10 mg/day to 50 mg/day (e.g., 10 mg/day, 15 mg/day, 20 mg/day, 25 mg/day, 30 mg/day, 35 mg/day, 40 mg/day, 45 mg/day, or 50 mg/day). When administered alone, dosing may need to be reduced or discontinued due to the development of cytopenias, but, when administered in combination with an ActRII signaling inhibitor, the subject may be able to remain on the same dose of ruxolitinib with few to no treatment discontinuations and may be able to receive a higher dose of ruxolitinib (e.g., by 5 mg or more, e.g., 5 mg, 10 mg, or 15 mg) compared to the dose of ruxolitinib when it is administered alone. Fedratinib may be taken at a dose of 400 mg or less once daily, such as 400 mg, 300 mg, 200 mg, or 100 mg once daily by subjects with myelofibrosis (e.g., for example, by subjects with intermediate-2 or high-risk primary or secondary myelofibrosis and greater than 50×10⁹/L platelets at baseline). When administered alone, dosing may need to be reduced or discontinued due to the development of cytopenias, but, when administered in combination with an ActRII signaling inhibitor, the subject may be able to remain on the same dose of fedratinib with few to no treatment discontinuations. Pacritinib may be taken at a dose of 200 mg twice daily by subjects with myelofibrosis (e.g., by subjects with intermediate or high-risk primary or secondary (post-polycythemia vera or post-essential thrombocythemia) myelofibrosis with a platelet count below 50×10⁹/L platelets at baseline), which may be reduced to 100 mg twice daily or 100 mg once daily if dose modification is needed for adverse reactions. When administered alone, dosing may need to be reduced or discontinued due to the development of cytopenias, but, when administered in combination with an ActRII signaling inhibitor, the subject may be able to remain on the same dose of pacritinib with few to no treatment discontinuations. Imetelstat may be administered by intravenous infusion at a dose of about 1.0 mg/kg to about 50 mg/kg (e.g., 1.0, 2.0, 3.0, 4.0, 5.0, 6.0, 7.0, 7.5, 8.0, 9.0, 9.4, 10.0, 11.0, 12.0, 13.0, 14.0, 15.0, 16.0, 17.0, 18.0, 19.0, 20.0, 25.0, 30.0, 35.0, 40.0, 45.0, or 50.0 mg/kg) once a week, once every two weeks, once every four weeks, once a month, once bimonthly, once every three months, once every four months, or once every six months. When administered alone, dosing may need to be reduced or discontinued due to the development of cytopenias, but, when administered in combination with an ActRII signaling inhibitor, the subject may be able to remain on the same dose of imetelstat with few to no treatment discontinuations and may be able to receive a higher dose of imetelstat (e.g., by 1.0 mg/kg or more, e.g., 1.0 mg/kg, 2.0, mg/kg, 3.0 mg/kg, 4.0 mg/kg, 5.0 mg/kg or more) compared to the dose of imetelstat when it is administered alone or receive imetelstat treatment less frequently. The ActRII signaling inhibitor can be administered by subcutaneous or intravenous injection or infusion at a dose of from about 0.01 to about 500 mg/kg (e.g., 0.01, 0.1, 0.2, 0.3, 0.325, 0.35, 0.375, 0.4, 0.5, 0.75, 1, 1.25, 1.5, 1.75, 2, 2.25, 2.5, 2.75, 3, 3.25, 3.5, 3.75, 4, 4.25, 4.5, 4.75, 5, 10, 15, 20, 25, 30, 35, 40, 45, 50, 100, 150, 200, 250, 300, 350, 400, 450, or 500 mg/kg) and, in a more specific embodiment, about 0.1 to about 30 mg/kg and, in a more specific embodiment, about 0.3 to about 30 mg/kg, once a week, once every two weeks, once every four weeks, once a month, once bimonthly, once every three months, once every four months, or once every six months, or once a year.

In some embodiments, combination therapy with an ActRII signaling inhibitor and a cytopenia-associated myelofibrosis treatment reduces the adverse reactions associated with the cytopenia-associated myelofibrosis treatment, such as the development of anemia, thrombocytopenia, and/or neutropenia that can lead to treatment interruptions and discontinuations. In some embodiments, combination therapy reduces or ameliorates anemia, thrombocytopenia, and/or neutropenia, or reduces the number of episodes of one or more of these cytopenias. In some embodiments, combination therapy improves adherence to treatment with the cytopenia-associated myelofibrosis treatment (e.g., the subject can remain on treatment for a longer period of time), improves tolerability of the cytopenia-associated myelofibrosis treatment (e.g., the subject can remain on the same dose or increase the dose of the cytopenia-associated myelofibrosis treatment), decreases transfusion burden, decreases bleeding events, decreases infections, or decreases interruptions or discontinuations in treatment with the cytopenia-associated myelofibrosis treatment.

### Kits

An ActRII signaling inhibitor and a cytopenia-associated myelofibrosis treatment described herein can be provided in a kit for use in treating myelofibrosis. Each agent may be provided in unit dosage form, optionally in a pharmaceutically acceptable excipient (e.g., saline), in an amount sufficient to treat myelofibrosis. The kit can further include a package insert that instructs a user of the kit, such as a physician, to perform the methods described herein. The kit may optionally include a syringe or other device for administering the ActRII signaling inhibitor or cytopenia-associated myelofibrosis treatment.

TABLE 18

| Row | Composition |
|---|---|
| 1 | A polypeptide comprising an extracellular activin receptor type IIa (ActRIIa) |
| | variant, the variant having a sequence of |
| | GAILGRSETQECLX₁X₂NANWX₃X₄X₅X₆TNQTGVEX₇CX₈GX₉X₁₀X₁₁X₁₂X₁₃X₁₄HCX₁₅ATWX₁₆NI |
| | SGSIEIVX₁₇X₁₈GCX₁₉X₂₀X₂₁DX₂₂NCYDRTDCVEX₂₃X₂₄X₂₅X₂₆PX₂₇VYFCCCEGNMCNEKFSYF |
| | PEMEVTQPTS (SEQ ID NO: 1), |
| | wherein X₁ is F or Y; X₂ is F or Y; X₃ is E or A; X₄ is K or L; X₅ is D or E; X₆ is R |
| | or A; X₇ is P or R; X₈ is Y or E; X₉ is D or E; X₁₀ is K or Q; X₁₁ is D or A; X₁₂ is K |
| | or A; X₁₃ is R or A; X₁₄ is R or L; X₁₅ is F or Y; X₁₆ is K, R, or A; X₁₇ is K, A, Y, |
| | F, or I; X₁₈ is Q or K; X₁₉ is W or A; X₂₀ is L or A; X₂₁ is D, K, R, A, F, G, M, N, |
| | or I; X₂₂ is I, F, or A; X₂₃ is K or T; X₂₄ is K or E; X₂₅ is D or E; X₂₆ is S or N; |
| | and X₂₇ is E or Q, and wherein the variant has at least one amino acid substitution |
| | relative to a wild-type extracellular ActRIIa having the sequence of SEQ ID NO: 73. |
| 2 | The polypeptide of row 1, wherein the variant has a sequence of |
| | GAILGRSETQECLFX₂NANWX₃X₄X₅X₆TNQTGVEX₇CX₈GX₉KX₁₁X₁₂X₁₃X₁₄HCX₁₅ATWX₁₆NIS |
| | GSIEIVX₁₇ |
| | X₁₈GCX₁₉X₂₀X₂₁DX₂₂NCYDRTDCVEX₂₃X₂₄X₂₅X₂₆PX₂₇VYFCCCEGNMCNEKFSYFPEMEVTQP |
| | TS (SEQ ID NO: 2). |
| 3 | The polypeptide of row 1 or 2, wherein the variant has a sequence of |
| | GAILGRSETQECLFX₂NANWEX₄X₅RTNQTGVEX₇CX₈GX₉KDKRX₁₄HCX₁₅ATWX₁₆NISGSIEIV |
| | KX₁₈GCWLDDX₂₂NCYDRTDCVEX₂₃X₂₄X₂₅X₂₆PX₂₇VYFCCCEGNMCNEKFSYFPEMEVTQPTS |
| | (SEQ ID NO: 3). |
| 4 | The polypeptide of any one of rows 1-3, wherein the variant has a sequence of |
| | GAILGRSETQECLFX₂NANWEX₄DRTNQTGVEX₇CX₈GX₉KDKRX₁₄HCX₁₅ATWX₁₆NISGSIEIV |
| | KX₁₈GCWLDDX₂₂NCYDRTDCVEX₂₃KX₂₅X₂₆PX₂₇VYFCCCEGNMCNEKFSYFPEMEVTQPTS |
| | (SEQ ID NO: 4). |
| 5 | The polypeptide of any one of rows 1-4, wherein the variant has a sequence of |
| | GAILGRSETQECLFX₂NANWEX₄DRTNQTGVEPCX₈GX₉KDKRX₁₄HCFATWKNISGSIEIVKX₁₈ |
| | GCWLDDINCYDRTDCVEX₂₃KX₂₅X₂₆PX₂₇VYFCCCEGNMCNEKFSYFPEMEVTQPTS (SEQ |
| | ID NO: 5). |
| 6 | The polypeptide of row 1, wherein X₁ is F. |
| 7 | The polypeptide of row 1, wherein X₁ is Y. |
| 8 | The polypeptide of row 1, 6, or 7 wherein X₁₀ is K. |
| 9 | The polypeptide of row 1, 6, or 7 wherein X₁₀ is Q. |
| 10 | The polypeptide of any one of rows 1-9, wherein X₂ is F. |
| 11 | The polypeptide of any one of rows 1-9, wherein X₂ is or Y. |
| 12 | The polypeptide of any one of rows 1, 2, and 6-11, wherein X₃ is E. |
| 13 | The polypeptide of any one of rows 1, 2, and 6-11, wherein X₃ is A. |
| 14 | The polypeptide of any one of rows 1-13, wherein X₄ is K. |
| 15 | The polypeptide of any one of rows 1-13, wherein X₄ is L. |
| 16 | The polypeptide of any one of rows 1, 2, 3, and 6-15, wherein X₅ is D. |
| 17 | The polypeptide of any one of rows 1, 2, 3, and 6-15, wherein X₅ is E. |
| 18 | The polypeptide of any one of rows 1, 2 and 6-17, wherein X₆ is R. |
| 19 | The polypeptide of any one of rows 1, 2 and 6-17, wherein X₆ is A. |
| 20 | The polypeptide of any one of rows 1-4 and 6-19, wherein X₇ is P. |
| 21 | The polypeptide of any one of rows 1-4 and 6-19, wherein X₇ is R. |
| 22 | The polypeptide of any one of rows 1-21, wherein X₈ is Y. |
| 23 | The polypeptide of any one of rows 1-21, wherein X₈ is E. |
| 24 | The polypeptide of any one of rows 1-23, wherein X₉ is D. |
| 25 | The polypeptide of any one of rows 1-23, wherein X₉ is E. |
| 26 | The polypeptide of any one of rows 1, 2 and 6-25, wherein X₁₁ is D. |
| 27 | The polypeptide of any one of rows 1, 2 and 6-25, wherein X₁₁ is A. |
| 28 | The polypeptide of any one of rows 1, 2 and 6-27, wherein X₁₂ is K. |
| 29 | The polypeptide of any one of rows 1, 2 and 6-27, wherein X₁₂ is A. |
| 30 | The polypeptide of any one of rows 1, 2 and 6-29, wherein X₁₃ is R. |
| 31 | The polypeptide of any one of rows 1, 2 and 6-29, wherein X₁₃ is A. |
| 32 | The polypeptide of any one of rows 1-31, wherein X₁₄ is R. |
| 33 | The polypeptide of any one of rows 1-31, wherein X₁₄ is L. |
| 34 | The polypeptide of any one of rows 1-4 and 6-33, wherein X₁₅ is F. |
| 35 | The polypeptide of any one of rows 1-4 and 6-33, wherein X₁₅ is Y. |
| 36 | The polypeptide of any one of rows 1-4 and 6-35, wherein X₁₆ is K. |
| 37 | The polypeptide of any one of rows 1-4 and 6-35, wherein X₁₆ is R. |
| 38 | The polypeptide of any one of rows 1-4 and 6-35, wherein X₁₆ is A. |
| 39 | The polypeptide of any one of rows 1, 2 and 6-38, wherein X₁₇ is K. |
| 40 | The polypeptide of any one of rows 1, 2 and 6-38, wherein X₁₇ is A. |
| 41 | The polypeptide of any one of rows 1, 2 and 6-38, wherein X₁₇ is Y. |
| 42 | The polypeptide of any one of rows 1, 2 and 6-38, wherein X₁₇ is F. |
| 43 | The polypeptide of any one of rows 1, 2 and 6-38, wherein X₁₇ is I. |
| 44 | The polypeptide of any one of rows 1-43, wherein X₁₈ is Q. |
| 45 | The polypeptide of any one of rows 1-43, wherein X₁₈ is K. |
| 46 | The polypeptide of any one of rows 1, 2 and 6-45, wherein X₁₉ is W. |
| 47 | The polypeptide of any one of rows 1, 2 and 6-45, wherein X₁₉ is A. |
| 48 | The polypeptide of any one of rows 1, 2 and 6-47, wherein X₂₀ is L. |
| 49 | The polypeptide of any one of rows 1, 2 and 6-47, wherein X₂₀ is A. |
| 50 | The polypeptide of any one of rows 1, 2 and 6-49, wherein X₂₁ is D. |
| 51 | The polypeptide of any one of rows 1, 2 and 6-49, wherein X₂₁ is K. |
| 52 | The polypeptide of any one of rows 1, 2 and 6-49, wherein X₂₁ is R. |
| 53 | The polypeptide of any one of rows 1, 2 and 6-49, wherein X₂₁ is A. |
| 54 | The polypeptide of any one of rows 1, 2 and 6-49, wherein X₂₁ is F. |
| 55 | The polypeptide of any one of rows 1, 2 and 6-49, wherein X₂₁ is G. |
| 56 | The polypeptide of any one of rows 1, 2 and 6-49, wherein X₂₁ is M. |
| 57 | The polypeptide of any one of rows 1, 2 and 6-49, wherein X₂₁ is N. |
| 58 | The polypeptide of any one of rows 1, 2 and 6-49, wherein X₂₁ is I. |
| 59 | The polypeptide of any one of rows 1-4 and 6-58, wherein X₂₂ is I. |
| 60 | The polypeptide of any one of rows 1-4 and 6-58, wherein X₂₂ is F. |
| 61 | The polypeptide of any one of rows 1-4 and 6-58, wherein X₂₂ is A. |
| 62 | The polypeptide of any one of rows 1-61, wherein X₂₃ is K. |
| 63 | The polypeptide of any one of rows 1-61, wherein X₂₃ is T. |
| 64 | The polypeptide of any one of rows 1, 2, 3, and 6-63, wherein X₂₄ is K. |
| 65 | The polypeptide of any one of rows 1, 2, 3, and 6-63, wherein X₂₄ is E. |
| 66 | The polypeptide of any one of rows 1-65, wherein X₂₅ is D. |
| 67 | The polypeptide of any one of rows 1-65, wherein X₂₅ is E. |
| 68 | The polypeptide of any one of rows 1-67, wherein X₂₆ is S. |
| 69 | The polypeptide of any one of rows 1-67, wherein X₂₆ is N. |
| 70 | The polypeptide of any one of rows 1-69, wherein X₂₇ is E. |
| 71 | The polypeptide of any one of rows 1-69, wherein X₂₇ is Q. |
| 72 | The polypeptide of any one of rows 1-71, wherein X₂₃ is T, X₂₄ is E, X₂₅ is E, and X₂₆ |
| | is N. |
| 73 | The polypeptide of any one of rows 1-71, wherein X₂₃ is T, X₂₄ is K, X₂₅ is E, and X₂₆ |
| | is N. |
| 74 | The polypeptide of any one of rows 1-73, wherein X₁₇ is K. |
| 75 | The polypeptide of row 1, wherein the variant has the sequence of any one of SEQ |
| | ID NOs: 6-72. |
| 76 | The polypeptide of row 75, wherein the variant has the sequence of SEQ ID NO: 69. |
| 77 | The polypeptide of row 75, wherein the variant has the sequence of SEQ ID NO: 58. |
| 78 | The polypeptide of row 75, wherein the variant has the sequence of SEQ ID NO: 6. |
| 79 | The polypeptide of row 75, wherein the variant has the sequence of SEQ ID NO: 38. |
| 80 | The polypeptide of row 75, wherein the variant has the sequence of SEQ ID NO: 41. |
| 81 | The polypeptide of row 75, wherein the variant has the sequence of SEQ ID NO: 44. |
| 82 | The polypeptide of row 75, wherein the variant has the sequence of SEQ ID NO: 70. |
| 83 | The polypeptide of row 75, wherein the variant has the sequence of SEQ ID NO: 71. |
| 84 | The polypeptide of row 75, wherein the variant has the sequence of SEQ ID NO: 72. |
| 85 | The polypeptide of any one of rows 1-84, wherein the amino acid at position X₂₄ is |
| | replaced with the amino acid K. |
| 86 | The polypeptide of any one of rows 1-85, wherein the amino acid at position X₂₄ is |
| | replaced with the amino acid E. |
| 87 | The polypeptide of any one of rows 1-86, further comprising a C-terminal extension of |
| | one or more amino acids. |
| 88 | The polypeptide of row 87, wherein the C-terminal extension is NP. |
| 89 | The polypeptide of row 87, wherein the C-terminal extension is NPVTPK (SEQ ID NO: 78). |
| 90 | The polypeptide of any one of rows 1-89, further comprising an Fc domain monomer |
| | fused to the C-terminus of the polypeptide by way of a linker. |
| 91 | The polypeptide of row 90, wherein the Fc domain monomer comprises the sequence of SEQ |
| | ID NO: 97. |
| 92 | The polypeptide of any one of rows 1-89, further comprising an Fc domain fused to the |
| | C-terminus of the polypeptide by way of a linker. |
| 93 | The polypeptide of row 92, wherein the Fc domain comprises or consists of the sequence |
| | of SEQ ID NO: 84 or SEQ ID NO: 79. |
| 94 | The polypeptide of row 93, wherein the Fc domain comprises or consists of the sequence |
| | of SEQ ID NO: 79. |
| 95 | The polypeptide of any one of rows 1-89, further comprising an albumin-binding peptide |
| | fused to the C-terminus of the polypeptide by way of a linker. |
| 96 | The polypeptide of row 95, wherein the albumin-binding peptide comprises the sequence |
| | of SEQ ID NO: 83. |
| 97 | The polypeptide of any one of rows 1-89, further comprising a fibronectin domain fused |
| | to the C-terminus of the polypeptide by way of a linker. |
| 98 | The polypeptide of row 97, wherein the fibronectin domain comprises the sequence of |
| | SEQ ID NO: 82. |
| 99 | The polypeptide of any one of rows 1-89, further comprising a human serum albumin |
| | fused to the C-terminus of the polypeptide by way of a linker. |
| 100 | The polypeptide of row 99, wherein the human serum albumin comprises the sequence of |
| | SEQ ID NO: 81. |
| 101 | The polypeptide of row 90 or 91, wherein the polypeptide forms a dimer. |
| 102 | The polypeptide of any one of rows 90-101, wherein the linker is an amino acid spacer. |
| 103 | The polypeptide of row 102, wherein the amino acid spacer is GGG, GGGA (SEQ ID NO: 98), |
| | GGGG (SEQ ID NO: 100), GGGAG (SEQ ID NO: 130), GGGAGG (SEQ ID NO: 131), or |
| | GGGAGGG (SEQ ID NO: 132). |
| 104 | The polypeptide of row 103, wherein the amino acid spacer is GGG. |
| 105 | The polypeptide of row 94 or 104, wherein the polypeptide has the sequence of SEQ ID |
| | NO: 80. |
| 106 | The polypeptide of row 102, wherein the amino acid spacer is GGGS (SEQ ID NO: 99), |
| | GGGGA (SEQ ID NO: 101), GGGGS (SEQ ID NO: 102), GGGGG (SEQ ID NO: 103), GGAG |
| | (SEQ ID NO: 104), GGSG (SEQ ID NO: 105), AGGG (SEQ ID NO: 106), SGGG (SEQ ID NO: |
| | 107), GAGA (SEQ ID NO: 108), GSGS (SEQ ID NO: 109), GAGAGA (SEQ ID NO: 110), |
| | GSGSGS (SEQ ID NO: 111), GAGAGAGA (SEQ ID NO: 112), GSGSGSGS (SEQ ID NO: |
| | 113), GAGAGAGAGA (SEQ ID NO: 114), GSGSGSGSGS (SEQ ID NO: 115), |
| | GAGAGAGAGAGA (SEQ ID NO: 116), and GSGSGSGSGSGS (SEQ ID NO: 117), GGAGGA |
| | (SEQ ID NO: 118), GGSGGS (SEQ ID NO: 119), GGAGGAGGA (SEQ ID NO: 120), |
| | GGSGGSGGS (SEQ ID NO: 121), GGAGGAGGAGGA (SEQ ID NO: 122), |
| | GGSGGSGGSGGS (SEQ ID NO: 123), GGAGGGAG (SEQ ID NO: 124), GGSGGGSG (SEQ |
| | ID NO: 125), GGAGGGAGGGAG (SEQ ID NO: 126), and GGSGGGSGGGSG (SEQ ID NO: |
| | 127), GGGGAGGGGAGGGGA (SEQ ID NO: 128), GGGGSGGGGSGGGGS (SEQ ID NO: |
| | 129), AAAL (SEQ ID NO: 133), AAAK (SEQ ID NO: 134), AAAR (SEQ ID NO: 135), |
| | EGKSSGSGSESKST (SEQ ID NO: 136), GSAGSAAGSGEF (SEQ ID NO: 137), |
| | AEAAAKEAAAKA (SEQ ID NO: 96), KESGSVSSEQLAQFRSLD (SEQ ID NO: 95), |
| | GENLYFQSGG (SEQ ID NO: 94), SACYCELS (SEQ ID NO: 93), RSIAT (SEQ ID NO: 92), |
| | RPACKIPNDLKQKVMNH (SEQ ID NO: 91), |
| | GGSAGGSGSGSSGGSSGASGTGTAGGTGSGSGTGSG (SEQ ID NO: 90), |
| | AAANSSIDLISVPVDSR (SEQ ID NO: 89), |
| | GGSGGGSEGGGSEGGGSEGGGSEGGGSEGGGSGGGS (SEQ ID NO: 88), EAAAK (SEQ |
| | ID NO: 87), or PAPAP(SEQ ID NO: 86). |
| 107 | The polypeptide of any one of rows 1-106, wherein the polypeptide has a serum half- |
| | life of at least 7 days. |
| 108 | The polypeptide of any one of rows 1-107, wherein the polypeptide binds to activin A, |
| | activin B, and/or myostatin and has reduced or weak binding to human BMP9. |
| 109 | The polypeptide of row 108, wherein the polypeptide does not substantially bind to |
| | human BMP9. |
| 110 | The polypeptide of any one of rows 1-109, wherein the polypeptide binds to human |
| | activin A with a KD of 800 pM or less. |
| 111 | The polypeptide of any one of rows 1-110, wherein the polypeptide binds to human |
| | activin B with a Kp of 800 pM or less. |
| 112 | The polypeptide of any one of rows 1-111, wherein the polypeptide binds to human |
| | GDF-11 with a Ko of 5 pM or higher. |
| 113 | A nucleic acid molecule encoding a polypeptide of any one of rows 1-112. |
| 114 | A vector comprising the nucleic acid molecule of row 113. |
| 115 | A host cell that expresses a polypeptide of any one of rows 1-112, wherein the host |
| | cell comprises a nucleic acid molecule of row 113 or a vector of row 114, wherein the |
| | nucleic acid molecule or vector is expressed in the host cell. |
| 116 | A pharmaceutical composition comprising a polypeptide of any one of rows 1-112, a |
| | nucleic acid molecule of row 113, or a vector of row 114, and one or more |
| | pharmaceutically acceptable carriers or excipients. |
| 117 | The pharmaceutical composition of row 116, wherein the polypeptide is in a |
| | therapeutically effective amount. |

TABLE 19

| Row | Composition |
|---|---|
| 1 | A polypeptide comprising an extracellular activin receptor type IIB (ActRIIB) variant, |
| | the variant having one or more amino acid substitutions relative to the sequence of |
| | GRGEAETRECIYYNANWELERTNQSGLERCEGEQDKRLHCYASWRNSSGTIELVKKGCWL |
| | DDFNCYDRQECVATEENPQVYFCCCEGNFCNERFTHLPEAGGPEVTYEPPPTAPT (SEQ ID |
| | NO: 74), wherein the variant comprises one or more amino acid substitutions that impart |
| | reduced BMP9 binding relative to wild type extracellular ActRIIB and one or more |
| | additional amino acid substitutions, wherein the substitutions that reduce BMP9 binding |
| | comprise one or more of: |
| | a) amino acid substitution E75K; |
| | b) amino acid substitutions Q69T and E70D; or |
| | c) amino acid substitutions Q69D and E70T, |
| | optionally wherein the variant is truncated from the N-terminus by deletion of 1, 2, 3, |
| | 4, 5, 6, or 7 amino acids. |
| 2 | The polypeptide of row 1, wherein the variant comprises one or more amino acid |
| | substitutions selected from the group consisting of I11L, Y12F, L19K, E20D, S25T, L27V, |
| | R29P, E31Y, E33D, Q34K, L38R, Y41F, R45K, S47I, S48T, T50S, I51L, L53I, K56Q, F63I, |
| | T74K, E76D, N77S, Q79E, and F89M. |
| 3 | The polypeptide of row 1 or 2, wherein the variant comprises amino acid substitutions |
| | E75K, E20D, and F631. |
| 4 | The polypeptide of row 1 or 2, wherein the variant comprises amino acid substitution |
| | E75K. |
| 5 | The polypeptide of row 4, wherein the variant comprises amino acid substitutions T74K, |
| | E76D, N77S, and Q79E. |
| 6 | The polypeptide of row 5, wherein the variant further comprises one or more additional |
| | amino acid substitutions. |
| 7 | The polypeptide of row 6, wherein the variant comprises amino acid substitutions Y41F, |
| | R45K, and K56Q. |
| 8 | The polypeptide of row 7, wherein the variant further comprises amino acid substitutions |
| | Y12F, L19K, E20D, R29P, E31Y, E33D, L38R, and F63I. |
| 9 | The polypeptide of row 6, wherein the variant comprises amino acid substitutions S25T and |
| | S47I. |
| 10 | The polypeptide of row 9, wherein the variant comprises amino acid substitution S48T. |
| 11 | The polypeptide of row 6, wherein the variant comprises amino acid substitution R29P. |
| 12 | The polypeptide of row 6, wherein the variant comprises amino acid substitutions E31Y, |
| | E33D, and Q34K. |
| 13 | The polypeptide of row 6, wherein the variant comprises amino acid substitutions Y12F, |
| | L19K, and E20D. |
| 14 | The polypeptide of row 6, wherein the variant comprises amino acid substitutions E31Y, |
| | E33D, and L38R. |
| 15 | The polypeptide of row 1 or 2, wherein the variant comprises amino acid substitutions |
| | Q69T and E70D. |
| 16 | The polypeptide of any one of rows 1, 2, and 15, wherein the variant comprises amino acid |
| | substitutions Q69T and E70D and additional amino acid substitutions I11L, L27V, Q34K, |
| | T50S, I51L, L53I, and F89M. |
| 17 | The polypeptide of row 1 or 2, wherein the variant comprises amino acid substitutions |
| | Q69D and E70T. |
| 18 | The polypeptide of any one of rows 1, 2, and 17, wherein the variant comprises amino |
| | acid substitutions Q69D and E70T and additional amino acid substitutions I11L, L27V, |
| | Q34K, T50S, I51L, L53I, and F89M. |
| 19 | The polypeptide of any one of rows 15-18, wherein the variant comprises amino acid |
| | substitution E75K. |
| 20 | A polypeptide comprising an ActRIIB variant, the variant having a sequence of |
| | GRGEAETRECX₁X₂YNANWEX₃X₄RTNQX₅GX₆EX₇CX₈GX₉X₁₀DKRX₁₁HCX₁₂ASWX₁₃NX₁₄X₁₅ |
| | GX₁₆X₁₇EX₁₈VKX₁₉GCWLDDX₂₀NCYDRX₂₁X₂₂CVAX₂₃X₂₄X₂₅X₂₆PX₂₇VYFCCCEGNX₂₈CNERF |
| | THLPEAGGPEVTYEPPPTAPT (SEQ ID NO: 730), wherein X₁ is I or L; X₂ is F or Y; X₃ is L or |
| | K; X₄ is D or E; X₅ is T or S; X₆ is L or V; X₇ is P or R; X₈ is Y or E; X₉ is D or E; X₁₀ |
| | is K or Q; X₁₁ is R or L; X₁₂ is Y or F; X₁₃ is R or K; X₁₄ is S or I; X₁₅ is S or T; X₁₆ |
| | is S or T; X₁₇ is I or L; X₁₈ is I or L; X₁₉ is K or Q; X₂₀ is F or I; X₂₁ is Q, T, or D; |
| | X₂₂ is E, D, or T; X₂₃ is K or T; X₂₄ is K or E; X₂₅ is D or E; X₂₆ is S or N; X₂₇ is E or Q; |
| | and X₂₈ is F or M, and wherein X₂₄ is E and/or either X₂₁ is T and X₂₂ is D or X₂₁ is D and |
| | X₂₂ is T, and wherein the variant has at least one amino acid substitution relative to a |
| | wild-type extracellular ActRIIB having the sequence of SEQ ID NO: 74, optionally wherein |
| | the variant is truncated from the N-terminus by deletion of 1, 2, 3, 4, 5, 6, or 7 amino |
| | acids. |
| 21 | The polypeptide of row 20, wherein X₁ is I. |
| 22 | The polypeptide of row 20, wherein X₁ is L. |
| 23 | The polypeptide of any one of rows 20-22, wherein X₂ is F. |
| 24 | The polypeptide of any one of rows 20-22, wherein X₂ is Y. |
| 25 | The polypeptide of any one of rows 20-24, wherein X₃ is L. |
| 26 | The polypeptide of any one of rows 20-24, wherein X₃ is K. |
| 27 | The polypeptide of any one of rows 20-26, wherein X₄ is D. |
| 28 | The polypeptide of any one of rows 20-26, wherein X₄ is E. |
| 29 | The polypeptide of any one of rows 20-28, wherein X₅ is T. |
| 30 | The polypeptide of any one of rows 20-28, wherein X₅ is S. |
| 31 | The polypeptide of any one of rows 20-30, wherein X₆ is L. |
| 32 | The polypeptide of any one of rows 20-30, wherein X₆ is V. |
| 33 | The polypeptide of any one of rows 20-32, wherein X₇ is P. |
| 34 | The polypeptide of any one of rows 20-32, wherein X₇ is R. |
| 35 | The polypeptide of any one of rows 20-34, wherein X₈ is Y. |
| 36 | The polypeptide of any one of rows 20-34, wherein X₈ is E. |
| 37 | The polypeptide of any one of rows 20-36, wherein X₉ is D. |
| 38 | The polypeptide of any one of rows 20-36, wherein X₉ is E. |
| 39 | The polypeptide of any one of rows 20-38, wherein X₁₀ is K. |
| 40 | The polypeptide of any one of rows 20-38, wherein X₁₀ is Q. |
| 41 | The polypeptide of any one of rows 20-40, wherein X₁₁ is R. |
| 42 | The polypeptide of any one of rows 20-40, wherein X₁₁ is L. |
| 43 | The polypeptide of any one of rows 20-42, wherein X₁₂ is Y. |
| 44 | The polypeptide of any one of rows 20-42, wherein X₁₂ is F. |
| 45 | The polypeptide of any one of rows 20-44, wherein X₁₃ is R. |
| 46 | The polypeptide of any one of rows 20-44, wherein X₁₃ is K. |
| 47 | The polypeptide of any one of rows 20-46, wherein X₁₄ is S. |
| 48 | The polypeptide of any one of rows 20-46, wherein X₁₄ is I. |
| 49 | The polypeptide of any one of rows 20-48, wherein X₁₅ is S. |
| 50 | The polypeptide of any one of rows 20-48, wherein X₁₅ is T. |
| 51 | The polypeptide of any one of rows 20-50, wherein X₁₆ is S. |
| 52 | The polypeptide of any one of rows 20-50, wherein X₁₆ is T. |
| 53 | The polypeptide of any one of rows 20-52, wherein X₁₇ is I. |
| 54 | The polypeptide of any one of rows 20-52, wherein X₁₇ is L. |
| 55 | The polypeptide of any one of rows 20-54, wherein X₁₈ is I. |
| 56 | The polypeptide of any one of rows 20-54, wherein X₁₈ is L. |
| 57 | The polypeptide of any one of rows 20-56, wherein X₁₉ is K. |
| 58 | The polypeptide of any one of rows 20-56, wherein X₁₉ is Q. |
| 59 | The polypeptide of any one of rows 20-58, wherein X₂₀ is F. |
| 60 | The polypeptide of any one of rows 20-58, wherein X₂₀ is I. |
| 61 | The polypeptide of any one of rows 20-60, wherein X₂₁ is Q. |
| 62 | The polypeptide of any one of rows 20-60, wherein X₂₁ is T. |
| 63 | The polypeptide of any one of rows 20-60, wherein X₂₁ is D. |
| 64 | The polypeptide of any one of rows 20-61, wherein X₂₂ is E. |
| 65 | The polypeptide of any one of rows 20-60 and 62, wherein X₂₂ is D. |
| 66 | The polypeptide of any one of rows 20-60 and 63, wherein X₂₂ is T. |
| 67 | The polypeptide of any one of rows 20-66, wherein X₂₃ is K. |
| 68 | The polypeptide of any one of rows 20-66, wherein X₂₃ is T. |
| 69 | The polypeptide of any one of rows 20-68, wherein X₂₄ is K. |
| 70 | The polypeptide of any one of rows 20-60, 62, 63, and 65-68, wherein X₂₄ is E. |
| 71 | The polypeptide of any one of rows 20-70, wherein X₂₅ is D. |
| 72 | The polypeptide of any one of rows 20-70, wherein X₂₅ is E. |
| 73 | The polypeptide of any one of rows 20-72, wherein X₂₆ is S. |
| 74 | The polypeptide of any one of rows 20-72, wherein X₂₆ is N. |
| 75 | The polypeptide of any one of rows 20-74, wherein X₂₇ is E. |
| 76 | The polypeptide of any one of rows 20-74, wherein X₂₇ is Q. |
| 77 | The polypeptide of any one of rows 20-76, wherein X₂₈ is F. |
| 78 | The polypeptide of any one of rows 20-76, wherein X₂₈ is M. |
| 79 | The polypeptide of any one of rows 20-78, wherein X₂₃ is T, X₂₄ is K, X₂₅ is E, and X₂₆ |
| | is N. |
| 80 | The polypeptide of any one of rows 20-78, wherein X₂₃ is T, X₂₄ is E, X₂₅ is E, and X₂₆ |
| | is N. |
| 81 | The polypeptide of any one of rows 20-78, wherein X₂₃ is K, X₂₄ is K, X₂₅ is D, and X₂₆ |
| | is S. |
| 82 | The polypeptide of any one of rows 1-81, wherein the variant has the sequence of any |
| | one of SEQ ID NOs: 731-744. |
| 83 | The polypeptide of row 82, wherein the variant has the sequence of SEQ ID NO: 732. |
| 84 | The polypeptide of row 82, wherein the variant has the sequence of SEQ ID NO: 738. |
| 85 | The polypeptide of row 82, wherein the variant has the sequence of SEQ ID NO: 741. |
| 86 | The polypeptide of row 82, wherein the variant has the sequence of SEQ ID NO: 742. |
| 87 | The polypeptide of row 82, wherein the variant has the sequence of SEQ ID NO: 743. |
| 88 | The polypeptide of row 82, wherein the variant has the sequence of SEQ ID NO: 744. |
| 89 | The polypeptide of any one of rows 1-88, wherein the amino acid at position X₂₄ is |
| | replaced with the amino acid K. |
| 90 | The polypeptide of any one of rows 1-88, wherein the amino acid at position X₂₄ is |
| | replaced with the amino acid E. |
| 91 | The polypeptide of any one of rows 1-90, wherein the variant is truncated from the N- |
| | terminus by deletion of one amino acid. |
| 92 | The polypeptide of any one of rows 1-90, wherein the variant is truncated from the N- |
| | terminus by deletion of two amino acids. |
| 93 | The polypeptide of any one of rows 1-90, wherein the variant is truncated from the N- |
| | terminus by deletion of three amino acids. |
| 94 | The polypeptide of any one of rows 1-90, wherein the variant is truncated from the N- |
| | terminus by deletion of four amino acids. |
| 95 | The polypeptide of any one of rows 1-90, wherein the variant is truncated from the N- |
| | terminus by deletion of five amino acids. |
| 96 | The polypeptide of any one of rows 1-90, wherein the variant is truncated from the N- |
| | terminus by deletion of six amino acids. |
| 97 | The polypeptide of any one of rows 1-90, wherein the variant is truncated from the N- |
| | terminus by deletion of seven amino acids. |
| 98 | The polypeptide of any one of rows 1-97, further comprising an Fc domain monomer fused |
| | to the C-terminus of the polypeptide by way of a linker. |
| 99 | The polypeptide of row 98, wherein the Fc domain monomer comprises the sequence of SEQ |
| | ID NO: 97. |
| 100 | The polypeptide of row 98 or 99, wherein the polypeptide forms a dimer. |
| 101 | The polypeptide of any one of rows 1-97, further comprising an Fc domain fused to the C- |
| | terminus of the polypeptide by way of a linker. |
| 102 | The polypeptide of row 101, wherein the Fc domain comprises the sequence of SEQ ID NO: |
| | 84 or SEQ ID NO: 79. |
| 103 | The polypeptide of row 102, wherein the Fc domain comprises the sequence of SEQ ID NO: |
| | 84. |
| 104 | The polypeptide of row 102, wherein the Fc domain comprises the sequence of SEQ ID NO: |
| | 79. |
| 105 | The polypeptide of any one of rows 1-97, further comprising an albumin-binding peptide |
| | fused to the C-terminus of the polypeptide by way of a linker. |
| 106 | The polypeptide of row 105, wherein the albumin-binding peptide comprises the sequence |
| | of SEQ ID NO: 83. |
| 107 | The polypeptide of any one of rows 1-97, further comprising a fibronectin domain fused |
| | to the C-terminus of the polypeptide by way of a linker. |
| 108 | The polypeptide of row 107, wherein the fibronectin domain comprises the sequence of |
| | SEQ ID NO: 82. |
| 109 | The polypeptide of any one of rows 1-97, further comprising a human serum albumin fused |
| | to the C-terminus of the polypeptide by way of a linker. |
| 110 | The polypeptide of row 109, wherein the human serum albumin comprises the sequence of |
| | SEQ ID NO: 81. |
| 111 | The polypeptide of any one of rows 98-110, wherein the linker is an amino acid spacer. |
| 112 | The polypeptide of row 111, wherein the amino acid spacer is GGG, GGGA (SEQ ID NO: 98), |
| | GGGG (SEQ ID NO: 100), GGGAG (SEQ ID NO: 130), GGGAGG (SEQ ID NO: 131), or |
| | GGGAGGG (SEQ ID NO: 132). |
| 113 | The polypeptide of row 112, wherein the amino acid spacer is GGG. |
| 114 | The polypeptide of row 111, wherein the amino acid spacer is GA, GS, GG, GGA, GGS, GGG, |
| | GGGS (SEQ ID NO: 99), GGGGA (SEQ ID NO: 101), GGGGS (SEQ ID NO: 102), GGGGG |
| | (SEQ ID NO: 103), GGAG (SEQ ID NO: 104), GGSG (SEQ ID NO: 105), AGGG (SEQ ID NO: |
| | 106), SGGG (SEQ ID NO: 107), GAGA (SEQ ID NO: 108), GSGS (SEQ ID NO: 109), |
| | GAGAGA (SEQ ID NO: 110), GSGSGS (SEQ ID NO: 111), GAGAGAGA (SEQ ID NO: 112), |
| | GSGSGSGS (SEQ ID NO: 113), GAGAGAGAGA (SEQ ID NO: 114), GSGSGSGSGS (SEQ |
| | ID NO: 115), GAGAGAGAGAGA (SEQ ID NO: 116), and GSGSGSGSGSGS (SEQ ID NO: |
| | 117), GGAGGA (SEQ ID NO: 118), GGSGGS (SEQ ID NO: 119), GGAGGAGGA (SEQ ID |
| | NO: 120), GGSGGSGGS (SEQ ID NO: 121), GGAGGAGGAGGA (SEQ ID NO: 122), |
| | GGSGGSGGSGGS (SEQ ID NO: 123), GGAGGGAG (SEQ ID NO: 124), GGSGGGSG (SEQ |
| | ID NO: 125), GGAGGGAGGGAG (SEQ ID NO: 126), and GGSGGGSGGGSG (SEQ ID NO: |
| | 127), GGGGAGGGGAGGGGA (SEQ ID NO: 128), GGGGSGGGGSGGGGS (SEQ ID NO: |
| | 129), AAAL (SEQ ID NO: 133), AAAK (SEQ ID NO: 134), AAAR (SEQ ID NO: 135), |
| | EGKSSGSGSESKST (SEQ ID NO: 136), GSAGSAAGSGEF (SEQ ID NO: 137), |
| | AEAAAKEAAAKA (SEQ ID NO: 96), KESGSVSSEQLAQFRSLD (SEQ ID NO: 95), |
| | GENLYFQSGG (SEQ ID NO: 94), SACYCELS (SEQ ID NO: 93), RSIAT (SEQ ID NO: 92), |
| | RPACKIPNDLKQKVMNH (SEQ ID NO: 91), |
| | GGSAGGSGSGSSGGSSGASGTGTAGGTGSGSGTGSG (SEQ ID NO: 90), |
| | AAANSSIDLISVPVDSR (SEQ ID NO: 89), |
| | GGSGGGSEGGGSEGGGSEGGGSEGGGSEGGGSGGGS (SEQ ID NO: 88), EAAAK (SEQ |
| | ID NO: 87), or PAPAP(SEQ ID NO: 86). |
| 115 | The polypeptide of any one of rows 1-114, wherein the polypeptide has a serum half-life |
| | of at least 7 days. |
| 116 | The polypeptide of any one of rows 1-115, wherein the polypeptide binds to activin A, |
| | activin B, and/or myostatin and has reduced or weak binding to human BMP9. |
| 117 | The polypeptide of row 116, wherein the polypeptide does not substantially bind to human |
| | BMP9. |
| 118 | The polypeptide of any one of rows 1-117, wherein the polypeptide binds to human activin |
| | A with a Kp of 800 pM or less. |
| 119 | The polypeptide of any one of rows 1-118, wherein the polypeptide binds to human activin |
| | B with a Kp of 800 pM or less. |
| 120 | The polypeptide of any one of rows 1-119, wherein the polypeptide binds to human GDF-11 |
| | with a Ko of 5 pM or higher. |
| 121 | A nucleic acid molecule encoding a polypeptide of any one of rows 1-120. |
| 122 | A vector comprising the nucleic acid molecule of row 121. |
| 123 | A host cell that expresses a polypeptide of any one of rows 1-120, wherein the host cell |
| | comprises a nucleic acid molecule of row 121 or a vector of row 122, wherein the nucleic |
| | acid molecule or vector is expressed in the host cell. |
| 124 | A pharmaceutical composition comprising a polypeptide of any one of rows 1-120, a |
| | nucleic acid molecule of row 121, or a vector of row 122, and one or more |
| | pharmaceutically acceptable carriers or excipients. |
| 125 | The pharmaceutical composition of row 124, wherein the polypeptide is in a |
| | therapeutically effective amount. |
| 126 | A construct comprising two identical polypeptides (e.g., a homodimer), each comprising |
| | an extracellular ActRIIB variant of any one of rows 1-97 (e.g., an ActRIIB variant |
| | having a sequence of any one of SEQ ID NOs: 730-744) fused (e.g., linked using an amino |
| | acid spacer) to the N- or C-terminus of an Fc domain monomer (e.g., the sequence of SEQ |
| | ID NO: 97). The two Fc domain monomers in the two polypeptides interact to form an Fc |
| | domain in the construct. |
| 127 | A construct comprising two different polypeptides (e.g., a heterodimer), each comprising |
| | an extracellular ActRIIB variant of any one of rows 1-97 (e.g., an ActRIIB variant |
| | having a sequence of any one of SEQ ID NOs: 730-744) fused (e.g., linked using an amino |
| | acid spacer) to the N- or C-terminus of an Fc domain monomer (e.g., the sequence of SEQ |
| | ID NO: 97). The two Fc domain monomers in the two polypeptides interact to form an Fc |
| | domain in the construct. |

TABLE 20

| Row | Composition |
|---|---|
| 1 | A polypeptide comprising an extracellular activin receptor type II (ActRII) chimera, |
| | the chimera having a sequence of any one of |
| | GAILGRAETRECIYYNANWELERTNQSGLERCEGEQX₁KRRHCFATWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 751), |
| | GAILGRAETRECIYYNANWELERTNQSGLERCEGEQX₁KRLHCFATWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 752), |
| | GAILGRAETRECIYYNANWELERTNQSGLERCEGEQX₁KRLHCYASWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 753), |
| | GAILGRAETRECIYYNANWELERTNQSGLERCEGEQX₁KRLHCYASWRNSSGSIEIVKQGCWLD |
| | DX₂X₃CYDRTDCVX₄X₅X₆X₇X₈PX₉VYFCCCEGNMCNEKFSYFPEMEVTQPTS (SEQ ID NO: 754), |
| | GAILGRAETRECIYYNANWELERTNQSGLERCEGEQX₁KRLHCYASWRNSSGTIEIVKQGCWLD |
| | DX₂X₃CYDRTDCVX₄X₅X₆X₇X₈PX₉VYFCCCEGNMCNEKFSYFPEMEVTQPTS (SEQ ID NO: 755), |
| | GAILGRAETRECIYYNANWELERTNQSGLERCEGEQX₁KRLHCYASWRNSSGTIELVKKGCWLD |
| | DX₂X₃CYDRTDCVX₄X₅X₆X₇X₈PX₉VYFCCCEGNMCNEKFSYFPEMEVTQPTS (SEQ ID NO: 756), |
| | GAILGRAETRECIYYNANWELERTNQSGLERCEGEQX₁KRLHCYASWRNSSGTIELVKKGCWLD |
| | DX₂X₃CYDRQECVX₄X₅X₆X₇X₈PX₉VYFCCCEGNMCNEKFSYFPEMEVTQPTS (SEQ ID NO: 757), |
| | GRGEAETRECIYYNANWELERTNQSGLERCEGEQX₁KRRHCFATWKNISGSIEIVKQGCWLDDX₂ |
| | X₃CYDRTDCVX₄X₅X₆X₇X₈PX₉VYFCCCEGNMCNEKFSYFPEMEVTQPTS (SEQ ID NO: 758), |
| | GRGEAETRECIYYNANWELERTNQSGLERCEGEQX₁KRLHCFATWKNISGSIEIVKQGCWLDDX₂ |
| | X₃CYDRTDCVX₄X₅X₆X₇X₈PX₉VYFCCCEGNMCNEKFSYFPEMEVTQPTS (SEQ ID NO: 759), |
| | GRGEAETRECIYYNANWELERTNQSGLERCEGEQX₁KRLHCYASWKNISGSIEIVKQGCWLDDX₂ |
| | X₃CYDRTDCVX₄X₅X₆X₇X₈PX₉VYFCCCEGNMCNEKFSYFPEMEVTQPTS (SEQ ID NO: 760), |
| | GRGEAETRECIYYNANWELERTNQSGLERCEGEQX₁KRLHCYASWRNSSGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 761), |
| | GRGEAETRECIYYNANWELERTNQSGLERCEGEQX₁KRLHCYASWRNSSGTIEIVKQGCWLDD |
| | X₂X₃CYDRTDCVX₄X₅X₆X₇X₈PX₉VYFCCCEGNMCNEKFSYFPEMEVTQPTS (SEQ ID NO: 762), |
| | GRGEAETRECIYYNANWELERTNQSGLERCEGEQX₁KRLHCYASWRNSSGTIELVKKGCWLDD |
| | X₂X₃CYDRTDCVX₄X₅X₆X₇X₈PX₉VYFCCCEGNMCNEKFSYFPEMEVTQPTS (SEQ ID NO: 763), |
| | and |
| | GRGEAETRECIYYNANWELERTNQSGLERCEGEQX₁KRLHCYASWRNSSGTIELVKKGCWLDD |
| | X₂X₃CYDRQECVX₄X₅X₆X₇X₈PX₉VYFCCCEGNMCNEKFSYFPEMEVTQPTS (SEQ ID NO: 764), |
| | wherein X₁ is D or R, X₂ is I, F, E, D, Y, S, N, Q, or T, X₃ is N or T, X₄ is A or E, |
| | X₅ is T or K, X₆ is E or K, X₇ is E or D, X₈ is N or S, and X₉ is Q, E, K, R, D, or N, |
| | optionally wherein the chimera is truncated from the N-terminus by deletion of 1, 2, |
| | 3, 4, 5, 6, 7, 8, or 9 amino acids, wherein the chimera retains the two amino acids |
| | before the first cysteine. |
| 2 | A polypeptide containing an extracellular ActRII chimera, the chimera having a sequence |
| | of any one of |
| | GAILGRSETQECIYYNANWELERTNQSGLERCEGEQX₁KRRHCFATWKNISGSIEIVKQGCWLD |
| | DX₂X₃CYDRTDCVX₄X₅X₆X₇X₈PX₉VYFCCCEGNMCNEKFSYFPEMEVTQPTS (SEQ ID NO: 765), |
| | GAILGRSETQECIYYNANWELERTNQSGLERCEGEQX₁KRLHCFATWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 766), |
| | GAILGRSETQECIYYNANWELERTNQSGLERCEGEQX₁KRLHCYASWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 767), |
| | GAILGRSETQECIYYNANWELERTNQSGLERCEGEQX₁KRLHCYASWRNSSGSIEIVKQGCWLD |
| | DX₂X₃CYDRTDCVX₄X₅X₆X₇X₈PX₉VYFCCCEGNMCNEKFSYFPEMEVTQPTS (SEQ ID NO: 768), |
| | GAILGRSETQECIYYNANWELERTNQSGLERCEGEQX₁KRLHCYASWRNSSGTIEIVKQGCWLD |
| | DX₂X₃CYDRTDCVX₄X₅X₆X₇X₈PX₉VYFCCCEGNMCNEKFSYFPEMEVTQPTS (SEQ ID NO: 769), |
| | GAILGRSETQECIYYNANWELERTNQSGLERCEGEQX₁KRLHCYASWRNSSGTIELVKKGCWLD |
| | DX₂X₃CYDRTDCVX₄X₅X₆X₇X₈PX₉VYFCCCEGNMCNEKFSYFPEMEVTQPTS (SEQ ID NO: 770), |
| | and |
| | GAILGRSETQECIYYNANWELERTNQSGLERCEGEQX₁KRLHCYASWRNSSGTIELVKKGCWLD |
| | DX₂X₃CYDRQECVX₄X₅X₆X₇X₈PX₉VYFCCCEGNMCNEKFSYFPEMEVTQPTS (SEQ ID NO: 771), |
| | in which X₁ is D or R, X₂ is I, F, E, D, Y, S, N, Q, or T, X₃ is N or T, X₄ is A or E, |
| | X₅ is T or K, X₆ is E or K, X₇ is E or D, X₈ is N or S, and X₉ is Q, E, K, R, D, or N, |
| | optionally wherein the chimera is truncated from the N-terminus by deletion of 1, 2, |
| | 3, 4, 5, 6, 7, 8, or 9 amino acids, wherein the chimera retains the two amino acids |
| | before the first cysteine. |
| 3 | The polypeptide of row 1, wherein the chimera has the sequence of |
| | GAILGRAETRECIYYNANWELERTNQSGLERCEGEQX₁KRRHCFATWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 751). |
| 4 | The polypeptide of row 1, wherein the chimera has the sequence of |
| | GAILGRAETRECIYYNANWELERTNQSGLERCEGEQX₁KRLHCFATWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 752). |
| 5 | The polypeptide of row 1, wherein the chimera has the sequence of |
| | GAILGRAETRECIYYNANWELERTNQSGLERCEGEQX₁KRLHCYASWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 753). |
| 6 | The polypeptide of row 1, wherein the chimera has the sequence of |
| | GAILGRAETRECIYYNANWELERTNQSGLERCEGEQX₁KRLHCYASWRNSSGSIEIVKQGCWLD |
| | DX₂X₃CYDRTDCVX₄X₅X₆X₇X₈PX₉VYFCCCEGNMCNEKFSYFPEMEVTQPTS (SEQ ID NO: 754). |
| 7 | The polypeptide of row 1, wherein the chimera has the sequence of |
| | GAILGRAETRECIYYNANWELERTNQSGLERCEGEQX₁KRLHCYASWRNSSGTIEIVKQGCWLD |
| | DX₂X₃CYDRTDCVX₄X₅X₆X₇X₈PX₉VYFCCCEGNMCNEKFSYFPEMEVTQPTS (SEQ ID NO: 755). |
| 8 | The polypeptide of row 1, wherein the chimera has the sequence of |
| | GAILGRAETRECIYYNANWELERTNQSGLERCEGEQX₁KRLHCYASWRNSSGTIELVKKGCWLD |
| | DX₂X₃CYDRTDCVX₄X₅X₆X₇X₈PX₉VYFCCCEGNMCNEKFSYFPEMEVTQPTS (SEQ ID NO: 756). |
| 9 | The polypeptide of row 1, wherein the chimera has the sequence of |
| | GAILGRAETRECIYYNANWELERTNQSGLERCEGEQX₁KRLHCYASWRNSSGTIELVKKGCWLD |
| | DX₂X₃CYDRQECVX₄X₅X₆X₇X₈PX₉VYFCCCEGNMCNEKFSYFPEMEVTQPTS (SEQ ID NO: 757). |
| 10 | The polypeptide of row 1, wherein the chimera has the sequence of |
| | GRGEAETRECIYYNANWELERTNQSGLERCEGEQX₁KRRHCFATWKNISGSIEIVKQGCWLDDX₂ |
| | X₃CYDRTDCVX₄X₅X₆X₇X₈PX₉VYFCCCEGNMCNEKFSYFPEMEVTQPTS (SEQ ID NO: 758). |
| 11 | The polypeptide of row 1, wherein the chimera has the sequence of |
| | GRGEAETRECIYYNANWELERTNQSGLERCEGEQX₁KRLHCFATWKNISGSIEIVKQGCWLDDX₂ |
| | X₃CYDRTDCVX₄X₅X₆X₇X₈PX₉VYFCCCEGNMCNEKFSYFPEMEVTQPTS (SEQ ID NO: 759). |
| 12 | The polypeptide of row 1, wherein the chimera has the sequence of |
| | GRGEAETRECIYYNANWELERTNQSGLERCEGEQX₁KRLHCYASWKNISGSIEIVKQGCWLDDX₂ |
| | X₃CYDRTDCVX₄X₅X₆X₇X₈PX₉VYFCCCEGNMCNEKFSYFPEMEVTQPTS (SEQ ID NO: 760). |
| 13 | The polypeptide of row 1, wherein the chimera has the sequence of |
| | GRGEAETRECIYYNANWELERTNQSGLERCEGEQX₁KRLHCYASWRNSSGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 761). |
| 14 | The polypeptide of row 1, wherein the chimera has the sequence of |
| | GRGEAETRECIYYNANWELERTNQSGLERCEGEQX₁KRLHCYASWRNSSGTIEIVKQGCWLDD |
| | X₂X₃CYDRTDCVX₄X₅X₆X₇X₈PX₉VYFCCCEGNMCNEKFSYFPEMEVTQPTS (SEQ ID NO: 762). |
| 15 | The polypeptide of row 1, wherein the chimera has the sequence of |
| | GRGEAETRECIYYNANWELERTNQSGLERCEGEQX₁KRLHCYASWRNSSGTIELVKKGCWLDD |
| | X₂X₃CYDRTDCVX₄X₅X₆X₇X₈PX₉VYFCCCEGNMCNEKFSYFPEMEVTQPTS (SEQ ID NO: 763). |
| 16 | The polypeptide of row 1, wherein the chimera has the sequence of |
| | GRGEAETRECIYYNANWELERTNQSGLERCEGEQX₁KRLHCYASWRNSSGTIELVKKGCWLDD |
| | X₂X₃CYDRQECVX₄X₅X₆X₇X₈PX₉VYFCCCEGNMCNEKFSYFPEMEVTQPTS (SEQ ID NO: 764). |
| 17 | The polypeptide of row 2, wherein the chimera has the sequence of |
| | GAILGRSETQECIYYNANWELERTNQSGLERCEGEQX₁KRRHCFATWKNISGSIEIVKQGCWLD |
| | DX₂X₃CYDRTDCVX₄X₅X₆X₇X₈PX₉VYFCCCEGNMCNEKFSYFPEMEVTQPTS (SEQ ID NO: 765). |
| 18 | The polypeptide of row 2, wherein the chimera has the sequence of |
| | GAILGRSETQECIYYNANWELERTNQSGLERCEGEQX₁KRLHCFATWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 766). |
| 19 | The polypeptide of row 2, wherein the chimera has the sequence of |
| | GAILGRSETQECIYYNANWELERTNQSGLERCEGEQX₁KRLHCYASWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 767). |
| 20 | The polypeptide of row 2, wherein the chimera has the sequence of |
| | GAILGRSETQECIYYNANWELERTNQSGLERCEGEQX₁KRLHCYASWRNSSGSIEIVKQGCWLD |
| | DX₂X₃CYDRTDCVX₄X₅X₆X₇X₈PX₉VYFCCCEGNMCNEKFSYFPEMEVTQPTS (SEQ ID NO: 768). |
| 21 | The polypeptide of row 2, wherein the chimera has the sequence of |
| | GAILGRSETQECIYYNANWELERTNQSGLERCEGEQX₁KRLHCYASWRNSSGTIEIVKQGCWLD |
| | DX₂X₃CYDRTDCVX₄X₅X₆X₇X₈PX₉VYFCCCEGNMCNEKFSYFPEMEVTQPTS (SEQ ID NO: 769). |
| 22 | The polypeptide of row 2, wherein the chimera has the sequence of |
| | GAILGRSETQECIYYNANWELERTNQSGLERCEGEQX₁KRLHCYASWRNSSGTIELVKKGCWLD |
| | DX₂X₃CYDRTDCVX₄X₅X₆X₇X₈PX₉VYFCCCEGNMCNEKFSYFPEMEVTQPTS (SEQ ID NO: 770). |
| 23 | The polypeptide of row 2, wherein the chimera has the sequence of |
| | GAILGRSETQECIYYNANWELERTNQSGLERCEGEQX₁KRLHCYASWRNSSGTIELVKKGCWLD |
| | DX₂X₃CYDRQECVX₄X₅X₆X₇X₈PX₉VYFCCCEGNMCNEKFSYFPEMEVTQPTS (SEQ ID NO: 771). |
| 24 | The polypeptide of any one of rows 1-23, wherein X₁ is D. |
| 25 | The polypeptide of any one of rows 1-23, wherein X₁ is R. |
| 26 | The polypeptide of any one of rows 1-25, wherein X₂ is I. |
| 27 | The polypeptide of any one of rows 1-25, wherein X₂ is F. |
| 28 | The polypeptide of any one of rows 1-25, wherein X₂ is E. |
| 29 | The polypeptide of any one of rows 1-25, wherein X₂ is D. |
| 30 | The polypeptide of any one of rows 1-25, wherein X₂ is Y. |
| 31 | The polypeptide of any one of rows 1-25, wherein X₂ is S. |
| 32 | The polypeptide of any one of rows 1-25, wherein X₂ is N. |
| 33 | The polypeptide of any one of rows 1-25, wherein X₂ is Q. |
| 34 | The polypeptide of any one of rows 1-25, wherein X₂ is T. |
| 35 | The polypeptide of any one of rows 1-34, wherein X₃ is N. |
| 36 | The polypeptide of any one of rows 1-34, wherein X₃ is T. |
| 37 | The polypeptide of any one of rows 1-36, wherein X₄ is A. |
| 38 | The polypeptide of any one of rows 1-36, wherein X₄ is E. |
| 39 | The polypeptide of any one of rows 1-38, wherein X₅ is T. |
| 40 | The polypeptide of any one of rows 1-38, wherein X₅ is K. |
| 41 | The polypeptide of any one of rows 1-40, wherein X₆ is E. |
| 42 | The polypeptide of any one of rows 1-40, wherein X₆ is K. |
| 43 | The polypeptide of any one of rows 1-42, wherein X₇ is E. |
| 44 | The polypeptide of any one of rows 1-42, wherein X₇ is D. |
| 45 | The polypeptide of any one of rows 1-44, wherein X₈ is N. |
| 46 | The polypeptide of any one of rows 1-44, wherein X₈ is S. |
| 47 | The polypeptide of any one of rows 1-46, wherein X₉ is Q. |
| 48 | The polypeptide of any one of rows 1-46, wherein X₉ is E. |
| 49 | The polypeptide of any one of rows 1-46, wherein X₉ is K. |
| 50 | The polypeptide of any one of rows 1-46, wherein X₉ is R. |
| 51 | The polypeptide of any one of rows 1-46, wherein X₉ is D. |
| 52 | The polypeptide of any one of rows 1-46, wherein X₉ is N. |
| 53 | The polypeptide of any one of rows 1-52, wherein X₅ is T, X₆ is E, X₇ is E, and X₈ is N. |
| 54 | The polypeptide of any one of rows 1-52, wherein X₅ is T, X₆ is K, X₇ is E, and X₈ is N. |
| 55 | The polypeptide of any one of rows 1-54, wherein X₂ is E and X₃ is T. |
| 56 | The polypeptide of any one of rows 1-54, wherein X₂ is I or F and X₃ is N. |
| 57 | The polypeptide of row 56, wherein X₂ is I. |
| 58 | The polypeptide of row 56, wherein X₂ is F. |
| 59 | The polypeptide of row 1 or row 2, wherein the chimera has the sequence of any one of |
| | SEQ ID NOs: 772-793. |
| 60 | The polypeptide of row 59, wherein the chimera has the sequence of SEQ ID NO: 772. |
| 61 | The polypeptide of row 59, wherein the chimera has the sequence of SEQ ID NO: 773. |
| 62 | The polypeptide of row 59, wherein the chimera has the sequence of SEQ ID NO: 774. |
| 63 | The polypeptide of row 59, wherein the chimera has the sequence of SEQ ID NO: 775. |
| 64 | The polypeptide of row 59, wherein the chimera has the sequence of SEQ ID NO: 790. |
| 65 | The polypeptide of row 59, wherein the chimera has the sequence of SEQ ID NO: 791. |
| 66 | The polypeptide of row 59, wherein the chimera has the sequence of SEQ ID NO: 778. |
| 67 | The polypeptide of row 59, wherein the chimera has the sequence of SEQ ID NO: 792. |
| 68 | The polypeptide of row 59, wherein the chimera has the sequence of SEQ ID NO: 793. |
| 69 | The polypeptide of row 59, wherein the chimera has the sequence of SEQ ID NO: 787. |
| 70 | The polypeptide of any one of rows 1-69, wherein the chimera is truncated from the N- |
| | terminus by deletion of one amino acid. |
| 71 | The polypeptide of any one of rows 1-69, wherein the chimera is truncated from the N- |
| | terminus by deletion of two amino acids. |
| 72 | The polypeptide of any one of rows 1-69, wherein the chimera is truncated from the N- |
| | terminus by deletion of three amino acids. |
| 73 | The polypeptide of any one of rows 1-69, wherein the chimera is truncated from the N- |
| | terminus by deletion of four amino acids. |
| 74 | The polypeptide of any one of rows 1-69, wherein the chimera is truncated from the N- |
| | terminus by deletion of five amino acids. |
| 75 | The polypeptide of any one of rows 1-69, wherein the chimera is truncated from the N- |
| | terminus by deletion of six amino acids. |
| 76 | The polypeptide of any one of rows 1-69, wherein the chimera is truncated from the N- |
| | terminus by deletion of seven amino acids. |
| 77 | The polypeptide of any one of rows 1-69, wherein the chimera is truncated from the N- |
| | terminus by deletion of eight amino acids. |
| 78 | The polypeptide of any one of rows 1-69, wherein the chimera is truncated from the N- |
| | terminus by deletion of nine amino acids. |
| 79 | The polypeptide of any one of rows 1-78, wherein the polypeptide (e.g., the chimera) |
| | further includes a C-terminal extension of one or more amino acids (e.g., 1, 2, 3, 4, |
| | 5, 6, or more amino acids from wild-type extracellular ActRIIA or ActRIIB). |
| 80 | The polypeptide of row 79, wherein the C-terminal extension is NP. |
| 81 | The polypeptide of row 79, wherein the C-terminal extension is NPVTPK (SEQ ID NO: 78). |
| 82 | The polypeptide of any one of rows 1-81, wherein the polypeptide further includes an |
| | Fc domain monomer fused to the C-terminus of the polypeptide (e.g., the C-terminus of |
| | the chimera) by way of a linker. |
| 83 | The polypeptide of row 82, wherein the Fc domain monomer has the sequence of SEQ ID NO: |
| | 97. |
| 84 | The polypeptide of row 82 or 83, wherein the polypeptide forms a dimer. |
| 85 | The polypeptide of any one of rows 1-81, wherein the polypeptide further includes an |
| | Fc domain fused to the C-terminus of the polypeptide (e.g., the C-terminus of the |
| | chimera) by way of a linker. |
| 86 | The polypeptide of row 85, wherein the Fc domain comprises the sequence of SEQ ID NO: |
| | 84 or SEQ ID NO: 79. |
| 87 | The polypeptide of any one of rows 1-81, wherein the polypeptide further includes an |
| | albumin-binding peptide fused to the C-terminus of the polypeptide (e.g., the C- |
| | terminus of the chimera) by way of a linker. |
| 88 | The polypeptide of row 87, wherein the albumin-binding peptide has the sequence of SEQ |
| | ID NO: 83. |
| 89 | The polypeptide of any one of rows 1-81, wherein the polypeptide further includes a |
| | fibronectin domain fused to the C-terminus of the polypeptide (e.g., the C-terminus of |
| | the chimera) by way of a linker. |
| 90 | The polypeptide of row 89, wherein the fibronectin domain has the sequence of SEQ ID |
| | NO: 82. |
| 91 | The polypeptide of any one of rows 1-81, wherein the polypeptide further includes a |
| | human serum albumin fused to the C-terminus of the polypeptide (e.g., the C-terminus |
| | of the chimera) by way of a linker. |
| 92 | The polypeptide of row 91, wherein the human serum albumin has the sequence of SEQ ID |
| | NO: 81. |
| 93 | The polypeptide of any one of rows 82-92, wherein the linker is an amino acid spacer. |
| 94 | The polypeptide of row 93, wherein the amino acid spacer is GGG, GGGA (SEQ ID NO: 98), |
| | GGGG (SEQ ID NO: 100), GGGAG (SEQ ID NO: 130), GGGAGG (SEQ ID NO: 131), or |
| | GGGAGGG (SEQ ID NO: 132). |
| 95 | The polypeptide of row 93, wherein the amino acid spacer is GGS, GGGS (SEQ ID NO: 99), |
| | GGGGS (SEQ ID NO: 102), GGSG (SEQ ID NO: 105), or SGGG (SEQ ID NO: 107). |
| 96 | The polypeptide of row 93, wherein the amino acid spacer is GA, GS, GG, GGA, GGS, GGG, |
| | GGGS (SEQ ID NO: 99), GGGGA (SEQ ID NO: 101), GGGGS (SEQ ID NO: 102), GGGGG |
| | (SEQ ID NO: 103), GGAG (SEQ ID NO: 104), GGSG (SEQ ID NO: 105), AGGG (SEQ ID NO: |
| | 106), SGGG (SEQ ID NO: 107), GAGA (SEQ ID NO: 108), GSGS (SEQ ID NO: 109), GAGAGA |
| | (SEQ ID NO: 110), GSGSGS (SEQ ID NO: 111), GAGAGAGA (SEQ ID NO: 112), GSGSGSGS |
| | (SEQ ID NO: 113), GAGAGAGAGA (SEQ ID NO: 114), GSGSGSGSGS (SEQ ID NO: 115), |
| | GAGAGAGAGAGA (SEQ ID NO: 116), and GSGSGSGSGSGS (SEQ ID NO: 117), GGAGGA |
| | (SEQ ID NO: 118), GGSGGS (SEQ ID NO: 119), GGAGGAGGA (SEQ ID NO: 120), |
| | GGSGGSGGS (SEQ ID NO: 121), GGAGGAGGAGGA (SEQ ID NO: 122), GGSGGSGGSGGS |
| | (SEQ ID NO: 123), GGAGGGAG (SEQ ID NO: 124), GGSGGGSG (SEQ ID NO: 125), |
| | GGAGGGAGGGAG (SEQ ID NO: 126), and GGSGGGSGGGSG (SEQ ID NO: 127), |
| | GGGGAGGGGAGGGGA (SEQ ID NO: 128), GGGGSGGGGSGGGGS (SEQ ID NO: 129), AAAL |
| | (SEQ ID NO: 133), AAAK (SEQ ID NO: 134), AAAR (SEQ ID NO: 135), EGKSSGSGSESKST |
| | (SEQ ID NO: 136), GSAGSAAGSGEF (SEQ ID NO: 137), AEAAAKEAAAKA (SEQ ID NO: 96), |
| | KESGSVSSEQLAQFRSLD (SEQ ID NO: 95), GENLYFQSGG (SEQ ID NO: 94), SACYCELS |
| | (SEQ ID NO: 93), RSIAT (SEQ ID NO: 92), RPACKIPNDLKQKVMNH (SEQ ID NO: 91), |
| | GGSAGGSGSGSSGGSSGASGTGTAGGTGSGSGTGSG (SEQ ID NO: 90), |
| | AAANSSIDLISVPVDSR (SEQ ID NO: 89), |
| | GGSGGGSEGGGSEGGGSEGGGSEGGGSEGGGSGGGS (SEQ ID NO: 88), EAAAK (SEQ ID |
| | NO: 87), or PAPAP(SEQ ID NO: 86). |
| 97 | The polypeptide of any one of rows 1-96, wherein the polypeptide (e.g., an ActRII |
| | chimera-Fc fusion protein) has a serum half-life of at least seven days. |
| 98 | The polypeptide of any one of rows 1-97, wherein the polypeptide has increased binding |
| | to one or more an ActRII ligands (e.g., activin A, activin B, myostatin, and/or GDF-11) |
| | compared to wild-type ActRIIA and/or wild-type ActRIIB (e.g., wild-type extracellular |
| | ActRIIA and/or ActRIIB). |
| 99 | The polypeptide of any one of rows 1-98, wherein the polypeptide has decreased binding |
| | to bone morphogenetic protein 9 (BMP9, e.g., human BMP9) compared to wild-type ActRIIB |
| | (e.g., wild-type extracellular ActRIIB). |
| 100 | The polypeptide of any one of rows 1-99, wherein the polypeptide binds to activin A, |
| | activin B, and/or myostatin and has reduced or weak binding to human BMP9 (e.g., |
| | compared to wild-type extracellular ActRIIB). |
| 101 | The polypeptide of any one of rows 1-100, wherein the polypeptide does not |
| | substantially bind to human BMP9. |
| 102 | The polypeptide of any one of rows 1-101, wherein the polypeptide binds to human |
| | activin A with a KD of 800 pM or less. |
| 103 | The polypeptide of any one of rows 1-102, wherein the polypeptide binds to human |
| | activin B with a KD of 800 pM or less. |
| 104 | The polypeptide of any one of rows 1-103, wherein the polypeptide binds to human |
| | GDF-11 with a KD of 5 pM or higher. |
| 105 | A nucleic acid molecule encoding the polypeptide of any one of rows 1-104. |
| 106 | A vector comprising the nucleic acid molecule of row 105. |
| 107 | A host cell that expresses the polypeptide of any one of rows 1-104, wherein the host |
| | cell comprises the nucleic acid molecule of row 105 or the vector of row 106, wherein |
| | the nucleic acid molecule or vector is expressed in the host cell. |
| 108 | A pharmaceutical composition comprising the polypeptide of any one of rows 1-104, the |
| | nucleic acid molecule of row 105, or the vector of row 106 and one or more |
| | pharmaceutically acceptable carriers or excipients. |
| 109 | The pharmaceutical composition of row 108, wherein the polypeptide, nucleic acid |
| | molecule, or vector is in a therapeutically effective amount. |
| 110 | A construct comprising two identical polypeptides (e.g., a homodimer), each comprising |
| | an extracellular ActRII chimera of any one of rows 1-81 fused (e.g., linked using an |
| | amino acid spacer) to the N- or C-terminus of an Fc domain monomer (e.g., the sequence |
| | of SEQ ID NO: 97). The two Fc domain monomers in the two polypeptides interact to form |
| | an Fc domain in the construct. |
| 111 | A construct comprising two different polypeptides (e.g., a heterodimer), each comprising |
| | an extracellular ActRII chimera of any one of rows 1-81 fused (e.g., linked using an |
| | amino acid spacer) to the N- or C-terminus of an Fc domain monomer (e.g., the sequence |
| | of SEQ ID NO: 97). The two Fc domain monomers in the two polypeptides interact to form |
| | an Fc domain in the construct. |

TABLE 21

| Row | Composition |
|---|---|
| 1 | A polypeptide comprising an extracellular activin receptor type II (ActRII) chimera, the |
| | chimera having a sequence of X₁β₁X₂β₂X₃β₃X₄β₄X₅β₅X₆β₆X₇β₇X₈, wherein: |
| | X₁ is GAILGRSETQ (SEQ ID NO: 794) or GRGEAETR (SEQ ID NO: 795); |
| | β₁ is ECLFFN (β₁ₐ) (SEQ ID NO: 796) or ECIYYN (β_{1b}) (SEQ ID NO: 797); |
| | X₂ is ANWEKDRTN (SEQ ID NO: 798) or ANWELERTN (SEQ ID NO: 799); |
| | β₂ is QTGVEPC (ß₂ₐ) (SEQ ID NO: 800) or QSGLERC (β_{2b}) (SEQ ID NO: 801); |
| | X₃ is YGDKDKR (SEQ ID NO: 802) or EGEQDKR (SEQ ID NO: 803); |
| | β₃ is RHCFATWKNI (β₃ₐ) (SEQ ID NO: 804) or a portion thereof that comprises HCFATWK (SEQ |
| | ID NO: 805) or LHCYASWRNS (β_{3b}) (SEQ ID NO: 806) or a portion thereof that comprises |
| | HCYASWR (SEQ ID NO: 807), wherein when β₃ is HCFATWK or HCYASWR, the chimera |
| | comprises contiguous amino acids from ActRIIA or ActRIIB connecting β₃ to X₃ and X₄; |
| | X₄ is SG; |
| | β₄ is SIEIVKQGCW (β₄ₐ) (SEQ ID NO: 808) or a portion thereof that comprises EIVKQGCW (SEQ |
| | ID NO: 809) or TIELVKKGCW (β_{4b}) (SEQ ID NO: 810) or a portion thereof that comprises |
| | ELVKKGCW (SEQ ID NO: 811), wherein when β₄ is EIVKQGCW or ELVKKGCW, the chimera |
| | comprises contiguous amino acids from ActRIIA or ActRIIB connecting β₄ to X₄; |
| | X₅ is LDDINCYDRTDC (SEQ ID NO: 812) or LDDFNCYDRQEC (SEQ ID NO: 813); |
| | ß₅ is VEK (β₅ₐ) or a portion thereof that comprises VE or VAT (β_{5b}) or a portion thereof |
| | that comprises V, wherein when β₅ is VE or V, the chimera comprises contiguous amino |
| | acids from ActRIIA or ActRIIB connecting β₅ to X₆; |
| | X₆ is KDSPEV (SEQ ID NO: 814) or EENPQV (SEQ ID NO: 815); |
| | β₆ is YFCCCE (SEQ ID NO: 816); |
| | X₇ is GNMCNE (SEQ ID NO: 817) or GNFCNE (SEQ ID NO: 818); |
| | β₇ is KFSYF (β₇ₐ) (SEQ ID NO: 819) or a portion thereof that comprises SYF or RFTHL (β_{7b}) |
| | (SEQ ID NO: 820) or a portion thereof that comprises T, wherein when β₇ is SYF or T, the |
| | chimera comprises contiguous amino acids from ActRIIA or ActRIIB connecting β₇ to X₇ and |
| | X₈; and X₈ is PEMEVTQPTS (SEQ ID NO: 821) or PEAGGPEVTYEPPPTAPT (SEQ ID NO: 822), |
| | wherein at least one of β₁ₐ, β₂ₐ, β₃ₐ, β₄ₐ, β₅ₐ, or β₇ₐ and at least one of β_{1b}, β_{2b}, β_{3b}, β_{4b}, |
| | β_{5b}, or β_{7b} is present in the chimera, optionally wherein the chimera is truncated from the |
| | N-terminus by deletion of 1, 2, 3, 4, 5, 6, 7, 8, or 9 amino acids, wherein the chimera |
| | retains the two amino acids before the first cysteine. |
| 2 | The polypeptide of row 1, wherein X₁ is GAILGRSETQ. |
| 3 | The polypeptide of row 1, wherein X₁ is GRGEAETR. |
| 4 | The polypeptide of any one of rows 1-3, wherein β₁ is ECLFFN (β₁ₐ). |
| 5 | The polypeptide of any one of rows 1-3, wherein β₁ is ECIYYN (β_{1b}). |
| 6 | The polypeptide of any one of rows 1-5, wherein X₂ is ANWEKDRTN. |
| 7 | The polypeptide of any one of rows 1-5, wherein X₂ is ANWELERTN. |
| 8 | The polypeptide of any one of rows 1-7, wherein β₂ is QTGVEPC (β₂ₐ). |
| 9 | The polypeptide of any one of rows 1-7, wherein β₂ is QSGLERC (β_{2b}). |
| 10 | The polypeptide of any one of rows 1-9, wherein X₃ is YGDKDKR. |
| 11 | The polypeptide of any one of rows 1-9, wherein X₃ is EGEQDKR. |
| 12 | The polypeptide of any one of rows 1-11, wherein β₃ is RHCFATWKNI (β₃ₐ). |
| 13 | The polypeptide of any one of rows 1-11, wherein β₃ is LHCYASWRNS (β_{3b}). |
| 14 | The polypeptide of any one of rows 1-11, wherein β₃ is HCFATWK, wherein the chimera |
| | comprises contiguous amino acids from ActRIIA or ActRIIB connecting β₃ to X₃ and X₄ |
| 15 | The polypeptide of any one of rows 1-11, wherein β₃ is HCYASWR, wherein the chimera |
| | comprises contiguous amino acids from ActRIIA or ActRIIB connecting β₃ to X₃ and X₄. |
| 16 | The polypeptide of row 14 or 15, wherein the contiguous amino acids connecting β₃ to X₃ |
| | are from ActRIIA. |
| 17 | The polypeptide of row 14 or 15, wherein the contiguous amino acids connecting β₃ to X₃ |
| | are from ActRIIB. |
| 18 | The polypeptide of any one of rows 14-17, wherein the contiguous amino acids connecting |
| | β₃ to X₄ are from ActRIIA. |
| 19 | The polypeptide of any one of rows 14-17, wherein the contiguous amino acids connecting |
| | β₃ to X₄ are from ActRIIB. |
| 20 | The polypeptide of any one of rows 1-19, wherein β₄ is SIEIVKQGCW (β₄ₐ). |
| 21 | The polypeptide of any one of rows 1-19, wherein β₄ is TIELVKKGCW (β_{4b}). |
| 22 | The polypeptide of any one of rows 1-19, wherein β₄ is EIVKQGCW, wherein the chimera |
| | comprises contiguous amino acids from ActRIIA or ActRIIB connecting β₄ to X₄. |
| 23 | The polypeptide of any one of rows 1-19, wherein β₄ is ELVKKGCW, wherein the chimera |
| | comprises contiguous amino acids from ActRIIA or ActRIIB connecting β₄ to X₄. |
| 24 | The polypeptide of row 22 or 23, wherein the contiguous amino acids connecting β₄ to X₄ |
| | are from ActRIIA. |
| 25 | The polypeptide of row 22 or 23, wherein the contiguous amino acids connecting β₄ to X₄ |
| | are from ActRIIB. |
| 26 | The polypeptide of any one of rows 1-25, wherein X₅ is LDDINCYDRTDC. |
| 27 | The polypeptide of any one of rows 1-25, wherein X₅ is LDDFNCYDRQEC. |
| 28 | The polypeptide of any one of rows 1-27, wherein β₅ is VEK (β₅ₐ). |
| 29 | The polypeptide of any one of rows 1-27, wherein β₅ is VAT (β_{5b}). |
| 30 | The polypeptide of any one of rows 1-27, wherein β₅ is VE, wherein the chimera comprises |
| | contiguous amino acids from ActRIIA or ActRIIB connecting β₅ to X₆. |
| 31 | The polypeptide of any one of rows 1-27, wherein B₅ is V, wherein the chimera comprises |
| | contiguous amino acids from ActRIIA or ActRIIB connecting β₅ to X₆. |
| 32 | The polypeptide of row 30 or 31, wherein the contiguous amino acids connecting β₅ to X₆ |
| | are from ActRIIA. |
| 33 | The polypeptide of row 30 or 31, wherein the contiguous amino acids connecting β₅ to X₆ |
| | are from ActRIIB. |
| 34 | The polypeptide of any one of rows 1-33, wherein X₆ is KDSPEV. |
| 35 | The polypeptide of any one of rows 1-33, wherein X₆ is EENPQV. |
| 36 | The polypeptide of any one of rows 1-35, wherein X₇ is GNMCNE. |
| 37 | The polypeptide of any one of rows 1-35, wherein X₇ is GNFCNE. |
| 38 | The polypeptide of any one of rows 1-37, wherein β₇ is KFSYF (B₇ₐ). |
| 39 | The polypeptide of any one of rows 1-37, wherein β₇ is RFTHL (B_{7b}). |
| 40 | The polypeptide of any one of rows 1-37, wherein β₇ is SYF, wherein the chimera comprises |
| | contiguous amino acids from ActRIIA or ActRIIB connecting β₇ to X₇. |
| 41 | The polypeptide of any one of rows 1-37, wherein β₇ is T, wherein the chimera comprises |
| | contiguous amino acids from ActRIIA or ActRIIB connecting β₇ to X₇ and X₈. |
| 42 | The polypeptide of row 40 or 41, wherein the contiguous amino acids connecting β₇ to X₇ |
| | are from ActRIIA. |
| 43 | The polypeptide of row 40 or 41, wherein the contiguous amino acids connecting β₇ to X₇ |
| | are from ActRIIB. |
| 44 | The polypeptide of any one of rows 41-43, wherein the contiguous amino acids connecting |
| | β₇ to X₈ are from ActRIIA. |
| 45 | The polypeptide of any one of rows 41-43, wherein the contiguous amino acids connecting |
| | β₇ to X₈ are from ActRIIB. |
| 46 | The polypeptide of any one of rows 1-45, wherein X₈ is PEMEVTQPTS. |
| 47 | The polypeptide of any one of rows 1-45, wherein X₈ is PEAGGPEVTYEPPPTAPT. |
| 48 | The polypeptide of any one of rows 1-47, wherein the chimera is truncated from the N- |
| | terminus by deletion of one amino acid. |
| 49 | The polypeptide of any one of rows 1-47, wherein the chimera is truncated from the N- |
| | terminus by deletion of two amino acids. |
| 50 | The polypeptide of any one of rows 1-47, wherein the chimera is truncated from the N- |
| | terminus by deletion of three amino acids. |
| 51 | The polypeptide of any one of rows 1-47, wherein the chimera is truncated from the N- |
| | terminus by deletion of four amino acids. |
| 52 | The polypeptide of any one of rows 1-47, wherein the chimera is truncated from the N- |
| | terminus by deletion of five amino acids. |
| 53 | The polypeptide of any one of rows 1-47, wherein the chimera is truncated from the N- |
| | terminus by deletion of six amino acids. |
| 54 | The polypeptide of any one of rows 1-47, wherein the chimera is truncated from the N- |
| | terminus by deletion of seven amino acids. |
| 55 | The polypeptide of any one of rows 1-47, wherein the chimera is truncated from the N- |
| | terminus by deletion of eight amino acids |
| 56 | The polypeptide of any one of rows 1-47, wherein the chimera is truncated from the N- |
| | terminus by deletion of nine amino acids. |
| 57 | The polypeptide of any one of rows 1-56, further comprising a C-terminal extension of one |
| | or more amino acids. |
| 58 | The polypeptide of row 57, wherein the C-terminal extension is NP. |
| 59 | The polypeptide of row 57, wherein the C-terminal extension is NPVTPK (SEQ ID NO: 78). |
| 60 | The polypeptide of any one of rows 1-59, wherein the polypeptide further includes an Fc |
| | domain monomer fused to the C-terminus of the polypeptide (e.g., the C-terminus of the |
| | chimera) by way of a linker. |
| 61 | The polypeptide of row 60, wherein the Fc domain monomer comprises the sequence of SEQ ID |
| | NO: 97. |
| 62 | The polypeptide of row 60 or 61, wherein the polypeptide forms a dimer. |
| 63 | The polypeptide of any one of rows 1-59, wherein the polypeptide further includes an Fc |
| | domain fused to the C-terminus of the polypeptide (e.g., the C-terminus of the chimera) |
| | by way of a linker. |
| 64 | The polypeptide of row 63, wherein the Fc domain comprises the sequence of SEQ ID NO: 84 |
| | or SEQ ID NO: 79. |
| 65 | The polypeptide of any one of rows 1-59, wherein the polypeptide further includes an |
| | albumin-binding peptide fused to the C-terminus of the polypeptide (e.g., the C-terminus |
| | of the chimera) by way of a linker. |
| 66 | The polypeptide of row 65, wherein the albumin-binding peptide has the sequence of SEQ ID |
| | NO: 83. |
| 67 | The polypeptide of any one of rows 1-59, wherein the polypeptide further includes a |
| | fibronectin domain fused to the C-terminus of the polypeptide (e.g., the C-terminus of |
| | the chimera) by way of a linker. |
| 68 | The polypeptide of row 67, wherein the fibronectin domain has the sequence of SEQ ID |
| | NO: 82. |
| 69 | The polypeptide of any one of rows 1-59, wherein the polypeptide further includes a human |
| | serum albumin fused to the C-terminus of the polypeptide (e.g., the C-terminus of the |
| | chimera) by way of a linker. |
| 70 | The polypeptide of row 69, wherein the human serum albumin has the sequence of SEQ ID NO: |
| | 81. |
| 71 | The polypeptide of any one of rows 60-70, wherein the linker is an amino acid spacer. |
| 72 | The polypeptide of row 71, wherein the amino acid spacer is GGG, GGGA (SEQ ID NO: 98), |
| | GGGG (SEQ ID NO: 100), GGGAG (SEQ ID NO: 130), GGGAGG (SEQ ID NO: 131), or |
| | GGGAGGG (SEQ ID NO: 132). |
| 73 | The polypeptide of row 71, wherein the amino acid spacer is GGS, GGGS (SEQ ID NO: 99), |
| | GGGGS (SEQ ID NO: 102), GGSG (SEQ ID NO: 105), or SGGG (SEQ ID NO: 107). |
| 74 | The polypeptide of row 71, wherein the amino acid spacer is GA, GS, GG, GGA, GGS, GGG, |
| | GGGS (SEQ ID NO: 99), GGGGA (SEQ ID NO: 101), GGGGS (SEQ ID NO: 102), GGGGG (SEQ |
| | ID NO: 103), GGAG (SEQ ID NO: 104), GGSG (SEQ ID NO: 105), AGGG (SEQ ID NO: 106), |
| | SGGG (SEQ ID NO: 107), GAGA (SEQ ID NO: 108), GSGS (SEQ ID NO: 109), GAGAGA (SEQ |
| | ID NO: 110), GSGSGS (SEQ ID NO: 111), GAGAGAGA (SEQ ID NO: 112), GSGSGSGS (SEQ |
| | ID NO: 113), GAGAGAGAGA (SEQ ID NO: 114), GSGSGSGSGS (SEQ ID NO: 115), |
| | GAGAGAGAGAGA (SEQ ID NO: 116), and GSGSGSGSGSGS (SEQ ID NO: 117), GGAGGA |
| | (SEQ ID NO: 118), GGSGGS (SEQ ID NO: 119), GGAGGAGGA (SEQ ID NO: 120), |
| | GGSGGSGGS (SEQ ID NO: 121), GGAGGAGGAGGA (SEQ ID NO: 122), GGSGGSGGSGGS |
| | (SEQ ID NO: 123), GGAGGGAG (SEQ ID NO: 124), GGSGGGSG (SEQ ID NO: 125), |
| | GGAGGGAGGGAG (SEQ ID NO: 126), and GGSGGGSGGGSG (SEQ ID NO: 127), |
| | GGGGAGGGGAGGGGA (SEQ ID NO: 128), GGGGSGGGGSGGGGS (SEQ ID NO: 129), AAAL |
| | (SEQ ID NO: 133), AAAK (SEQ ID NO: 134), AAAR (SEQ ID NO: 135), EGKSSGSGSESKST |
| | (SEQ ID NO: 136), GSAGSAAGSGEF (SEQ ID NO: 137), AEAAAKEAAAKA (SEQ ID NO: 96), |
| | KESGSVSSEQLAQFRSLD (SEQ ID NO: 95), GENLYFQSGG (SEQ ID NO: 94), SACYCELS |
| | (SEQ ID NO: 93), RSIAT (SEQ ID NO: 92), RPACKIPNDLKQKVMNH (SEQ ID NO: 91), |
| | GGSAGGSGSGSSGGSSGASGTGTAGGTGSGSGTGSG (SEQ ID NO: 90), |
| | AAANSSIDLISVPVDSR (SEQ ID NO: 89), |
| | GGSGGGSEGGGSEGGGSEGGGSEGGGSEGGGSGGGS (SEQ ID NO: 88), EAAAK (SEQ ID |
| | NO: 87), or PAPAP(SEQ ID NO: 86). |
| 75 | The polypeptide of any one of rows 1-74, wherein the polypeptide (e.g., an ActRII |
| | chimera-Fc fusion protein) has a serum half-life of at least seven days. |
| 76 | The polypeptide of any one of rows 1-75, wherein the polypeptide binds to activin A, |
| | activin B, and/or myostatin and has reduced or weak binding to human BMP9 (e.g., compared |
| | to wild-type extracellular ActRIIB). |
| 77 | The polypeptide of any one of rows 1-76, wherein the polypeptide does not substantially |
| | bind to human BMP9. |
| 78 | The polypeptide of any one of rows 1-77, wherein the polypeptide binds to human activin |
| | A with a KD of 800 pM or less. |
| 79 | The polypeptide of any one of rows 1-78, wherein the polypeptide binds to human activin |
| | B with a KD of 800 pM or less. |
| 80 | The polypeptide of any one of rows 1-79, wherein the polypeptide binds to human GDF-11 |
| | with a KD of 5 pM or higher. |
| 81 | A nucleic acid molecule encoding the polypeptide of any one of rows 1-80. |
| 82 | A vector comprising the nucleic acid molecule of row 81. |
| 83 | A host cell that expresses the polypeptide of any one of rows 1-80, wherein the host cell |
| | comprises the nucleic acid molecule of row 81 or the vector of row 82, wherein the nucleic |
| | acid molecule or vector is expressed in the host cell. |
| 84 | A pharmaceutical composition comprising the polypeptide of any one of rows 1-80, the |
| | nucleic acid molecule of row 81, or the vector of row 82 and one or more pharmaceutically |
| | acceptable carriers or excipients. |
| 85 | The pharmaceutical composition of row 84, wherein the polypeptide, nucleic acid molecule, |
| | or vector is in a therapeutically effective amount. |

### EXAMPLES

The following examples are provided to further illustrate some embodiments of the present invention, but are not intended to limit the scope of the invention; it will be understood by their exemplary nature that other procedures, methodologies, or techniques known to those skilled in the art may alternatively be used.

### Example 1—Effect of ActRIIA/B-mFc on Platelets

Eleven-week-old C57BI/6 mice were dosed with either TBS (Vehicle) or ActRIIA/B-mFc (10 mg/kg) via intraperitoneal (IP) administration. Twelve hours post-dose whole blood was sampled, and platelet counts were determined using a veterinary hematology analyzer (Heska Element HT5). Mice were then euthanized, and bone marrow extracted from the femurs. Bone marrow cells were stained with antibodies against Lineage (Perc-Cy5), sca1 (BV525), cKit (Alexa750), CD41 (APC) and CD150 (Pcy7) and analyzed on flow cytometer (Cytoflex, Beckman coulter). Megakaryocyte progenitors were gated on Lin−; sca1−; ckit+; CD150+; CD41+ cells.

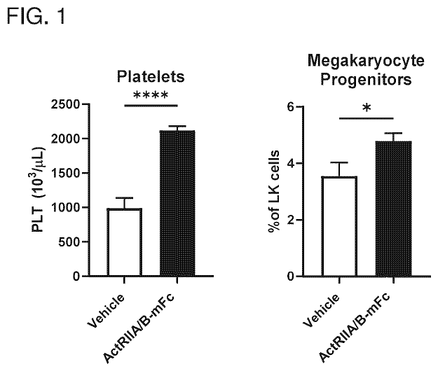

FIG. 1 shows the effect of ActRIIA/B-mFc on thrombopoiesis. A single dose of ActRIIA/B-mFc increased circulating platelet numbers and bone marrow megakaryocyte progenitors within 12 hours post-dose. The timing of the effect on platelets is suggestive of a direct effect of ActRIIA/B-mFc on the terminal maturation of proplatelets to platelets and the megakaryocyte progenitor data demonstrate that that ActRIIA/B-mFc affects earlier stages of the platelet formation process. Data are represented as mean±SEM. Statistical analysis was performed with a Student T-Test; * p<0.05; ** p<0.01; *** p<0.001; *** p<0.0001.

### Example 2—Effect of ActRIIA/B-mFc on Megakaryocyte Maturation

Eleven-week-old C57BI/6 mice were dosed with either TBS (Vehicle) or ActRIIA/B-mFc (10 mg/kg) via IP administration. Twelve and twenty-four hour later mice were euthanized and bone marrow extracted from the femurs. Bone marrow cells were fixed in ethanol following by a staining with Propidium lodide (PI) and anti CD41 (FITC, Efferet) antibody in parallel with RNAse treatment. Samples were analyzed by flow cytometer (Cytoflex, Beckman coulter). Ploidy of CD41+ nucleated cells (PI+ cells) was analyzed. Graphs represent % cells at each ploidy stage. For 12HR N=3 and for 24HR N=2. For CD41+ cells at 12HR time point a t-test was performed for statistical analysis. Data are presented as mean±SEM.

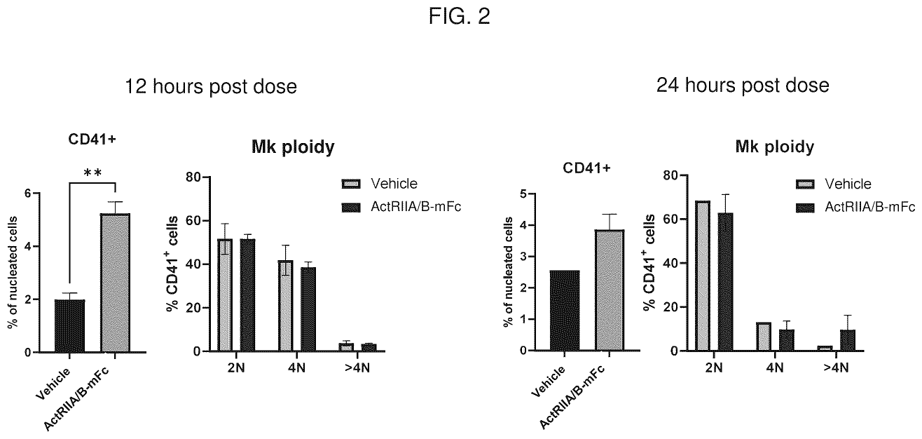

FIG. 2 shows that ActRIIA/B-mFc treatment exhibits a direct effect on megakaryocyte differentiation and maturation as shown by an increase in the number of CD41+ megakaryocyte progenitors at 12 hours after treatment and an increase in number of polyploid megakaryocytes by 24 hours. These data indicate an early effect on progenitors of the thrombopoiesis pathway, indicate that ActRIIA/B-mFc increased differentiation of megakaryocyte precursors toward later stages of maturation, and demonstrate that ActRIIA/B-mFc treatment results in a greater number of megakaryocytes that are potentially primed for more proplatelet production.

### Example 3—Effect of ActRIIA/B-mFc on Platelet Recovery Following Depletion

Twelve-week-old mice were treated with either anti-GP1bα (0.08 mg/kg, Efferet) or IgG control. At day 4 after treatment, the anti-GP1bα treated group was further divided to receive either vehicle or ActRIIA/B-mFc (7.5 mg/kg) treatment. Platelets were measured at indicated time points post anti-GP1bα dosing. At day 10 post-treatment, mice were euthanized and bone marrow cells were harvested. Bone marrow cells were fixed in ice-cold 100% ethanol and stained with Propidium lodide (PI) (200 μg/mL, Sigma-Aldrich) and anti-CD41 (FITC conjugated, Emfret Analytics) antibody in parallel with RNase treatment (2 mg/ml, Invitrogen). Samples were analyzed by flow cytometer (Cytoflex, Beckman coulter) and % CD41+ nucleated cells (PI+ cells) was measured.

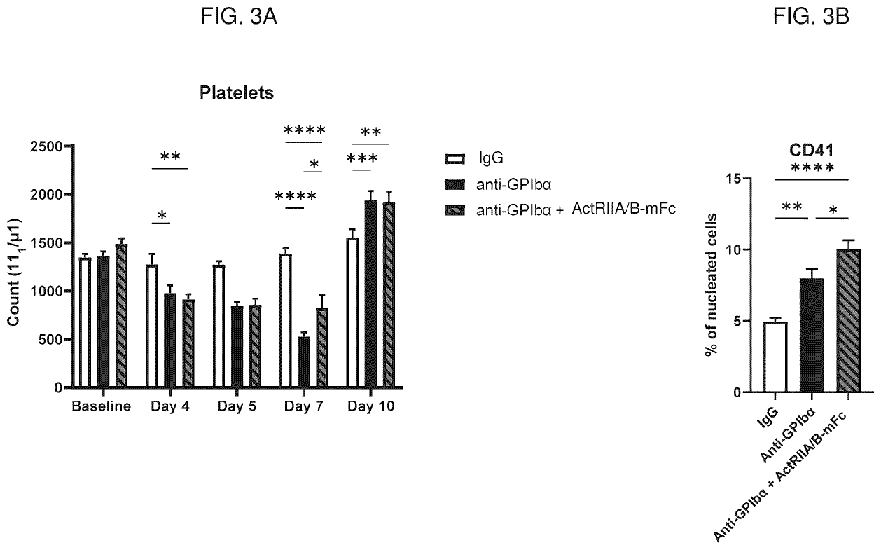

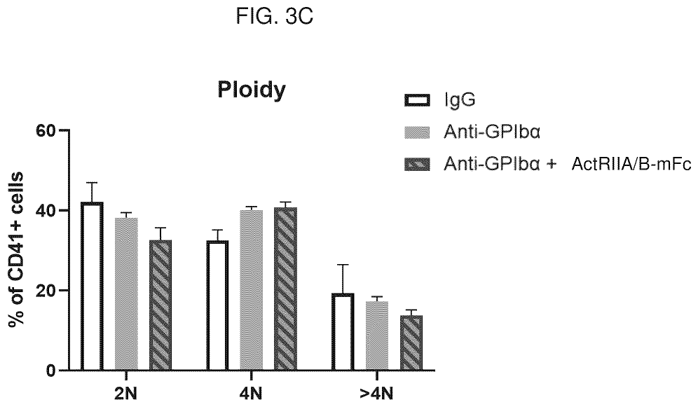

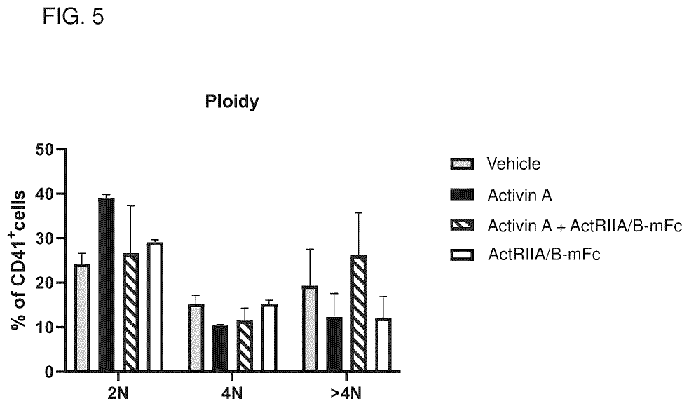

As shown in FIG. 3A, mice treated with ActRIIA/B-mFc exhibited accelerated recovery of platelet numbers following platelet depletion compared to vehicle treated mice in a mouse model of immune thrombocytopenia. These data suggest that ActRIIA/B-mFc could potentially promote faster recovery from thrombocytopenia. Additionally, as shown in FIGS. 5B-5C, there was a 25% increase in the number of CD41⁺ megakaryocyte progenitors in the bone marrow of the ActRIIA/B-mFc-treated group compared to the vehicle-treated group at day 10 after platelet depletion and a higher 4N ploidy level, suggesting that under acute thrombocytopenia, ActRIIA/B-mFc treatment promotes differentiation of megakaryocytes, potentially by accelerating maturation of megakaryocyte precursors, and contributes to accelerated recovery in mice. For platelet data, statistical was analysis conducted with repeat measures mixed effect modeling. Individual comparisons shown are from a Tukey post test. For CD41 data, statistical analysis was performed with 1-Way ANOVA and individual comparisons calculated using a Tukey post test. *p<0.05; ** p<0.01; *** p<0.001; **** p<0.0001. N=9/group.

### Example 4—Effect of a Single Dose of ActRIIA/B-mFc on Platelet Numbers

Eleven-week-old C57BI/6 mice were given a single dose of either TBS (vehicle) or ActRIIA/B-mFc (10 mg/kg) via subcutaneous administration. Separate cohorts of mice from both dosing groups were sampled for whole blood at study day 37, 51 and 85, and platelet counts were determined using a veterinary hematology analyzer (Heska Element HT5).

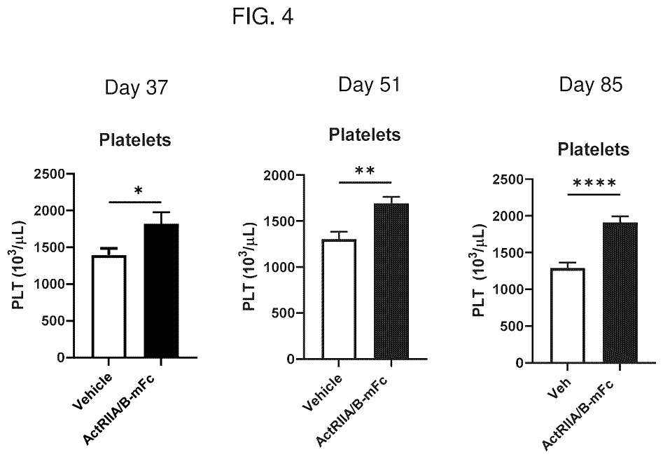

As shown in FIG. 4, a single dose of ActRIIA/B-mFc resulted in increased circulating platelets at day 37, 51, and 85. Data are shown as mean±SEM. Statistical analysis was performed using a Student T-Test; * p≤0.05; ** p≤0.01; *** p≤0.001; **** p≤0.0001.

### Example 5—Effect of ActRIIA/B-mFc on Megakaryocyte Precursors Ex-Vivo

Bone marrow cells from 11-week-old C57BI/6 mice were isolated and treated with Activin A (5 mg/kg), ActRIIA/B-mFc (10 mg/kg), or a combination of both for six days. Cells were harvested after six days and analyzed using flow cytometry (N=2).

As shown in FIG. 5, ex vivo activin A treatment increased 2N ploidy levels and reduced the extent of polyploidy (decreased higher ploidy levels), an indication that activin A was acting to prevent maturation of these cells. Ex-vivo treatment with ActRIIA/B-mFc reversed activin-mediated changes in megakaryocyte precursors, indicating that ActRIIA/B-mFc inhibited the effects of Activin A on ploidy. Higher polyploid levels appeared in the Activin A+ActRIIA/B-mFc treated group compared to the Activin A group. Data are represented as mean±SEM.

### Example 6—Effect of an Anti-Activin a Antibody on Platelet Numbers

Ten-week-old C57BI/6 male mice were dosed with either TBS (vehicle), anti-activin A antibody (described in WO2008031061A2, 5 mg/kg), or ActRIIA/B-mFc (10 mg/kg) via intraperitoneal administration. Twenty-four hours post-dose whole blood was sampled, and platelet counts were determined using a veterinary hematology analyzer (Hematrue).

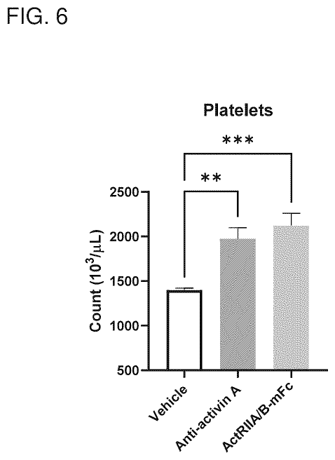

As shown in FIG. 6, treatment with an anti-activin A antibody and with ActRIIA/B-mFc increased platelets in wild-type mice. Observed increases in platelets with the anti-activin A antibody suggests that inhibition of activin A may be at least partly responsible for the increase in platelet levels observed with ActRIIA/B-mFc. Data are represented as mean±SEM. Data were analyzed using Prism 9 (GraphPad Software, San Diego, CA, USA) using one-way ANOVA with Fisher's LSD. * p<0.05, ** p<0.01, *** p<0.001, **** p<0.0001.

### Example 7—Effect of ActRIIA/B-mFc on Platelets, Red Blood Cell Parameters, Spleen Weight, and Immune Cells in a TPO^{high} Model of Myelofibrosis

The TPO^{high} model of myelofibrosis induces a myelofibrotic-like pathology through elevated exposure to thrombopoietin, the native endocrine inducer of megakaryocyte progenitor proliferation and development. Seven-week-old C57BI/6 albino mice (B6(Cg)-Tyr, Jackson Laboratory) were teil-vein injected with 0.75 mg/kg thrombopoietin (TPO) expressing plasmid cloned into pLEV113 plasmid (Lake Pharma). The injection was done in hydrodynamic approach in which 100 ml/kg volume is injected in a short period of time (6-10 seconds). On day 3 after TPO injection mice were divided into 2 groups receiving either vehicle (TBS) or ActRIIA/B-mFc (7.5 mg/kg), twice weekly. Mice were sacrificed on day 14 after TPO injection. Hematological parameters were measured using the Heska Element HT5 veterinary hematology analyzer.

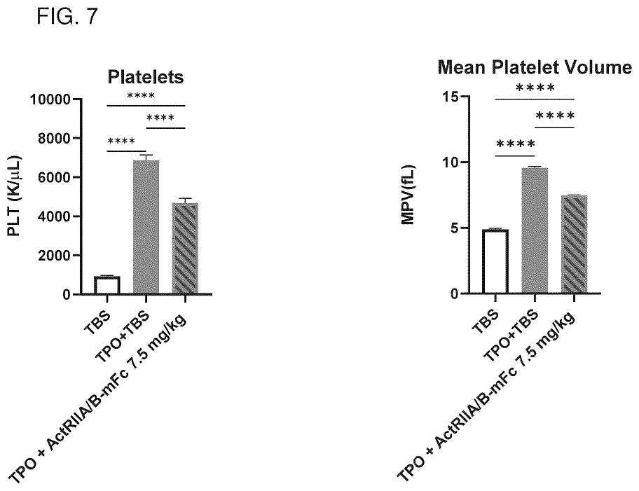

As shown in FIG. 7, TPO HDI increased platelet number and volume. Treatment with ActRIIA/B-mFc was associated with a significant attenuation in the expansion of platelets. High platelet levels are associated with thrombocythemia, which can lead to secondary myelofibrosis. These data suggest that ActRIIA/B-mFc is rebalancing the number of cells committed to the megakaryocyte lineage. N=10-12 mice/group. Results are presented as mean±SEM. Statistical analysis was performed by one-way ANOVA. ****=p<0.0001.

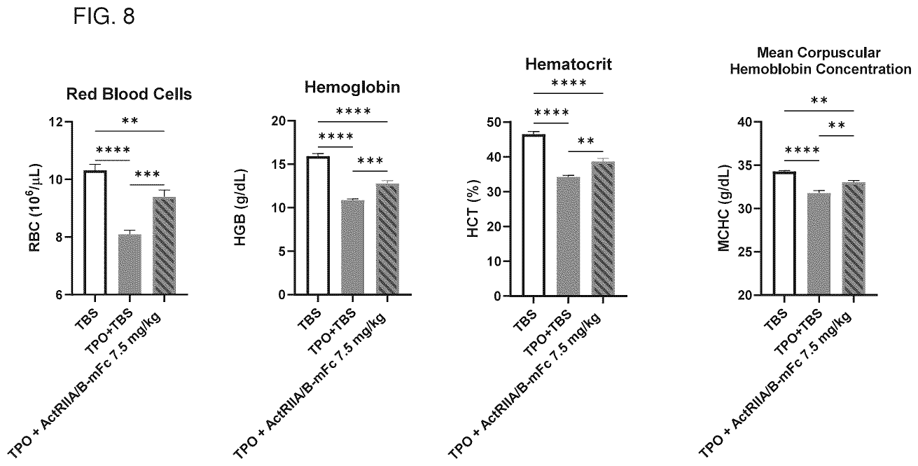

Data confirmed that the TPO^{high} myelofibrosis model was anemic after 14 days of TPO overexpression (FIG. 8). Treatment with ActRIIA/B-mFc was associated with a significant improvement in RBC metrics and appeared to reduce the development of anemia in this model. N=10-12 mice/group. Results are presented as mean±SEM. Statistical analysis was performed by one-way ANOVA. **=p<0.01; ***=p<0.001; ****=p<0.0001.

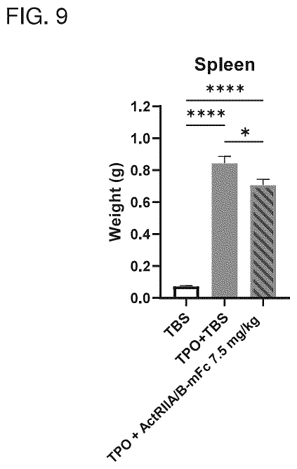

The expansion in megakaryocyte growth and proliferation reduces the capacity of bone marrow for hematopoiesis, inducing compensatory extra medullary hematopoiesis in the liver and spleen (FIG. 9). These data show a significant reduction in splenomegaly in mice treated with ActRIIA/B-mFc, indicating reduced splenic extra medullary hematopoiesis, likely due to a reduced requirement for this compensatory process. N=10-12 mice/group. Results are presented as mean±SEM. Statistical analysis was performed by one-way ANOVA. *=p<0.05; ****=p<0.0001.

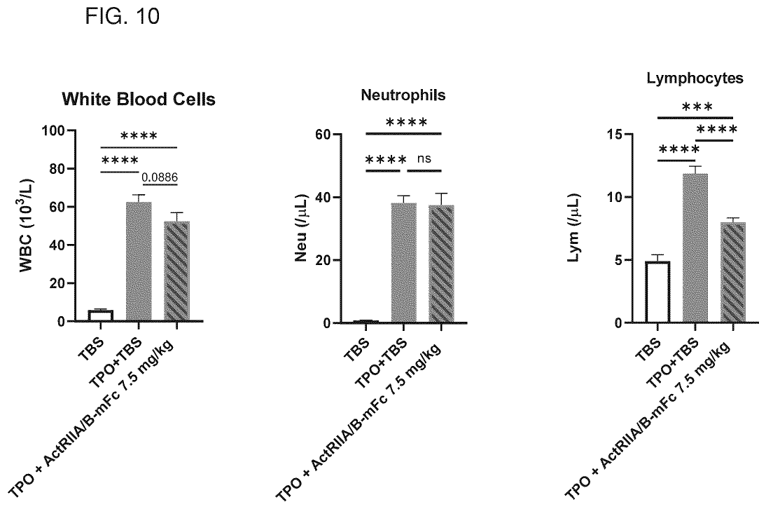

Finally, as shown in FIG. 10, TPO HDI led to an increase in white blood cells, neutrophils, and lymphocytes and treatment with ActRIIA/B-mFc reduced the TPO-mediated increase in white blood cells and lymphocytes. TPO signals through the JAK2 pathway. The JAK2 pathway is proliferative and activating mutations are associated with neoplastic syndromes and a predisposition for the development of myeloid leukemias. N=10-12 mice/group. Results are presented as mean±SEM. Statistical analysis was performed by one-way ANOVA. ***=p<0.001; ****=p<0.0001.

### Example 8—Effect of administering ActRIIA/B-mFc in combination with ruxolitinib

To examine whether treatment with ActRIIA/B-mFc can overcome ruxolitinib-induced anemia, a pharmacological assessment was conducted in mice. Ten- to twelve-week-old, female, C57BI/6 mice were dosed orally with either vehicle (n=10) or ruxolitinib at 90 mg/kg (n=20) or 120 mg/kg (n=20) twice daily. After 37 days of ruxolitinib therapy, blood was sampled via cheek bleed for hematological assessment. On Day 41, mice from each ruxolitinib dose were divided into two groups that received either TBS (vehicle) (n=10) or ActRIIA/B-mFc (7.5 mg/kg, n=10 per group), IP twice weekly for 14 days concomitant to continued ruxolitinib dosing. Hematological parameters were assessed in whole blood with the use of a Heska Element HT5 veterinary hematology analyzer.

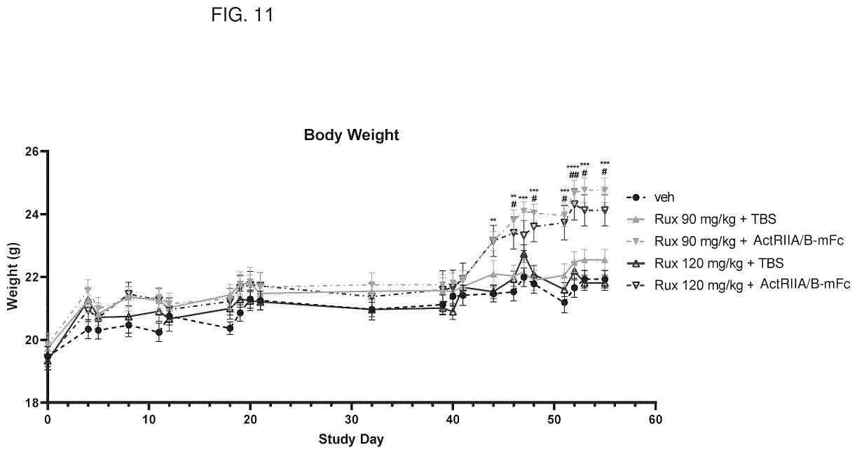

As shown in FIG. 11, ActRIIA/B-mFc increased body weight in mice treated with ruxolitinib. N=10. Data are presented as mean±SEM. A two-way ANOVA was used for statistical analysis. **=p<0.01 between Veh and Rux 90 mg/kg+ActRIIA/B-mFc at each time point. ***=p<0.005 between Veh and Rux 90 mg/kg+ActRIIA/B-mFc at each time point. ****=p<0.0001 between Veh and Rux 90 mg/kg+ActRIIA/B-mFc at each time point. #=p<0.05 between Veh and RUX 120 mg/kg+ActRIIA/B-mFc at each time point. ##=p<0.01 between Veh and RUX 120 mg/kg+ActRIIA/B-mFc at each time point. This demonstrates that ruxolitinib+ActRIIA/B-mFc combination therapy did not have a negative effect on bodyweight, a common marker of tolerability. Moreover, the ability to increase body weight could be a potential benefit in elderly myelofibrosis patients.

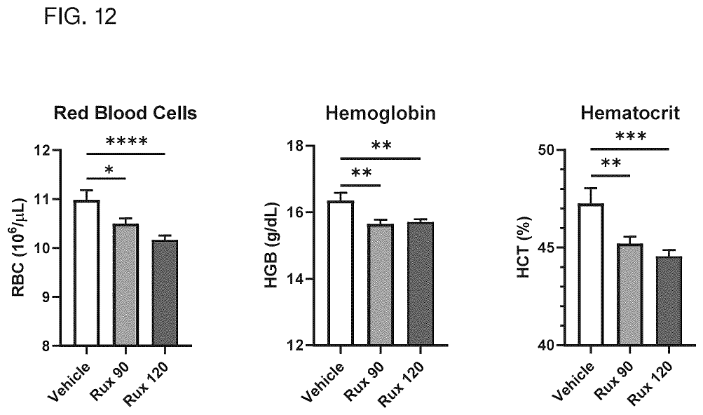

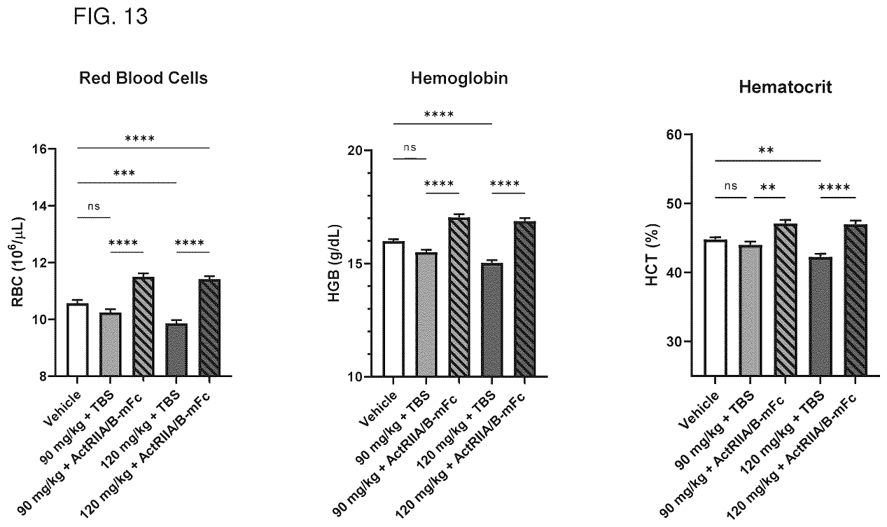

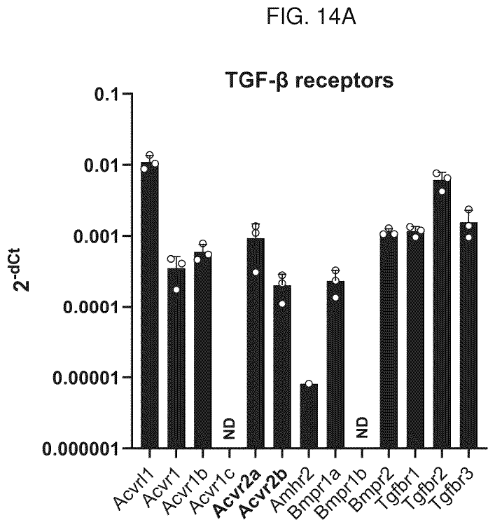

As shown in FIG. 12, treatment with ruxolitinib alone reduced RBC volume, hemoglobin, and hematocrit. Data are represented as mean±SEM. Vehicle N=10, Rux 90 N=20, Rux 120 N=20. One-way ANOVA followed by Dunnett post hoc test was used for statistical analysis. * p≤0.05; ** p≤0.01; *** p≤0.001; **** p≤0.0001. However, administration of ActRIIA/B-mFc abrogated the ruxolitinib-associated reductions in RBC volume, hemoglobin, and hematocrit (FIG. 13). This indicates that ActRIIA/B-mFc functions independent of the JAK/STAT pathway and suggests that ActRIIA/B-Fc could be a potential treatment option for ineffective hematopoiesis resulting from defective JAK/STAT signaling in myelofibrosis patients. Treatment with ActRIIA/B-Fc has the potential to mitigate the dose limiting effects of ruxolitinib and enhance duration of therapy in myelofibrosis patients. Data are represented as mean±SEM. All groups N=10. One-way ANOVA followed by Tukey post hoc test was used for analysis. ns—not significant; * p≤0.05; ** p≤0.01; *** p≤0.001; **** p≤0.0001.

### Example 9—Megakaryocyte Precursor Cells Express Activins, GDFs, BMPs and TGF-β Ligands and their Cognate Receptors

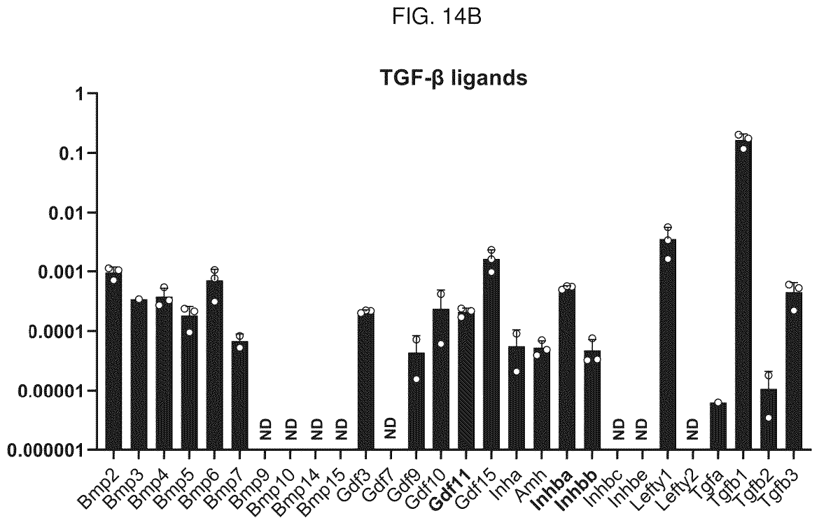

Bone marrow from three untreated mice was pooled and selected for megakaryocyte marker CD41. Once cells underwent positive selection using Rat anti-mouse CD41 (clone-MWReg30), RNA was extracted via Zymo Research Direct-zol RNA MicroPrep Kit. Total RNA was then converted to cDNA (QuantiTect Reverse Transcription Kit) and at least 20 ng per well was added to a mouse specific TGF-β family pathway TaqMan gene expression array (ThermoFisher Custom Gene Array). Quantitative real time PCR was performed, and results were plotted using 2\hat{ }delta Ct values. Housekeeping genes including 18s, Gusb, Gapdh, Actb, and Ubc were used to normalize TGF-β gene expression. The results are shown in FIGS. 14A-14B and represent 3 independent experimental repeats. As shown in FIGS. 14A-14B, murine bone marrow megakaryocyte precursors expressed activins, GDFs, BMPs and TGF-β ligands and their cognate receptors, including activin receptors IIA and IIB. These data demonstrate the capability of the TGF-β pathway to be involved in the differentiation of megakaryocytes and in normal megakaryocyte function. The gene that encodes the activin A protein, INHBA, was moderately expressed compared to other family member ligands. Receptors and ligands directly related to ActRIIA/B-mFc are in bold. ND, not detected.

### Example 10—Treatment of Anemia in a Subject Receiving Ruxolitinib for Myelofibrosis by Co-Administration of an ActRIIA Ligand Trap Containing an Extracellular ActRIIA Variant

According to the methods disclosed herein, a physician of skill in the art can treat a subject, such as a human patient, receiving ruxolitinib for the treatment of myelofibrosis (e.g., PMF, post-ET MF, and post-PV MF) and having anemia so as to increase red blood cell count, increase hemoglobin levels, increase hematocrit, decrease RBC transfusions, promote transfusion independence, and/or treat anemia. The method of treatment can include diagnosing or identifying a subject as a candidate for treatment by measuring hemoglobin levels. To treat the subject, a physician of skill in the art can administer to the subject a composition containing an ActRIIA ligand trap containing an extracellular ActRIIA variant (e.g., an extracellular ActRIIA variant having the sequence of any one of SEQ ID NOs: 1-72 (e.g., SEQ ID NOs: 6-72)). The composition containing the ActRIIA ligand trap containing an extracellular ActRIIA variant may be administered to the subject, for example, by parenteral injection (e.g., intravenous or subcutaneous injection) in combination with ruxolitinib, which is administered orally one to two times per day. The ActRIIA ligand trap containing an extracellular ActRIIA variant (e.g., an extracellular ActRIIA variant having the sequence of any one of SEQ ID NOs: 1-72 (e.g., SEQ ID NOs: 6-72)) is administered in a therapeutically effective amount, such as from 0.01 to 500 mg/kg (e.g., 0.01, 0.1, 0.2, 0.3, 0.325, 0.35, 0.375, 0.4, 0.5, 0.75, 1, 1.25, 1.5, 1.75, 2, 2.25, 2.5, 2.75, 3, 3.25, 3.5, 3.75, 4, 4.25, 4.5, 4.75, 5, 10, 15, 20, 25, 30, 35, 40, 45, 50, 100, 150, 200, 250, 300, 350, 400, 450, or 500 mg/kg). In some embodiments, the extracellular ActRIIA variant is administered bimonthly, once a month, once every four weeks, once every two weeks, or at least once a week or more (e.g., 1, 2, 3, 4, 5, 6, or 7 times a week or more). The ActRIIA ligand trap containing an extracellular ActRIIA variant is administered in an amount sufficient to increase red blood cell count, increase hemoglobin levels, increase hematocrit, decrease RBC transfusions, promote transfusion independence, and/or treat anemia.

Following administration of the composition to a patient, a practitioner of skill in the art can monitor the patient's improvement in response to the therapy by a variety of methods. For example, a physician can monitor the patient's red blood cell count, hemoglobin levels, or hematocrit using a blood test. A finding that the patient's red blood cell count, hemoglobin levels, or hematocrit are increased following administration of the composition compared to test results prior to administration of the composition indicates that the patient is responding favorably to the treatment. Subsequent doses can be determined and administered as needed.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims. Other embodiments are within the claims.

1. A method of treating a subject having myelofibrosis, the method comprising the step of administering in combination to the subject an effective amount of a cytopenia-associated myelofibrosis treatment and an ActRII signaling inhibitor.

2. The method of claim 1, wherein the cytopenia-associated myelofibrosis treatment and the ActRII signaling inhibitor are administered in combination after the subject has been identified as having a cytopenia.

3. The method of claim 1, wherein the cytopenia-associated myelofibrosis treatment and the ActRII signaling inhibitor are administered in combination before the subject develops a cytopenia.

4. A method of treating a subject with a myelofibrosis that has been identified as having a cytopenia, the method comprising the step of administering in combination to the subject an effective amount of a cytopenia-associated myelofibrosis treatment and an ActRII signaling inhibitor.

5. A method of treating a cytopenia in a subject diagnosed as having myelofibrosis, the method comprising the step of administering in combination to the subject an effective amount of a cytopenia-associated myelofibrosis treatment and an ActRII signaling inhibitor.

6. A method of treating a subject having polycythemia vera, the method comprising the step of administering in combination to the subject an effective amount of a cytopenia-associated myelofibrosis treatment and an ActRII signaling inhibitor.

7. A method of treating a subject having steroid-refractory graft-versus-host disease, the method comprising the step of administering in combination to the subject an effective amount of a cytopenia-associated myelofibrosis treatment and an ActRII signaling inhibitor.

8. The method of claim 6 or 7, wherein the cytopenia-associated myelofibrosis treatment and the ActRII signaling inhibitor are administered in combination after the subject has been identified as having a cytopenia.

9. The method of claim 6 or 7, wherein the cytopenia-associated myelofibrosis treatment and the ActRII signaling inhibitor are administered in combination before the subject develops a cytopenia.

10. A method of treating a cytopenia in a subject diagnosed as having polycythemia vera, the method comprising the step of administering in combination to the subject an effective amount of a cytopenia-associated myelofibrosis treatment and an ActRII signaling inhibitor.

11. A method of treating a cytopenia in a subject diagnosed as having steroid-refractory graft-versus-host disease, the method comprising the step of administering in combination to the subject an effective amount of a cytopenia-associated myelofibrosis treatment and an ActRII signaling inhibitor.

12. A method of treating a subject receiving treatment with a cytopenia-associated myelofibrosis treatment, the method comprising the step of administering in combination to the subject an effective amount of a cytopenia-associated myelofibrosis treatment and an ActRII signaling inhibitor.

13. A method of improving adherence to treatment with a cytopenia-associated myelofibrosis treatment in a subject in need thereof, the method comprising the step of administering in combination the cytopenia-associated myelofibrosis treatment and an ActRII signaling inhibitor.

14. A method of increasing the dose of a cytopenia-associated myelofibrosis treatment administered to a subject in need thereof, the method comprising the step of administering in combination the cytopenia-associated myelofibrosis treatment and an ActRII signaling inhibitor.

15. A method of increasing treatment duration for a cytopenia-associated myelofibrosis treatment in a subject in need thereof, the method comprising the step of administering in combination the cytopenia-associated myelofibrosis treatment and an ActRII signaling inhibitor.

16. A method of maintaining dose intensity for a cytopenia-associated myelofibrosis treatment in a subject in need thereof, the method comprising the step of administering in combination the cytopenia-associated myelofibrosis treatment and an ActRII signaling inhibitor.

17. A method of decreasing episodes of cytopenia associated with a cytopenia-associated myelofibrosis treatment in a subject in need thereof, the method comprising the step of administering in combination the cytopenia-associated myelofibrosis treatment and an ActRII signaling inhibitor.

18. A method of decreasing transfusion burden in a subject treated with a cytopenia-associated myelofibrosis treatment, the method comprising the step of administering in combination the cytopenia-associated myelofibrosis treatment and an ActRII signaling inhibitor.

19. A method of decreasing bleeding events in a subject treated with a cytopenia-associated myelofibrosis treatment, the method comprising the step of administering in combination the cytopenia-associated myelofibrosis treatment and an ActRII signaling inhibitor.

20. A method of decreasing infections in a subject treated with a cytopenia-associated myelofibrosis treatment, the method comprising the step of administering in combination the cytopenia-associated myelofibrosis treatment and an ActRII signaling inhibitor.

21. A method of decreasing treatment interruptions or discontinuations for a cytopenia-associated myelofibrosis treatment in a subject in need thereof, the method comprising the step of administering in combination the cytopenia-associated myelofibrosis treatment and an ActRII signaling inhibitor.

22. A method of resuming treatment with a cytopenia-associated myelofibrosis treatment in a subject who developed a myelofibrosis treatment-associated cytopenia, the method comprising the step of administering in combination the cytopenia-associated myelofibrosis treatment and an ActRII signaling inhibitor.

23. The method of any one of claims 1-5 and 12-22, wherein the subject has medium- or high-risk myelofibrosis.

24. The method of any one of claims 1-5 and 12-23, wherein the subject has primary myelofibrosis (PMF), post-essential thrombocythemia myelofibrosis (post-ET MF), or post-polycythemia vera myelofibrosis (post-PV MF).

25. The method of any one of claims 6, 8-10, and 12-23, wherein the subject has polycythemia vera.

26. The method of any one of claims 7-9 and 11-23, wherein the subject has steroid-refractory graft-versus-host disease.

27. The method of any one of claims 1-26, wherein the cytopenia-associated myelofibrosis treatment is a JAK inhibitor or Imetelstat.

28. The method of claim 27, wherein the JAK inhibitor is Ruxolitinib, Fedratinib, or Pacritinib.

29. The method of any one of claims 1, 6, 7, and 12-28, wherein the subject has a cytopenia.

30. The method of any one of claims 1, 6, 7, and 12-29, wherein the subject is identified as having a cytopenia prior to the administering of the ActRII signaling inhibitor.

31. The method of any one of claims 1, 6, 7, and 12-29, wherein the method further comprises identifying the subject as having a cytopenia prior to the administering of the ActRII signaling inhibitor.

32. The method of any one of claims 2-5, 8-11, 17, and 29-31, wherein the cytopenia is anemia.

33. The method of any one of claims 2-5, 8-11, 17, and 29-32, wherein the cytopenia is thrombocytopenia.

34. The method of any one of claims 2-5, 8-11, 17, and 29-33, wherein the cytopenia is neutropenia.

35. The method of any one of claims 1-34, wherein the ActRII signaling inhibitor is an activin A antibody or an antigen binding fragment thereof.

36. The method of claim 35, wherein the activin A antibody is garetosmab.

37. The method of any one of claims 1-34, wherein the ActRII signaling inhibitor is a myostatin antibody or an antigen binding fragment thereof.

38. The method of claim 37, wherein the myostatin antibody is domagrozumab, landogrozumab, trevogrumab, or SRK-015.

39. The method of any one of claims 1-34, wherein the ActRII signaling inhibitor is an ActRII antibody or an antigen binding fragment thereof.

40. The method of claim 39, wherein the ActRII antibody is bimagrumab, CSJ089, CQ1876, or CDD861.

41. The method of any one of claims 1-34, wherein the ActRII signaling inhibitor is an ActRII ligand trap.

42. The method of claim 41, wherein the ActRII ligand trap is an ActRIIA ligand trap.

43. The method of claim 42, wherein the ActRIIA ligand trap is a composition of Table 18.

44. The method of claim 42, wherein the ActRIIA ligand trap is sotatercept.

45. The method of claim 41, wherein the ActRII ligand trap is an ActRIIB ligand trap.

46. The method of claim 45, wherein the ActRIIB ligand trap is BIIB110, ALG-802, luspatercept, ramatercept, or ACE-2494.

47. The method of claim 45, wherein the ActRIIB ligand trap is a composition of Table 19.

48. The method of claim 41, wherein the ActRII ligand trap is an ActRII chimera ligand trap.

49. The method of claim 48, wherein the ActRII chimera ligand trap is a composition of Table 20 or Table 21.

50. The method of any one of claims 1-34, wherein the ActRII signaling inhibitor is an activin B antibody or an antigen binding fragment thereof.

51. The method of any one of claims 1-34, wherein the ActRII signaling inhibitor is a GDF-11 antibody or an antigen binding fragment thereof.

52. The method of any one of claims 1-51, wherein the method further includes evaluating red cell or platelet parameters after the administering of the ActRII signaling inhibitor.


SEQUENCE LISTING (828 sequences)

SEQ ID NO: 1 - protein, 109 aa, synthetic construct
  REGION 1..109: note = Synthetic Construct
  VARIANT 14: note = Xaa is Phe or Tyr
  VARIANT 15: note = Xaa is Phe or Tyr
  VARIANT 20: note = Xaa is Glu or Ala
  VARIANT 21: note = Xaa is Lys or Leu
  VARIANT 22: note = Xaa is Asp or Glu
  VARIANT 23: note = Xaa is Arg or Ala
  VARIANT 31: note = Xaa is Pro or Arg
  VARIANT 33: note = Xaa is Tyr or Glu
  VARIANT 35: note = Xaa is Asp or Glu
  VARIANT 36: note = Xaa is Lys or Gln
  VARIANT 37: note = Xaa is Asp or Ala
  VARIANT 38: note = Xaa is Lys or Ala
  VARIANT 39: note = Xaa is Arg or Ala
  VARIANT 40: note = Xaa is Arg or Leu
  VARIANT 43: note = Xaa is Phe or Tyr
  VARIANT 47: note = Xaa is Lys, Arg, or Ala
  VARIANT 57: note = Xaa is Lys, Ala, Tyr, Phe, or Ile
  VARIANT 58: note = Xaa is Gln or Lys
  VARIANT 61: note = Xaa is Trp or Ala
  VARIANT 62: note = Xaa is Leu or Ala
  VARIANT 63: note = Xaa is Asp, Lys, Arg, Ala, Phe, Gly, Met, Asn, or Ile
  VARIANT 65: note = Xaa is Ile, Phe, or Ala
  VARIANT 76: note = Xaa is Lys or Thr
  VARIANT 77: note = Xaa is Lys or Glu
  VARIANT 78: note = Xaa is Asp or Glu
  VARIANT 79: note = Xaa is Ser or Asn
  VARIANT 81: note = Xaa is Glu or Gln
GAILGRSETQECLXXNANWXXXXTNQTGVEXCXGXXXXXXHCXATWXNISGSIEIVXXGCXXXDXNCYDRTDCVEXXXXPXVYFCCCEGNMCNEKFSYFPEMEVTQPTS

SEQ ID NO: 2 - protein, 109 aa, synthetic construct
  REGION 1..109: note = Synthetic Construct
  VARIANT 15: note = Xaa is Phe or Tyr
  VARIANT 20: note = Xaa is Glu or Ala
  VARIANT 21: note = Xaa is Lys or Leu
  VARIANT 22: note = Xaa is Asp or Glu
  VARIANT 23: note = Xaa is Arg or Ala
  VARIANT 31: note = Xaa is Pro or Arg
  VARIANT 33: note = Xaa is Tyr or Glu
  VARIANT 35: note = Xaa is Asp or Glu
  VARIANT 37: note = Xaa is Asp or Ala
  VARIANT 38: note = Xaa is Lys or Ala
  VARIANT 39: note = Xaa is Arg or Ala
  VARIANT 40: note = Xaa is Arg or Leu
  VARIANT 43: note = Xaa is Phe or Tyr
  VARIANT 47: note = Xaa is Lys, Arg, or Ala
  VARIANT 57: note = Xaa is Lys, Ala, Tyr, Phe, or Ile
  VARIANT 58: note = Xaa is Gln or Lys
  VARIANT 61: note = Xaa is Trp or Ala
  VARIANT 62: note = Xaa is Leu or Ala
  VARIANT 63: note = Xaa is Asp, Lys, Arg, Ala, Phe, Gly, Met, Asn, or Ile
  VARIANT 65: note = Xaa is Ile, Phe, or Ala
  VARIANT 76: note = Xaa is Lys or Thr
  VARIANT 77: note = Xaa is Lys or Glu
  VARIANT 78: note = Xaa is Asp or Glu
  VARIANT 79: note = Xaa is Ser or Asn
  VARIANT 81: note = Xaa is Glu or Gln
GAILGRSETQECLFXNANWXXXXTNQTGVEXCXGXKXXXXHCXATWXNISGSIEIVXXGCXXXDXNCYDRTDCVEXXXXPXVYFCCCEGNMCNEKFSYFPEMEVTQPTS

SEQ ID NO: 3 - protein, 109 aa, synthetic construct
  REGION 1..109: note = Synthetic Construct
  VARIANT 15: note = Xaa is Phe or Tyr
  VARIANT 21: note = Xaa is Lys or Leu
  VARIANT 22: note = Xaa is Asp or Glu
  VARIANT 31: note = Xaa is Pro or Arg
  VARIANT 33: note = Xaa is Tyr or Glu
  VARIANT 35: note = Xaa is Asp or Glu
  VARIANT 40: note = Xaa is Arg or Leu
  VARIANT 43: note = Xaa is Phe or Tyr
  VARIANT 47: note = Xaa is Lys, Arg, or Ala
  VARIANT 58: note = Xaa is Gln or Lys
  VARIANT 65: note = Xaa is Ile, Phe, or Ala
  VARIANT 76: note = Xaa is Lys or Thr
  VARIANT 77: note = Xaa is Lys or Glu
  VARIANT 78: note = Xaa is Asp or Glu
  VARIANT 79: note = Xaa is Ser or Asn
  VARIANT 81: note = Xaa is Glu or Gln
GAILGRSETQECLFXNANWEXXRTNQTGVEXCXGXKDKRXHCXATWXNISGSIEIVKXGCWLDDXNCYDRTDCVEXXXXPXVYFCCCEGNMCNEKFSYFPEMEVTQPTS

SEQ ID NO: 4 - protein, 109 aa, synthetic construct
  REGION 1..109: note = Synthetic Construct
  VARIANT 15: note = Xaa is Phe or Tyr
  VARIANT 21: note = Xaa is Lys or Leu
  VARIANT 31: note = Xaa is Pro or Arg
  VARIANT 33: note = Xaa is Tyr or Glu
  VARIANT 35: note = Xaa is Asp or Glu
  VARIANT 40: note = Xaa is Arg or Leu
  VARIANT 43: note = Xaa is Phe or Tyr
  VARIANT 47: note = Xaa is Lys, Arg, or Ala
  VARIANT 58: note = Xaa is Gln or Lys
  VARIANT 65: note = Xaa is Ile, Phe, or Ala
  VARIANT 76: note = Xaa is Lys or Thr
  VARIANT 78: note = Xaa is Asp or Glu
  VARIANT 79: note = Xaa is Ser or Asn
  VARIANT 81: note = Xaa is Glu or Gln
GAILGRSETQECLFXNANWEXDRTNQTGVEXCXGXKDKRXHCXATWXNISGSIEIVKXGCWLDDXNCYDRTDCVEXKXXPXVYFCCCEGNMCNEKFSYFPEMEVTQPTS

SEQ ID NO: 5 - protein, 109 aa, synthetic construct
  REGION 1..109: note = Synthetic Construct
  VARIANT 15: note = Xaa is Phe or Tyr
  VARIANT 21: note = Xaa is Lys or Leu
  VARIANT 33: note = Xaa is Tyr or Glu
  VARIANT 35: note = Xaa is Asp or Glu
  VARIANT 40: note = Xaa is Arg or Leu
  VARIANT 58: note = Xaa is Gln or Lys
  VARIANT 76: note = Xaa is Lys or Thr
  VARIANT 78: note = Xaa is Asp or Glu
  VARIANT 79: note = Xaa is Ser or Asn
  VARIANT 81: note = Xaa is Glu or Gln
GAILGRSETQECLFXNANWEXDRTNQTGVEPCXGXKDKRXHCFATWKNISGSIEIVKXGCWLDDINCYDRTDCVEXKXXPXVYFCCCEGNMCNEKFSYFPEMEVTQPTS

SEQ ID NO: 6 - protein, 109 aa, synthetic construct
  REGION 1..109: note = Synthetic Construct
GAILGRSETQECLFYNANWELDRTNQTGVEPCEGEKDKRLHCFATWKNISGSIEIVKKGCWLDDINCYDRTDCVETKENPQVYFCCCEGNMCNEKFSYFPEMEVTQPTS

SEQ ID NO: 7 - protein, 109 aa, synthetic construct
  REGION 1..109: note = Synthetic Construct
GAILGRSETQECLFYNANWELERTNQTGVEPCEGEKDKRLHCFATWKNISGSIEIVKKGCWLDDINCYDRTDCVETKENPQVYFCCCEGNMCNEKFSYFPEMEVTQPTS

SEQ ID NO: 8 - protein, 109 aa, synthetic construct
  REGION 1..109: note = Synthetic Construct
GAILGRSETQECLFYNANWELDRTNQTGVERCEGEKDKRLHCFATWKNISGSIEIVKKGCWLDDINCYDRTDCVETKENPQVYFCCCEGNMCNEKFSYFPEMEVTQPTS

SEQ ID NO: 9 - protein, 109 aa, synthetic construct
  REGION 1..109: note = Synthetic Construct
GAILGRSETQECLFYNANWELDRTNQTGVEPCEGEKDKRLHCYATWKNISGSIEIVKKGCWLDDINCYDRTDCVETKENPQVYFCCCEGNMCNEKFSYFPEMEVTQPTS

SEQ ID NO: 10 - protein, 109 aa, synthetic construct
  REGION 1..109: note = Synthetic Construct
GAILGRSETQECLFYNANWELDRTNQTGVEPCEGEKDKRLHCFATWRNISGSIEIVKKGCWLDDINCYDRTDCVETKENPQVYFCCCEGNMCNEKFSYFPEMEVTQPTS

SEQ ID NO: 11 - protein, 109 aa, synthetic construct
  REGION 1..109: note = Synthetic Construct
GAILGRSETQECLFYNANWELDRTNQTGVEPCEGEKDKRLHCFATWKNISGSIEIVKKGCWLDDFNCYDRTDCVETKENPQVYFCCCEGNMCNEKFSYFPEMEVTQPTS

SEQ ID NO: 12 - protein, 109 aa, synthetic construct
  REGION 1..109: note = Synthetic Construct
GAILGRSETQECLFYNANWELDRTNQTGVEPCEGEKDKRLHCFATWKNISGSIEIVKKGCWLDDINCYDRTDCVETEENPQVYFCCCEGNMCNEKFSYFPEMEVTQPTS

SEQ ID NO: 13 - protein, 109 aa, synthetic construct
  REGION 1..109: note = Synthetic Construct
GAILGRSETQECLFYNANWELERTNQTGVERCEGEKDKRLHCFATWKNISGSIEIVKKGCWLDDINCYDRTDCVETKENPQVYFCCCEGNMCNEKFSYFPEMEVTQPTS

SEQ ID NO: 14 - protein, 109 aa, synthetic construct
  REGION 1..109: note = Synthetic Construct
GAILGRSETQECLFYNANWELERTNQTGVEPCEGEKDKRLHCYATWKNISGSIEIVKKGCWLDDINCYDRTDCVETKENPQVYFCCCEGNMCNEKFSYFPEMEVTQPTS

SEQ ID NO: 15 - protein, 109 aa, synthetic construct
  REGION 1..109: note = Synthetic Construct
GAILGRSETQECLFYNANWELERTNQTGVEPCEGEKDKRLHCFATWRNISGSIEIVKKGCWLDDINCYDRTDCVETKENPQVYFCCCEGNMCNEKFSYFPEMEVTQPTS

SEQ ID NO: 16 - protein, 109 aa, synthetic construct
  REGION 1..109: note = Synthetic Construct
GAILGRSETQECLFYNANWELERTNQTGVEPCEGEKDKRLHCFATWKNISGSIEIVKKGCWLDDFNCYDRTDCVETKENPQVYFCCCEGNMCNEKFSYFPEMEVTQPTS

SEQ ID NO: 17 - protein, 109 aa, synthetic construct
  REGION 1..109: note = Synthetic Construct
GAILGRSETQECLFYNANWELERTNQTGVEPCEGEKDKRLHCFATWKNISGSIEIVKKGCWLDDINCYDRTDCVETEENPQVYFCCCEGNMCNEKFSYFPEMEVTQPTS

SEQ ID NO: 18 - protein, 109 aa, synthetic construct
  REGION 1..109: note = Synthetic Construct
GAILGRSETQECLFYNANWELDRTNQTGVERCEGEKDKRLHCYATWKNISGSIEIVKKGCWLDDINCYDRTDCVETKENPQVYFCCCEGNMCNEKFSYFPEMEVTQPTS

SEQ ID NO: 19 - protein, 109 aa, synthetic construct
  REGION 1..109: note = Synthetic Construct
GAILGRSETQECLFYNANWELDRTNQTGVERCEGEKDKRLHCFATWRNISGSIEIVKKGCWLDDINCYDRTDCVETKENPQVYFCCCEGNMCNEKFSYFPEMEVTQPTS

SEQ ID NO: 20 - protein, 109 aa, synthetic construct
  REGION 1..109: note = Synthetic Construct
GAILGRSETQECLFYNANWELDRTNQTGVERCEGEKDKRLHCFATWKNISGSIEIVKKGCWLDDFNCYDRTDCVETKENPQVYFCCCEGNMCNEKFSYFPEMEVTQPTS

SEQ ID NO: 21 - protein, 109 aa, synthetic construct
  REGION 1..109: note = Synthetic Construct
GAILGRSETQECLFYNANWELDRTNQTGVERCEGEKDKRLHCFATWKNISGSIEIVKKGCWLDDINCYDRTDCVETEENPQVYFCCCEGNMCNEKFSYFPEMEVTQPTS

SEQ ID NO: 22 - protein, 109 aa, synthetic construct
  REGION 1..109: note = Synthetic Construct
GAILGRSETQECLFYNANWELDRTNQTGVEPCEGEKDKRLHCYATWRNISGSIEIVKKGCWLDDINCYDRTDCVETKENPQVYFCCCEGNMCNEKFSYFPEMEVTQPTS

SEQ ID NO: 23 - protein, 109 aa, synthetic construct
  REGION 1..109: note = Synthetic Construct
GAILGRSETQECLFYNANWELDRTNQTGVEPCEGEKDKRLHCYATWKNISGSIEIVKKGCWLDDFNCYDRTDCVETKENPQVYFCCCEGNMCNEKFSYFPEMEVTQPTS

SEQ ID NO: 24 - protein, 109 aa, synthetic construct
  REGION 1..109: note = Synthetic Construct
GAILGRSETQECLFYNANWELDRTNQTGVEPCEGEKDKRLHCYATWKNISGSIEIVKKGCWLDDINCYDRTDCVETEENPQVYFCCCEGNMCNEKFSYFPEMEVTQPTS

SEQ ID NO: 25 - protein, 109 aa, synthetic construct
  REGION 1..109: note = Synthetic Construct
GAILGRSETQECLFYNANWELDRTNQTGVEPCEGEKDKRLHCFATWRNISGSIEIVKKGCWLDDFNCYDRTDCVETKENPQVYFCCCEGNMCNEKFSYFPEMEVTQPTS

SEQ ID NO: 26 - protein, 109 aa, synthetic construct
  REGION 1..109: note = Synthetic Construct
GAILGRSETQECLFYNANWELDRTNQTGVEPCEGEKDKRLHCFATWRNISGSIEIVKKGCWLDDINCYDRTDCVETEENPQVYFCCCEGNMCNEKFSYFPEMEVTQPTS

SEQ ID NO: 27 - protein, 109 aa, synthetic construct
  REGION 1..109: note = Synthetic Construct
GAILGRSETQECLFYNANWELDRTNQTGVEPCEGEKDKRLHCFATWKNISGSIEIVKKGCWLDDFNCYDRTDCVETEENPQVYFCCCEGNMCNEKFSYFPEMEVTQPTS

SEQ ID NO: 28 - protein, 109 aa, synthetic construct
  REGION 1..109: note = Synthetic Construct
GAILGRSETQECLFYNANWELERTNQTGVERCEGEKDKRLHCYATWKNISGSIEIVKKGCWLDDINCYDRTDCVETKENPQVYFCCCEGNMCNEKFSYFPEMEVTQPTS

SEQ ID NO: 29 - protein, 109 aa, synthetic construct
  REGION 1..109: note = Synthetic Construct
GAILGRSETQECLFYNANWELERTNQTGVERCEGEKDKRLHCFATWRNISGSIEIVKKGCWLDDINCYDRTDCVETKENPQVYFCCCEGNMCNEKFSYFPEMEVTQPTS

SEQ ID NO: 30 - protein, 109 aa, synthetic construct
  REGION 1..109: note = Synthetic Construct
GAILGRSETQECLFYNANWELERTNQTGVERCEGEKDKRLHCFATWKNISGSIEIVKKGCWLDDFNCYDRTDCVETKENPQVYFCCCEGNMCNEKFSYFPEMEVTQPTS

SEQ ID NO: 31 - protein, 109 aa, synthetic construct
  REGION 1..109: note = Synthetic Construct
GAILGRSETQECLFYNANWELERTNQTGVERCEGEKDKRLHCFATWKNISGSIEIVKKGCWLDDINCYDRTDCVETEENPQVYFCCCEGNMCNEKFSYFPEMEVTQPTS

SEQ ID NO: 32 - protein, 109 aa, synthetic construct
  REGION 1..109: note = Synthetic Construct
GAILGRSETQECLFYNANWELERTNQTGVEPCEGEKDKRLHCYATWRNISGSIEIVKKGCWLDDINCYDRTDCVETKENPQVYFCCCEGNMCNEKFSYFPEMEVTQPTS

SEQ ID NO: 33 - protein, 109 aa, synthetic construct
  REGION 1..109: note = Synthetic Construct
GAILGRSETQECLFYNANWELERTNQTGVEPCEGEKDKRLHCYATWKNISGSIEIVKKGCWLDDFNCYDRTDCVETKENPQVYFCCCEGNMCNEKFSYFPEMEVTQPTS

SEQ ID NO: 34 - protein, 109 aa, synthetic construct
  REGION 1..109: note = Synthetic Construct
GAILGRSETQECLFYNANWELERTNQTGVEPCEGEKDKRLHCYATWKNISGSIEIVKKGCWLDDINCYDRTDCVETEENPQVYFCCCEGNMCNEKFSYFPEMEVTQPTS

SEQ ID NO: 35 - protein, 109 aa, synthetic construct
  REGION 1..109: note = Synthetic Construct
GAILGRSETQECLFYNANWELERTNQTGVEPCEGEKDKRLHCFATWRNISGSIEIVKKGCWLDDFNCYDRTDCVETKENPQVYFCCCEGNMCNEKFSYFPEMEVTQPTS

SEQ ID NO: 36 - protein, 109 aa, synthetic construct
  REGION 1..109: note = Synthetic Construct
GAILGRSETQECLFYNANWELERTNQTGVEPCEGEKDKRLHCFATWRNISGSIEIVKKGCWLDDINCYDRTDCVETEENPQVYFCCCEGNMCNEKFSYFPEMEVTQPTS

SEQ ID NO: 37 - protein, 109 aa, synthetic construct
  REGION 1..109: note = Synthetic Construct
GAILGRSETQECLFYNANWELERTNQTGVEPCEGEKDKRLHCFATWKNISGSIEIVKKGCWLDDFNCYDRTDCVETEENPQVYFCCCEGNMCNEKFSYFPEMEVTQPTS

SEQ ID NO: 38 - protein, 109 aa, synthetic construct
  REGION 1..109: note = Synthetic Construct
GAILGRSETQECLFYNANWELDRTNQTGVERCEGEKDKRLHCYATWRNISGSIEIVKKGCWLDDINCYDRTDCVETKENPQVYFCCCEGNMCNEKFSYFPEMEVTQPTS

SEQ ID NO: 39 - protein, 109 aa, synthetic construct
  REGION 1..109: note = Synthetic Construct
GAILGRSETQECLFYNANWELDRTNQTGVERCEGEKDKRLHCYATWKNISGSIEIVKKGCWLDDFNCYDRTDCVETKENPQVYFCCCEGNMCNEKFSYFPEMEVTQPTS

SEQ ID NO: 40 - protein, 109 aa, synthetic construct
  REGION 1..109: note = Synthetic Construct
GAILGRSETQECLFYNANWELDRTNQTGVERCEGEKDKRLHCYATWKNISGSIEIVKKGCWLDDINCYDRTDCVETEENPQVYFCCCEGNMCNEKFSYFPEMEVTQPTS

SEQ ID NO: 41 - protein, 109 aa, synthetic construct
  REGION 1..109: note = Synthetic Construct
GAILGRSETQECLFYNANWELDRTNQTGVERCEGEKDKRLHCFATWRNISGSIEIVKKGCWLDDFNCYDRTDCVETKENPQVYFCCCEGNMCNEKFSYFPEMEVTQPTS

SEQ ID NO: 42 - protein, 109 aa, synthetic construct
  REGION 1..109: note = Synthetic Construct
GAILGRSETQECLFYNANWELDRTNQTGVERCEGEKDKRLHCFATWRNISGSIEIVKKGCWLDDINCYDRTDCVETEENPQVYFCCCEGNMCNEKFSYFPEMEVTQPTS

SEQ ID NO: 43 - protein, 109 aa, synthetic construct
  REGION 1..109: note = Synthetic Construct
GAILGRSETQECLFYNANWELDRTNQTGVERCEGEKDKRLHCFATWKNISGSIEIVKKGCWLDDFNCYDRTDCVETEENPQVYFCCCEGNMCNEKFSYFPEMEVTQPTS

SEQ ID NO: 44 - protein, 109 aa, synthetic construct
  REGION 1..109: note = Synthetic Construct
GAILGRSETQECLFYNANWELDRTNQTGVEPCEGEKDKRLHCYATWRNISGSIEIVKKGCWLDDFNCYDRTDCVETKENPQVYFCCCEGNMCNEKFSYFPEMEVTQPTS

SEQ ID NO: 45 - protein, 109 aa, synthetic construct
  REGION 1..109: note = Synthetic Construct
GAILGRSETQECLFYNANWELDRTNQTGVEPCEGEKDKRLHCYATWRNISGSIEIVKKGCWLDDINCYDRTDCVETEENPQVYFCCCEGNMCNEKFSYFPEMEVTQPTS

SEQ ID NO: 46 - protein, 109 aa, synthetic construct
  REGION 1..109: note = Synthetic Construct
GAILGRSETQECLFYNANWELDRTNQTGVEPCEGEKDKRLHCYATWKNISGSIEIVKKGCWLDDFNCYDRTDCVETEENPQVYFCCCEGNMCNEKFSYFPEMEVTQPTS

SEQ ID NO: 47 - protein, 109 aa, synthetic construct
  REGION 1..109: note = Synthetic Construct
GAILGRSETQECLFYNANWELDRTNQTGVEPCEGEKDKRLHCFATWRNISGSIEIVKKGCWLDDFNCYDRTDCVETEENPQVYFCCCEGNMCNEKFSYFPEMEVTQPTS

SEQ ID NO: 48 - protein, 109 aa, synthetic construct
  REGION 1..109: note = Synthetic Construct
GAILGRSETQECLFYNANWELERTNQTGVERCEGEKDKRLHCYATWRNISGSIEIVKKGCWLDDINCYDRTDCVETKENPQVYFCCCEGNMCNEKFSYFPEMEVTQPTS

SEQ ID NO: 49 - protein, 109 aa, synthetic construct
  REGION 1..109: note = Synthetic Construct
GAILGRSETQECLFYNANWELERTNQTGVERCEGEKDKRLHCYATWKNISGSIEIVKKGCWLDDFNCYDRTDCVETKENPQVYFCCCEGNMCNEKFSYFPEMEVTQPTS

SEQ ID NO: 50 - protein, 109 aa, synthetic construct
  REGION 1..109: note = Synthetic Construct
GAILGRSETQECLFYNANWELERTNQTGVERCEGEKDKRLHCYATWKNISGSIEIVKKGCWLDDINCYDRTDCVETEENPQVYFCCCEGNMCNEKFSYFPEMEVTQPTS

SEQ ID NO: 51 - protein, 109 aa, synthetic construct
  REGION 1..109: note = Synthetic Construct
GAILGRSETQECLFYNANWELERTNQTGVERCEGEKDKRLHCFATWRNISGSIEIVKKGCWLDDFNCYDRTDCVETKENPQVYFCCCEGNMCNEKFSYFPEMEVTQPTS

SEQ ID NO: 52 - protein, 109 aa, synthetic construct
  REGION 1..109: note = Synthetic Construct
GAILGRSETQECLFYNANWELERTNQTGVERCEGEKDKRLHCFATWRNISGSIEIVKKGCWLDDINCYDRTDCVETEENPQVYFCCCEGNMCNEKFSYFPEMEVTQPTS

SEQ ID NO: 53 - protein, 109 aa, synthetic construct
  REGION 1..109: note = Synthetic Construct
GAILGRSETQECLFYNANWELERTNQTGVERCEGEKDKRLHCFATWKNISGSIEIVKKGCWLDDFNCYDRTDCVETEENPQVYFCCCEGNMCNEKFSYFPEMEVTQPTS

SEQ ID NO: 54 - protein, 109 aa, synthetic construct
  REGION 1..109: note = Synthetic Construct
GAILGRSETQECLFYNANWELERTNQTGVEPCEGEKDKRLHCYATWRNISGSIEIVKKGCWLDDFNCYDRTDCVETKENPQVYFCCCEGNMCNEKFSYFPEMEVTQPTS

SEQ ID NO: 55 - protein, 109 aa, synthetic construct
  REGION 1..109: note = Synthetic Construct
GAILGRSETQECLFYNANWELERTNQTGVEPCEGEKDKRLHCYATWRNISGSIEIVKKGCWLDDINCYDRTDCVETEENPQVYFCCCEGNMCNEKFSYFPEMEVTQPTS

SEQ ID NO: 56 - protein, 109 aa, synthetic construct
  REGION 1..109: note = Synthetic Construct
GAILGRSETQECLFYNANWELERTNQTGVEPCEGEKDKRLHCYATWKNISGSIEIVKKGCWLDDFNCYDRTDCVETEENPQVYFCCCEGNMCNEKFSYFPEMEVTQPTS

SEQ ID NO: 57 - protein, 109 aa, synthetic construct
  REGION 1..109: note = Synthetic Construct
GAILGRSETQECLFYNANWELERTNQTGVEPCEGEKDKRLHCFATWRNISGSIEIVKKGCWLDDFNCYDRTDCVETEENPQVYFCCCEGNMCNEKFSYFPEMEVTQPTS

SEQ ID NO: 58 - protein, 109 aa, synthetic construct
  REGION 1..109: note = Synthetic Construct
GAILGRSETQECLFYNANWELDRTNQTGVERCEGEKDKRLHCYATWRNISGSIEIVKKGCWLDDFNCYDRTDCVETKENPQVYFCCCEGNMCNEKFSYFPEMEVTQPTS

SEQ ID NO: 59 - protein, 109 aa, synthetic construct
  REGION 1..109: note = Synthetic Construct
GAILGRSETQECLFYNANWELDRTNQTGVERCEGEKDKRLHCYATWRNISGSIEIVKKGCWLDDINCYDRTDCVETEENPQVYFCCCEGNMCNEKFSYFPEMEVTQPTS

SEQ ID NO: 60 - protein, 109 aa, synthetic construct
  REGION 1..109: note = Synthetic Construct
GAILGRSETQECLFYNANWELDRTNQTGVERCEGEKDKRLHCYATWKNISGSIEIVKKGCWLDDFNCYDRTDCVETEENPQVYFCCCEGNMCNEKFSYFPEMEVTQPTS

SEQ ID NO: 61 - protein, 109 aa, synthetic construct
  REGION 1..109: note = Synthetic Construct
GAILGRSETQECLFYNANWELDRTNQTGVERCEGEKDKRLHCFATWRNISGSIEIVKKGCWLDDFNCYDRTDCVETEENPQVYFCCCEGNMCNEKFSYFPEMEVTQPTS

SEQ ID NO: 62 - protein, 109 aa, synthetic construct
  REGION 1..109: note = Synthetic Construct
GAILGRSETQECLFYNANWELDRTNQTGVEPCEGEKDKRLHCYATWRNISGSIEIVKKGCWLDDFNCYDRTDCVETEENPQVYFCCCEGNMCNEKFSYFPEMEVTQPTS

SEQ ID NO: 63 - protein, 109 aa, synthetic construct
  REGION 1..109: note = Synthetic Construct
GAILGRSETQECLFYNANWELERTNQTGVERCEGEKDKRLHCYATWRNISGSIEIVKKGCWLDDFNCYDRTDCVETKENPQVYFCCCEGNMCNEKFSYFPEMEVTQPTS

SEQ ID NO: 64 - protein, 109 aa, synthetic construct
  REGION 1..109: note = Synthetic Construct
GAILGRSETQECLFYNANWELERTNQTGVERCEGEKDKRLHCYATWRNISGSIEIVKKGCWLDDINCYDRTDCVETEENPQVYFCCCEGNMCNEKFSYFPEMEVTQPTS

SEQ ID NO: 65 - protein, 109 aa, synthetic construct
  REGION 1..109: note = Synthetic Construct
GAILGRSETQECLFYNANWELERTNQTGVERCEGEKDKRLHCYATWKNISGSIEIVKKGCWLDDFNCYDRTDCVETEENPQVYFCCCEGNMCNEKFSYFPEMEVTQPTS

SEQ ID NO: 66 - protein, 109 aa, synthetic construct
  REGION 1..109: note = Synthetic Construct
GAILGRSETQECLFYNANWELERTNQTGVERCEGEKDKRLHCFATWRNISGSIEIVKKGCWLDDFNCYDRTDCVETEENPQVYFCCCEGNMCNEKFSYFPEMEVTQPTS

SEQ ID NO: 67 - protein, 109 aa, synthetic construct
  REGION 1..109: note = Synthetic Construct
GAILGRSETQECLFYNANWELDRTNQTGVERCEGEKDKRLHCYATWRNISGSIEIVKKGCWLDDFNCYDRTDCVETEENPQVYFCCCEGNMCNEKFSYFPEMEVTQPTS

SEQ ID NO: 68 - protein, 109 aa, synthetic construct
  REGION 1..109: note = Synthetic Construct
GAILGRSETQECLFYNANWELERTNQTGVEPCEGEKDKRLHCYATWRNISGSIEIVKKGCWLDDFNCYDRTDCVETEENPQVYFCCCEGNMCNEKFSYFPEMEVTQPTS

SEQ ID NO: 69 - protein, 109 aa, synthetic construct
  REGION 1..109: note = Synthetic Construct
GAILGRSETQECLFYNANWELERTNQTGVERCEGEKDKRLHCYATWRNISGSIEIVKKGCWLDDFNCYDRTDCVETEENPQVYFCCCEGNMCNEKFSYFPEMEVTQPTS

SEQ ID NO: 70 - protein, 109 aa, synthetic construct
  REGION 1..109: note = Synthetic Construct
GAILGRSETQECLYYNANWELERTNQTGVERCEGEQDKRLHCYATWRNISGSIEIVKKGCWLDDFNCYDRTDCVETEENPQVYFCCCEGNMCNEKFSYFPEMEVTQPTS

SEQ ID NO: 71 - protein, 111 aa, synthetic construct
  REGION 1..111: note = Synthetic Construct
GAILGRSETQECLFYNANWELERTNQTGVERCEGEKDKRLHCYATWRNISGSIEIVKKGCWLDDFNCYDRTDCVETEENPQVYFCCCEGNMCNEKFSYFPEMEVTQPTSNP

SEQ ID NO: 72 - protein, 115 aa, synthetic construct
  REGION 1..115: note = Synthetic Construct
GAILGRSETQECLFYNANWELERTNQTGVERCEGEKDKRLHCYATWRNISGSIEIVKKGCWLDDFNCYDRTDCVETEENPQVYFCCCEGNMCNEKFSYFPEMEVTQPTSNPVTPK

SEQ ID NO: 73 - protein, 109 aa, Homo sapiens
GAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTS

SEQ ID NO: 74 - protein, 115 aa, Homo sapiens
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 75 - protein, 513 aa, Homo sapiens
MGAAAKLAFAVFLISCSSGAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPYYNILLYSLVPLMLIAGIVICAFWVYRHHKMAYPPVLVPTQDPGPPPPSPLLGLKPLQLLEVKARGRFGCVWKAQLLNEYVAVKIFPIQDKQSWQNEYEVYSLPGMKHENILQFIGAEKRGTSVDVDLWLITAFHEKGSLSDFLKANVVSWNELCHIAETMARGLAYLHEDIPGLKDGHKPAISHRDIKSKNVLLKNNLTACIADFGLALKFEAGKSAGDTHGQVGTRRYMAPEVLEGAINFQRDAFLRIDMYAMGLVLWELASRCTAADGPVDEYMLPFEEEIGQHPSLEDMQEVVVHKKKRPVLRDYWQKHAGMAMLCETIEECWDHDAEARLSAGCVGERITQMQRLTNIITTEDIVTVVTMVTNVDFPPKESSL

SEQ ID NO: 76 - protein, 4 aa, Homo sapiens
TEEN

SEQ ID NO: 77 - protein, 4 aa, synthetic construct
  REGION 1..4: note = Synthetic Construct
TKEN

SEQ ID NO: 78 - protein, 6 aa, Homo sapiens
NPVTPK

SEQ ID NO: 79 - protein, 226 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 80 - protein, 338 aa, synthetic construct
  REGION 1..338: note = Synthetic Construct
GAILGRSETQECLFYNANWELERTNQTGVERCEGEKDKRLHCYATWRNISGSIEIVKKGCWLDDFNCYDRTDCVETEENPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 81 - protein, 609 aa, Homo sapiens [sequence omitted]

SEQ ID NO: 82 - protein, 93 aa, Homo sapiens
GPVEVFITETPSQPNSHPIQWNAPQPSHISKYILRWRPKNSVGRWKEATIPGHLNSYTIKGLKPGVVYEGQLISIQQYGHQEVTRFDFTTTST

SEQ ID NO: 83 - protein, 11 aa, synthetic construct
  REGION 1..11: note = Synthetic Construct
DICLPRWGCLW

SEQ ID NO: 84 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 109 aa, synthetic construct
  REGION 1..109: note = Synthetic Construct
GAILGRSETQECLFYNANWELERTNQTGVERCEGEKDKRLHCYATWRNISGSIEIVAKGCWLDDFNCYDRTDCVETEENPQVYFCCCEGNMCNEKFSYFPEMEVTQPTS

SEQ ID NO: 86 - protein, 5 aa, synthetic construct
  REGION 1..5: note = Synthetic Construct
PAPAP

SEQ ID NO: 87 - protein, 5 aa, synthetic construct
  REGION 1..5: note = Synthetic Construct
EAAAK

SEQ ID NO: 88 - protein, 36 aa, synthetic construct
  REGION 1..36: note = Synthetic Construct
GGSGGGSEGGGSEGGGSEGGGSEGGGSEGGGSGGGS

SEQ ID NO: 89 - protein, 17 aa, synthetic construct
  REGION 1..17: note = Synthetic Construct
AAANSSIDLISVPVDSR

SEQ ID NO: 90 - protein, 36 aa, synthetic construct
  REGION 1..36: note = Synthetic Construct
GGSAGGSGSGSSGGSSGASGTGTAGGTGSGSGTGSG

SEQ ID NO: 91 - protein, 17 aa, synthetic construct
  REGION 1..17: note = Synthetic Construct
RPACKIPNDLKQKVMNH

SEQ ID NO: 92 - protein, 5 aa, synthetic construct
  REGION 1..5: note = Synthetic Construct
RSIAT

SEQ ID NO: 93 - protein, 8 aa, synthetic construct
  REGION 1..8: note = Synthetic Construct
SACYCELS

SEQ ID NO: 94 - protein, 10 aa, synthetic construct
  REGION 1..10: note = Synthetic Construct
GENLYFQSGG

SEQ ID NO: 95 - protein, 18 aa, synthetic construct
  REGION 1..18: note = Synthetic Construct
KESGSVSSEQLAQFRSLD

SEQ ID NO: 96 - protein, 12 aa, synthetic construct
  REGION 1..12: note = Synthetic Construct
AEAAAKEAAAKA

SEQ ID NO: 97 - protein, 225 aa, Homo sapiens
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGPFFLYSKLTVDKSRWQQGNVFSCSVMHEALHNHYTQKSLSLSPGK

SEQ ID NO: 98 - protein, 4 aa, synthetic construct
  REGION 1..4: note = Synthetic Construct
GGGA

SEQ ID NO: 99 - protein, 4 aa, synthetic construct
  REGION 1..4: note = Synthetic Construct
GGGS

SEQ ID NO: 100 - protein, 4 aa, synthetic construct
  REGION 1..4: note = Synthetic Construct
GGGG

SEQ ID NO: 101 - protein, 5 aa, synthetic construct
  REGION 1..5: note = Synthetic Construct
GGGGA

SEQ ID NO: 102 - protein, 5 aa, synthetic construct
  REGION 1..5: note = Synthetic Construct
GGGGS

SEQ ID NO: 103 - protein, 5 aa, synthetic construct
  REGION 1..5: note = Synthetic Construct
GGGGG

SEQ ID NO: 104 - protein, 4 aa, synthetic construct
  REGION 1..4: note = Synthetic Construct
GGAG

SEQ ID NO: 105 - protein, 4 aa, synthetic construct
  REGION 1..4: note = Synthetic Construct
GGSG

SEQ ID NO: 106 - protein, 4 aa, synthetic construct
  REGION 1..4: note = Synthetic Construct
AGGG

SEQ ID NO: 107 - protein, 4 aa, synthetic construct
  REGION 1..4: note = Synthetic Construct
SGGG

SEQ ID NO: 108 - protein, 4 aa, synthetic construct
  REGION 1..4: note = Synthetic Construct
GAGA

SEQ ID NO: 109 - protein, 4 aa, synthetic construct
  REGION 1..4: note = Synthetic Construct
GSGS

SEQ ID NO: 110 - protein, 6 aa, synthetic construct
  REGION 1..6: note = Synthetic Construct
GAGAGA

SEQ ID NO: 111 - protein, 6 aa, synthetic construct
  REGION 1..6: note = Synthetic Construct
GSGSGS

SEQ ID NO: 112 - protein, 8 aa, synthetic construct
  REGION 1..8: note = Synthetic Construct
GAGAGAGA

SEQ ID NO: 113 - protein, 8 aa, synthetic construct
  REGION 1..8: note = Synthetic Construct
GSGSGSGS

SEQ ID NO: 114 - protein, 10 aa, synthetic construct
  REGION 1..10: note = Synthetic Construct
GAGAGAGAGA

SEQ ID NO: 115 - protein, 10 aa, synthetic construct
  REGION 1..10: note = Synthetic Construct
GSGSGSGSGS

SEQ ID NO: 116 - protein, 12 aa, synthetic construct
  REGION 1..12: note = Synthetic Construct
GAGAGAGAGAGA

SEQ ID NO: 117 - protein, 12 aa, synthetic construct
  REGION 1..12: note = Synthetic Construct
GSGSGSGSGSGS

SEQ ID NO: 118 - protein, 6 aa, synthetic construct
  REGION 1..6: note = Synthetic Construct
GGAGGA

SEQ ID NO: 119 - protein, 6 aa, synthetic construct
  REGION 1..6: note = Synthetic Construct
GGSGGS

SEQ ID NO: 120 - protein, 9 aa, synthetic construct
  REGION 1..9: note = Synthetic Construct
GGAGGAGGA

SEQ ID NO: 121 - protein, 9 aa, synthetic construct
  REGION 1..9: note = Synthetic Construct
GGSGGSGGS

SEQ ID NO: 122 - protein, 12 aa, synthetic construct
  REGION 1..12: note = Synthetic Construct
GGAGGAGGAGGA

SEQ ID NO: 123 - protein, 12 aa, synthetic construct
  REGION 1..12: note = Synthetic Construct
GGSGGSGGSGGS

SEQ ID NO: 124 - protein, 8 aa, synthetic construct
  REGION 1..8: note = Synthetic Construct
GGAGGGAG

SEQ ID NO: 125 - protein, 8 aa, synthetic construct
  REGION 1..8: note = Synthetic Construct
GGSGGGSG

SEQ ID NO: 126 - protein, 12 aa, synthetic construct
  REGION 1..12: note = Synthetic Construct
GGAGGGAGGGAG

SEQ ID NO: 127 - protein, 12 aa, synthetic construct
  REGION 1..12: note = Synthetic Construct
GGSGGGSGGGSG

SEQ ID NO: 128 - protein, 15 aa, synthetic construct
  REGION 1..15: note = Synthetic Construct
GGGGAGGGGAGGGGA

SEQ ID NO: 129 - protein, 15 aa, synthetic construct
  REGION 1..15: note = Synthetic Construct
GGGGSGGGGSGGGGS

SEQ ID NO: 130 - protein, 5 aa, synthetic construct
  REGION 1..5: note = Synthetic Construct
GGGAG

SEQ ID NO: 131 - protein, 6 aa, synthetic construct
  REGION 1..6: note = Synthetic Construct
GGGAGG

SEQ ID NO: 132 - protein, 7 aa, synthetic construct
  REGION 1..7: note = Synthetic Construct
GGGAGGG

SEQ ID NO: 133 - protein, 4 aa, synthetic construct
  REGION 1..4: note = Synthetic Construct
AAAL

SEQ ID NO: 134 - protein, 4 aa, synthetic construct
  REGION 1..4: note = Synthetic Construct
AAAK

SEQ ID NO: 135 - protein, 4 aa, synthetic construct
  REGION 1..4: note = Synthetic Construct
AAAR

SEQ ID NO: 136 - protein, 14 aa, synthetic construct
  REGION 1..14: note = Synthetic Construct
EGKSSGSGSESKST

SEQ ID NO: 137 - protein, 12 aa, synthetic construct
  REGION 1..12: note = Synthetic Construct
GSAGSAAGSGEF

SEQ ID NO: 138 - protein, 126 aa, synthetic construct
  REGION 1..126: note = Synthetic Construct
QVQLQESGPGLVKPSETLSLTCTVSGGSFSSHFWSWIRQPPGKGLEWIGYILYTGGTSFNPSLKSRVSMSVGTSKNQFSLKLSSVTAADTAVYYCARARSGITFTGIIVPGSFDIWGQGTMVTVSS

SEQ ID NO: 139 - protein, 108 aa, synthetic construct
  REGION 1..108: note = Synthetic Construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 140 - protein, 122 aa, synthetic construct
  REGION 1..122: note = Synthetic Construct
QVQLQQSGPGLLKPSQTLSLTCAISGDSVSSNSAAWSWIRQSPSRGLEWLGRTYYRANWFNDYALSVKSRITINPVTSTNHFSLQLHSVTPEDTAVYYCAREGALGYYFDSWGQGTLVTVSS

SEQ ID NO: 141 - protein, 113 aa, synthetic construct
  REGION 1..113: note = Synthetic Construct
DIVMTQSPDSLAVSLGERATINCKSSQSVLYSSNNKNYLAWYQQKPGQPPTLLIYWASTRESGVPDRFSGSGSGTDFTLTISSLQAEDVAIYYCHQYFITPLTFGGGTKVEIK

SEQ ID NO: 142 - protein, 126 aa, synthetic construct
  REGION 1..126: note = Synthetic Construct
QVQLQESGPGLVKPSETLSITCTVSGGSFSSHFWTWIRQPPGKGLEWIGYLHYSGGTSYNPSLKSRVIISVDTSKNQFSLKLNSVTAADTAVYYCARARSGITFGGLIVPGSFDIWGQGTMVTVSS

SEQ ID NO: 143 - protein, 126 aa, synthetic construct
  REGION 1..126: note = Synthetic Construct
QVQLQESGPGLVKPSETLSLTCTVSGGSFSSHFWSWIRQPPGKGLEWIGYIYYSGGTHYNPSLESRVTISVDTSKNQFSLKLNSVTAADTAVYYCARARSGITFGGLIVPGSFDIWGQGTMVTVSS

SEQ ID NO: 144 - protein, 125 aa, synthetic construct
  REGION 1..125: note = Synthetic Construct
EVQLVESGGGLVQPGRSLRLSCKASGFAFDDFAMHWVRQAPGKGLEWVSGIVWNSGDIGYADSVKGRFTISRDNAKNSLYLQLNSLRTEDTALYFCVKDMVRGLMGFNYYGMDVWGQGTTVTVSS

SEQ ID NO: 145 - protein, 107 aa, synthetic construct
  REGION 1..107: note = Synthetic Construct
EIVLTQSPATLSLSPGERATLSCRASQTISTYLVWYRQRPGQAPSLLIYDASNRATDIPARFSGSGSGTDFTLTISSLEPEDFAVYYCQQRSNWPITFGQGTRLEIK

SEQ ID NO: 146 - protein, 117 aa, synthetic construct
  REGION 1..117: note = Synthetic Construct
QVQLVESGGGVVQPGRSLRLSCVASGFTVSSYGIHWVRQAPGKGLEWVSVIWYDGRNKDYADSVKGRFTISRDNSKNTVYLEMKGLRAEDTALYYCARDKTGDFDSWGQGTLVTVSS

SEQ ID NO: 147 - protein, 107 aa, synthetic construct
  REGION 1..107: note = Synthetic Construct
DIQMTQSPSSLSASVGDRVTITCRASQNINSFLSWYQQKPGKAPKFLIYDASSIQSGAPSRFSGSGSGTDFTLTISSLQPEDFATYYCQQSYSSPFTFGQGTKLEIK

SEQ ID NO: 148 - protein, 123 aa, synthetic construct
  REGION 1..123: note = Synthetic Construct
EVQLVESGGGLVQPGRSLTLSCAVSGFTFDDHAMHWVRQAPGKGLEWVSGISWNSVSIGYADSVKGRFTISRDNAKTSLYLQMNSLRVDDTALYYCVQDLNDILTGYPLFDFWGQGTLVTVSS

SEQ ID NO: 149 - protein, 108 aa, synthetic construct
  REGION 1..108: note = Synthetic Construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 150 - protein, 117 aa, synthetic construct
  REGION 1..117: note = Synthetic Construct
QVQLVESGGGVVQPGRSLRLTCVASGFTVSSYGMHWVRQAPGKGLEWVAVMFYDESKKYYADSVKGRFTISRDNSKNTLYLQMNSLRVEDTAVYYCARDEQLDFEYWGQGTLVTVSS

SEQ ID NO: 151 - protein, 126 aa, synthetic construct
  REGION 1..126: note = Synthetic Construct
EVQLVESGGGVVQPGGSLRLSCAASTFTFDDFAMHWVRQAPGKGLEWVSLITGDGGSTYYADPVKGRFTISRDNSKNSLYLQMNSLRTEDTALYYCVKDWWIAARPDYYYYGMDVWGQGTTVTVSS

SEQ ID NO: 152 - protein, 8 aa, synthetic construct
  REGION 1..8: note = Synthetic Construct
GGSFSSHF

SEQ ID NO: 153 - protein, 7 aa, synthetic construct
  REGION 1..7: note = Synthetic Construct
ILYTGGT

SEQ ID NO: 154 - protein, 20 aa, synthetic construct
  REGION 1..20: note = Synthetic Construct
ARARSGITFTGIIVPGSFDI

SEQ ID NO: 155 - protein, 7 aa, synthetic construct
  REGION 1..7: note = Synthetic Construct
QSVSSSY

SEQ ID NO: 156 - skipped

SEQ ID NO: 157 - protein, 9 aa, synthetic construct
  REGION 1..9: note = Synthetic Construct
QQYGSSPWT

SEQ ID NO: 158 - protein, 8 aa, synthetic construct
  REGION 1..8: note = Synthetic Construct
GFAFDDFA

SEQ ID NO: 159 - protein, 8 aa, synthetic construct
  REGION 1..8: note = Synthetic Construct
IVWNSGDI

SEQ ID NO: 160 - protein, 18 aa, synthetic construct
  REGION 1..18: note = Synthetic Construct
VKDMVRGLMGFNYYGMDV

SEQ ID NO: 161 - protein, 6 aa, synthetic construct
  REGION 1..6: note = Synthetic Construct
QTISTY

SEQ ID NO: 162 - skipped

SEQ ID NO: 163 - protein, 9 aa, synthetic construct
  REGION 1..9: note = Synthetic Construct
QQRSNWPIT

SEQ ID NO: 164 - protein, 120 aa, synthetic construct
  REGION 1..120: note = Synthetic Construct
EVQVLESGGDLVQPGGSLRLSCAASGFTFSAYAMTWVRQAPGKGLEWVSAISGSGGSAYYADSVKGRFTISRDNSKNTVYLQMNSLRAEDTAVYYCAKDGAWKMSGLDVWGQGTTVIVSS

SEQ ID NO: 165 - protein, 107 aa, synthetic construct
  REGION 1..107: note = Synthetic Construct
DIQMTQSPASLSASVGDRVTITCRASQDISDYLAWYQQKPGKIPRLLIYTTSTLQSGVPSRFRGSGSGTDFTLTISSLQPEDVATYYCQKYDSAPLTFGGGTKVEIK

SEQ ID NO: 166 - protein, 8 aa, synthetic construct
  REGION 1..8: note = Synthetic Construct
GFTFSAYA

SEQ ID NO: 167 - protein, 8 aa, synthetic construct
  REGION 1..8: note = Synthetic Construct
ISGSGGSA

SEQ ID NO: 168 - protein, 13 aa, synthetic construct
  REGION 1..13: note = Synthetic Construct
AKDGAWKMSGLDV

SEQ ID NO: 169 - protein, 6 aa, synthetic construct
  REGION 1..6: note = Synthetic Construct
QDISDY

SEQ ID NO: 170 - skipped

SEQ ID NO: 171 - protein, 9 aa, synthetic construct
  REGION 1..9: note = Synthetic Construct
QKYDSAPLT

SEQ ID NO: 172 - protein, 122 aa, synthetic construct
  REGION 1..122: note = Synthetic Construct
QVQLVQSGAEVKKPGASVKVSCKASGYTFTSYGLSWVRQAPGQGLEWMGWIIPYNGNTNSAQKLQGRVTMTTDTSTSTAYMELRSLRSDDTAVYFCARDRDYGVNYDAFDIWGQGTMVTVSS

SEQ ID NO: 173 - protein, 106 aa, synthetic construct
  REGION 1..106: note = Synthetic Construct
SYEVTQAPSVSVSPGQTASITCSGDKLGDKYACWYQQKPGQSPVLVIYQDSKRPSGIPERFSGSNSGNTATLTISGTQAMDEADYYCQAWDSSTAVFGGGTKLTVL

SEQ ID NO: 174 - protein, 123 aa, synthetic construct
  REGION 1..123: note = Synthetic Construct
EVQLVESGGGLVQPGGSLRLSCAASGFTFSSYWMSWVRQAPGKGLECVANIKQDGSEEYYVDSVKGRFTISRDNAKNSLYLQMNSLRAEDTAVYYCARGSSSWYYYNYGMDVWGQGTTVTVSS

SEQ ID NO: 175 - protein, 107 aa, synthetic construct
  REGION 1..107: note = Synthetic Construct
DIQMTQSPSSLSASVGDRVTITCRASQGIRNDLGWYQQKPGKAPKRLIYAASSLQSGVPSRFSGSGSGTEFTLTISSLQPEDFATYYCRQQNTYPLTFGGGTKVEIK

SEQ ID NO: 176 - protein, 10 aa, synthetic construct
  REGION 1..10: note = Synthetic Construct
GYTFTSYGLS

SEQ ID NO: 177 - protein, 17 aa, synthetic construct
  REGION 1..17: note = Synthetic Construct
WIIPYNGNTNSAQKLQG

SEQ ID NO: 178 - protein, 13 aa, synthetic construct
  REGION 1..13: note = Synthetic Construct
DRDYGVNYDAFDI

SEQ ID NO: 179 - protein, 11 aa, synthetic construct
  REGION 1..11: note = Synthetic Construct
SGDKLGDKYAC

SEQ ID NO: 180 - protein, 7 aa, synthetic construct
  REGION 1..7: note = Synthetic Construct
QDSKRPS

SEQ ID NO: 181 - protein, 9 aa, synthetic construct
  REGION 1..9: note = Synthetic Construct
QAWDSSTAV

SEQ ID NO: 182 - protein, 10 aa, synthetic construct
  REGION 1..10: note = Synthetic Construct
GFTFSSYWMS

SEQ ID NO: 183 - protein, 17 aa, synthetic construct
  REGION 1..17: note = Synthetic Construct
NIKQDGSEEYYVDSVKG

SEQ ID NO: 184 - protein, 14 aa, synthetic construct
  REGION 1..14: note = Synthetic Construct
GSSSWYYYNYGMDV

SEQ ID NO: 185 - protein, 11 aa, synthetic construct
  REGION 1..11: note = Synthetic Construct
RASQGIRNDLG

SEQ ID NO: 186 - protein, 7 aa, synthetic construct
  REGION 1..7: note = Synthetic Construct
AASSLQS

SEQ ID NO: 187 - protein, 9 aa, synthetic construct
  REGION 1..9: note = Synthetic Construct
RQQNTYPLT

SEQ ID NO: 188 - protein, 113 aa, synthetic construct
  REGION 1..113: note = Synthetic Construct
EVQLVESGGGLVQPGGSLRLSCAASGLTFSRYPMSWVRQAPGKGLVWVSAITSSGGSTYYSDTVKGRFTISRDNAKNTLYLQMNSLRAEDTAVYYCARLPDYWGQGTLVTVSS

SEQ ID NO: 189 - protein, 108 aa, synthetic construct
  REGION 1..108: note = Synthetic Construct
EIVLTQSPGTLSLSPGERATLSCRASSSVSSSYLHWYQQKPGQAPRLLIYSTSNLVAGIPDRFSGSGSGTDFTLTISRLEPEDFAVYYCQHHSGYHFTFGGGTKVEIK

SEQ ID NO: 190 - protein, 10 aa, synthetic construct
  REGION 1..10: note = Synthetic Construct
  VARIANT 3: note = Xaa is Thr or Asn
  VARIANT 8: note = Xaa is Gly, Thr or Pro
GLXFSRYXMS

SEQ ID NO: 191 - protein, 17 aa, synthetic construct
  REGION 1..17: note = Synthetic Construct
  VARIANT 1: note = Xaa is Ala, Arg or His
  VARIANT 2: note = Xaa is IIe or Gln
  VARIANT 3: note = Xaa is Asn, Thr or Lys
  VARIANT 4: note = Xaa is Ser or Ala
  VARIANT 5: note = Xaa is His, Ser, Val, Leu, Met, Asn, Gly, Asp, Trp, Arg, Ala,Tyr, Lys, Pro, Thr, IIe or Glu
  VARIANT 7: note = Xaa is Gly or Ser
  VARIANT 10: note = Xaa is Tyr or Lys
XXXXXGXSTXYSDTVKG

SEQ ID NO: 192 - protein, 4 aa, synthetic construct
  REGION 1..4: note = Synthetic Construct
LPDY

SEQ ID NO: 193 - protein, 12 aa, synthetic construct
  REGION 1..12: note = Synthetic Construct
  VARIANT 3: note = Xaa is Ser or Leu
  VARIANT 4: note = Xaa is Ser or Gln
RAXXSVSSSYLH

SEQ ID NO: 194 - protein, 7 aa, synthetic construct
  REGION 1..7: note = Synthetic Construct
  VARIANT 6: note = Xaa is Ala, Val, Thr, or Met
  VARIANT 7: note = Xaa is Ser, Ala, Thr, Asn, Trp, Asp or Tyr
STSNLXX

SEQ ID NO: 195 - protein, 9 aa, synthetic construct
  REGION 1..9: note = Synthetic Construct
  VARIANT 2: note = Xaa is Gln, Asn, His, Leu, Pro or Trp
  VARIANT 3: note = Xaa is Tyr or His
  VARIANT 4: note = Xaa is Ser or Leu
  VARIANT 7: note = Xaa is His, Phe, Gln or Thr
  VARIANT 8: note = Xaa is Phe or Trp
QXXXGYXXT

SEQ ID NO: 196 - protein, 10 aa, synthetic construct
  REGION 1..10: note = Synthetic Construct
GLTFSRYPMS

SEQ ID NO: 197 - protein, 17 aa, synthetic construct
  REGION 1..17: note = Synthetic Construct
AITSSGGSTYYSDTVKG

SEQ ID NO: 198 - protein, 12 aa, synthetic construct
  REGION 1..12: note = Synthetic Construct
RASSSVSSSYLH

SEQ ID NO: 199 - protein, 7 aa, synthetic construct
  REGION 1..7: note = Synthetic Construct
STSNLVA

SEQ ID NO: 200 - protein, 9 aa, synthetic construct
  REGION 1..9: note = Synthetic Construct
QHHSGYHFT

SEQ ID NO: 201 - protein, 124 aa, synthetic construct
  REGION 1..124: note = Synthetic Construct
QVTLRESGPALVKPTQTLTLTCTFSGFSLRKVGSSVSWIRQPPGKALEWLAHIYWDDDKRYNPSLRNRLTISKDTSKNQVVLTMTNMDPVDTATYYCARRAITTVIGGGTFDYWGQGTTVTVSS

SEQ ID NO: 202 - protein, 106 aa, synthetic construct
  REGION 1..106: note = Synthetic Construct
DIQMTQSPSSLSASVGDRVTITCSASSSISYMHWYQQKPGKAPKLLIYDTSKLAVGVPSRFSGSGSGTDFTLTISSLQPEDFATYYCQQWYRNPLTFGGGTKVEIK

SEQ ID NO: 203 - protein, 106 aa, synthetic construct
  REGION 1..106: note = Synthetic Construct
DIQMTQSPSSLSASVGDRVTITCSASSSISYMHWYQQKPGKAPKLLIYDTSKLARGVPSRFSGSGSGTDFTLTISSLQPEDFATYYCQQWYRNPLTFGGGTKVEIK

SEQ ID NO: 204 - protein, 124 aa, synthetic construct
  REGION 1..124: note = Synthetic Construct
QVTLRESGPALVKPTQTLTLTCTFSGFSLRKVGSSVSWIRQPPGKALEWLAHIYWDDDKRYNPSLRNRLTISKDTSKNQVVLTMTNMDPVDTATYYCARRAITTVIGGGTMDYWGQGTTVTVSS

SEQ ID NO: 205 - protein, 123 aa, synthetic construct
  REGION 1..123: note = Synthetic Construct
QVTLRESGPALVKPTQTLTLTCTFSGFSLRKVGRSVSWIRQPPGKALEWLAHIYWDDDKRYNPSLRNRLTISKDTSKNQVVLTMTNMDPVDTATYYCARRAITTVIGGGTFDYWGQGTTVVSS

SEQ ID NO: 206 - protein, 124 aa, synthetic construct
  REGION 1..124: note = Synthetic Construct
QVTLRESGPALVKPTQTLTLTCTFSGFSWRKVGSSVSWIRQPPGKALEWLAHIYWDDDKRYNPSLRNRLTISKDTSKNQVVLTMTNMDPVDTATYYCARRAITTVIGGGTFDYWGQGTTVTVSS

SEQ ID NO: 207 - protein, 124 aa, synthetic construct
  REGION 1..124: note = Synthetic Construct
QVTLRESGPALVKPTQTLTLTCTFSGFSLRKVGSSVSWIRQPPGKALEWLAHIYWDDDLRYNPSLRNRLTISKDTSKNQVVLTMTNMDPVDTATYYCARRAITTVIGGGTFDYWGQGTTVTVSS

SEQ ID NO: 208 - protein, 124 aa, synthetic construct
  REGION 1..124: note = Synthetic Construct
QVTLRESGPALVKPTQTLTLTCTFSGFSMRKVGSSVSWIRQPPGKALEWLAHIYWDDDKRYNPSLRNRLTISKDTSKNQVVLTMTNMDPVDTATYYCARRAITTVIGGGTFDYWGQGTTVTVSS

SEQ ID NO: 209 - protein, 124 aa, synthetic construct
  REGION 1..124: note = Synthetic Construct
QVTLRESGPALVKPTQTLTLTCTFSGFSLRKVGSSISWIRQPPGKALEWLAHIYWDDDKRYNPSLRNRLTISKDTSKNQVVLTMTNMDPVDTATYYCARRAITTVIGGGTFDYWGQGTTVTVSS

SEQ ID NO: 210 - protein, 124 aa, synthetic construct
  REGION 1..124: note = Synthetic Construct
QVTLRESGPALVKPTQTLTLTCTFSGFSLRKVGSSVSWIRQPPGKALEWLAHIYWDDDKRYNPSLRNRLTISKDTSKNQVVLTMTNMDPVDTATYYCARRKITTVIGGGTFDYWGQGTTVTVSS

SEQ ID NO: 211-220 - skipped (10 entries)

SEQ ID NO: 221 - protein, 106 aa, synthetic construct
  REGION 1..106: note = Synthetic Construct
DIQMTQSPSSLSASVGDRVTITCSASSSISYMHWYQQKPGKAPKLLIYDTSKLARGVPSRFSGSGSGTDFTLTISSLQPEDFATYYCQQWYSNPLTFGGGTKVEIK

SEQ ID NO: 222 - protein, 124 aa, synthetic construct
  REGION 1..124: note = Synthetic Construct
QLQLQESGPGLVKPSETLSLTCTVSGFSLRRVGSSVSWIRQPPGKGLEWIGHIYWDDDKRYNPSLRNRVTISVDTSKNQFSLKLSSVTAADTAVYYCARRAITTVIGGGTFDYWGQGTLVTVSS

SEQ ID NO: 223 - protein, 124 aa, synthetic construct
  REGION 1..124: note = Synthetic Construct
QLQLQESGPGLVKPSETLSLTCTVSGFSLRMVGSSVSWIRQPPGKGLEWIGHIYWDDDKRLNPSLRNRVTISVDTSKNQFSLKLSSVTAADTAVYYCARRAITTVIGGGTFDYWGQGTLVTVSS

SEQ ID NO: 224 - protein, 124 aa, synthetic construct
  REGION 1..124: note = Synthetic Construct
QLQLQESGPGLVKPSETLSLTCTVSGFSLRKVGSSVSWIRQPPGKGLEWIGHIYWDDDLRYNPSLRNRVTISVDTSKNQFSLKLSSVTAADTAVYYCARRAITTVIGGGTFDYWGQGTLVTVSS

SEQ ID NO: 225 - protein, 124 aa, synthetic construct
  REGION 1..124: note = Synthetic Construct
QLQLQESGPGLVKPSETLSLTCTVSGFSLRKVGSSVSWIRQPPGKGLEWIGHIYWDDDKRYNPSLRNRVTISVDTSKNQFSLKLSSVTAADTAVYYCARRAITTVIGGGTFDMWGQGTLVTVSS

SEQ ID NO: 226 - protein, 124 aa, synthetic construct
  REGION 1..124: note = Synthetic Construct
QLQLQESGPGLVKPSETLSLTCTVSGFSLRKVGSSVSWIRQPPGKGLEWIGHIYWDDDKRYNPSLRNRVTISVDTSKNQFSLKLSSVTAADTAVYYCARRAITTVIGGGTFDLWGQGTLVTVSS

SEQ ID NO: 227 - protein, 124 aa, synthetic construct
  REGION 1..124: note = Synthetic Construct
QLQLQESGPGLVKPSETLSLTCTVSGFSLRKVGSSVSWIRQPPGKGLEWIGHIYWDDDKRLNPSLRNRVTISVDTSKNQFSLKLSSVTAADTAVYYCARRAITTVIGGGTFDYWGQGTLVTVSS

SEQ ID NO: 228 - protein, 124 aa, synthetic construct
  REGION 1..124: note = Synthetic Construct
QLQLQESGPGLVKPSETLSLTCTVSGFSLRKVGSSVSWIRQPPGKGLEWIGHIYWDDDKRYNPSLRNRVTISVDTSKNQFSLKLSSVTAADTAVYYCARRAITTVIGGGTFDYWGQGTLVTVSS

SEQ ID NO: 229 - protein, 106 aa, synthetic construct
  REGION 1..106: note = Synthetic Construct
DIQMTQSPSSLSASVGDRVTITCSASSSISYSHWYQQKPGKAPKLLIYDTSKLARGVPSRFSGSGSGTDFTLTISSLQPEDFATYYCQQWYSNPLTFGGGTKVEIK

SEQ ID NO: 230 - protein, 106 aa, synthetic construct
  REGION 1..106: note = Synthetic Construct
DIQMTQSPSSLSASVGDRVTITCSASSSISYAHWYQQKPGKAPKLLIYDTSKLARGVPSRFSGSGSGTDFTLTISSLQPEDFATYYCQQWYSNPLTFGGGTKVEIK

SEQ ID NO: 231 - protein, 106 aa, synthetic construct
  REGION 1..106: note = Synthetic Construct
DIQMTQSPSSLSASVGDRVTITCSASSSISYMHWYQQKPGKAPKLLIYDTSKLARGVPSRFSGSGSGTDFTLTISSLQPEDFATYYCQQWYLNPLTFGGGTKVEIK

SEQ ID NO: 232 - protein, 106 aa, synthetic construct
  REGION 1..106: note = Synthetic Construct
DIQMTQSPSSLSASVGDRVTITCSASSSISYMHWYQQKPGKAPKLLIYDTSKLARGVPSRFSGSGSGTDFTLTISSLQPEDFATYYCQQWYENPLTFGGGTKVEIK

SEQ ID NO: 233 - protein, 106 aa, synthetic construct
  REGION 1..106: note = Synthetic Construct
DIQMTQSPSSLSASVGDRVTITCSASSSISYMHWYQQKPGKAPKLLIYDTSKLARGVPSRFSGSGSGTDFTLTISSLQPEDFATYYCQQWYFNPLTFGGGTKVEIK

SEQ ID NO: 234 - protein, 124 aa, synthetic construct
  REGION 1..124: note = Synthetic Construct
QLQLQESGPGLVKPSETLSLTCTVSGFSLRKVGSSVSWIRQPPGKGLEWIGHIYWDDDKRLNPSLRNRVTISVDTSKNQFSLKLSSVTAADTAVYYCARRAITTVIGGGTFDLWGQGTLVTVSS

SEQ ID NO: 235 - protein, 12 aa, synthetic construct
  REGION 1..12: note = Synthetic Construct
  VARIANT 4: note = Xaa is Leu, Trp, or Met
  VARIANT 9: note = Xaa is Ser or Arg
  VARIANT 6: note = Xaa is Lys, Arg, or Met
GFSXRXVGXSVS

SEQ ID NO: 236 - protein, 16 aa, synthetic construct
  REGION 1..16: note = Synthetic Construct
  VARIANT 8: note = Xaa is Lys or Leu
  VARIANT 10: note = Xaa is Tyr or Leu
HIYWDDDXRXNPSLRN

SEQ ID NO: 237 - protein, 14 aa, synthetic construct
  REGION 1..14: note = Synthetic Construct
  VARIANT 2: note = Xaa is Ala or Lys
  VARIANT 12: note = Xaa is Met or Phe
  VARIANT 14: note = Xaa is Met or Leu
RXITTVIGGGTXDX

SEQ ID NO: 238 - protein, 10 aa, synthetic construct
  REGION 1..10: note = Synthetic Construct
SASSSISYMH

SEQ ID NO: 239 - protein, 7 aa, synthetic construct
  REGION 1..7: note = Synthetic Construct
DTSKLAR

SEQ ID NO: 240 - protein, 9 aa, synthetic construct
  REGION 1..9: note = Synthetic Construct
  VARIANT 5: note = Xaa is Ser, Arg, Leu, Glu, or Phe
QQWYXNPLT

SEQ ID NO: 241 - protein, 10 aa, synthetic construct
  REGION 1..10: note = Synthetic Construct
SASSSISYSH

SEQ ID NO: 242 - protein, 12 aa, synthetic construct
  REGION 1..12: note = Synthetic Construct
GFSLRKVGRSVS

SEQ ID NO: 243 - protein, 16 aa, synthetic construct
  REGION 1..16: note = Synthetic Construct
HIYWDDDKRYNPSLRN

SEQ ID NO: 244 - protein, 14 aa, synthetic construct
  REGION 1..14: note = Synthetic Construct
RAITTVIGGGTFDY

SEQ ID NO: 245 - protein, 9 aa, synthetic construct
  REGION 1..9: note = Synthetic Construct
QQWYRNPLT

SEQ ID NO: 246 - protein, 12 aa, synthetic construct
  REGION 1..12: note = Synthetic Construct
GFSLRKVGSSVS

SEQ ID NO: 247 - protein, 16 aa, synthetic construct
  REGION 1..16: note = Synthetic Construct
HIYWDDDKRLNPSLRN

SEQ ID NO: 248 - protein, 14 aa, synthetic construct
  REGION 1..14: note = Synthetic Construct
RAITTVIGGGTFDL

SEQ ID NO: 249 - protein, 9 aa, synthetic construct
  REGION 1..9: note = Synthetic Construct
QQWYLNPLT

SEQ ID NO: 250 - protein, 119 aa, synthetic construct
  REGION 1..119: note = Synthetic Construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDRGYSYGLDYWGQGTLVTVSS

SEQ ID NO: 251 - protein, 107 aa, synthetic construct
  REGION 1..107: note = Synthetic Construct
DIQMTQSPSSLSASVGDRVTITCRASQGIRNALGWYQQKPGKAPQRLIYAASSLQSGVPSRFSGSGSGTEFTLTISSLQPEDFATYYCLQHNSYPRTFGQGTKVEIK

SEQ ID NO: 252 - protein, 123 aa, synthetic construct
  REGION 1..123: note = Synthetic Construct
QVQLVQSGAEVKKPGASVKVSCKASGYTFTGYYMHWVRQAPGQGLEWMGWINPNNGGTNYAQKFQGRVTMTRDTSISTAYMELSRLRSDDTAVYHCARNTVGSGYYYYGMDVWGQGTTVTVSS

SEQ ID NO: 253 - protein, 112 aa, synthetic construct
  REGION 1..112: note = Synthetic Construct
DIVMTQSPLSLPVTPGEPASISCRSSQSLLHSNGYNYLDWYLQKSGQSPQLLIHLGSNRASGVPDRFSGSGSGTDFTLKISRVEAEDVGVYYCMQVLQTPLTFGGGTKVEIK

SEQ ID NO: 254 - protein, 119 aa, synthetic construct
  REGION 1..119: note = Synthetic Construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDRGYSYGIDYWGQGTLVTVSS

SEQ ID NO: 255 - protein, 107 aa, synthetic construct
  REGION 1..107: note = Synthetic Construct
DIQMTQSPSSLSASVGDRVTITCRASQGIRNALGWYQQKPGKAPKRLIYAASSLQSGVPSRFSGSGSGTEFTLTISSLQPEDFATYYCLQHNSYPRTFGQGTKVEIK

SEQ ID NO: 256 - protein, 119 aa, synthetic construct
  REGION 1..119: note = Synthetic Construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDRGYYYGMDVWGQGTTVTVSS

SEQ ID NO: 257 - protein, 107 aa, synthetic construct
  REGION 1..107: note = Synthetic Construct
DIQMTQSPSSLSASVGDRVTLTCRASQGIRNALGWYQQKPGKAPKRLIYGASSLQSGVPSRFSGSGSGTEFTLTISSLQPEDFATYYCLQHNSYPFTFGPGTKVDIK

SEQ ID NO: 258 - protein, 119 aa, synthetic construct
  REGION 1..119: note = Synthetic Construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDRGSYFLFDYWGQGTLVTVSS

SEQ ID NO: 259 - protein, 107 aa, synthetic construct
  REGION 1..107: note = Synthetic Construct
DMQMTQSPSSLSASVGDRVTITCRASQGIRNDLGWYQQKPGKAPKRLIYGASSLQSGVPSRFSGSGSGTEFTLTISSLQPEDSATYYCLQHNSYPLTFGGGTKVEIK

SEQ ID NO: 260 - protein, 119 aa, synthetic construct
  REGION 1..119: note = Synthetic Construct
EVQLVESGGGLVKPGGSLRLSCAASGFTFSSYSMNWVRQAPGKGLEWVSSISSSSSYIYYADSVKGRFTISRDNAKNSLYLQMNSLRAEDTTVYYCARDRGYLYGMDVWGQGTTVTVSS

SEQ ID NO: 261 - protein, 107 aa, synthetic construct
  REGION 1..107: note = Synthetic Construct
DIQMTQSPSSLSASVGDRVTITCRASQAIRNDLGWYQQKPGKAPKRLIYAASSLQSGVPSRFSGSGSGTEFTLTISSLQPEDFATYYCLQHNSYPRTFGQGTKVEIK

SEQ ID NO: 262 - protein, 123 aa, synthetic construct
  REGION 1..123: note = Synthetic Construct
QVQLVQSGAEVKKPGASVKVSCKASGYTFTGYYMHWVRQAPGQGLEWMGWINPNSGGTNYAQKFQGRVTMTRDTSISTAYLELSRLRSDDTAVYYCARNTVGTGYYYYGMDVWGQGTTVTVSS

SEQ ID NO: 263 - protein, 112 aa, synthetic construct
  REGION 1..112: note = Synthetic Construct
DIVMTQSPLSLPVTPGEPASISCRSSQSLLHSNGYNYLDWYLQKPGQSPQVLIYLVSNRASGVPDRFSGSGSGTDFTLKISRVEAEDVGVYYCMQALQTPPTFGGGTKVEIK

SEQ ID NO: 264 - protein, 119 aa, synthetic construct; identical to SEQ ID NO: 250
  REGION 1..119: note = Synthetic Construct

SEQ ID NO: 265 - protein, 107 aa, synthetic construct
  REGION 1..107: note = Synthetic Construct
DIQMTQSPSSLSASVGDRVTITCRASQGIRNDLGWYQQKPGKAPKRLIYAASSLQSGVPSRFSGSGSGTEFTLTISSLQPEDFATYYCLQLNSYPFTFGPGTKVDIK

SEQ ID NO: 266 - protein, 122 aa, synthetic construct
  REGION 1..122: note = Synthetic Construct
EVQLLESGGGLIQPGGSLRLSCAASGFTFSSFAMSWVRQAPGKGLEWVSTISGSGGYTFYADSVKGRFTISRDNSKNTLYLQMNSLRAEDTAVYYCAKDGRYNWNYGAFDIWGQGTMVTVSS

SEQ ID NO: 267 - protein, 107 aa, synthetic construct
  REGION 1..107: note = Synthetic Construct
EIVMTQSPATLSVSPGERATLSCRASQSVSSNLAWYQQKPGQAPRLLIYGASTRATGFPARFSGSGSGTEFTLTISSLQSEDFAIYYCQQYNNWPLTFGGGTKVEIK

SEQ ID NO: 268 - protein, 122 aa, synthetic construct
  REGION 1..122: note = Synthetic Construct
EVQLLESGGGLIQPGGSLRLSCAASGFTFSSFAMSWVRQAPGKGLEWVSTISGSGGSTYYADFVKGRFTISRDNSKNTLYLQMNSLRAEDTAVYYCAKDGRYNWNYGAFDIWGQGTMVTVSS

SEQ ID NO: 269 - protein, 107 aa, synthetic construct
  REGION 1..107: note = Synthetic Construct
EIVMTQSPATLSVSPGERATLSCRASQSVRSNLAWFQQKPGQAPRLLIYGASTRATGIPARFSGSGSGTEFTLTISSLQSEDFAVYYCQQYNNWPLTFGGGTKVEIK

SEQ ID NO: 270 - protein, 122 aa, synthetic construct
  REGION 1..122: note = Synthetic Construct
EVQLLESGGGLVQPGGSLRLSCAASGFTFSSYAMSWVRQAPGKGLEWVSAISGSGGSTFYADSVTGRFTISRDNSKNTLFLQLNSLRAEDTAVYHCAKDGRFNWNYGASDIWGQGTMVTVSS

SEQ ID NO: 271 - protein, 107 aa, synthetic construct
  REGION 1..107: note = Synthetic Construct
EIVMTQSPATLSVSPGERATLSCRTSQSVSSNLAWYQQKPGQAPRLLIYGASTRATGIPARFSGSGSGTEFTLTISSLQSEDFAVYYCQQYNNWPLTFGGGTKVEIK

SEQ ID NO: 272 - protein, 122 aa, synthetic construct
  REGION 1..122: note = Synthetic Construct
EVQLLESGGGLVQPGGSLRLSCAASGFTFSSFAMSWVRQAPGKGLEWVSTISGSGGYTFYADSVKGRFTISRDNSKNTLYLQMNSLRAEDTAVYYCAKDGRYNWNYGAFDIWGQGTMVTVSS

SEQ ID NO: 273 - protein, 107 aa, synthetic construct
  REGION 1..107: note = Synthetic Construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPLTFGGGTKVEIK

SEQ ID NO: 274 - protein, 445 aa, synthetic construct
  REGION 1..445: note = Synthetic Construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDRGYSYGLDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 275 - protein, 214 aa, synthetic construct
  REGION 1..214: note = Synthetic Construct
DIQMTQSPSSLSASVGDRVTITCRASQGIRNALGWYQQKPGKAPQRLIYAASSLQSGVPSRFSGSGSGTEFTLTISSLQPEDFATYYCLQHNSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 276 - protein, 449 aa, synthetic construct
  REGION 1..449: note = Synthetic Construct
QVQLVQSGAEVKKPGASVKVSCKASGYTFTGYYMHWVRQAPGQGLEWMGWINPNNGGTNYAQKFQGRVTMTRDTSISTAYMELSRLRSDDTAVYHCARNTVGSGYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 277 - protein, 219 aa, synthetic construct
  REGION 1..219: note = Synthetic Construct
DIVMTQSPLSLPVTPGEPASISCRSSQSLLHSNGYNYLDWYLQKSGQSPQLLIHLGSNRASGVPDRFSGSGSGTDFTLKISRVEAEDVGVYYCMQVLQTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 278 - protein, 445 aa, synthetic construct
  REGION 1..445: note = Synthetic Construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDRGYSYGIDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 279 - protein, 214 aa, synthetic construct
  REGION 1..214: note = Synthetic Construct
DIQMTQSPSSLSASVGDRVTITCRASQGIRNALGWYQQKPGKAPKRLIYAASSLQSGVPSRFSGSGSGTEFTLTISSLQPEDFATYYCLQHNSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 280 - protein, 445 aa, synthetic construct
  REGION 1..445: note = Synthetic Construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDRGYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 281 - protein, 214 aa, synthetic construct
  REGION 1..214: note = Synthetic Construct
DIQMTQSPSSLSASVGDRVTLTCRASQGIRNALGWYQQKPGKAPKRLIYGASSLQSGVPSRFSGSGSGTEFTLTISSLQPEDFATYYCLQHNSYPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 282 - protein, 445 aa, synthetic construct
  REGION 1..445: note = Synthetic Construct
EVQLVESGGGLVKPGGSLRLSCAASGFTFSSYSMNWVRQAPGKGLEWVSSISSSSSYIYYADSVKGRFTISRDNAKNSLYLQMNSLRAEDTTVYYCARDRGYLYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 283 - protein, 214 aa, synthetic construct
  REGION 1..214: note = Synthetic Construct
DIQMTQSPSSLSASVGDRVTITCRASQAIRNDLGWYQQKPGKAPKRLIYAASSLQSGVPSRFSGSGSGTEFTLTISSLQPEDFATYYCLQHNSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 284 - protein, 449 aa, synthetic construct
  REGION 1..449: note = Synthetic Construct
QVQLVQSGAEVKKPGASVKVSCKASGYTFTGYYMHWVRQAPGQGLEWMGWINPNSGGTNYAQKFQGRVTMTRDTSISTAYLELSRLRSDDTAVYYCARNTVGTGYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 285 - protein, 219 aa, synthetic construct
  REGION 1..219: note = Synthetic Construct
DIVMTQSPLSLPVTPGEPASISCRSSQSLLHSNGYNYLDWYLQKPGQSPQVLIYLVSNRASGVPDRFSGSGSGTDFTLKISRVEAEDVGVYYCMQALQTPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 286 - protein, 449 aa, synthetic construct
  REGION 1..449: note = Synthetic Construct
EVQLLESGGGLIQPGGSLRLSCAASGFTFSSFAMSWVRQAPGKGLEWVSTISGSGGYTFYADSVKGRFTISRDNSKNTLYLQMNSLRAEDTAVYYCAKDGRYNWNYGAFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 287 - protein, 214 aa, synthetic construct
  REGION 1..214: note = Synthetic Construct
EIVMTQSPATLSVSPGERATLSCRASQSVSSNLAWYQQKPGQAPRLLIYGASTRATGFPARFSGSGSGTEFTLTISSLQSEDFAIYYCQQYNNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 288 - protein, 445 aa, synthetic construct
  REGION 1..445: note = Synthetic Construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDRGSYFLFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 289 - protein, 214 aa, synthetic construct
  REGION 1..214: note = Synthetic Construct
DMQMTQSPSSLSASVGDRVTITCRASQGIRNDLGWYQQKPGKAPKRLIYGASSLQSGVPSRFSGSGSGTEFTLTISSLQPEDSATYYCLQHNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 290 - protein, 445 aa, synthetic construct; identical to SEQ ID NO: 274
  REGION 1..445: note = Synthetic Construct

SEQ ID NO: 291 - protein, 214 aa, synthetic construct
  REGION 1..214: note = Synthetic Construct
DIQMTQSPSSLSASVGDRVTITCRASQGIRNDLGWYQQKPGKAPKRLIYAASSLQSGVPSRFSGSGSGTEFTLTISSLQPEDFATYYCLQLNSYPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 292 - protein, 449 aa, synthetic construct
  REGION 1..449: note = Synthetic Construct
EVQLLESGGGLIQPGGSLRLSCAASGFTFSSFAMSWVRQAPGKGLEWVSTISGSGGSTYYADFVKGRFTISRDNSKNTLYLQMNSLRAEDTAVYYCAKDGRYNWNYGAFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 293 - protein, 214 aa, synthetic construct
  REGION 1..214: note = Synthetic Construct
EIVMTQSPATLSVSPGERATLSCRASQSVRSNLAWFQQKPGQAPRLLIYGASTRATGIPARFSGSGSGTEFTLTISSLQSEDFAVYYCQQYNNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 294 - protein, 449 aa, synthetic construct
  REGION 1..449: note = Synthetic Construct
EVQLLESGGGLVQPGGSLRLSCAASGFTFSSYAMSWVRQAPGKGLEWVSAISGSGGSTFYADSVTGRFTISRDNSKNTLFLQLNSLRAEDTAVYHCAKDGRFNWNYGASDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 295 - protein, 214 aa, synthetic construct
  REGION 1..214: note = Synthetic Construct
EIVMTQSPATLSVSPGERATLSCRTSQSVSSNLAWYQQKPGQAPRLLIYGASTRATGIPARFSGSGSGTEFTLTISSLQSEDFAVYYCQQYNNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 296 - protein, 448 aa, synthetic construct
  REGION 1..448: note = Synthetic Construct
EVQLLESGGGLVQPGGSLRLSCAASGFTFSSFAMSWVRQAPGKGLEWVSTISGSGGYTFYADSVKGRFTISRDNSKNTLYLQMNSLRAEDTAVYYCAKDGRYNWNYGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 297 - protein, 214 aa, synthetic construct
  REGION 1..214: note = Synthetic Construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 298 - protein, 115 aa, synthetic construct
  REGION 1..115: note = Synthetic Construct
EVQLVESGGGLVQPGGSLRLSCAASGFTFSRYWMNWVRQAPGKGLEWVAQIRLKSDNYATHYAESVKGRFTISRDNAKNSLYLQMNSLRAEDTAVYYCTEGLDYWGQGTTVTVSS

SEQ ID NO: 299 - protein, 107 aa, synthetic construct
  REGION 1..107: note = Synthetic Construct
DIQMTQSPSSLSASVGDRVTITCKASQDINKYVAWYQQKPGKAPKLLIYYTSWLQPGVPSRFSGSGSGTDFTFTISSLQPEDIATYYCLQYDNLLYTFGQGTKLEIK

SEQ ID NO: 300 - protein, 5 aa, synthetic construct
  REGION 1..5: note = Synthetic Construct
RYWMN

SEQ ID NO: 301 - protein, 19 aa, synthetic construct
  REGION 1..19: note = Synthetic Construct
QIRLKSDNYATHYAESVKG

SEQ ID NO: 302 - protein, 4 aa, synthetic construct
  REGION 1..4: note = Synthetic Construct
GLDY

SEQ ID NO: 303 - protein, 11 aa, synthetic construct
  REGION 1..11: note = Synthetic Construct
KASQDINKYVA

SEQ ID NO: 304 - protein, 7 aa, synthetic construct
  REGION 1..7: note = Synthetic Construct
YTSWLQP

SEQ ID NO: 305 - protein, 9 aa, synthetic construct
  REGION 1..9: note = Synthetic Construct
LQYDNLLYT

SEQ ID NO: 306 - protein, 126 aa, synthetic construct
  REGION 1..126: note = Synthetic Construct
QIQLVQSGGGVVQPGRSLRLSCAASGFTFSSYGMHWVRQAPGKGLEWVAVISYDGSNKYYADSVKGRFTISRDNSKNTLYLQMNSLRAEDTAVYYCARDLLVRFLEWSHYYGMDVWGQGTTVTVSS

SEQ ID NO: 307 - protein, 109 aa, synthetic construct
  REGION 1..109: note = Synthetic Construct
QPVLTQPPSASGTPGQRVTISCSGSSSNIGSNTVHWYQQLPGTAPKLLIYSDNQRPSGVPDRFSGSKSGTSASLVISGLQSDDEADYYCAAWDDSLNGVFGGGTKLTVL

SEQ ID NO: 308 - protein, 126 aa, synthetic construct
  REGION 1..126: note = Synthetic Construct
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARDLLVRFLEWSHYYGMDVWGQGTTVTVSS

SEQ ID NO: 309 - protein, 109 aa, synthetic construct
  REGION 1..109: note = Synthetic Construct
QSVLTQPPSASGTPGQRVTISCSGSSSNIGSNTVHWYQQLPGTAPKLLIYSDNQRPSGVPDRFSGSKSGTSASLAISGLQSEDEADYYCAAWDDSLNGVFGGGTKLTVL

SEQ ID NO: 310 - protein, 131 aa, synthetic construct
  REGION 1..131: note = Synthetic Construct
QVQLQQSGGGLVQPGGSLRLSCAASGFTFSSYAMTWVRQAPGKGLEWVSAISGGGGATYYADSVEGRFTISRDNSKNTLYLQMNSLRAEDTAIYYCASPAGHICSGGSCQYYYYGMDVWGQGTTVTVSSGS

SEQ ID NO: 311 - protein, 116 aa, synthetic construct
  REGION 1..116: note = Synthetic Construct
DIQMTQSPSFLSASVGDRVTITCRASQGISSYLAWYQQKPGKAPKLLIYAASSLQSGVPSRFSGSGSGTDFTLTISSLQPEDFATYYCQQSYSTPLTFGGGTRVDMARTVAAPSVF

SEQ ID NO: 312 - protein, 128 aa, synthetic construct
  REGION 1..128: note = Synthetic Construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDRHLYRRGYSSVDGMDVWGQGTTVTVSSGS

SEQ ID NO: 313 - protein, 117 aa, synthetic construct
  REGION 1..117: note = Synthetic Construct
EIVMTQSPGTLSLSPGERATLSCRASQSVSSGYLAWYQQKAGQAPRLLIYGGSARAPGIPARFSGSGSGTDFTLTISSLEPEDFAVYYCQQRSIWPLTFGGGTKVEIKRTVAAPSVF

SEQ ID NO: 314 - protein, 120 aa, synthetic construct
  REGION 1..120: note = Synthetic Construct
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARHPIGSGYDYWGQGTLVTVSSGS

SEQ ID NO: 315 - protein, 121 aa, synthetic construct
  REGION 1..121: note = Synthetic Construct
DIVMTQSPVSLPVTLGQPASISCRSSQSVVYRDGNTYLSWFLQRPGQSPRRLIYKVSNRDSGVPDRFSGSGSATDFTLKISRVEAEDVGVYYCMQGSSWPYTFGQGTKLEIKRTVAAPSVF

SEQ ID NO: 316 - protein, 122 aa, synthetic construct
  REGION 1..122: note = Synthetic Construct
QVQLVQSGGGVVRPGGSLRLSCAASGFTFDDYGMSWVRQAPGKGLEWVSGISWNSGSIGYADSVKGRFTISRDNAKNSLYLQMNSLRAEDTALYYCAKEISDTAMGFDYWGQGTLVTVSSGS

SEQ ID NO: 317 - protein, 126 aa, synthetic construct
  REGION 1..126: note = Synthetic Construct
QSALTQPASVSGSPGQSITISCTGTSSDVGGYNYISWYQQYPGKAPKLMIYDVSKRPSGVSNRFSGSKSGNTASLTISGLQAEDEADYYCNSYTSSRTVVFGGGTKLTVLGQPKAAPSVTLFPPSS

SEQ ID NO: 318 - protein, 121 aa, synthetic construct
  REGION 1..121: note = Synthetic Construct
QVQLVQSGGGLVQPGGSLRLSCAASGFTFSNYWMYWVRQAPGKGLVWVSNINSDGSSTSYADSVKGRFTISRDNAKNSLYLQMNSLRDEDTAVYYCAREGYNAHYFDYWGQGTLVTVSSGS

SEQ ID NO: 319 - protein, 131 aa, synthetic construct
  REGION 1..131: note = Synthetic Construct
QSALTQPASVSGSPGQSITISCTGTSSDVGGYNYVSWYQQHPGKAPKLLIYDVTKRPSGVSNRFSGSKSGNTASLTISGLQAEDEADYYCSSYTSSSTVVFGGGTKLTVLGQPKAAPSVTLFPPSSKASGA

SEQ ID NO: 320 - protein, 121 aa, synthetic construct
  REGION 1..121: note = Synthetic Construct
QIQLVQSGGGVVQPGRSLRLSCAASGFTFSSYGMHWVRQAPGKGLEWVAVIWYDGSNKYYADSVKGRFTISRDNSKNTLYLQMNSLRAEDTAVYYCAKGLGYHPLFDYWGQGTLVTVSSGS

SEQ ID NO: 321 - protein, 126 aa, synthetic construct
  REGION 1..126: note = Synthetic Construct
DIVMTQSPDSLAVSLGERATINCKSSQSVLYSSDNKNYLAWYQQKPGQPPKLLIYWASTRESGVPDRFSGSGSGTDFTLTISSLQAEDVAVYYCQQYYSTPSFGPGTKVDIKRTVAAPSVFKASGA

SEQ ID NO: 322 - protein, 6 aa, synthetic construct
  REGION 1..6: note = Synthetic Construct
SSYGMH

SEQ ID NO: 323 - protein, 17 aa, synthetic construct
  REGION 1..17: note = Synthetic Construct
VISYDGSNKYYADSVKG

SEQ ID NO: 324 - protein, 17 aa, synthetic construct
  REGION 1..17: note = Synthetic Construct
DLLVRFLEWSHYYGMDV

SEQ ID NO: 325 - protein, 13 aa, synthetic construct
  REGION 1..13: note = Synthetic Construct
SGSSSNIGSNTVH

SEQ ID NO: 326 - protein, 7 aa, synthetic construct
  REGION 1..7: note = Synthetic Construct
SDNQRPS

SEQ ID NO: 327 - protein, 10 aa, synthetic construct
  REGION 1..10: note = Synthetic Construct
AAWDDSLNGV

SEQ ID NO: 328 - protein, 10 aa, synthetic construct
  REGION 1..10: note = Synthetic Construct
GFTFSSYGMH

SEQ ID NO: 329 - protein, 7 aa, synthetic construct
  REGION 1..7: note = Synthetic Construct
ISYDGSN

SEQ ID NO: 330 - protein, 8 aa, synthetic construct
  REGION 1..8: note = Synthetic Construct
SSNIGSNT

SEQ ID NO: 331 - skipped

SEQ ID NO: 332 - protein, 8 aa, synthetic construct
  REGION 1..8: note = Synthetic Construct
GFTFSSYA

SEQ ID NO: 333 - protein, 8 aa, synthetic construct
  REGION 1..8: note = Synthetic Construct
GFTFDDYG

SEQ ID NO: 334 - protein, 8 aa, synthetic construct
  REGION 1..8: note = Synthetic Construct
GFTFSNYW

SEQ ID NO: 335 - protein, 8 aa, synthetic construct
  REGION 1..8: note = Synthetic Construct
ISGGGGAT

SEQ ID NO: 336 - protein, 8 aa, synthetic construct
  REGION 1..8: note = Synthetic Construct
INSDGSST

SEQ ID NO: 337 - protein, 22 aa, synthetic construct
  REGION 1..22: note = Synthetic Construct
ASPAGHICSGGSCQYYYYGMDV

SEQ ID NO: 338 - protein, 19 aa, synthetic construct
  REGION 1..19: note = Synthetic Construct
ARDRHLYRRGYSSVDGMDV

SEQ ID NO: 339 - protein, 11 aa, synthetic construct
  REGION 1..11: note = Synthetic Construct
ARHPIGSGYDY

SEQ ID NO: 340 - protein, 13 aa, synthetic construct
  REGION 1..13: note = Synthetic Construct
AKEISDTAMGFDY

SEQ ID NO: 341 - protein, 12 aa, synthetic construct
  REGION 1..12: note = Synthetic Construct
AREGYNAHYFDY

SEQ ID NO: 342 - protein, 12 aa, synthetic construct
  REGION 1..12: note = Synthetic Construct
AKGLGYHPLFDY

SEQ ID NO: 343 - protein, 6 aa, synthetic construct
  REGION 1..6: note = Synthetic Construct
QGISSY

SEQ ID NO: 344 - protein, 7 aa, synthetic construct
  REGION 1..7: note = Synthetic Construct
QSVSSGY

SEQ ID NO: 345 - protein, 11 aa, synthetic construct
  REGION 1..11: note = Synthetic Construct
QSVVYRDGNTY

SEQ ID NO: 346 - protein, 12 aa, synthetic construct
  REGION 1..12: note = Synthetic Construct
QSVLYSSDNKNY

SEQ ID NO: 347-349 - skipped (3 entries)

SEQ ID NO: 350 - protein, 9 aa, synthetic construct
  REGION 1..9: note = Synthetic Construct
QQSYSTPLT

SEQ ID NO: 351 - protein, 9 aa, synthetic construct
  REGION 1..9: note = Synthetic Construct
QQRSIWPLT

SEQ ID NO: 352 - protein, 9 aa, synthetic construct
  REGION 1..9: note = Synthetic Construct
MQGSSWPYT

SEQ ID NO: 353 - protein, 10 aa, synthetic construct
  REGION 1..10: note = Synthetic Construct
NSYTSSRTVV

SEQ ID NO: 354 - protein, 10 aa, synthetic construct
  REGION 1..10: note = Synthetic Construct
SSYTSSSTVV

SEQ ID NO: 355 - protein, 8 aa, synthetic construct
  REGION 1..8: note = Synthetic Construct
QQYYSTPS

SEQ ID NO: 356 - protein, 116 aa, synthetic construct
  REGION 1..116: note = Synthetic Construct
EVQLLESGGGLVQPGGSLRLSCAASGFTFSSYAMSWVRQAPGKGLEWVSTISSGGSYTSYPDSVKGRFTISRDNSKNTLYLQMNSLRAEDTAVYYCAKQDYAMNYWGQGTLVTVSS

SEQ ID NO: 357 - protein, 107 aa, synthetic construct
  REGION 1..107: note = Synthetic Construct
DIQMTQSPSSLSASVGDRVTITCKASQDVSTAVAWYQQKPGKAPKLLIYSASYRYTGVPSRFSGSGSGTDFTLTISSLQPEDFATYYCQQHYSTPWTFGGGTKVEIK

SEQ ID NO: 358 - protein, 107 aa, synthetic construct
  REGION 1..107: note = Synthetic Construct
DIQMTQSPSSLSASVGDRVTITCKASQDVSTAVAWYQQKPGKAPKLLIYSASYRYTGVPSRFSGSGSGTDFTLTISSLQPEDFATYYCQQHYSTPWTFGQGTKVEIK

SEQ ID NO: 359 - protein, 5 aa, synthetic construct
  REGION 1..5: note = Synthetic Construct
SYAMS

SEQ ID NO: 360 - protein, 17 aa, synthetic construct
  REGION 1..17: note = Synthetic Construct
TISSGGSYTSYPDSVKG

SEQ ID NO: 361 - protein, 7 aa, synthetic construct
  REGION 1..7: note = Synthetic Construct
QDYAMNY

SEQ ID NO: 362 - protein, 11 aa, synthetic construct
  REGION 1..11: note = Synthetic Construct
KASQDVSTAVA

SEQ ID NO: 363 - protein, 7 aa, synthetic construct
  REGION 1..7: note = Synthetic Construct
SASYRYT

SEQ ID NO: 364 - protein, 9 aa, synthetic construct
  REGION 1..9: note = Synthetic Construct
QQHYSTPWT

SEQ ID NO: 365 - protein, 10 aa, synthetic construct
  REGION 1..10: note = Synthetic Construct
TISSGGSYTS

SEQ ID NO: 366 - protein, 10 aa, synthetic construct
  REGION 1..10: note = Synthetic Construct
GFTFSSYAMS

SEQ ID NO: 367 - protein, 330 aa, synthetic construct
  REGION 1..330: note = Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 368 - protein, 107 aa, synthetic construct [sequence omitted]

SEQ ID NO: 369 - protein, 446 aa, synthetic construct
  REGION 1..446: note = Synthetic Construct
EVQLLESGGGLVQPGGSLRLSCAASGFTFSSYAMSWVRQAPGKGLEWVSTISSGGSYTSYPDSVKGRFTISRDNSKNTLYLQMNSLRAEDTAVYYCAKQDYAMNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 370 - protein, 214 aa, synthetic construct
  REGION 1..214: note = Synthetic Construct
DIQMTQSPSSLSASVGDRVTITCKASQDVSTAVAWYQQKPGKAPKLLIYSASYRYTGVPSRFSGSGSGTDFTLTISSLQPEDFATYYCQQHYSTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 371 - protein, 120 aa, synthetic construct
  REGION 1..120: note = Synthetic Construct
QVQLVQSGAEVKKPGASVKVSCKASGYTFTSYWMQWVRQAPGQGLEWMGAIYPGDGDTRYTQKFKGRVTMTRDTSTSTVYMELSSLRSEDTAVYYCARMGGYDRYYFDYWGQGTLVTVSS

SEQ ID NO: 372 - protein, 113 aa, synthetic construct
  REGION 1..113: note = Synthetic Construct
DIVMTQSPDSLAVSLGERATINCKSSQSLLNSANQKNYLAWYQQKPGQPPKLLIYFASTRESGVPDRFSGSGSGTDFTLTISSLQAEDVAVYYCQQHYNTPLTFGGGTKVEIK

SEQ ID NO: 373 - protein, 120 aa, synthetic construct
  REGION 1..120: note = Synthetic Construct
QVQLQQSGAELARPGASVKLSCKASGYTFTSYWMQWVKQRPGQGLEWIGAIYPGDGDTRYTQKFKGKATLTADKSSSTAYMQLSSLASEDSAVYYCARMGGYDRYYFDYWGQGTTLTVSS

SEQ ID NO: 374 - protein, 113 aa, synthetic construct
  REGION 1..113: note = Synthetic Construct
DIVMTQSPSSLAMSVGQKVTMSCKSSQSLLNSANQKNYLAWYQQKPGQSPKLLVYFASTRESGVPDRFIGSGSGTDFTLTISSVQAEDLADYFCQQHYNTPLTFGAGTKLELK

SEQ ID NO: 375 - protein, 5 aa, synthetic construct
  REGION 1..5: note = Synthetic Construct
SYWMQ

SEQ ID NO: 376 - protein, 17 aa, synthetic construct
  REGION 1..17: note = Synthetic Construct
AIYPGDGDTRYTQKFKG

SEQ ID NO: 377 - protein, 13 aa, synthetic construct
  REGION 1..13: note = Synthetic Construct
ARMGGYDRYYFDY

SEQ ID NO: 378 - protein, 17 aa, synthetic construct
  REGION 1..17: note = Synthetic Construct
KSSQSLLNSANQKNYLA

SEQ ID NO: 379 - protein, 7 aa, synthetic construct
  REGION 1..7: note = Synthetic Construct
FASTRES

SEQ ID NO: 380 - protein, 9 aa, synthetic construct
  REGION 1..9: note = Synthetic Construct
QQHYNTPLT

SEQ ID NO: 381 - protein, 10 aa, synthetic construct
  REGION 1..10: note = Synthetic Construct
GYTFTSYWMQ

SEQ ID NO: 382 - protein, 9 aa, synthetic construct
  REGION 1..9: note = Synthetic Construct
AIYPGDGDT

SEQ ID NO: 383 - protein, 13 aa, synthetic construct
  REGION 1..13: note = Synthetic Construct
ARMGGYDRYYFDY

SEQ ID NO: 384 - protein, 17 aa, synthetic construct
  REGION 1..17: note = Synthetic Construct
KSSQSLLNSANQKNYLA

SEQ ID NO: 385 - protein, 7 aa, synthetic construct
  REGION 1..7: note = Synthetic Construct
FASTRES

SEQ ID NO: 386 - protein, 9 aa, synthetic construct
  REGION 1..9: note = Synthetic Construct
QQHYNTPLT

SEQ ID NO: 387 - protein, 118 aa, synthetic construct
  REGION 1..118: note = Synthetic Construct
QVQLVESGGGVVQPGRSLRLSCAASGFTFSRYGIHWVRQAPGKGLEWVAVISYDGSDEYYVDSVKGRFSISRDNSKNTLYLQMNSLRPADSAVYYCVKGDLELGFDYWGQGTLVTVSS

SEQ ID NO: 388 - protein, 112 aa, synthetic construct
  REGION 1..112: note = Synthetic Construct
DIVMTQAAPSIPVIPGESVSMSCRSSKSLLYSNGHTYVYWFVQRPGQSPQLLIYRMSNLASGVPDRFSGSGSGTAFTLRISRVEAEDVGVYYCMQNLEFPLTFGAGTKLELK

SEQ ID NO: 389 - protein, 120 aa, synthetic construct; identical to SEQ ID NO: 164
  REGION 1..120: note = Synthetic Construct

SEQ ID NO: 390 - protein, 107 aa, synthetic construct; identical to SEQ ID NO: 165
  REGION 1..107: note = Synthetic Construct

SEQ ID NO: 391 - protein, 121 aa, synthetic construct
  REGION 1..121: note = Synthetic Construct
QVQLVESGGGVVQPGKSLRLSCAASGFTFSSFGMHWVRQAPGKGLEWVAVIGYDGGNEYYADSVKGRFTISRDNSKNTLNLQMSSLRAEDTAVYYCSTISHYDILSGMDVWGRGTTVTVSS

SEQ ID NO: 392 - protein, 108 aa, synthetic construct
  REGION 1..108: note = Synthetic Construct
DIQMTQSPSSVSASVGDRVTITCRASQGISNWLAWYQQKPGKAPKLLIFAASSLQSGVPSRFSGSASGTDFTLTINSLQPEDFATYYCQQANSFPLTFGGGTKVEIKR

SEQ ID NO: 393 - protein, 8 aa, synthetic construct
  REGION 1..8: note = Synthetic Construct
GFTFSAYA

SEQ ID NO: 394 - protein, 8 aa, synthetic construct
  REGION 1..8: note = Synthetic Construct
ISGSGGSA

SEQ ID NO: 395 - protein, 13 aa, synthetic construct
  REGION 1..13: note = Synthetic Construct
AKDGAWKMSGLDV

SEQ ID NO: 396 - protein, 6 aa, synthetic construct
  REGION 1..6: note = Synthetic Construct
QDISDY

SEQ ID NO: 397 - skipped

SEQ ID NO: 398 - protein, 9 aa, synthetic construct
  REGION 1..9: note = Synthetic Construct
QKYDSAPLT

SEQ ID NO: 399 - protein, 8 aa, synthetic construct
  REGION 1..8: note = Synthetic Construct
GFTFSSFG

SEQ ID NO: 400 - protein, 8 aa, synthetic construct
  REGION 1..8: note = Synthetic Construct
IGYDGGNE

SEQ ID NO: 401 - protein, 14 aa, synthetic construct
  REGION 1..14: note = Synthetic Construct
STISHYDILSGMDV

SEQ ID NO: 402 - protein, 6 aa, synthetic construct
  REGION 1..6: note = Synthetic Construct
QGISNW

SEQ ID NO: 403 - skipped

SEQ ID NO: 404 - protein, 9 aa, synthetic construct
  REGION 1..9: note = Synthetic Construct
QQANSFPLT

SEQ ID NO: 405 - protein, 122 aa, synthetic construct
  REGION 1..122: note = Synthetic Construct
QVQLVQSGGGLVQPGGSLRLSCAASGFTFNTYAISWVRQAPGKGLEWVSTITGSGYNTYYADSVKGRFTISRDNSKNTLYLQMSSLRAEDTAVFYCAKDSRYNWNYGIFDYWGQGTTVTVSS

SEQ ID NO: 406 - protein, 108 aa, synthetic construct
  REGION 1..108: note = Synthetic Construct
DIQMTQSPATLSVSPGERATLSCRASQSVSSNLAWYHQKPGQAPRLLIYGVSTRATGIPARFSGNGSGTEFTLTISSLQSEDFAVYYCQQHNNWPLTFGGGTKVEIKR

SEQ ID NO: 407 - protein, 125 aa, synthetic construct
  REGION 1..125: note = Synthetic Construct
QVQLVQSGPGLVKPSETLSLTCTVSGGSISNSNYYWGWIRQPPGKGLEWIGTTYYSGTTYYNPSLKSRVTISVDTSKNQFSLKLSSVTAADTAVYYCARDYYDSSGYYYNWFDPWGQGTTVTVSS

SEQ ID NO: 408 - protein, 108 aa, synthetic construct
  REGION 1..108: note = Synthetic Construct
DIQLTQSPSSLSASVGDRVTITCRASQDIRNDLGWYQQKPGKAPKRLIYAASSLQSGVPSRFSGSGSGTEFSLTLSSLQPEDFATYFCLQHHIYPWTFGQGTKLEIKR

SEQ ID NO: 409 - protein, 125 aa, synthetic construct
  REGION 1..125: note = Synthetic Construct
QVQLVESGPGLVKPSETLSLTCTVYGGSISSGNYYWGWIRQPPGKGLEWIGTIYYSGSAYYNPSLKSRVTMSVDTSKNQFSLKLSSVTAADTAVYYCVRDYYDSSGHYYNWFDPWGQGTTVTVSS

SEQ ID NO: 410 - protein, 108 aa, synthetic construct
  REGION 1..108: note = Synthetic Construct
AIQMTQSPSSLSASVGDRVTITCRASQDIRHDLGWYQQKPGKAPKRLIYAASSLQSGVPSRFSGSGSGTEFTLTISSLQPEDFATYYCLQHNTYPWTFGQGTKVEIKR

SEQ ID NO: 411 - protein, 119 aa, synthetic construct
  REGION 1..119: note = Synthetic Construct
QVQLVQSGGGLVKPGGSLRLSCAASGFTFSSYSMNWVRQAPGKGLEWVSSISSSSSYIYYADSVKGRFTISRDNAKNSLYLQMNSLRAEDTAVYYCARDRGYTFGVDYWGQGTTVTVSS

SEQ ID NO: 412 - protein, 108 aa, synthetic construct
  REGION 1..108: note = Synthetic Construct
AIQMTQSPSSLSASVGDRVTITCRASQGIRNDLGWYQQKPGKAPKRLIYAASSLQSGVPSRFSGSGSGTEFTLTISSLQPEDFATYYCLQHNSYPYTFGQGTKVEIKR

SEQ ID NO: 413 - protein, 125 aa, synthetic construct
  REGION 1..125: note = Synthetic Construct
QVQLVQSGPGLVKPSETLSLTCTVSGGSIITYSYYWGWIRQPPGKGLEWIGTIHHSGSTYYNPSLKSRVTISVDTSKNQFSLTLSSVTAADTAVYYCARDYYDSSGYYYNWFDPWGQGTMVTVSS

SEQ ID NO: 414 - protein, 108 aa, synthetic construct
  REGION 1..108: note = Synthetic Construct
AIQMTQSPSSLSASVGDRVTITCRASQGIRNDLGWYQQKPGKAPKRLIYAASSLQSGVPSRFSGSGSGTEFTLTISSLQPEDFATYYCLQHNSSPWTFGQGTKVEIKR

SEQ ID NO: 415 - protein, 123 aa, synthetic construct
  REGION 1..123: note = Synthetic Construct
QVHLKESGPTLVKPTQTLTLTCTFSGFSLSTSGVGVGWIRQPPGKALEWLALIYWNDDKRYSPSLKSRLTITKDTSKNQVVLTMTNMDPVDTATYYCTHTSRYNWHYGFLDYWGQGTTVTVSS

SEQ ID NO: 416 - protein, 108 aa, synthetic construct
  REGION 1..108: note = Synthetic Construct
AIQMTQSPSSLSASVGDRVTITCRASQGISNYLAWFQQKPGKAPKSLIYAASSLQSGVPSKFSGSGSGTDFTLTISSLQPEDFATYYCQQYNSYPLTFGGGTKVEIKR

SEQ ID NO: 417 - protein, 125 aa, synthetic construct
  REGION 1..125: note = Synthetic Construct
EVQLVQLGPGLVKPSETLSLTCTVSGGSISNSNYYWGWIRQPPGKGLEWIGTTYYSGTTYYNPSLKSRVTISVDTSKNQFSLKLSSVTAADTAVYYCARDYYDSSGYYYNWFDPWGQGTTVTVSS

SEQ ID NO: 418 - protein, 108 aa, synthetic construct
  REGION 1..108: note = Synthetic Construct
AIQMTQSPSSLSASVGDRVTITCRASQDIRNDLGWYQQKPGKAPKRLIYAASSLQSGVPSRFSGSGSGTEFSLTLSSLQPEDFATYFCLQHHIYPWTFGQGTKVEIKR

SEQ ID NO: 419 - protein, 118 aa, synthetic construct
  REGION 1..118: note = Synthetic Construct
QVQLVEAGGGVVQPGRSLRLSCAASGFTFSRYGIHWVRQAPGKGLEWVAVISYDGTDEYYADSVKGRFTISRDNSKNTLYLQMNSLRPADSAVYYCAKGDLELGFDYWGQGTLVTVSS

SEQ ID NO: 420 - protein, 107 aa, synthetic construct
  REGION 1..107: note = Synthetic Construct
AIRLTQSPSSVSASVGDRVTITCRASQDISIWLAWYQQSPGKAPKLLINVASRLQSGVPSRFSGSGSGTDFTLTINGLQPEDFVTYYCQQANSFPITFGQGTRLATK

SEQ ID NO: 421 - protein, 119 aa, synthetic construct
  REGION 1..119: note = Synthetic Construct
QVQLVQSGGGLVKPGGSLRLSCAASGFIFNTYTMNWVRQAPGKGLEWVSSITSRGTYIFYSDSLKGRFTISRDNANNSLFLQMNSLRVEDTAVYYCSRDRGYTFGPDYWGQGTLVTVSS

SEQ ID NO: 422 - protein, 107 aa, synthetic construct
  REGION 1..107: note = Synthetic Construct
DIQMTQSPSSLSASVGDRVTITCRASQDIRNDLGWYQQKPGKAPKGLIYAASSLQSGVPSRFSGSGSGTEFTLTISSLQPEDFATYHCLHYDFHPRTFGQGTKVEIK

SEQ ID NO: 423 - protein, 130 aa, synthetic construct
  REGION 1..130: note = Synthetic Construct
QVQLVESGGGVVQPGRSLRLSCAASGFTFSSYAMHWVRQAPGKGLEWVAVIWYDGTNYYYADSVKGRFTISRDNSKNTLYLQMNSLRAEDTAVYYCARDPLYYDILTGYSPDYYYGMDVWGQGTTVTVSS

SEQ ID NO: 424 - protein, 107 aa, synthetic construct
  REGION 1..107: note = Synthetic Construct
EIVMTQSPATLSVSPGERATLSCRASQTFSSNLAWYQQKPGQAPRLLIYGASTRATGIPARFSGSGSGTEFTLTISSLQSEDFAVYYCQQYNKWPLTFGGGTKVEIK

SEQ ID NO: 425 - protein, 122 aa, synthetic construct
  REGION 1..122: note = Synthetic Construct
EVQLLESGGGLVQPGESLRLSCAASGFTFSSYAMNWVRQAPGKGLEWVSTISGSGGYIYYADSVKGRFTISRDNSKNTLYLQMNSLRAEDTAVYFCAKDSRYNWNYGNFDYWGQGTLVTVSS

SEQ ID NO: 426 - protein, 107 aa, synthetic construct
  REGION 1..107: note = Synthetic Construct
EIVMTQSPATLSVSPGERATLSCRASQNVSSNLAWNKQKPGQAPRLLIYATSTRATGVPARFSASGSGTDFALTINSLQSEDFAVYYCQQYNNWPLTFGGGTKVEIK

SEQ ID NO: 427 - protein, 122 aa, synthetic construct
  REGION 1..122: note = Synthetic Construct
EVQLLESGGGLVQPGGSLRLSCVASRFTFSSNAMSWVRQAPGTGLEWVSAITGSGSRTYYADSVKGRFTISRDNSKNTVYLQMNSLRGEDTAVYYCAKDQGGTWNYGDFDYWGQGTLVTVSS

SEQ ID NO: 428 - protein, 107 aa, synthetic construct
  REGION 1..107: note = Synthetic Construct
EIVMTQSPATLSVSPGERATLSCRASQSVSSNLAWYQQKPGQAPRLLIYGASTRATGLPARFSGSGSGTDFTLTISSLQSEDFAVYYCQQYNNWPLTFGGGTKVEIK

SEQ ID NO: 429 - protein, 124 aa, synthetic construct
  REGION 1..124: note = Synthetic Construct
QVQLVESGGDVVQPGRSLRLSCTASGFTFSSYGMHWVRQAPGRGLEWVAVISFDGKNKYYADSVKGRFTVSRDNSKNTLFLQMNSLRAEDTALYYCAKRIAATGYYYFYGLDVWGQGTTVTVSS

SEQ ID NO: 430 - protein, 107 aa, synthetic construct
  REGION 1..107: note = Synthetic Construct
EIMMTQSPATLSVSPGERGTLSCRASQSVSSNLAWYQQKPGQAPRLLIYGASTRASGIPARFSGSGSGTEFTLTVSSLQSEDFAVYYCQQYNNWPLTFGGGTKVEIK

SEQ ID NO: 431 - protein, 122 aa, synthetic construct
  REGION 1..122: note = Synthetic Construct
EVQLLESGGGLVQPGGSLRLSCAASGFTSITYAMSWVRQAPGKGLEWVSAISVSGTNTYYADSVKGRFTISRDKSKNMLYLQMNSLRAEDTAVYYCAKDLLHNWKYGTFDIWGQGTMVTVSS

SEQ ID NO: 432 - protein, 107 aa, synthetic construct
  REGION 1..107: note = Synthetic Construct
EIVMTQSPATLSVSPGERATLSCRASQSVDSNLVWYQQKPGQVPRLLIYGASTRATGIPARFSGSGSGTEFTLTISSLQSEDFAVYYCQQYNKWPLTFGGGTKVEIK

SEQ ID NO: 433 - protein, 122 aa, synthetic construct; identical to SEQ ID NO: 431
  REGION 1..122: note = Synthetic Construct

SEQ ID NO: 434 - protein, 108 aa, synthetic construct
  REGION 1..108: note = Synthetic Construct
EIVMTQSPATLSVSPGERATLSCRASQSVSSNLAWNQQKPGQAPRLLIYGASTRATGIPARFSGSGSGTEFTLTISSLQSEDFAVYYCQQYNNWPMYTFGQGTKLEIK

SEQ ID NO: 435 - protein, 120 aa, synthetic construct
  REGION 1..120: note = Synthetic Construct
QVQLVESGGGVVQPGRSLRLSCEASGFTFSSSGMHWVRQAPGKGLQWVAVISYDGNNKFYEDSVKGRLTISRDNSNNTLWLQMNSLRVEDTAVYYCAKSGGRVGAAFAYWGQGTLVTVSS

SEQ ID NO: 436 - protein, 112 aa, synthetic construct
  REGION 1..112: note = Synthetic Construct
DIVNTQSPLSLPVTPGEPASISCRSSQSLLYGNGYNYLDWYLQKPGHSPQLLIYLGSNRGSGVPDRFSGSGSGTDFTLKISRVEAEDVGIYYCMQTLQTPFTFGPGTKMYIK

SEQ ID NO: 437 - protein, 125 aa, synthetic construct
  REGION 1..125: note = Synthetic Construct
EVQLLESGGGLVQPGGSLRLSCAGSGITFSSYAMSWVRQAPGKGLEWVSAISGNGGTTNYADSVKGRFTISRDNSKNTLYLQMNSLRAEDTAVYYCAKERILTSSWTRYGIMDVWGQGTTVTVSS

SEQ ID NO: 438 - protein, 107 aa, synthetic construct
  REGION 1..107: note = Synthetic Construct
EIVMTQSPATLSMSPGERATLSCRASQSVSSNLAWYQQKPGQAPRLLIYGASTRATGIPARFSGSGSGTEFTLTISSLQSEDFAVYYCQQYNNWPLTFGGGTKLEIK

SEQ ID NO: 439 - protein, 119 aa, synthetic construct
  REGION 1..119: note = Synthetic Construct
QVQLQQWGAGLLKPSETLSLTCAVYGGSFSGYYWSWIRQPPGKGLEWIGEINHSGNTNYNPSLKSRVTISVDTSKNQFSLKLNSVTAADTAVYYCARREATVTPYFDYWGQGTLVTVSS

SEQ ID NO: 440 - protein, 107 aa, synthetic construct
  REGION 1..107: note = Synthetic Construct
DIQLTQSPSFLSASVGDRVTITCWASQGISSYLAWYQQKPGKAPKLLIYAASTLQSGVPSRFSGSGSGTEFTLTISSLQPEDFATYYCQQLNSYPLTFGGGTKVEIK

SEQ ID NO: 441 - protein, 118 aa, synthetic construct
  REGION 1..118: note = Synthetic Construct
QVQLVESGGGVVQPGRSLRLSCVVSGFNFSRNGIHWVRQAPGKGLEWVAVISYDGRNKFYVESVKGRFTISRDNSKNTLYLQMNSLRVEDTAVYYCAKSSIGGFFEYWGQGTLVTVSS

SEQ ID NO: 442 - protein, 112 aa, synthetic construct
  REGION 1..112: note = Synthetic Construct
DIVMTQSPLSLPVTPGEPASISCRSSQSLLHSNGYNYLDWYLQKPGQSPQLMIYLGSHRASGVPDRFSGSGSGTDFTLKISRVEAEDVGVYYCIQVQQTPITFGQGTRLEIK

SEQ ID NO: 443 - protein, 112 aa, synthetic construct
  REGION 1..112: note = Synthetic Construct
DIVMTQTPLSSPVTLGQPASISCRSSQSLVHSDGNTYLSWLQQRPGQPPRLLIYKISNRFSGVPDRFSGSGAGTDFTLKISRVEAEDVGVYFCMQATQFPYTFGQGTKLEIK

SEQ ID NO: 444 - protein, 116 aa, synthetic construct
  REGION 1..116: note = Synthetic Construct
EVQLLESGGGLVQPGGSLRLSCAASGFTFSSYAMSWVRQAPGKGLEWVSTISSGGSYTSYPDSVKGRFTISRDNSKNTLYLQMNSLRAEDTAVYYCAKQDYAMNYWGQGTMVTVSS

SEQ ID NO: 445 - protein, 107 aa, synthetic construct; identical to SEQ ID NO: 358
  REGION 1..107: note = Synthetic Construct

SEQ ID NO: 446 - protein, 116 aa, synthetic construct
  REGION 1..116: note = Synthetic Construct
EVQLVESGGGLVKPGGSLKLSCAASGFTFSSYAMSWVRQTPEKRLEWVATISSGGSYTSYPDSVKGRFTISRDNAKNTLYLQMSSLRSEDTAMYYCARQDYAMNYWGQGTSVTVSS

SEQ ID NO: 447 - protein, 107 aa, synthetic construct
  REGION 1..107: note = Synthetic Construct
DIEMTQSHKFMSTSVGDRVSITCKASQDVSTAVAWYQQKPGQSPKLLLYSASYRYTGVPDRFTGSGSGTDFTFTISSVQAEDLAVYYCQQHYSTPWTFGGGTKLEIK

SEQ ID NO: 448 - protein, 119 aa, synthetic construct
  REGION 1..119: note = Synthetic Construct
QVTLKESGPVLVKPTETLTLTCTVSGIDLSSYDISWVRQAPGKGLEWVGIISYAGSTYYASWAKGRLTISKDTSKNQVVLTMTNMDPVDTATYYCARGVPAYSTGGDLWGQGTLVTVSS

SEQ ID NO: 449 - protein, 119 aa, synthetic construct
  REGION 1..119: note = Synthetic Construct
QVTLKESGPVLVKPTETLTLTCTVSGIDLSHYDISWVRQAPGKGLEWVGIISYAGSTYYASWAKGRLTISKDTSKNQVVLTMTNMDPVDTATYYCARGVPAYSTGGDLWGQGTLVTVSS

SEQ ID NO: 450 - protein, 119 aa, synthetic construct
  REGION 1..119: note = Synthetic Construct
QVTLKESGPVLVKPTETLTLTCTVSGIDLSSYDISWVRQAPGKGLEWVGIISHAGSTYYASWAKGRLTISKDTSKNQVVLTMTNMDPVDTATYYCARGVPAYSTGGDLWGQGTLVTVSS

SEQ ID NO: 451 - protein, 119 aa, synthetic construct
  REGION 1..119: note = Synthetic Construct
QVTLKESGPVLVKPTETLTLTCTVSGIDLSSYDISWVRQAPGKGLEWVGIISYAGSTYYASWAKGRLTISKDTSKNQVVLTMTNMDPVDTATYYCARGVPAHSTGGDLWGQGTLVTVSS

SEQ ID NO: 452 - protein, 119 aa, synthetic construct
  REGION 1..119: note = Synthetic Construct
QVTLKESGPVLVKPTETLTLTCTVSGIDLSSYDISWVRQAPGKGLEWVGIISYAGSTYYASWAKGRLTISKDTSKNQVVLTMTNMDPVDTATYYCARGVPAYSHGGDLWGQGTLVTVSS

SEQ ID NO: 453 - protein, 119 aa, synthetic construct
  REGION 1..119: note = Synthetic Construct
QVTLKESGPVLVKPTETLTLTCTVSGIDLSHYDISWVRQAPGKGLEWVGIISHAGSTYYASWAKGRLTISKDTSKNQVVLTMTNMDPVDTATYYCARGVPAYSTGGDLWGQGTLVTVSS

SEQ ID NO: 454 - protein, 119 aa, synthetic construct
  REGION 1..119: note = Synthetic Construct
QVTLKESGPVLVKPTETLTLTCTVSGIDLSSYDISWVRQAPGKGLEWVGIISHAGSTYYASWAKGRLTISKDTSKNQVVLTMTNMDPVDTATYYCARGVPAHSTGGDLWGQGTLVTVSS

SEQ ID NO: 455 - protein, 119 aa, synthetic construct
  REGION 1..119: note = Synthetic Construct
QVTLKESGPVLVKPTETLTLTCTVSGIDLSHYDISWVRQAPGKGLEWVGIISYAGSTYYASWAKGRLTISKDTSKNQVVLTMTNMDPVDTATYYCARGVPAHSTGGDLWGQGTLVTVSS

SEQ ID NO: 456 - protein, 119 aa, synthetic construct
  REGION 1..119: note = Synthetic Construct
QVTLKESGPVLVKPTETLTLTCTVSGIDLSHYDISWVRQAPGKGLEWVGIISHAGSTYYASWAKGRLTISKDTSKNQVVLTMTNMDPVDTATYYCARGVPAHSTGGDLWGQGTLVTVSS

SEQ ID NO: 457 - protein, 119 aa, synthetic construct
  REGION 1..119: note = Synthetic Construct
QVTLKESGPVLVKPTETLTLTCTVSGIDLSHYDISWVRQAPGKGLEWVGIISYAGSTYYASWAKGRLTISKDTSKNQVVLTMTNMDPVDTATYYCARGVPAYSHGGDLWGQGTLVTVSS

SEQ ID NO: 458 - protein, 119 aa, synthetic construct
  REGION 1..119: note = Synthetic Construct
QVTLKESGPVLVKPTETLTLTCTVSGIDLSSYDISWVRQAPGKGLEWVGIISHAGSTYYASWAKGRLTISKDTSKNQVVLTMTNMDPVDTATYYCARGVPAYSHGGDLWGQGTLVTVSS

SEQ ID NO: 459 - protein, 119 aa, synthetic construct
  REGION 1..119: note = Synthetic Construct
QVTLKESGPVLVKPTETLTLTCTVSGIDLSSYDISWVRQAPGKGLEWVGIISYAGSTYYASWAKGRLTISKDTSKNQVVLTMTNMDPVDTATYYCARGVPAHSHGGDLWGQGTLVTVSS

SEQ ID NO: 460 - protein, 119 aa, synthetic construct
  REGION 1..119: note = Synthetic Construct
QVTLKESGPVLVKPTETLTLTCTVSGIDLSHYDISWVRQAPGKGLEWVGIISHAGSTYYASWAKGRLTISKDTSKNQVVLTMTNMDPVDTATYYCARGVPAYSHGGDLWGQGTLVTVSS

SEQ ID NO: 461 - protein, 119 aa, synthetic construct
  REGION 1..119: note = Synthetic Construct
QVTLKESGPVLVKPTETLTLTCTVSGIDLSSYDISWVRQAPGKGLEWVGIISHAGSTYYASWAKGRLTISKDTSKNQVVLTMTNMDPVDTATYYCARGVPAHSHGGDLWGQGTLVTVSS

SEQ ID NO: 462 - protein, 119 aa, synthetic construct
  REGION 1..119: note = Synthetic Construct
QVTLKESGPVLVKPTETLTLTCTVSGIDLSHYDISWVRQAPGKGLEWVGIISYAGSTYYASWAKGRLTISKDTSKNQVVLTMTNMDPVDTATYYCARGVPAHSHGGDLWGQGTLVTVSS

SEQ ID NO: 463 - protein, 119 aa, synthetic construct
  REGION 1..119: note = Synthetic Construct
QVTLKESGPVLVKPTETLTLTCTVSGIDLSHYDISWVRQAPGKGLEWVGIISHAGSTYYASWAKGRLTISKDTSKNQVVLTMTNMDPVDTATYYCARGVPAHSHGGDLWGQGTLVTVSS

SEQ ID NO: 464 - protein, 119 aa, synthetic construct
  REGION 1..119: note = Synthetic Construct
QVTLKESGPVLVKPTETLTLTCTVSGIDLSHTDISWVRQAPGKGLEWVGIISYAGSTYYASWAKGRLTISKDTSKNQVVLTMTNMDPVDTATYYCARGVPAYSHGGDLWGQGTLVTVSS

SEQ ID NO: 465 - protein, 119 aa, synthetic construct
  REGION 1..119: note = Synthetic Construct
QVTLKESGPVLVKPTETLTLTCTVSGIDLSHTDISWVRQAPGKGLEWVGIISYAGSTYYMSWAKGRLTISKDTSKNQVVLTMTNMDPVDTATYYCARGVPAYSHGGDLWGQGTLVTVSS

SEQ ID NO: 466 - protein, 119 aa, synthetic construct; identical to SEQ ID NO: 457
  REGION 1..119: note = Synthetic Construct

SEQ ID NO: 467 - protein, 119 aa, synthetic construct
  REGION 1..119: note = Synthetic Construct
QVTLKESGPVLVKPTETLTLTCTVSGIDLSHDDISWVRQAPGKGLEWVGIISYAGSTYYASWAKGRLTISKDTSKNQVVLTMTNMDPVDTATYYCARGVPAYSHGGDLWGQGTLVTVSS

SEQ ID NO: 468 - protein, 119 aa, synthetic construct
  REGION 1..119: note = Synthetic Construct
QVTLKESGPVLVKPTETLTLTCTVSGIDLSHDDISWVRQAPGKGLEWVGIISYAGSMYYASWAKGRLTISKDTSKNQVVLTMTNMDPVDTATYYCARGVPAYSHGGDKWGQGTLVTVSS

SEQ ID NO: 469 - protein, 119 aa, synthetic construct
  REGION 1..119: note = Synthetic Construct
QVTLKESGPVLVKPTETLTLTCTVSGIDLSHTDISWVRQAPGKGLEWVGIISYAGKTYYASWAKKRLTISKDTSKNQVVLTMTNMDPVDTATYYCARGVPAYSHGGDLWGQGTLVTVSS

SEQ ID NO: 470 - protein, 119 aa, synthetic construct
  REGION 1..119: note = Synthetic Construct
QVTLKESGPVLVKPTETLTLTCTVSGIDLSHTDISWVRQAPGKGLEWVGIISYAGKKYYASWAKGRLTISKDTSKNQVVLTMTNMDPVDTATYYCARGVPAYSHGGDLWGQGTLVTVSS

SEQ ID NO: 471 - protein, 119 aa, synthetic construct
  REGION 1..119: note = Synthetic Construct
QVTLKESGPVLVKPTETLTLTCTVSGIDLSHTDISWVRQAPGKGLEWVGIISYAGSKYKASWAKGRLTISKDTSKNQVVLTMTNMDPVDTATYYCARGVPAYSHGGDLWGQGTLVTVSS

SEQ ID NO: 472 - protein, 119 aa, synthetic construct
  REGION 1..119: note = Synthetic Construct
QVTLKESGPVLVKPTETLTLTCTVSGIDLSHDDISWVRQAPGKGLEWVGIISYAGKKYYASWAKGRLTISKDTSKNQVVLTMTNMDPVDTATYYCARGVPAYSHGGDLWGQGTLVTVSS

SEQ ID NO: 473 - protein, 119 aa, synthetic construct
  REGION 1..119: note = Synthetic Construct
QVQLVESGGGLVQPGGSLRLSCAVSGIDLSHDDISWVRQAPGKGLEWVSIISYAGSTYYASWAKGRLTISKDTSKNQVVLTMTNMDPVDTATYYCARGVPAYSHGGDLWGQGTLVTVSS

SEQ ID NO: 474 - protein, 119 aa, synthetic construct
  REGION 1..119: note = Synthetic Construct
QVTLKESGPVLVKPTETLTLTCKVSGIDLSHDDISWVRQAPGKGLEWVSIISYAGSTYYASWAKGRLTISKDTSKNQVVLTMTNMDPVDTATYYCARGVPAYSHGGDLWGQGTLVTVSS

SEQ ID NO: 475 - protein, 119 aa, synthetic construct
  REGION 1..119: note = Synthetic Construct
QVTLKESGPVLVKPTETLTLTCKVSGIDLSHEDISWVRQAPGKGLEWVSIISYAGSTYYASWAKGRLTISKDTSKNQVVLTMTNMDPVDTATYYCARGVPAYSHGGDLWGQGTLVTVSS

SEQ ID NO: 476 - protein, 119 aa, synthetic construct
  REGION 1..119: note = Synthetic Construct
QVQLVESGGGLVQPGGSLRLSCAVSGIDLSHEDISWVRQAPGKGLEWVSIISYAGSKYYASWAKGRLTISKDTSKNQVVLTMTNMDPVDTATYYCARGVPAYSHGGDLWGQGTLVTVSS

SEQ ID NO: 477 - protein, 110 aa, synthetic construct
  REGION 1..110: note = Synthetic Construct
DIVMTQSPATLSLSPGERATLSCQSSQSVYDNNWLSWFQQKPGQPPKLLIYWASTLASGVPSRFSGSGSGTDFTLTISSLQPEDAATYYCAGGYGGGLYAFGQGTKVEIK

SEQ ID NO: 478 - protein, 110 aa, synthetic construct
  REGION 1..110: note = Synthetic Construct
DIVMTQSPATLSLSPGERATLSCQSSQSVYHNNWLSWFQQKPGQPPKLLIYWASTLASGVPSRFSGSGSGTDFTLTISSLQPEDAATYYCAGGYGGGLYAFGQGTKVEIK

SEQ ID NO: 479 - protein, 110 aa, synthetic construct
  REGION 1..110: note = Synthetic Construct
DIVMTQSPATLSLSPGERATLSCTSSQSVYHNNWLSWFQQKPGQPPKLLIYWASTLAYGVPSRFSGSGSGTDFTLTISSLQPEDAATYYCAGGYGGGRYAFGQGTKVEIK

SEQ ID NO: 480 - protein, 110 aa, synthetic construct
  REGION 1..110: note = Synthetic Construct
DIVMTQSPATLSLSPGERATLSCQSSQSVYHNNWLSWFQQKPGQPPKLLIYWASTLAFGVPSRFSGSGSGTDFTLTISSLQPEDAATYYCAGGYGGGLYAFGQGTKVEIK

SEQ ID NO: 481 - protein, 110 aa, synthetic construct
  REGION 1..110: note = Synthetic Construct
DIVMTQSPATLSLSPGERATLSCQTSQSVYHDNWLSWFQQKPGQPPKLLIYWASTLASGVPSRFSGSGSGTDFTLTISSLQPEDAATYYCAGGYGGGRYAFGQGTKVEIK

SEQ ID NO: 482 - protein, 110 aa, synthetic construct
  REGION 1..110: note = Synthetic Construct
DIVMTQSPATLSLSPGERATLSCTSSQSVYHDNWLSWFQQKPGQPPKLLIYWASTLAYGVPSRFSGSGSGTDFTLTISSLQPEDAATYYCAGGYGGGLYAFGQGTKVEIK

SEQ ID NO: 483 - protein, 110 aa, synthetic construct
  REGION 1..110: note = Synthetic Construct
DIVMTQSPATLSLSPGERATLSCTSSQSVYHANWLSWFQQKPGQPPKLLIYWASTLAYGVPSRFSGSGSGTDFTLTISSLQPEDAATYYCAGGYGGGRYAFGQGTKVEIK

SEQ ID NO: 484 - protein, 110 aa, synthetic construct
  REGION 1..110: note = Synthetic Construct
DIVMTQSPATLSLSPGERATLSCTTSQSVYHENWLSWFQQKPGQPPKLLIYWASTLAYGVPSRFSGSGSGTDFTLTISSLQPEDAATYYCAGGYGGGRYAFGQGTKVEIK

SEQ ID NO: 485 - protein, 110 aa, synthetic construct
  REGION 1..110: note = Synthetic Construct
DIVMTQSPATLSLSPGERATLSCTSSQSVFHENWLSWFQQKPGQPPKLLIYWASTLAWGVPSRFSGSGSGTDFTLTISSLQPEDAATYYCAGGYGGGRYAFGQGTKVEIK

SEQ ID NO: 486 - protein, 110 aa, synthetic construct
  REGION 1..110: note = Synthetic Construct
DIVMTQSPATLSLSPGERATLSCTTSQSVYHENWLSWYQQKPGQPPKLLIYWASTLAYGVPSRFSGSGSGTDFTLTISSLQPEDVATYYCAGGYGGGRYAFGQGTKVEIK

SEQ ID NO: 487 - protein, 5 aa, synthetic construct
  REGION 1..5: note = Synthetic Construct
HDDIS

SEQ ID NO: 488 - protein, 16 aa, synthetic construct
  REGION 1..16: note = Synthetic Construct
IISYAGSTYYASWAKG

SEQ ID NO: 489 - protein, 11 aa, synthetic construct
  REGION 1..11: note = Synthetic Construct
GVPAYSHGGDL

SEQ ID NO: 490 - protein, 13 aa, synthetic construct
  REGION 1..13: note = Synthetic Construct
TSSQSVYHANWLS

SEQ ID NO: 491 - protein, 7 aa, synthetic construct
  REGION 1..7: note = Synthetic Construct
WASTLAY

SEQ ID NO: 492 - protein, 10 aa, synthetic construct
  REGION 1..10: note = Synthetic Construct
AGGYGGGRYA

SEQ ID NO: 493 - protein, 13 aa, synthetic construct
  REGION 1..13: note = Synthetic Construct
TTSQSVYHENWLS

SEQ ID NO: 494 - protein, 157 aa, synthetic construct
  REGION 1..157: note = Synthetic Construct
MKCSWIMFFLVATATGVHSQVQLQQPGAELVKPGASVKLSCKASGYTFTNYWMYWVKQRPGQGLEWIGMIHPNSGSTNYNGKFKTGATLTVDKSSSTVYMQLSSLTSEDSAVYYCARWGYGGNYDYAMDYWGQGTSVTVSSAKTTPPSVYPLAPGSL

SEQ ID NO: 495 - protein, 165 aa, synthetic construct
  REGION 1..165: note = Synthetic Construct
MDFQVQIFSFLLISASVIMSRGQIVLTQSPAIMSASLGERVTMTCTASSSVSSSYFHWYQQKPGSSPKLWIYSTSNLASGVPARFSGSGSGTSYSLTISTMEAEDAVTYYCHQYHRSPWTFGGGTKLEIKRADAAPTVSIFPPSSEQLTSGGASVVCFLNNFYPK

SEQ ID NO: 496 - protein, 5 aa, synthetic construct
  REGION 1..5: note = Synthetic Construct
NSAMS

SEQ ID NO: 497 - protein, 18 aa, synthetic construct
  REGION 1..18: note = Synthetic Construct
GISVTGGSTFYTDSVKGR

SEQ ID NO: 498 - protein, 9 aa, synthetic construct
  REGION 1..9: note = Synthetic Construct
VYYYSFFDY

SEQ ID NO: 499 - protein, 14 aa, synthetic construct
  REGION 1..14: note = Synthetic Construct
GFNSGSVSTSYWPS

SEQ ID NO: 500 - protein, 7 aa, synthetic construct
  REGION 1..7: note = Synthetic Construct
NTNTRSS

SEQ ID NO: 501 - protein, 10 aa, synthetic construct
  REGION 1..10: note = Synthetic Construct
VLWMGSGIWV

SEQ ID NO: 502 - protein, 5 aa, synthetic construct
  REGION 1..5: note = Synthetic Construct
NYAMS

SEQ ID NO: 503 - protein, 18 aa, synthetic construct
  REGION 1..18: note = Synthetic Construct
GISVTGGSTYYTDSVKGR

SEQ ID NO: 504 - protein, 9 aa, synthetic construct
  REGION 1..9: note = Synthetic Construct
VYYSSFFDY

SEQ ID NO: 505 - protein, 14 aa, synthetic construct
  REGION 1..14: note = Synthetic Construct
GFNSGSVSTSYYPS

SEQ ID NO: 506 - protein, 7 aa, synthetic construct
  REGION 1..7: note = Synthetic Construct
NTNTRSS

SEQ ID NO: 507 - protein, 10 aa, synthetic construct
  REGION 1..10: note = Synthetic Construct
VLYMGSGIWV

SEQ ID NO: 508 - protein, 444 aa, synthetic construct
  REGION 1..444: note = Synthetic Construct
EVQLLESGGGLVQPGGSLRLSCAASGFTFRNSAMSWVRQAPGKGLEWVSGISVTGGSTFYTDSVKGRFTVSRDNSRNTLYLQMNSLRAEDTAVYYCAKVYYYSFFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 509 - protein, 216 aa, synthetic construct
  REGION 1..216: note = Synthetic Construct
QTVVTQEPSFSVSPGGTVTLTCGFNSGSVSTSYWPSWYQQTPGQAPRTLIYNTNTRSSGVPDRFSGSILGNKAALTITGAQADDESDYYCVLWMGSGIWVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 510 - protein, 444 aa, synthetic construct
  REGION 1..444: note = Synthetic Construct
EVQLLESGGGLVQPGGSLRLSCAASGFTFRNDAMSWVRQAPGKGLEWVSGISVTGGSTFYTDSVKGRFTVSRDNSRNTLYLQMNSLRAEDTAVYYCAKVYYTSFFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 511 - protein, 216 aa, synthetic construct
  REGION 1..216: note = Synthetic Construct
QTVVTQEPSFSVSPGGTVTLTCGFNSGSVSTSYYPSWYQQTPGQAPRTLIYNTNTRSSGVPDRFSGSILGNKAALTITGAQADDESDYYCVLYMGSGIWVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 512 - protein, 115 aa, synthetic construct
  REGION 1..115: note = Synthetic Construct
QVQLVQSGAEVKKPGASVKVSCKASGYTFTSSYINWVRQAPGQGLEWMGTINPVSGSTSYAQKFQGRVTMTRDTSISTAYMELSSLRSEDTAVYYCARGGWFDYWGQGTLVTVSS

SEQ ID NO: 513 - protein, 113 aa, synthetic construct
  REGION 1..113: note = Synthetic Construct
DIALTQPASVSGSPGQSITISCTGTSSDVGSYNYVNWYQQHPGKAPKLMIYGVSKRPSGVSNRFSGSKSGNTASLTISGLQAEDEADYYCGTFAGGSYYGVFGGGTKLTVLGQ

SEQ ID NO: 514 - protein, 115 aa, synthetic construct
  REGION 1..115: note = Synthetic Construct
QVQLVQSGAEVKKPGASVKVSCKASGYTFTSSYINWVRQAPGQGLEWMGTINPVSGNTSYAQKFQGRVTMTRDTSISTAYMELSSLRSEDTAVYYCARGGWFDYWGQGTLVTVSS

SEQ ID NO: 515 - protein, 112 aa, synthetic construct
  REGION 1..112: note = Synthetic Construct
DIALTQPASVSGSPGQSITISCTGTSSDVGSYNYVNWYQQHPGKAPKLMIYGVSKRPSGVSNRFSGSKSGNTASLTISGLQAEDEADYYCQAWTSKMAGVFGGGTKLTVLGQ

SEQ ID NO: 516 - protein, 115 aa, synthetic construct; identical to SEQ ID NO: 514
  REGION 1..115: note = Synthetic Construct

SEQ ID NO: 517 - protein, 112 aa, synthetic construct
  REGION 1..112: note = Synthetic Construct
DIALTQPASVSGSPGQSITISCTGTSSDVGSYNYVNWYQQHPGKAPKLMIYGVSKRPSGVSNRFSGSKSGNTASLTISGLQAEDEADYYCSSYTRMGHPVFGGGTKLTVLGQ

SEQ ID NO: 518 - protein, 115 aa, synthetic construct; identical to SEQ ID NO: 514
  REGION 1..115: note = Synthetic Construct

SEQ ID NO: 519 - protein, 113 aa, synthetic construct
  REGION 1..113: note = Synthetic Construct
DIALTQPASVSGSPGQSITISCTGTSSDVGSYNYVNWYQQHPGKAPKLMIYGVSKRPSGVSNRFSGSKSGNTASLTISGLQAEDEADYYCATYGKGVTPPVFGGGTKLTVLGQ

SEQ ID NO: 520 - protein, 115 aa, synthetic construct; identical to SEQ ID NO: 514
  REGION 1..115: note = Synthetic Construct

SEQ ID NO: 521 - protein, 113 aa, synthetic construct; identical to SEQ ID NO: 513
  REGION 1..113: note = Synthetic Construct

SEQ ID NO: 522 - protein, 115 aa, synthetic construct
  REGION 1..115: note = Synthetic Construct
QVQLVQSGAEVKKPGASVKVSCKASGYTFTSSYINWVRQAPGQGLEWMGMINAPIGTTRYAQKFQGRVTMTRDTSISTAYMELSSLRSEDTAVYYCARGGWFDYWGQGTLVTVSS

SEQ ID NO: 523 - protein, 112 aa, synthetic construct; identical to SEQ ID NO: 515
  REGION 1..112: note = Synthetic Construct

SEQ ID NO: 524 - protein, 115 aa, synthetic construct
  REGION 1..115: note = Synthetic Construct
QVQLVQSGAEVKKPGASVKVSCKASGYTFTSSYINWVRQAPGQGLEWMGQINAASGMTRYAQKFQGRVTMTRDTSISTAYMELSSLRSEDTAVYYCARGGWFDYWGQGTLVTVSS

SEQ ID NO: 525 - protein, 112 aa, synthetic construct; identical to SEQ ID NO: 515
  REGION 1..112: note = Synthetic Construct

SEQ ID NO: 526 - protein, 115 aa, synthetic construct; identical to SEQ ID NO: 522
  REGION 1..115: note = Synthetic Construct

SEQ ID NO: 527 - protein, 113 aa, synthetic construct; identical to SEQ ID NO: 513
  REGION 1..113: note = Synthetic Construct

SEQ ID NO: 528 - protein, 115 aa, synthetic construct
  REGION 1..115: note = Synthetic Construct
QVQLVQSGAEVKKPGASVKVSCKASGYTFTSSYINWVRQAPGQGLEWMGTINPVSGNTRYAQKFQGRVTMTRDTSISTAYMELSSLRSEDTAVYYCARGGWFDYWGQGTLVTVSS

SEQ ID NO: 529 - protein, 113 aa, synthetic construct; identical to SEQ ID NO: 513
  REGION 1..113: note = Synthetic Construct

SEQ ID NO: 530 - protein, 115 aa, synthetic construct; identical to SEQ ID NO: 524
  REGION 1..115: note = Synthetic Construct

SEQ ID NO: 531 - protein, 113 aa, synthetic construct; identical to SEQ ID NO: 513
  REGION 1..113: note = Synthetic Construct

SEQ ID NO: 532 - protein, 115 aa, synthetic construct
  REGION 1..115: note = Synthetic Construct
QVQLVQSGAEVKKPGASVKVSCKASGYTFTSSYINWVRQAPGQGLEWMGNINAAAGITLYAQKFQGRVTMTRDTSISTAYMELSSLRSEDTAVYYCARGGWFDYWGQGTLVTVSS

SEQ ID NO: 533 - protein, 113 aa, synthetic construct; identical to SEQ ID NO: 513
  REGION 1..113: note = Synthetic Construct

SEQ ID NO: 534 - protein, 115 aa, synthetic construct
  REGION 1..115: note = Synthetic Construct
QVQLVQSGAEVKKPGASVKVSCKASGYTFTSSYINWVRQAPGQGLEWMGTINPPTGGTYYAQKFQGRVTMTRDTSISTAYMELSSLRSEDTAVYYCARGGWFDYWGQGTLVTVSS

SEQ ID NO: 535 - protein, 113 aa, synthetic construct; identical to SEQ ID NO: 513
  REGION 1..113: note = Synthetic Construct

SEQ ID NO: 536 - protein, 115 aa, synthetic construct
  REGION 1..115: note = Synthetic Construct
QVQLVQSGAEVKKPGASVKVSCKASGYTFTSSYINWVRQAPGQGLEWMGGINPPAGTTSYAQKFQGRVTMTRDTSISTAYMELSSLRSEDTAVYYCARGGWFDYWGQGTLVTVSS

SEQ ID NO: 537 - protein, 113 aa, synthetic construct; identical to SEQ ID NO: 513
  REGION 1..113: note = Synthetic Construct

SEQ ID NO: 538 - protein, 115 aa, synthetic construct
  REGION 1..115: note = Synthetic Construct
QVQLVQSGAEVKKPGASVKVSCKASGYTFTSSYINWVRQAPGQGLEWMGNINPATGHADYAQKFQGRVTMTRDTSISTAYMELSSLRSEDTAVYYCARGGWFDYWGQGTLVTVSS

SEQ ID NO: 539 - protein, 113 aa, synthetic construct; identical to SEQ ID NO: 513
  REGION 1..113: note = Synthetic Construct

SEQ ID NO: 540 - protein, 10 aa, synthetic construct
  REGION 1..10: note = Synthetic Construct
GYTFTSSYIN

SEQ ID NO: 541 - protein, 17 aa, synthetic construct
  REGION 1..17: note = Synthetic Construct
TINPVSGSTSYAQKFQG

SEQ ID NO: 542 - protein, 6 aa, synthetic construct
  REGION 1..6: note = Synthetic Construct
GGWFDY

SEQ ID NO: 543 - protein, 14 aa, synthetic construct
  REGION 1..14: note = Synthetic Construct
TGTSSDVGSYNYVN

SEQ ID NO: 544 - protein, 11 aa, synthetic construct
  REGION 1..11: note = Synthetic Construct
LMIYGVSKRPS

SEQ ID NO: 545 - protein, 10 aa, synthetic construct
  REGION 1..10: note = Synthetic Construct
GTFAGGSYYG

SEQ ID NO: 546 - protein, 17 aa, synthetic construct
  REGION 1..17: note = Synthetic Construct
TINPVSGNTSYAQKFQG

SEQ ID NO: 547 - protein, 9 aa, synthetic construct
  REGION 1..9: note = Synthetic Construct
QAWTSKMAG

SEQ ID NO: 548 - protein, 9 aa, synthetic construct
  REGION 1..9: note = Synthetic Construct
SSYTRMGHP

SEQ ID NO: 549 - protein, 10 aa, synthetic construct
  REGION 1..10: note = Synthetic Construct
ATYGKGVTPP

SEQ ID NO: 550 - protein, 17 aa, synthetic construct
  REGION 1..17: note = Synthetic Construct
MINAPIGTTRYAQKFQG

SEQ ID NO: 551 - protein, 17 aa, synthetic construct
  REGION 1..17: note = Synthetic Construct
QINAASGMTRYAQKFQG

SEQ ID NO: 552 - protein, 17 aa, synthetic construct
  REGION 1..17: note = Synthetic Construct
TINPVSGNTRYAQKFQG

SEQ ID NO: 553 - protein, 17 aa, synthetic construct
  REGION 1..17: note = Synthetic Construct
NINAAAGITLYAQKFQG

SEQ ID NO: 554 - protein, 17 aa, synthetic construct
  REGION 1..17: note = Synthetic Construct
TINPPTGGTYYAQKFQG

SEQ ID NO: 555 - protein, 17 aa, synthetic construct
  REGION 1..17: note = Synthetic Construct
GINPPAGTTSYAQKFQG

SEQ ID NO: 556 - protein, 17 aa, synthetic construct
  REGION 1..17: note = Synthetic Construct
NINPATGHADYAQKFQG

SEQ ID NO: 557 - protein, 445 aa, synthetic construct
  REGION 1..445: note = Synthetic Construct
QVQLVQSGAEVKKPGASVKVSCKASGYTFTSSYINWVRQAPGQGLEWMGTINPVSGSTSYAQKFQGRVTMTRDTSISTAYMELSRLRSDDTAVYYCARGGWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 558 - protein, 217 aa, synthetic construct
  REGION 1..217: note = Synthetic Construct
QSALTQPASVSGSPGQSITISCTGTSSDVGSYNYVNWYQQHPGKAPKLMIYGVSKRPSGVSNRFSGSKSGNTASLTISGLQAEDEADYYCGTFAGGSYYGVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 559 - protein, 445 aa, synthetic construct
  REGION 1..445: note = Synthetic Construct
QVQLVQSGAEVKKPGASVKVSCKASGYTFTSSYINWVRQAPGQGLEWMGQINAASGMTRYAQKFQGRVTMTRDTSISTAYMELSRLRSDDTAVYYCARGGWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 560 - protein, 217 aa, synthetic construct; identical to SEQ ID NO: 558
  REGION 1..217: note = Synthetic Construct

SEQ ID NO: 561 - protein, 445 aa, synthetic construct
  REGION 1..445: note = Synthetic Construct
QVQLVQSGAEVKKPGASVKVSCKASGYTFTSSYINWVRQAPGQGLEWMGNINAAAGITLYAQKFQGRVTMTRDTSISTAYMELSRLRSDDTAVYYCARGGWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 562 - protein, 217 aa, synthetic construct; identical to SEQ ID NO: 558
  REGION 1..217: note = Synthetic Construct

SEQ ID NO: 563 - protein, 445 aa, synthetic construct
  REGION 1..445: note = Synthetic Construct
QVQLVQSGAEVKKPGASVKVSCKASGYTFTSSYINWVRQAPGQGLEWMGGINPPAGTTSYAQKFQGRVTMTRDTSISTAYMELSRLRSDDTAVYYCARGGWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 564 - protein, 217 aa, synthetic construct; identical to SEQ ID NO: 558
  REGION 1..217: note = Synthetic Construct

SEQ ID NO: 565 - protein, 445 aa, synthetic construct
  REGION 1..445: note = Synthetic Construct
QVQLVQSGAEVKKPGASVKVSCKASGYTFTSSYINWVRQAPGQGLEWMGNINPATGHADYAQKFQGRVTMTRDTSISTAYMELSRLRSDDTAVYYCARGGWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 566 - protein, 217 aa, synthetic construct; identical to SEQ ID NO: 558
  REGION 1..217: note = Synthetic Construct

SEQ ID NO: 567 - protein, 441 aa, synthetic construct
  REGION 1..441: note = Synthetic Construct
QVQLVQSGAEVKKPGASVKVSCKASGYTFTSSYINWVRQAPGQGLEWMGTINPVSGSTSYAQKFQGRVTMTRDTSISTAYMELSRLRSDDTAVYYCARGGWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 568 - protein, 217 aa, synthetic construct; identical to SEQ ID NO: 558
  REGION 1..217: note = Synthetic Construct

SEQ ID NO: 569 - protein, 441 aa, synthetic construct
  REGION 1..441: note = Synthetic Construct
QVQLVQSGAEVKKPGASVKVSCKASGYTFTSSYINWVRQAPGQGLEWMGQINAASGMTRYAQKFQGRVTMTRDTSISTAYMELSRLRSDDTAVYYCARGGWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 570 - protein, 217 aa, synthetic construct; identical to SEQ ID NO: 558
  REGION 1..217: note = Synthetic Construct

SEQ ID NO: 571 - protein, 441 aa, synthetic construct
  REGION 1..441: note = Synthetic Construct
QVQLVQSGAEVKKPGASVKVSCKASGYTFTSSYINWVRQAPGQGLEWMGNINAAAGITLYAQKFQGRVTMTRDTSISTAYMELSRLRSDDTAVYYCARGGWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 572 - protein, 217 aa, synthetic construct; identical to SEQ ID NO: 558
  REGION 1..217: note = Synthetic Construct

SEQ ID NO: 573 - protein, 441 aa, synthetic construct
  REGION 1..441: note = Synthetic Construct
QVQLVQSGAEVKKPGASVKVSCKASGYTFTSSYINWVRQAPGQGLEWMGGINPPAGTTSYAQKFQGRVTMTRDTSISTAYMELSRLRSDDTAVYYCARGGWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 574 - protein, 217 aa, synthetic construct; identical to SEQ ID NO: 558
  REGION 1..217: note = Synthetic Construct

SEQ ID NO: 575 - protein, 441 aa, synthetic construct
  REGION 1..441: note = Synthetic Construct
QVQLVQSGAEVKKPGASVKVSCKASGYTFTSSYINWVRQAPGQGLEWMGNINPATGHADYAQKFQGRVTMTRDTSISTAYMELSRLRSDDTAVYYCARGGWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 576 - protein, 217 aa, synthetic construct; identical to SEQ ID NO: 558
  REGION 1..217: note = Synthetic Construct

SEQ ID NO: 577 - protein, 5 aa, synthetic construct
  REGION 1..5: note = Synthetic Construct
NYWMG

SEQ ID NO: 578 - protein, 17 aa, synthetic construct
  REGION 1..17: note = Synthetic Construct
YIRSSGGLTHYADSVKG

SEQ ID NO: 579 - protein, 7 aa, synthetic construct
  REGION 1..7: note = Synthetic Construct
GLYSFDY

SEQ ID NO: 580 - protein, 11 aa, synthetic construct
  REGION 1..11: note = Synthetic Construct
RASQGVNNFLA

SEQ ID NO: 581 - protein, 7 aa, synthetic construct
  REGION 1..7: note = Synthetic Construct
AASTLQS

SEQ ID NO: 582 - protein, 9 aa, synthetic construct
  REGION 1..9: note = Synthetic Construct
QQSYSTPRG

SEQ ID NO: 583 - protein, 120 aa, synthetic construct
  REGION 1..120: note = Synthetic Construct
DVQLQESGPGLVKPSQSLSLTCSVTGYSITSGYYWNWIRQFPGNKLEWMGYISYDGSNNYNPSLINRISITRDTSKNQFFLKLNSVTTEDTATYFCASYAYRNDVRFAYWGQGTLVTVSA

SEQ ID NO: 584 - protein, 107 aa, synthetic construct
  REGION 1..107: note = Synthetic Construct
DIQMTQTTSSLSASLGDRVTISCRASQDISNFLNWYQQKPDGTVKLLIYFTSRLHSGVPSRFSGSGSGTDYSLTITNLEQEDIATYFCQQGNTLPWTFGGGTKLEIK

SEQ ID NO: 585 - protein, 10 aa, synthetic construct
  REGION 1..10: note = Synthetic Construct
YSITSGYYWN

SEQ ID NO: 586 - protein, 16 aa, synthetic construct
  REGION 1..16: note = Synthetic Construct
YISYDGSNNYNPSLIN

SEQ ID NO: 587 - protein, 11 aa, synthetic construct
  REGION 1..11: note = Synthetic Construct
YAYRNDVRFAY

SEQ ID NO: 588 - protein, 11 aa, synthetic construct
  REGION 1..11: note = Synthetic Construct
RASQDISNFLN

SEQ ID NO: 589 - protein, 7 aa, synthetic construct
  REGION 1..7: note = Synthetic Construct
FTSRLHS

SEQ ID NO: 590 - protein, 9 aa, synthetic construct
  REGION 1..9: note = Synthetic Construct
QQGNTLPWT

SEQ ID NO: 591 - protein, 120 aa, synthetic construct
  REGION 1..120: note = Synthetic Construct
QVQLVQSGAEVKKPGASVKVSCKASGYTFTSYRMHWVRQAPGQGLEWMGFIVPSGGSTGYAQKFQGRVTMTRDTSTSTVYMELSSLRSEDTAVYYCARVSRYAPEPMDVWGQGTTVTVSS

SEQ ID NO: 592 - protein, 107 aa, synthetic construct
  REGION 1..107: note = Synthetic Construct
DIQMTQSPSSVSASVGDRVTITCRASQGISRWLAWYQQKPGKAPKLLIYAASSLQSGVPSRFSGSGSGTDFTLTISSLQPEDFATYYCQQAFSHPWTFGGGTKVEIK

SEQ ID NO: 593 - protein, 130 aa, synthetic construct
  REGION 1..130: note = Synthetic Construct
QLQLQESGPGLVKPSETLSLTCTVSGGSISSSSYAWGWIRQPPGKGLEWIGSIYYSGSTYYNPSLKSRVTISVDTSKNQFSLKLSSVTAADTAVYYCARDSGIGYSYASSHGYYYYMDVWGKGTTVTVSS

SEQ ID NO: 594 - protein, 107 aa, synthetic construct
  REGION 1..107: note = Synthetic Construct
EIVLTQSPATLSVSPGERATLSCRASQSVGSNLAWYQQKPGQAPRLLIYGASTRATGIPARFSGSGSGTEFTLTISSLQSEDFAVYYCQQYFHFPLTFGGGTKVEIK

SEQ ID NO: 595 - protein, 130 aa, synthetic construct
  REGION 1..130: note = Synthetic Construct
QVQLQESGPGLVKPSQTLSLTCTVSGGSIGSGGYYWSWIRQHPGKGLEWIGGIYGSGSTYYNPSLKSRVTISVDTSKNQFSLKLSSVTAADTAVYYCARDSGIGYSYASSHGYYYYMDVWGKGTTVTVSS

SEQ ID NO: 596 - protein, 130 aa, synthetic construct
  REGION 1..130: note = Synthetic Construct
QVQLQESGPGLVKPSQTLSLTCTVSGGSIKSGGYYWSWIRQHPGKGLEWIGGIYPSGSTYYNPSLKSRVTISVDTSKNQFSLKLSSVTAADTAVYYCARDSGIGYSYASSHGYYYYMDVWGKGTTVTVSS

SEQ ID NO: 597 - protein, 130 aa, synthetic construct
  REGION 1..130: note = Synthetic Construct
QVQLQESGPGLVKPSQTLSLTCTVSGGSILSGGYYWSWIRQHPGKGLEWIGGIYYSGKTYYNPSLKSRVTISVDTSKNQFSLKLSSVTAADTAVYYCARDSGIGYSYASSHGYYYYMDVWGKGTTVTVSS

SEQ ID NO: 598 - protein, 130 aa, synthetic construct
  REGION 1..130: note = Synthetic Construct
QVQLQESGPGLVKPSQTLSLTCTVSGGSISSGGYFWSWIRQHPGKGLEWIGGIYYSGRTYYNPSLKSRVTISVDTSKNQFSLKLSSVTAADTAVYYCARDSGIGYSYASSHGYYYYMDVWGKGTTVTVSS

SEQ ID NO: 599 - protein, 130 aa, synthetic construct
  REGION 1..130: note = Synthetic Construct
QVQLQESGPGLVKPSQTLSLTCTVSGGSIESGGYYWSWIRQHPGKGLEWIGGIYGSGSTYYNPSLKSRVTISVDTSKNQFSLKLSSVTAADTAVYYCARDSGIGYSYASSHGYYYYMDVWGKGTTVTVSS

SEQ ID NO: 600 - protein, 121 aa, synthetic construct
  REGION 1..121: note = Synthetic Construct
QLQLQESGPGLVKPSETLSLTCTVSGGSISSSSYAWGWIRQPPGKGLEWIGSIYYSGSTYYNPSLKSRVTISVDTSKNQFSLKLSSVTAADTAVYYCARAGKYRWHGMDVWGQGTTVTVSS

SEQ ID NO: 601 - protein, 107 aa, synthetic construct
  REGION 1..107: note = Synthetic Construct
DIQMTQSPSSVSASVGDRVTITCRASQGISSWLAWYQQKPGKAPKLLIYAASNLQSGVPSRFSGSGSGTDFTLTISSLQPEDFATYYCQQAPDLPITFGGGTKVEIK

SEQ ID NO: 602 - protein, 120 aa, synthetic construct
  REGION 1..120: note = Synthetic Construct
QVQLQESGPGLVKPSETLSLTCAVSGYSISSGVYWMWIRQPPGKGLEWIGSIVHSGHTYYNPSLKSRVTISVDTSKNQFSLKLSSVTAADTAVYYCARAGKYRWHGMDVWGQGTTVTVSS

SEQ ID NO: 603 - protein, 121 aa, synthetic construct
  REGION 1..121: note = Synthetic Construct
EVQLLESGGGLVQPGGSLRLSCAASGFTFSSYAMSWVRQAPGKGLEWVSGISGSGGSTYYADSVKGRFTISRDNSKNTLYLQMNSLRAEDTAVYYCAKDPLSLLLGYFDYWGQGALVTVSS

SEQ ID NO: 604 - protein, 107 aa, synthetic construct
  REGION 1..107: note = Synthetic Construct
DIQMTQSPSTLSASVGDRVTITCRASQSISSWLAWYQQKPGKAPKLLIYDASSLESGVPSRFSGSGSGTEFTLTISSLQPDDFATYYCQQYNRHSPTFGGGTKVEIK

SEQ ID NO: 605 - protein, 121 aa, synthetic construct
  REGION 1..121: note = Synthetic Construct
EVQLLESGGGLVQPGGSLRLSCAASGFTFSRYAMSWVRQAPGKGLEWVSGISGSGGATYYADSVKGRFTISRDNSKNTLYLQMNSLRAEDTAVYYCAKDPLSLLLGYFDYWGQGALVTVSS

SEQ ID NO: 606 - protein, 117 aa, synthetic construct
  REGION 1..117: note = Synthetic Construct
EVQLLESGGGLVQPGGSLRLSCAASGFTFGSYGMTWVRQAPGKGLEWVSVISGSGGGTYYADSVKGRFTISRDNSKNTLYLQMNSLRAEDTAVYYCAKGPRIVGMDVWGQGTTVTVS

SEQ ID NO: 607 - protein, 107 aa, synthetic construct
  REGION 1..107: note = Synthetic Construct
DIQMTQSPSSVSASVGDRVTITCRASQGISSWLAWYQQKPGKAPKLLIYAASSLQSGVPSRFSGSGSGTDFTLTISSLQPEDFATYYCQQVFSYPLTFGGGTKVEIK

SEQ ID NO: 608 - protein, 120 aa, synthetic construct
  REGION 1..120: note = Synthetic Construct
QVQLVQSGAEVKKPGASVKVSCKASGYTFTSYGISWVRQAPGQGLEWMGWISPYNGNTNYAQKLQGRVTMTTDTSTSTAYMELRSLRSDDTAVYYCARVSMYAPEPMDVWGQGTTVTVSS

SEQ ID NO: 609 - protein, 107 aa, synthetic construct; identical to SEQ ID NO: 592
  REGION 1..107: note = Synthetic Construct

SEQ ID NO: 610 - protein, 120 aa, synthetic construct
  REGION 1..120: note = Synthetic Construct
QVQLVQSGAEVKKPGASVKVSCKASGYTFTGHKMHWVRQAPGQGLEWMGWINPASGWTNYAQKFQGRVTMTRDTSISTAYMELSRLRSDDTAVYYCARVSMYAPEPMDVWGQGTTVTVSS

SEQ ID NO: 611 - protein, 120 aa, synthetic construct
  REGION 1..120: note = Synthetic Construct
QVQLVQSGAEVKKPGASVKVSCKASGYTFTSYNMAWVRQAPGQGLEWMGIIRPSVGSTSYAQKFQGRVTMTRDTSTSTVYMELSSLRSEDTAVYYCARVSMYAPEPMDVWGQGTTVTVSS

SEQ ID NO: 612 - protein, 120 aa, synthetic construct
  REGION 1..120: note = Synthetic Construct
QVQLVQSGAEVKKPGASVKVSCKASGYTFTSYRMHWVRQAPGQGLEWMGFIVPSGGSTSYAQKFQGRVTMTRDTSTSTVYMELSSLRSEDTAVYYCARVSMYAPEPMDVWGQGTTVTVSS

SEQ ID NO: 613 - protein, 120 aa, synthetic construct; identical to SEQ ID NO: 591
  REGION 1..120: note = Synthetic Construct

SEQ ID NO: 614 - protein, 122 aa, synthetic construct
  REGION 1..122: note = Synthetic Construct
QVQLQESGPGLVKPSQTLSLTCTVSGGSISSGSYYWSWIRQHPGKGLEWIGYIYYSGSTYYNPSLKSRVTISVDTSKNQFSLKLSSVTAADTAVYYCARGLGMYYHVPFDIWGQGTMVTVSS

SEQ ID NO: 615 - protein, 107 aa, synthetic construct
  REGION 1..107: note = Synthetic Construct
EIVLTQSPATLSLSPGERATLSCRASQSVSSYLAWYQQKPGQAPRLLIYDASNRATGIPARFSGSGSGTDFTLTISSLEPEDFAVYYCQQYFHWPPTFGGGTKVEIK

SEQ ID NO: 616 - protein, 5 aa, synthetic construct
  REGION 1..5: note = Synthetic Construct
SYRMH

SEQ ID NO: 617 - protein, 17 aa, synthetic construct
  REGION 1..17: note = Synthetic Construct
FIVPSGGSTGYAQKFQG

SEQ ID NO: 618 - protein, 11 aa, synthetic construct
  REGION 1..11: note = Synthetic Construct
VSRYAPEPMDV

SEQ ID NO: 619 - protein, 11 aa, synthetic construct
  REGION 1..11: note = Synthetic Construct
RASQGISRWLA

SEQ ID NO: 620 - protein, 7 aa, synthetic construct
  REGION 1..7: note = Synthetic Construct
AASSLQS

SEQ ID NO: 621 - protein, 9 aa, synthetic construct
  REGION 1..9: note = Synthetic Construct
QQAFSHPWT

SEQ ID NO: 622 - protein, 9 aa, synthetic construct
  REGION 1..9: note = Synthetic Construct
GGSISSSSY

SEQ ID NO: 623 - protein, 5 aa, synthetic construct
  REGION 1..5: note = Synthetic Construct
YYSGS

SEQ ID NO: 624 - protein, 20 aa, synthetic construct
  REGION 1..20: note = Synthetic Construct
DSGIGYSYASSHGYYYYMDV

SEQ ID NO: 625 - protein, 11 aa, synthetic construct
  REGION 1..11: note = Synthetic Construct
RASQSVGSNLA

SEQ ID NO: 626 - protein, 7 aa, synthetic construct
  REGION 1..7: note = Synthetic Construct
GASTRAT

SEQ ID NO: 627 - protein, 9 aa, synthetic construct
  REGION 1..9: note = Synthetic Construct
QQYFHFPLT

SEQ ID NO: 628 - protein, 4 aa, synthetic construct
  REGION 1..4: note = Synthetic Construct
YGSG

SEQ ID NO: 629 - protein, 9 aa, synthetic construct
  REGION 1..9: note = Synthetic Construct
GGSIGSGGY

SEQ ID NO: 630 - protein, 7 aa, synthetic construct
  REGION 1..7: note = Synthetic Construct
GFTFSSY

SEQ ID NO: 631 - protein, 6 aa, synthetic construct
  REGION 1..6: note = Synthetic Construct
SGSGGS

SEQ ID NO: 632 - protein, 12 aa, synthetic construct
  REGION 1..12: note = Synthetic Construct
DPLSLLLGYFDY

SEQ ID NO: 633 - protein, 11 aa, synthetic construct
  REGION 1..11: note = Synthetic Construct
RASQSISSWLA

SEQ ID NO: 634 - protein, 7 aa, synthetic construct
  REGION 1..7: note = Synthetic Construct
DASSLES

SEQ ID NO: 635 - protein, 9 aa, synthetic construct
  REGION 1..9: note = Synthetic Construct
QQYNRHSPT

SEQ ID NO: 636 - protein, 12 aa, synthetic construct
  REGION 1..12: note = Synthetic Construct
DPLSLLLGYFDY

SEQ ID NO: 637 - protein, 6 aa, synthetic construct
  REGION 1..6: note = Synthetic Construct
SGSGGA

SEQ ID NO: 638 - protein, 7 aa, synthetic construct
  REGION 1..7: note = Synthetic Construct
GFTFSRY

SEQ ID NO: 639 - protein, 11 aa, synthetic construct
  REGION 1..11: note = Synthetic Construct
AGKYRWHGMDV

SEQ ID NO: 640 - protein, 11 aa, synthetic construct
  REGION 1..11: note = Synthetic Construct
RASQGISSWLA

SEQ ID NO: 641 - protein, 7 aa, synthetic construct
  REGION 1..7: note = Synthetic Construct
AASNLQS

SEQ ID NO: 642 - protein, 9 aa, synthetic construct
  REGION 1..9: note = Synthetic Construct
QQAPDLPIT

SEQ ID NO: 643 - protein, 5 aa, synthetic construct
  REGION 1..5: note = Synthetic Construct
VHSGH

SEQ ID NO: 644 - protein, 8 aa, synthetic construct
  REGION 1..8: note = Synthetic Construct
GYSISSGV

SEQ ID NO: 645 - protein, 5 aa, synthetic construct
  REGION 1..5: note = Synthetic Construct
SYGMT

SEQ ID NO: 646 - protein, 17 aa, synthetic construct
  REGION 1..17: note = Synthetic Construct
VISGSGGGTYYADSVKG

SEQ ID NO: 647 - protein, 9 aa, synthetic construct
  REGION 1..9: note = Synthetic Construct
GPRIVGMDV

SEQ ID NO: 648 - protein, 11 aa, synthetic construct
  REGION 1..11: note = Synthetic Construct
RASQGISSWLA

SEQ ID NO: 649 - protein, 7 aa, synthetic construct
  REGION 1..7: note = Synthetic Construct
AASSLQS

SEQ ID NO: 650 - protein, 9 aa, synthetic construct
  REGION 1..9: note = Synthetic Construct
QQVFSYPLT

SEQ ID NO: 651 - protein, 7 aa, synthetic construct
  REGION 1..7: note = Synthetic Construct
GYTFTSY

SEQ ID NO: 652 - protein, 6 aa, synthetic construct
  REGION 1..6: note = Synthetic Construct
SPYNGN

SEQ ID NO: 653 - protein, 11 aa, synthetic construct
  REGION 1..11: note = Synthetic Construct
VSMYAPEPMDV

SEQ ID NO: 654 - protein, 11 aa, synthetic construct
  REGION 1..11: note = Synthetic Construct
RASQGISRWLA

SEQ ID NO: 655 - protein, 6 aa, synthetic construct
  REGION 1..6: note = Synthetic Construct
ASSLQS

SEQ ID NO: 656 - protein, 9 aa, synthetic construct
  REGION 1..9: note = Synthetic Construct
QQAFSHPWT

SEQ ID NO: 657 - protein, 11 aa, synthetic construct
  REGION 1..11: note = Synthetic Construct
VSMYAPEPMDV

SEQ ID NO: 658 - protein, 6 aa, synthetic construct
  REGION 1..6: note = Synthetic Construct
NPASGW

SEQ ID NO: 659 - protein, 10 aa, synthetic construct
  REGION 1..10: note = Synthetic Construct
GYTFTGHKMH

SEQ ID NO: 660 - protein, 9 aa, synthetic construct
  REGION 1..9: note = Synthetic Construct
GGSISSGSY

SEQ ID NO: 661 - protein, 5 aa, synthetic construct
  REGION 1..5: note = Synthetic Construct
YYSGS

SEQ ID NO: 662 - protein, 12 aa, synthetic construct
  REGION 1..12: note = Synthetic Construct
GLGMYYHVPFDI

SEQ ID NO: 663 - protein, 11 aa, synthetic construct
  REGION 1..11: note = Synthetic Construct
RASQSVSSYLA

SEQ ID NO: 664 - protein, 7 aa, synthetic construct
  REGION 1..7: note = Synthetic Construct
DASNRAT

SEQ ID NO: 665 - protein, 9 aa, synthetic construct
  REGION 1..9: note = Synthetic Construct
QQYFHWPPT

SEQ ID NO: 666 - protein, 13 aa, synthetic construct
  REGION 1..13: note = Synthetic Construct
WIGGIYPSGSTYY

SEQ ID NO: 667 - protein, 5 aa, synthetic construct
  REGION 1..5: note = Synthetic Construct
YYSGK

SEQ ID NO: 668 - protein, 6 aa, synthetic construct
  REGION 1..6: note = Synthetic Construct
YYSGRT

SEQ ID NO: 669 - protein, 9 aa, synthetic construct
  REGION 1..9: note = Synthetic Construct
GGSIKSGGY

SEQ ID NO: 670 - protein, 9 aa, synthetic construct
  REGION 1..9: note = Synthetic Construct
GGSILSGGY

SEQ ID NO: 671 - protein, 9 aa, synthetic construct
  REGION 1..9: note = Synthetic Construct
GGSISSGGY

SEQ ID NO: 672 - protein, 9 aa, synthetic construct
  REGION 1..9: note = Synthetic Construct
GGSIESGGY

SEQ ID NO: 673 - protein, 11 aa, synthetic construct
  REGION 1..11: note = Synthetic Construct
VSMYAPEPMDV

SEQ ID NO: 674 - protein, 11 aa, synthetic construct
  REGION 1..11: note = Synthetic Construct
VSMYAPEPMDV

SEQ ID NO: 675 - protein, 11 aa, synthetic construct
  REGION 1..11: note = Synthetic Construct
VSRYAPEPMDV

SEQ ID NO: 676 - protein, 6 aa, synthetic construct
  REGION 1..6: note = Synthetic Construct
RPSVGS

SEQ ID NO: 677 - protein, 6 aa, synthetic construct
  REGION 1..6: note = Synthetic Construct
VPSGGS

SEQ ID NO: 678 - protein, 6 aa, synthetic construct
  REGION 1..6: note = Synthetic Construct
VPSGGS

SEQ ID NO: 679 - protein, 7 aa, synthetic construct
  REGION 1..7: note = Synthetic Construct
GYTFTSY

SEQ ID NO: 680 - protein, 7 aa, synthetic construct
  REGION 1..7: note = Synthetic Construct
GYTFTSY

SEQ ID NO: 681 - protein, 5 aa, synthetic construct
  REGION 1..5: note = Synthetic Construct
NYWMG

SEQ ID NO: 682 - protein, 17 aa, synthetic construct
  REGION 1..17: note = Synthetic Construct
YIRSSGGLTHYADSVKG

SEQ ID NO: 683 - protein, 7 aa, synthetic construct
  REGION 1..7: note = Synthetic Construct
GLYSFDY

SEQ ID NO: 684 - protein, 11 aa, synthetic construct
  REGION 1..11: note = Synthetic Construct
RASQGVNNFLA

SEQ ID NO: 685 - protein, 7 aa, synthetic construct
  REGION 1..7: note = Synthetic Construct
AASTLQS

SEQ ID NO: 686 - protein, 9 aa, synthetic construct
  REGION 1..9: note = Synthetic Construct
QQSYSTPRG

SEQ ID NO: 687 - protein, 115 aa, synthetic construct [sequence omitted]

SEQ ID NO: 688 - protein, 111 aa, synthetic construct [sequence omitted]

SEQ ID NO: 689 - protein, 119 aa, synthetic construct
  REGION 1..119: note = Synthetic Construct
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDSLRYGMDVWGQGTTVTVSS

SEQ ID NO: 690 - protein, 114 aa, synthetic construct
  REGION 1..114: note = Synthetic Construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYALAPPRTFGGGTKVEIK

SEQ ID NO: 691 - protein, 107 aa, synthetic construct
  REGION 1..107: note = Synthetic Construct
EIVMTQSPATLSVSPGERATLSCRASQSVSSNLAWYQQKPGQAPRLLIYGASTRATGIPARFSGSGSGTEFTLTISSLQSEDFAVYYCQVYNVWPRTFGGGTKVEIK

SEQ ID NO: 692 - protein, 118 aa, synthetic construct
  REGION 1..118: note = Synthetic Construct
EVQLLESGGGLVQPGGSLRLSCAASGFTFGSYGMTWVRQAPGKGLEWVSVISGSGGGTYYADSVKGRFTISRDNSKNTLYLQMNSLRAEDTAVYYCAKGPRVVGMDVWGQGTTVTVSS

SEQ ID NO: 693 - protein, 107 aa, synthetic construct
  REGION 1..107: note = Synthetic Construct
DIQMTQSPSSVSASVGDRVTITCRASQGISSWLAWYQQKPGKAPKLLIYAASSLQSGVPSRFSGSGSGTDYTLTISSLQPEDFATYYCQQVFSYPLTFGGGTKVEIK

SEQ ID NO: 694 - protein, 108 aa, synthetic construct
  REGION 1..108: note = Synthetic Construct
DIQMTQSPSTLSASVGDRVTITCRASQSIGSWLAWYQQKPGKAPKLLIYKASSLESGVPSRFSGSGSGTEFTLTISSLQPDDFATYYCQVYGSYSPRTFGGGTKVEIK

SEQ ID NO: 695 - protein, 119 aa, synthetic construct
  REGION 1..119: note = Synthetic Construct
EVQLLESGGGLVQPGGSLRLSCAASGFTFSSYAMSWVRQAPGKGLEWVSAISGSGGSTSYADSVKGRFTISRDNSKNTLYLQMNSLRAEDTAVYYCARLPQYSRPFDYWGQGTLVTVSS

SEQ ID NO: 696 - protein, 107 aa, synthetic construct
  REGION 1..107: note = Synthetic Construct
DIQMTQSPSSVSASVGDRVTITCRASQGISSWLAWYQQKPGKAPKLLIYAASSLQSGVPSRFSGSGSGTDFTLTISSLQPEDFATYYCQQGHSFPLTFGGGTKVEIK

SEQ ID NO: 697 - protein, 120 aa, synthetic construct
  REGION 1..120: note = Synthetic Construct
QVQLVQSGAEVKKPGASVKVSCKASGYTFTSYRMHWVRQAPGQGLEWMGFIVPSGGSTSYAQKFQGRVTMTRDTSTSTVYMELSSLRSEDTAVYYCARVSRYAPEPMDVWGQGTTVTVSS

SEQ ID NO: 698 - protein, 107 aa, synthetic construct; identical to SEQ ID NO: 592
  REGION 1..107: note = Synthetic Construct

SEQ ID NO: 699 - protein, 11 aa, synthetic construct
  REGION 1..11: note = Synthetic Construct
GSISSSSYYWG

SEQ ID NO: 700 - protein, 16 aa, synthetic construct
  REGION 1..16: note = Synthetic Construct
SISYSGSTYYNPSLKS

SEQ ID NO: 701 - protein, 11 aa, synthetic construct
  REGION 1..11: note = Synthetic Construct
ARDSLRYGMDV

SEQ ID NO: 702 - protein, 17 aa, synthetic construct
  REGION 1..17: note = Synthetic Construct
KSSQSVLYSSNNKNYLA

SEQ ID NO: 703 - protein, 7 aa, synthetic construct
  REGION 1..7: note = Synthetic Construct
WASTRES

SEQ ID NO: 704 - protein, 10 aa, synthetic construct
  REGION 1..10: note = Synthetic Construct
QQYALAPPRT

SEQ ID NO: 705 - protein, 11 aa, synthetic construct
  REGION 1..11: note = Synthetic Construct
RASQSVSSNLA

SEQ ID NO: 706 - protein, 7 aa, synthetic construct
  REGION 1..7: note = Synthetic Construct
GASTRAT

SEQ ID NO: 707 - protein, 9 aa, synthetic construct
  REGION 1..9: note = Synthetic Construct
QVYNVWPRT

SEQ ID NO: 708 - protein, 5 aa, synthetic construct
  REGION 1..5: note = Synthetic Construct
SYGMT

SEQ ID NO: 709 - protein, 17 aa, synthetic construct
  REGION 1..17: note = Synthetic Construct
VISGSGGGTYYADSVKG

SEQ ID NO: 710 - protein, 11 aa, synthetic construct
  REGION 1..11: note = Synthetic Construct
AKGPRVVGMDV

SEQ ID NO: 711 - protein, 11 aa, synthetic construct
  REGION 1..11: note = Synthetic Construct
RASQGISSWLA

SEQ ID NO: 712 - protein, 7 aa, synthetic construct
  REGION 1..7: note = Synthetic Construct
AASSLQS

SEQ ID NO: 713 - protein, 9 aa, synthetic construct
  REGION 1..9: note = Synthetic Construct
QQVFSYPLT

SEQ ID NO: 714 - protein, 11 aa, synthetic construct
  REGION 1..11: note = Synthetic Construct
RASQSIGSWLA

SEQ ID NO: 715 - protein, 7 aa, synthetic construct
  REGION 1..7: note = Synthetic Construct
KASSLES

SEQ ID NO: 716 - protein, 10 aa, synthetic construct
  REGION 1..10: note = Synthetic Construct
QVYGSYSPRT

SEQ ID NO: 717 - protein, 7 aa, synthetic construct
  REGION 1..7: note = Synthetic Construct
GFTFSSY

SEQ ID NO: 718 - protein, 17 aa, synthetic construct
  REGION 1..17: note = Synthetic Construct
AISGSGGSTSYADSVKG

SEQ ID NO: 719 - protein, 12 aa, synthetic construct
  REGION 1..12: note = Synthetic Construct
ARLPQYSRPFDY

SEQ ID NO: 720 - protein, 11 aa, synthetic construct
RASQGISSWLA

SEQ ID NO: 721 - protein, 7 aa, synthetic construct
  REGION 1..7: note = Synthetic Construct
AASSLQS

SEQ ID NO: 722 - protein, 9 aa, synthetic construct
  REGION 1..9: note = Synthetic Construct
QQGHSFPLT

SEQ ID NO: 723 - protein, 5 aa, synthetic construct
  REGION 1..5: note = Synthetic Construct
SYRMH

SEQ ID NO: 724 - protein, 17 aa, synthetic construct
  REGION 1..17: note = Synthetic Construct
FIVPSGGSTSYAQKFQG

SEQ ID NO: 725 - protein, 11 aa, synthetic construct
  REGION 1..11: note = Synthetic Construct
VSRYAPEPMDV

SEQ ID NO: 726 - protein, 11 aa, synthetic construct
  REGION 1..11: note = Synthetic Construct
RASQGISRWLA

SEQ ID NO: 727 - protein, 7 aa, synthetic construct
  REGION 1..7: note = Synthetic Construct
AASSLQS

SEQ ID NO: 728 - protein, 9 aa, synthetic construct
  REGION 1..9: note = Synthetic Construct
QQAFSHPWT

SEQ ID NO: 729 - protein, 115 aa, Homo sapiens
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPP

SEQ ID NO: 730 - protein, 115 aa, synthetic construct
  REGION 1..115: note = Synthetic Construct
  VARIANT 11: note = Xaa is IIe or Leu
  VARIANT 12: note = Xaa is Phe or Tyr
  VARIANT 19: note = Xaa is Leu or Lys
  VARIANT 20: note = Xaa is Asp or Glu
  VARIANT 25: note = Xaa is Thr or Ser
  VARIANT 27: note = Xaa is Leu or Val
  VARIANT 29: note = Xaa is Pro or Arg
  VARIANT 31: note = Xaa is Tyr or Glu
  VARIANT 33: note = Xaa is Asp or Glu
  VARIANT 34: note = Xaa is Lys or Gln
  VARIANT 38: note = Xaa is Arg or Leu
  VARIANT 41: note = Xaa is Tyr or Phe
  VARIANT 45: note = Xaa is Arg or Lys
  VARIANT 47: note = Xaa is Ser or Ile
  VARIANT 48: note = Xaa is Ser or Thr
  VARIANT 50: note = Xaa is Ser or Thr
  VARIANT 51: note = Xaa is Ile or Leu
  VARIANT 53: note = Xaa is Ile or Leu
  VARIANT 56: note = Xaa is Lys or Gln
  VARIANT 63: note = Xaa is Phe or Ile
  VARIANT 69: note = Xaa is Gln, Thr, or Asp
  VARIANT 74: note = Xaa is Lys or Thr
  VARIANT 75: note = Xaa is Lys or Glu
  VARIANT 76: note = Xaa is Asp or Glu
  VARIANT 77: note = Xaa is Ser or Asn
  VARIANT 79: note = Xaa is Glu or Gln
  VARIANT 89: note = Xaa is Phe or Met
  VARIANT 70: note = Xaa is Glu, Asp, or Thr
GRGEAETRECXXYNANWEXXRTNQXGXEXCXGXXDKRXHCXASWXNXXGXXEXVKXGCWLDDXNCYDRXXCVAXXXXPXVYFCCCEGNXCNERFTHLPEAGGPEVTYEPPPTAPT

SEQ ID NO: 731 - protein, 115 aa, synthetic construct
  REGION 1..115: note = Synthetic Construct
GRGEAETRECIFYNANWEKDRTNQSGLEPCYGDQDKRRHCFASWKNSSGTIELVKQGCWLDDINCYDRQECVAKKDSPEVYFCCCEGNFCNERFTHLPEAGGPEVTYEPPPTAPT

SEQ ID NO: 732 - protein, 115 aa, synthetic construct
  REGION 1..115: note = Synthetic Construct
GRGEAETRECIYYNANWELDRTNQSGLERCEGEQDKRLHCYASWRNSSGTIELVKKGCWLDDINCYDRQECVATKENPQVYFCCCEGNFCNERFTHLPEAGGPEVTYEPPPTAPT

SEQ ID NO: 733 - protein, 115 aa, synthetic construct
  REGION 1..115: note = Synthetic Construct
GRGEAETRECIYYNANWELERTNQSGLERCEGEQDKRLHCYASWRNSSGTIELVKKGCWLDDFNCYDRQECVAKKDSPEVYFCCCEGNFCNERFTHLPEAGGPEVTYEPPPTAPT

SEQ ID NO: 734 - protein, 115 aa, synthetic construct
  REGION 1..115: note = Synthetic Construct
GRGEAETRECIYYNANWELERTNQTGLERCEGEQDKRLHCYASWRNISGTIELVKKGCWLDDFNCYDRQECVAKKDSPEVYFCCCEGNFCNERFTHLPEAGGPEVTYEPPPTAPT

SEQ ID NO: 735 - protein, 115 aa, synthetic construct
  REGION 1..115: note = Synthetic Construct
GRGEAETRECIYYNANWELERTNQTGLERCEGEQDKRLHCYASWRNITGTIELVKKGCWLDDFNCYDRQECVAKKDSPEVYFCCCEGNFCNERFTHLPEAGGPEVTYEPPPTAPT

SEQ ID NO: 736 - protein, 115 aa, synthetic construct
  REGION 1..115: note = Synthetic Construct
GRGEAETRECIYYNANWELERTNQSGLEPCEGEQDKRLHCYASWRNSSGTIELVKKGCWLDDFNCYDRQECVAKKDSPEVYFCCCEGNFCNERFTHLPEAGGPEVTYEPPPTAPT

SEQ ID NO: 737 - protein, 115 aa, synthetic construct
  REGION 1..115: note = Synthetic Construct
GRGEAETRECIYYNANWELERTNQSGLERCYGDKDKRLHCYASWRNSSGTIELVKKGCWLDDFNCYDRQECVAKKDSPEVYFCCCEGNFCNERFTHLPEAGGPEVTYEPPPTAPT

SEQ ID NO: 738 - protein, 115 aa, synthetic construct
  REGION 1..115: note = Synthetic Construct
GRGEAETRECIYYNANWELERTNQSGLERCEGEQDKRLHCFASWKNSSGTIELVKQGCWLDDFNCYDRQECVAKKDSPEVYFCCCEGNFCNERFTHLPEAGGPEVTYEPPPTAPT

SEQ ID NO: 739 - protein, 115 aa, synthetic construct
  REGION 1..115: note = Synthetic Construct
GRGEAETRECIFYNANWEKDRTNQSGLERCEGEQDKRLHCYASWRNSSGTIELVKKGCWLDDFNCYDRQECVAKKDSPEVYFCCCEGNFCNERFTHLPEAGGPEVTYEPPPTAPT

SEQ ID NO: 740 - protein, 115 aa, synthetic construct
  REGION 1..115: note = Synthetic Construct
GRGEAETRECIYYNANWELERTNQSGLERCYGDQDKRRHCYASWRNSSGTIELVKKGCWLDDFNCYDRQECVAKKDSPEVYFCCCEGNFCNERFTHLPEAGGPEVTYEPPPTAPT

SEQ ID NO: 741 - protein, 115 aa, synthetic construct
  REGION 1..115: note = Synthetic Construct
GRGEAETRECLYYNANWELERTNQSGVERCEGEKDKRLHCYASWRNSSGSLEIVKKGCWLDDFNCYDRTDCVATEENPQVYFCCCEGNMCNERFTHLPEAGGPEVTYEPPPTAPT

SEQ ID NO: 742 - protein, 115 aa, synthetic construct
  REGION 1..115: note = Synthetic Construct
GRGEAETRECLYYNANWELERTNQSGVERCEGEKDKRLHCYASWRNSSGSLEIVKKGCWLDDFNCYDRDTCVATEENPQVYFCCCEGNMCNERFTHLPEAGGPEVTYEPPPTAPT

SEQ ID NO: 743 - protein, 115 aa, synthetic construct
  REGION 1..115: note = Synthetic Construct
GRGEAETRECLYYNANWELERTNQSGVERCEGEKDKRLHCYASWRNSSGSLEIVKKGCWLDDFNCYDRTDCVATKENPQVYFCCCEGNMCNERFTHLPEAGGPEVTYEPPPTAPT

SEQ ID NO: 744 - protein, 115 aa, synthetic construct
  REGION 1..115: note = Synthetic Construct
GRGEAETRECLYYNANWELERTNQSGVERCEGEKDKRLHCYASWRNSSGSLEIVKKGCWLDDFNCYDRDTCVATKENPQVYFCCCEGNMCNERFTHLPEAGGPEVTYEPPPTAPT

SEQ ID NO: 745 - protein, 110 aa, synthetic construct
  REGION 1..110: note = Synthetic Construct
ETRECIYYNANWELERTNQSGLERCYGDKDKRRHCYASWRNSSGTIELVKKGCWLDDFNCYDRQECVATEENPQVYFCCCEGNFCNERFTHLPEAGGPEVTYEPPPTAPT

SEQ ID NO: 746 - protein, 110 aa, synthetic construct
  REGION 1..110: note = Synthetic Construct
ETRECIYYNANWELERTNQSGLERCEGDQDKRLHCYASWRNSSGTIELVKKGCWLDDINCYDRQECVATKENPQVYFCCCEGNFCNERFTHLPEAGGPEVTYEPPPTAPT

SEQ ID NO: 747 - protein, 107 aa, synthetic construct
  REGION 1..107: note = Synthetic Construct
ETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPPPT

SEQ ID NO: 748 - protein, 110 aa, synthetic construct
  REGION 1..110: note = Synthetic Construct
ETRWCIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 749 - protein, 110 aa, synthetic construct
  REGION 1..110: note = Synthetic Construct
ETRWCIYYNANWELERTNQTGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 750 - protein, 110 aa, synthetic construct
  REGION 1..110: note = Synthetic Construct
ETRYCIYYNANWELERTNQTGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 751 - protein, 109 aa, synthetic construct
  REGION 1..109: note = Synthetic Construct
  VARIANT 37: note = Xaa is Asp or Arg
  VARIANT 65: note = Xaa is Ile, Phe, Glu, Asp, Tyr, Ser, Asn, Gln, or Thr
  VARIANT 66: note = Xaa is Asn or Thr
  VARIANT 75: note = Xaa is Ala or Glu
  VARIANT 76: note = Xaa is Thr or Lys
  VARIANT 77: note = Xaa is Glu or Lys
  VARIANT 78: note = Xaa is Glu or Asp
  VARIANT 79: note = Xaa is Asn or Ser
  VARIANT 81: note = Xaa is Gln, Glu, Lys, Arg, Asp, or Asn
GAILGRAETRECIYYNANWELERTNQSGLERCEGEQXKRRHCFATWKNISGSIEIVKQGCWLDDXXCYDRTDCVXXXXXPXVYFCCCEGNMCNEKFSYFPEMEVTQPTS

SEQ ID NO: 752 - protein, 109 aa, synthetic construct
  REGION 1..109: note = Synthetic Construct
  VARIANT 37: note = Xaa is Asp or Arg
  VARIANT 65: note = Xaa is Ile, Phe, Glu, Asp, Tyr, Ser, Asn, Gln, or Thr
  VARIANT 66: note = Xaa is Asn or Thr
  VARIANT 75: note = Xaa is Ala or Glu
  VARIANT 76: note = Xaa is Thr or Lys
  VARIANT 77: note = Xaa is Glu or Lys
  VARIANT 78: note = Xaa is Glu or Asp
  VARIANT 79: note = Xaa is Asn or Ser
  VARIANT 81: note = Xaa is Gln, Glu, Lys, Arg, Asp, or Asn
GAILGRAETRECIYYNANWELERTNQSGLERCEGEQXKRLHCFATWKNISGSIEIVKQGCWLDDXXCYDRTDCVXXXXXPXVYFCCCEGNMCNEKFSYFPEMEVTQPTS

SEQ ID NO: 753 - protein, 109 aa, synthetic construct
  REGION 1..109: note = Synthetic Construct
  VARIANT 37: note = Xaa is Asp or Arg
  VARIANT 65: note = Xaa is Ile, Phe, Glu, Asp, Tyr, Ser, Asn, Gln, or Thr
  VARIANT 66: note = Xaa is Asn or Thr
  VARIANT 75: note = Xaa is Ala or Glu
  VARIANT 76: note = Xaa is Thr or Lys
  VARIANT 77: note = Xaa is Glu or Lys
  VARIANT 78: note = Xaa is Glu or Asp
  VARIANT 79: note = Xaa is Asn or Ser
  VARIANT 81: note = Xaa is Gln, Glu, Lys, Arg, Asp, or Asn
GAILGRAETRECIYYNANWELERTNQSGLERCEGEQXKRLHCYASWKNISGSIEIVKQGCWLDDXXCYDRTDCVXXXXXPXVYFCCCEGNMCNEKFSYFPEMEVTQPTS

SEQ ID NO: 754 - protein, 109 aa, synthetic construct
  REGION 1..109: note = Synthetic Construct
  VARIANT 37: note = Xaa is Asp or Arg
  VARIANT 65: note = Xaa is Ile, Phe, Glu, Asp, Tyr, Ser, Asn, Gln, or Thr
  VARIANT 66: note = Xaa is Asn or Thr
  VARIANT 75: note = Xaa is Ala or Glu
  VARIANT 76: note = Xaa is Thr or Lys
  VARIANT 77: note = Xaa is Glu or Lys
  VARIANT 78: note = Xaa is Glu or Asp
  VARIANT 79: note = Xaa is Asn or Ser
  VARIANT 81: note = Xaa is Gln, Glu, Lys, Arg, Asp, or Asn
GAILGRAETRECIYYNANWELERTNQSGLERCEGEQXKRLHCYASWRNSSGSIEIVKQGCWLDDXXCYDRTDCVXXXXXPXVYFCCCEGNMCNEKFSYFPEMEVTQPTS

SEQ ID NO: 755 - protein, 109 aa, synthetic construct
  REGION 1..109: note = Synthetic Construct
  VARIANT 37: note = Xaa is Asp or Arg
  VARIANT 65: note = Xaa is Ile, Phe, Glu, Asp, Tyr, Ser, Asn, Gln, or Thr
  VARIANT 66: note = Xaa is Asn or Thr
  VARIANT 75: note = Xaa is Ala or Glu
  VARIANT 76: note = Xaa is Thr or Lys
  VARIANT 78: note = Xaa is Glu or Asp
  VARIANT 79: note = Xaa is Asn or Ser
  VARIANT 81: note = Xaa is Gln, Glu, Lys, Arg, Asp, or Asn
  VARIANT 77: note = Xaa is Glu or Lys
GAILGRAETRECIYYNANWELERTNQSGLERCEGEQXKRLHCYASWRNSSGTIEIVKQGCWLDDXXCYDRTDCVXXXXXPXVYFCCCEGNMCNEKFSYFPEMEVTQPTS

SEQ ID NO: 756 - protein, 109 aa, synthetic construct
  REGION 1..109: note = Synthetic Construct
  VARIANT 37: note = Xaa is Asp or Arg
  VARIANT 65: note = Xaa is Ile, Phe, Glu, Asp, Tyr, Ser, Asn, Gln, or Thr
  VARIANT 66: note = Xaa is Asn or Thr
  VARIANT 75: note = Xaa is Ala or Glu
  VARIANT 76: note = Xaa is Thr or Lys
  VARIANT 77: note = Xaa is Glu or Lys
  VARIANT 78: note = Xaa is Glu or Asp
  VARIANT 79: note = Xaa is Asn or Ser
  VARIANT 81: note = Xaa is Gln, Glu, Lys, Arg, Asp, or Asn
GAILGRAETRECIYYNANWELERTNQSGLERCEGEQXKRLHCYASWRNSSGTIELVKKGCWLDDXXCYDRTDCVXXXXXPXVYFCCCEGNMCNEKFSYFPEMEVTQPTS

SEQ ID NO: 757 - protein, 109 aa, synthetic construct
  REGION 1..109: note = Synthetic Construct
  VARIANT 37: note = Xaa is Asp or Arg
  VARIANT 65: note = Xaa is Ile, Phe, Glu, Asp, Tyr, Ser, Asn, Gln, or Thr
  VARIANT 66: note = Xaa is Asn or Thr
  VARIANT 75: note = Xaa is Ala or Glu
  VARIANT 76: note = Xaa is Thr or Lys
  VARIANT 77: note = Xaa is Glu or Lys
  VARIANT 78: note = Xaa is Glu or Asp
  VARIANT 79: note = Xaa is Asn or Ser
  VARIANT 81: note = Xaa is Gln, Glu, Lys, Arg, Asp, or Asn
GAILGRAETRECIYYNANWELERTNQSGLERCEGEQXKRLHCYASWRNSSGTIELVKKGCWLDDXXCYDRQECVXXXXXPXVYFCCCEGNMCNEKFSYFPEMEVTQPTS

SEQ ID NO: 758 - protein, 107 aa, synthetic construct
  REGION 1..107: note = Synthetic Construct
  VARIANT 63: note = Xaa is Ile, Phe, Glu, Asp, Tyr, Ser, Asn, Gln, or Thr
  VARIANT 64: note = Xaa is Asn or Thr
  VARIANT 73: note = Xaa is Ala or Glu
  VARIANT 74: note = Xaa is Thr or Lys
  VARIANT 75: note = Xaa is Glu or Lys
  VARIANT 76: note = Xaa is Glu or Asp
  VARIANT 77: note = Xaa is Asn or Ser
  VARIANT 79: note = Xaa is Gln, Glu, Lys, Arg, Asp, or Asn
  VARIANT 35: note = Xaa is Asp or Arg
GRGEAETRECIYYNANWELERTNQSGLERCEGEQXKRRHCFATWKNISGSIEIVKQGCWLDDXXCYDRTDCVXXXXXPXVYFCCCEGNMCNEKFSYFPEMEVTQPTS

SEQ ID NO: 759 - protein, 107 aa, synthetic construct
  REGION 1..107: note = Synthetic Construct
  VARIANT 35: note = Xaa is Asp or Arg
  VARIANT 63: note = Xaa is Ile, Phe, Glu, Asp, Tyr, Ser, Asn, Gln, or Thr
  VARIANT 64: note = Xaa is Asn or Thr
  VARIANT 73: note = Xaa is Ala or Glu
  VARIANT 74: note = Xaa is Thr or Lys
  VARIANT 75: note = Xaa is Glu or Lys
  VARIANT 76: note = Xaa is Glu or Asp
  VARIANT 79: note = Xaa is Gln, Glu, Lys, Arg, Asp, or Asn
  VARIANT 77: note = Xaa is Asn or Ser
GRGEAETRECIYYNANWELERTNQSGLERCEGEQXKRLHCFATWKNISGSIEIVKQGCWLDDXXCYDRTDCVXXXXXPXVYFCCCEGNMCNEKFSYFPEMEVTQPTS

SEQ ID NO: 760 - protein, 107 aa, synthetic construct
  REGION 1..107: note = Synthetic Construct
  VARIANT 35: note = Xaa is Asp or Arg
  VARIANT 63: note = Xaa is Ile, Phe, Glu, Asp, Tyr, Ser, Asn, Gln, or Thr
  VARIANT 64: note = Xaa is Asn or Thr
  VARIANT 73: note = Xaa is Ala or Glu
  VARIANT 74: note = Xaa is Thr or Lys
  VARIANT 75: note = Xaa is Glu or Lys
  VARIANT 76: note = Xaa is Glu or Asp
  VARIANT 77: note = Xaa is Asn or Ser
  VARIANT 79: note = Xaa is Gln, Glu, Lys, Arg, Asp, or Asn
GRGEAETRECIYYNANWELERTNQSGLERCEGEQXKRLHCYASWKNISGSIEIVKQGCWLDDXXCYDRTDCVXXXXXPXVYFCCCEGNMCNEKFSYFPEMEVTQPTS

SEQ ID NO: 761 - protein, 107 aa, synthetic construct
  REGION 1..107: note = Synthetic Construct
  VARIANT 35: note = Xaa is Asp or Arg
  VARIANT 64: note = Xaa is Asn or Thr
  VARIANT 73: note = Xaa is Ala or Glu
  VARIANT 74: note = Xaa is Thr or Lys
  VARIANT 75: note = Xaa is Glu or Lys
  VARIANT 76: note = Xaa is Glu or Asp
  VARIANT 77: note = Xaa is Asn or Ser
  VARIANT 79: note = Xaa is Gln, Glu, Lys, Arg, Asp, or Asn
  VARIANT 63: note = Xaa is Ile, Phe, Glu, Asp, Tyr, Ser, Asn, Gln, or Thr
GRGEAETRECIYYNANWELERTNQSGLERCEGEQXKRLHCYASWRNSSGSIEIVKQGCWLDDXXCYDRTDCVXXXXXPXVYFCCCEGNMCNEKFSYFPEMEVTQPTS

SEQ ID NO: 762 - protein, 107 aa, synthetic construct
  REGION 1..107: note = Synthetic Construct
  VARIANT 35: note = Xaa is Asp or Arg
  VARIANT 63: note = Xaa is Ile, Phe, Glu, Asp, Tyr, Ser, Asn, Gln, or Thr
  VARIANT 64: note = Xaa is Asn or Thr
  VARIANT 73: note = Xaa is Ala or Glu
  VARIANT 74: note = Xaa is Thr or Lys
  VARIANT 76: note = Xaa is Glu or Asp
  VARIANT 77: note = Xaa is Asn or Ser
  VARIANT 79: note = Xaa is Gln, Glu, Lys, Arg, Asp, or Asn
  VARIANT 75: note = Xaa is Glu or Lys
GRGEAETRECIYYNANWELERTNQSGLERCEGEQXKRLHCYASWRNSSGTIEIVKQGCWLDDXXCYDRTDCVXXXXXPXVYFCCCEGNMCNEKFSYFPEMEVTQPTS

SEQ ID NO: 763 - protein, 107 aa, synthetic construct
  REGION 1..107: note = Synthetic Construct
  VARIANT 35: note = Xaa is Asp or Arg
  VARIANT 63: note = Xaa is Ile, Phe, Glu, Asp, Tyr, Ser, Asn, Gln, or Thr
  VARIANT 64: note = Xaa is Asn or Thr
  VARIANT 73: note = Xaa is Ala or Glu
  VARIANT 74: note = Xaa is Thr or Lys
  VARIANT 75: note = Xaa is Glu or Lys
  VARIANT 76: note = Xaa is Glu or Asp
  VARIANT 77: note = Xaa is Asn or Ser
  VARIANT 79: note = Xaa is Gln, Glu, Lys, Arg, Asp, or Asn
GRGEAETRECIYYNANWELERTNQSGLERCEGEQXKRLHCYASWRNSSGTIELVKKGCWLDDXXCYDRTDCVXXXXXPXVYFCCCEGNMCNEKFSYFPEMEVTQPTS

SEQ ID NO: 764 - protein, 107 aa, synthetic construct
  REGION 1..107: note = Synthetic Construct
  VARIANT 35: note = Xaa is Asp or Arg
  VARIANT 63: note = Xaa is Ile, Phe, Glu, Asp, Tyr, Ser, Asn, Gln, or Thr
  VARIANT 64: note = Xaa is Asn or Thr
  VARIANT 73: note = Xaa is Ala or Glu
  VARIANT 74: note = Xaa is Thr or Lys
  VARIANT 75: note = Xaa is Glu or Lys
  VARIANT 76: note = Xaa is Glu or Asp
  VARIANT 77: note = Xaa is Asn or Ser
  VARIANT 79: note = Xaa is Gln, Glu, Lys, Arg, Asp, or Asn
GRGEAETRECIYYNANWELERTNQSGLERCEGEQXKRLHCYASWRNSSGTIELVKKGCWLDDXXCYDRQECVXXXXXPXVYFCCCEGNMCNEKFSYFPEMEVTQPTS

SEQ ID NO: 765 - protein, 109 aa, synthetic construct
  REGION 1..109: note = Synthetic Construct
  VARIANT 37: note = Xaa is Asp or Arg
  VARIANT 65: note = Xaa is Ile, Phe, Glu, Asp, Tyr, Ser, Asn, Gln, or Thr
  VARIANT 66: note = Xaa is Asn or Thr
  VARIANT 76: note = Xaa is Thr or Lys
  VARIANT 77: note = Xaa is Glu or Lys
  VARIANT 78: note = Xaa is Glu or Asp
  VARIANT 79: note = Xaa is Asn or Ser
  VARIANT 81: note = Xaa is Gln, Glu, Lys, Arg, Asp, or Asn
  VARIANT 75: note = Xaa is Ala or Glu
GAILGRSETQECIYYNANWELERTNQSGLERCEGEQXKRRHCFATWKNISGSIEIVKQGCWLDDXXCYDRTDCVXXXXXPXVYFCCCEGNMCNEKFSYFPEMEVTQPTS

SEQ ID NO: 766 - protein, 109 aa, synthetic construct
  REGION 1..109: note = Synthetic Construct
  VARIANT 37: note = Xaa is Asp or Arg
  VARIANT 65: note = Xaa is Ile, Phe, Glu, Asp, Tyr, Ser, Asn, Gln, or Thr
  VARIANT 66: note = Xaa is Asn or Thr
  VARIANT 75: note = Xaa is Ala or Glu
  VARIANT 76: note = Xaa is Thr or Lys
  VARIANT 77: note = Xaa is Glu or Lys
  VARIANT 78: note = Xaa is Glu or Asp
  VARIANT 79: note = Xaa is Asn or Ser
  VARIANT 81: note = Xaa is Gln, Glu, Lys, Arg, Asp, or Asn
GAILGRSETQECIYYNANWELERTNQSGLERCEGEQXKRLHCFATWKNISGSIEIVKQGCWLDDXXCYDRTDCVXXXXXPXVYFCCCEGNMCNEKFSYFPEMEVTQPTS

SEQ ID NO: 767 - protein, 109 aa, synthetic construct
  REGION 1..109: note = Synthetic Construct
  VARIANT 37: note = Xaa is Asp or Arg
  VARIANT 65: note = Xaa is Ile, Phe, Glu, Asp, Tyr, Ser, Asn, Gln, or Thr
  VARIANT 66: note = Xaa is Asn or Thr
  VARIANT 75: note = Xaa is Ala or Glu
  VARIANT 76: note = Xaa is Thr or Lys
  VARIANT 77: note = Xaa is Glu or Lys
  VARIANT 78: note = Xaa is Glu or Asp
  VARIANT 79: note = Xaa is Asn or Ser
  VARIANT 81: note = Xaa is Gln, Glu, Lys, Arg, Asp, or Asn
GAILGRSETQECIYYNANWELERTNQSGLERCEGEQXKRLHCYASWKNISGSIEIVKQGCWLDDXXCYDRTDCVXXXXXPXVYFCCCEGNMCNEKFSYFPEMEVTQPTS

SEQ ID NO: 768 - protein, 109 aa, synthetic construct
  REGION 1..109: note = Synthetic Construct
  VARIANT 37: note = Xaa is Asp or Arg
  VARIANT 65: note = Xaa is Ile, Phe, Glu, Asp, Tyr, Ser, Asn, Gln, or Thr
  VARIANT 66: note = Xaa is Asn or Thr
  VARIANT 75: note = Xaa is Ala or Glu
  VARIANT 76: note = Xaa is Thr or Lys
  VARIANT 77: note = Xaa is Glu or Lys
  VARIANT 78: note = Xaa is Glu or Asp
  VARIANT 79: note = Xaa is Asn or Ser
  VARIANT 81: note = Xaa is Gln, Glu, Lys, Arg, Asp, or Asn
GAILGRSETQECIYYNANWELERTNQSGLERCEGEQXKRLHCYASWRNSSGSIEIVKQGCWLDDXXCYDRTDCVXXXXXPXVYFCCCEGNMCNEKFSYFPEMEVTQPTS

SEQ ID NO: 769 - protein, 109 aa, synthetic construct
  REGION 1..109: note = Synthetic Construct
  VARIANT 37: note = Xaa is Asp or Arg
  VARIANT 65: note = Xaa is Ile, Phe, Glu, Asp, Tyr, Ser, Asn, Gln, or Thr
  VARIANT 66: note = Xaa is Asn or Thr
  VARIANT 75: note = Xaa is Ala or Glu
  VARIANT 76: note = Xaa is Thr or Lys
  VARIANT 77: note = Xaa is Glu or Lys
  VARIANT 78: note = Xaa is Glu or Asp
  VARIANT 79: note = Xaa is Asn or Ser
  VARIANT 81: note = Xaa is Gln, Glu, Lys, Arg, Asp, or Asn
GAILGRSETQECIYYNANWELERTNQSGLERCEGEQXKRLHCYASWRNSSGTIEIVKQGCWLDDXXCYDRTDCVXXXXXPXVYFCCCEGNMCNEKFSYFPEMEVTQPTS

SEQ ID NO: 770 - protein, 109 aa, synthetic construct
  REGION 1..109: note = Synthetic Construct
  VARIANT 37: note = Xaa is Asp or Arg
  VARIANT 65: note = Xaa is Ile, Phe, Glu, Asp, Tyr, Ser, Asn, Gln, or Thr
  VARIANT 66: note = Xaa is Asn or Thr
  VARIANT 76: note = Xaa is Thr or Lys
  VARIANT 77: note = Xaa is Glu or Lys
  VARIANT 78: note = Xaa is Glu or Asp
  VARIANT 81: note = Xaa is Gln, Glu, Lys, Arg, Asp, or Asn
  VARIANT 79: note = Xaa is Asn or Ser
  VARIANT 75: note = Xaa is Ala or Glu
GAILGRSETQECIYYNANWELERTNQSGLERCEGEQXKRLHCYASWRNSSGTIELVKKGCWLDDXXCYDRTDCVXXXXXPXVYFCCCEGNMCNEKFSYFPEMEVTQPTS

SEQ ID NO: 771 - protein, 109 aa, synthetic construct
  REGION 1..109: note = Synthetic Construct
  VARIANT 37: note = Xaa is Asp or Arg
  VARIANT 65: note = Xaa is Ile, Phe, Glu, Asp, Tyr, Ser, Asn, Gln, or Thr
  VARIANT 66: note = Xaa is Asn or Thr
  VARIANT 75: note = Xaa is Ala or Glu
  VARIANT 76: note = Xaa is Thr or Lys
  VARIANT 77: note = Xaa is Glu or Lys
  VARIANT 78: note = Xaa is Glu or Asp
  VARIANT 79: note = Xaa is Asn or Ser
  VARIANT 81: note = Xaa is Gln, Glu, Lys, Arg, Asp, or Asn
GAILGRSETQECIYYNANWELERTNQSGLERCEGEQXKRLHCYASWRNSSGTIELVKKGCWLDDXXCYDRQECVXXXXXPXVYFCCCEGNMCNEKFSYFPEMEVTQPTS

SEQ ID NO: 772 - protein, 109 aa, synthetic construct
  REGION 1..109: note = Synthetic Construct
GAILGRAETRECIYYNANWELERTNQSGLERCEGEQDKRLHCYASWRNSSGTIEIVKQGCWLDDINCYDRTDCVATEENPQVYFCCCEGNMCNEKFSYFPEMEVTQPTS

SEQ ID NO: 773 - protein, 107 aa, synthetic construct
  REGION 1..107: note = Synthetic Construct
GRGEAETRECIYYNANWELERTNQSGLERCEGEQDKRLHCYASWRNSSGTIEIVKQGCWLDDINCYDRTDCVEKKDSPEVYFCCCEGNMCNEKFSYFPEMEVTQPTS

SEQ ID NO: 774 - protein, 107 aa, synthetic construct
  REGION 1..107: note = Synthetic Construct
GRGEAETRECIYYNANWELERTNQSGLERCEGEQDKRLHCYASWRNSSGTIEIVKQGCWLDDINCYDRTDCVEKKDSPQVYFCCCEGNMCNEKFSYFPEMEVTQPTS

SEQ ID NO: 775 - protein, 107 aa, synthetic construct
  REGION 1..107: note = Synthetic Construct
GRGEAETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 776 - protein, 109 aa, synthetic construct
  REGION 1..109: note = Synthetic Construct
GAILGRAETRECIYYNANWELERTNQSGLERCEGEQDKRLHCYASWRNSSGTIEIVKQGCWLDDFNCYDRTDCVATEENPQVYFCCCEGNMCNEKFSYFPEMEVTQPTS

SEQ ID NO: 777 - protein, 107 aa, synthetic construct
  REGION 1..107: note = Synthetic Construct
GRGEAETRECIYYNANWELERTNQSGLERCEGEQDKRLHCYASWRNSSGTIEIVKQGCWLDDFNCYDRTDCVEKKDSPEVYFCCCEGNMCNEKFSYFPEMEVTQPTS

SEQ ID NO: 778 - protein, 107 aa, synthetic construct
  REGION 1..107: note = Synthetic Construct
GRGEAETRECIYYNANWELERTNQSGLERCEGEQDKRRHCFATWKNISGSIEIVKQGCWLDDFNCYDRTDCVEKKDSPQVYFCCCEGNMCNEKFSYFPEMEVTQPTS

SEQ ID NO: 779 - protein, 107 aa, synthetic construct
  REGION 1..107: note = Synthetic Construct
GRGEAETRECIYYNANWELERTNQSGLERCEGEQDKRLHCFATWKNISGSIEIVKQGCWLDDFNCYDRTDCVEKKDSPQVYFCCCEGNMCNEKFSYFPEMEVTQPTS

SEQ ID NO: 780 - protein, 107 aa, synthetic construct
  REGION 1..107: note = Synthetic Construct
GRGEAETRECIYYNANWELERTNQSGLERCEGEQDKRLHCYASWKNISGSIEIVKQGCWLDDFNCYDRTDCVEKKDSPQVYFCCCEGNMCNEKFSYFPEMEVTQPTS

SEQ ID NO: 781 - protein, 107 aa, synthetic construct
  REGION 1..107: note = Synthetic Construct
GRGEAETRECIYYNANWELERTNQSGLERCEGEQDKRLHCYASWRNSSGSIEIVKQGCWLDDFNCYDRTDCVEKKDSPQVYFCCCEGNMCNEKFSYFPEMEVTQPTS

SEQ ID NO: 782 - protein, 107 aa, synthetic construct
  REGION 1..107: note = Synthetic Construct
GRGEAETRECIYYNANWELERTNQSGLERCEGEQDKRLHCYASWRNSSGTIELVKKGCWLDDFNCYDRTDCVEKKDSPQVYFCCCEGNMCNEKFSYFPEMEVTQPTS

SEQ ID NO: 783 - protein, 107 aa, synthetic construct
  REGION 1..107: note = Synthetic Construct
GRGEAETRECIYYNANWELERTNQSGLERCEGEQDKRLHCYASWRNSSGTIELVKKGCWLDDFNCYDRQECVATKDSPQVYFCCCEGNMCNEKFSYFPEMEVTQPTS

SEQ ID NO: 784 - protein, 107 aa, synthetic construct
  REGION 1..107: note = Synthetic Construct
GRGEAETRECIYYNANWELERTNQSGLERCEGEQDKRLHCYASWRNSSGTIELVKKGCWLDDFNCYDRQECVATEENPQVYFCCCEGNMCNEKFSYFPEMEVTQPTS

SEQ ID NO: 785 - protein, 107 aa, synthetic construct
  REGION 1..107: note = Synthetic Construct
GRGEAETRECIYYNANWELERTNQSGLERCEGEQDKRLHCYASWRNSSGTIEIVKQGCWLDDNNCYDRTDCVEKKDSPQVYFCCCEGNMCNEKFSYFPEMEVTQPTS

SEQ ID NO: 786 - protein, 107 aa, synthetic construct
  REGION 1..107: note = Synthetic Construct
GRGEAETRECIYYNANWELERTNQSGLERCEGEQDKRLHCYASWRNSSGTIEIVKQGCWLDDTNCYDRTDCVEKKDSPQVYFCCCEGNMCNEKFSYFPEMEVTQPTS

SEQ ID NO: 787 - protein, 107 aa, synthetic construct
  REGION 1..107: note = Synthetic Construct
GRGEAETRECIYYNANWELERTNQSGLERCEGEQDKRLHCYASWRNSSGTIEIVKQGCWLDDETCYDRTDCVEKKDSPQVYFCCCEGNMCNEKFSYFPEMEVTQPTS

SEQ ID NO: 788 - protein, 107 aa, synthetic construct
  REGION 1..107: note = Synthetic Construct
GRGEAETRECIYYNANWELERTNQSGLERCEGEQRKRLHCYASWRNSSGTIEIVKQGCWLDDFNCYDRTDCVEKKDSPQVYFCCCEGNMCNEKFSYFPEMEVTQPTS

SEQ ID NO: 789 - protein, 107 aa, synthetic construct
  REGION 1..107: note = Synthetic Construct
GRGEAETRECIYYNANWELERTNQSGLERCEGEQDKRLHCYASWRNSSGTIEIVKQGCWLDDFNCYDRTDCVETKDSPQVYFCCCEGNMCNEKFSYFPEMEVTQPTS

SEQ ID NO: 790 - protein, 109 aa, synthetic construct
  REGION 1..109: note = Synthetic Construct
GAILGRAETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 791 - protein, 109 aa, synthetic construct
  REGION 1..109: note = Synthetic Construct
GAILGRSETQECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 792 - protein, 107 aa, synthetic construct
  REGION 1..107: note = Synthetic Construct
GRGEAETRECIYYNANWELERTNQSGLERCEGEQDKRLHCYASWRNSSGTIELVKKGCWLDDFNCYDRQECVATKDSPEVYFCCCEGNMCNEKFSYFPEMEVTQPTS

SEQ ID NO: 793 - protein, 107 aa, synthetic construct
  REGION 1..107: note = Synthetic Construct
GRGEAETRECIYYNANWELERTNQSGLERCEGEQDKRLHCYASWRNSSGTIELVKKGCWLDDFNCYDRQECVATKENPQVYFCCCEGNMCNEKFSYFPEMEVTQPTS

SEQ ID NO: 794 - protein, 10 aa, synthetic construct
  REGION 1..10: note = Synthetic Construct
GAILGRSETQ

SEQ ID NO: 795 - protein, 8 aa, synthetic construct
  REGION 1..8: note = Synthetic Construct
GRGEAETR

SEQ ID NO: 796 - protein, 6 aa, synthetic construct
  REGION 1..6: note = Synthetic Construct
ECLFFN

SEQ ID NO: 797 - protein, 6 aa, synthetic construct
  REGION 1..6: note = Synthetic Construct
ECIYYN

SEQ ID NO: 798 - protein, 9 aa, synthetic construct
  REGION 1..9: note = Synthetic Construct
ANWEKDRTN

SEQ ID NO: 799 - protein, 9 aa, synthetic construct
  REGION 1..9: note = Synthetic Construct
ANWELERTN

SEQ ID NO: 800 - protein, 7 aa, synthetic construct
  REGION 1..7: note = Synthetic Construct
QTGVEPC

SEQ ID NO: 801 - protein, 7 aa, synthetic construct
  REGION 1..7: note = Synthetic Construct
QSGLERC

SEQ ID NO: 802 - protein, 7 aa, synthetic construct
  REGION 1..7: note = Synthetic Construct
YGDKDKR

SEQ ID NO: 803 - protein, 7 aa, synthetic construct
  REGION 1..7: note = Synthetic Construct
EGEQDKR

SEQ ID NO: 804 - protein, 10 aa, synthetic construct
  REGION 1..10: note = Synthetic Construct
RHCFATWKNI

SEQ ID NO: 805 - protein, 7 aa, synthetic construct
  REGION 1..7: note = Synthetic Construct
HCFATWK

SEQ ID NO: 806 - protein, 10 aa, synthetic construct
  REGION 1..10: note = Synthetic Construct
LHCYASWRNS

SEQ ID NO: 807 - protein, 7 aa, synthetic construct
  REGION 1..7: note = Synthetic Construct
HCYASWR

SEQ ID NO: 808 - protein, 10 aa, synthetic construct
  REGION 1..10: note = Synthetic Construct
SIEIVKQGCW

SEQ ID NO: 809 - protein, 8 aa, synthetic construct
  REGION 1..8: note = Synthetic Construct
EIVKQGCW

SEQ ID NO: 810 - protein, 10 aa, synthetic construct
  REGION 1..10: note = Synthetic Construct
TIELVKKGCW

SEQ ID NO: 811 - protein, 8 aa, synthetic construct
  REGION 1..8: note = Synthetic Construct
ELVKKGCW

SEQ ID NO: 812 - protein, 12 aa, synthetic construct
  REGION 1..12: note = Synthetic Construct
LDDINCYDRTDC

SEQ ID NO: 813 - protein, 12 aa, synthetic construct
  REGION 1..12: note = Synthetic Construct
LDDFNCYDRQEC

SEQ ID NO: 814 - protein, 6 aa, synthetic construct
  REGION 1..6: note = Synthetic Construct
KDSPEV

SEQ ID NO: 815 - protein, 6 aa, synthetic construct
  REGION 1..6: note = Synthetic Construct
EENPQV

SEQ ID NO: 816 - protein, 6 aa, synthetic construct
  REGION 1..6: note = Synthetic Construct
YFCCCE

SEQ ID NO: 817 - protein, 6 aa, synthetic construct
  REGION 1..6: note = Synthetic Construct
GNMCNE

SEQ ID NO: 818 - protein, 6 aa, synthetic construct
  REGION 1..6: note = Synthetic Construct
GNFCNE

SEQ ID NO: 819 - protein, 5 aa, synthetic construct
  REGION 1..5: note = Synthetic Construct
KFSYF

SEQ ID NO: 820 - protein, 5 aa, synthetic construct
  REGION 1..5: note = Synthetic Construct
RFTHL

SEQ ID NO: 821 - protein, 10 aa, synthetic construct
  REGION 1..10: note = Synthetic Construct
PEMEVTQPTS

SEQ ID NO: 822 - protein, 18 aa, synthetic construct
  REGION 1..18: note = Synthetic Construct
PEAGGPEVTYEPPPTAPT

SEQ ID NO: 823 - skipped

SEQ ID NO: 824 - protein, 16 aa, synthetic construct
  REGION 1..16: note = Synthetic Construct
  VARIANT 4: note = Xaa is Tyr or His
  VARIANT 7: note = Xaa is Ser or Lys
  VARIANT 8: note = Xaa is Thr, Met or Lys
  VARIANT 10: note = Xaa is Tyr or Lys
  VARIANT 12: note = Xaa is Ser or Glu
  VARIANT 16: note = Xaa is Gly or Lys
  VARIANT 11: note = Xaa is Ala, Met or Glu
IISXAGXXYXXXWAKX

SEQ ID NO: 825 - protein, 11 aa, synthetic construct
  REGION 1..11: note = Synthetic Construct
  VARIANT 5: note = Xaa is Tyr or His
  VARIANT 7: note = Xaa is Thr or His
  VARIANT 11: note = Xaa is Leu or Lys
GVPAXSXGGDX

SEQ ID NO: 826 - protein, 13 aa, synthetic construct
  REGION 1..13: note = Synthetic Construct
  VARIANT 1: note = Xaa is Gln or Thr
  VARIANT 2: note = Xaa is Ser or Thr
  VARIANT 5: note = Xaa is Ser or Glu
  VARIANT 7: note = Xaa is Tyr or Phe
  VARIANT 8: note = Xaa is Asp or His
  VARIANT 9: note = Xaa is Asn, Asp, Ala or Glu
XXSQXVXXXNWLS

SEQ ID NO: 827 - protein, 7 aa, synthetic construct
  REGION 1..7: note = Synthetic Construct
  VARIANT 3: note = Xaa is Ser or Glu
  VARIANT 7: note = Xaa is Ser, Tyr, Phe or Trp
WAXTLAX

SEQ ID NO: 828 - protein, 10 aa, synthetic construct
  REGION 1..10: note = Synthetic Construct
  VARIANT 8: note = Xaa is Leu or Arg
AGGYGGGXYA